(19) United States
(12) Patent Application Publication
(10) Pub. No.: US 2010/0009970 A1
(43) Pub. Date: Jan. 14, 2010

(54) COMPOSITIONS AND METHODS FOR TREATMENT OF VIRAL DISEASES

(75) Inventors: Lisa M. Johansen, Belmont, MA (US); Christopher M. Owens, Cambridge, MA (US); Christina Mawhinney, Jamaica Plain, MA (US); Todd W. Chappell, Boston, MA (US); Alexander T. Brown, Watertown, MA (US); Michael G. Frank, Boston, MA (US); Michael A. Foley, Chestnut hill, MA (US); Ralf Altmeyer, Singapore (SG); Yu Chen, Singapore (SG)

Correspondence Address: Gearhart Law LLC, 4 Femdale Avenue, Chatham, NJ, 07928, US

(73) Assignee: Combinatorx (Singapore) Pte. Ltd., Singapore (SG)

(21) Appl. No.: 12/406,626

(22) Filed: Mar. 18, 2009

Related U.S. Application Data
(60) Provisional application No. 61/070,047, filed on Mar. 19, 2008.
(60) Provisional application No. 61/089,850, filed on Aug. 18, 2008.

(51) Int. Cl. A61K 31/136; A61K 31/404; A61K 31/366; A61K 31/505; A61K 31/495; C07C 211/33; C07C 217/56; A61P 25/00; A61K 31/445; A61K 31/551; A61K 31/5375; A61K 31/4965

(52) U.S. Cl. 514/218; 514/647; 514/419; 514/460; 514/275; 514/255.04; 564/367; 514/603; 514/655; 564/384; 514/331; 514/239.5; 514/255.03

(57) ABSTRACT

The present invention features compositions, methods, and kits useful in the treatment of viral diseases. In certain embodiments, the viral disease is caused by a single stranded RNA virus, a flaviviridae virus, or a hepatic virus. In particular embodiments, the viral disease is viral hepatitis (e.g., hepatitis A, hepatitis B, hepatitis C, hepatitis D, hepatitis E) and the agent or combination of agents includes sertraline, a sertraline analog, UK-416244, or a UK-416244 analog. Also featured are screening methods for identification of novel compounds that may be used to treat a viral disease.

### CROSS-REFERENCE TO RELATED APPLICATIONS

This application claims the benefit of U.S. Provisional Application No. 61/070,047, filed Mar. 19, 2008 and U.S. Provisional Application No. 61/089,850, filed Aug. 18, 2008, each of which is hereby incorporated by reference.

### BACKGROUND OF THE INVENTION

The invention relates to the treatment of diseases caused by a virus.

Diseases caused by viruses are major health problems worldwide, and include many potentially fatal or disabilitating illnesses. Viral diseases include diseases caused by single stranded RNA viruses, flaviviridae viruses, and hepatic viruses. In one example, viral hepatitis (e.g., hepatitis A, hepatitis B, hepatitis C, hepatitis D, and hepatitis E) can result in chronic or acute hepatitis. While vaccines protective against hepatitis A and hepatitis B exist, no cures for many viruses, including hepatitis B, C, D, or E, are available.

With regard to the hepatitis C virus (HCV), the Center for Disease Control estimates that 4.1 million Americans (1.6%) have been infected with this virus. Of those infected, 3.2 million are chronically infected, and HCV is the leading cause of death from liver disease in the United States. Hepatitis C is a major risk factor for developing liver cirrhosis and hepatocellular carcinoma, and the World Health Organization indicates that hepatitis C is responsible for two thirds of liver transplants. Worldwide, an estimated 180 million people, or about 3% of the world's population, are infected with HCV. No vaccine for hepatitis C is presently available, and the currently recommended therapy, a combination of pegylated interferon and ribavirin, is effective in only about 50% of those infected with HCV genotype 1. Further, both interferon and ribavirin have potentially serious side effects, which include seizures, acute heart or kidney failure, and anemia.

Given the lack of safe, efficacious treatments for many viral diseases, there exists a need for improved therapies.

### SUMMARY OF THE INVENTION

Based on the results of our screen identifying compounds and combinations of compounds having antiviral activity, the present invention features compositions, methods, and kits for the treatment of viral disease (e.g., caused by the viruses described herein). In certain embodiments, the viral disease may be caused by a virus which is a member of one or more of the following groups: single stranded RNA viruses, flaviviridae viruses (e.g., a hepacivirus such as HCV, flavivirus, pestivirus, or hepatitis G virus), and hepatic viruses. HCV, for example, is a single stranded RNA virus, a flaviviridae virus, and a hepatic virus. In certain embodiments, the viral disease is caused by the hepatitis C virus. Additional exemplary viruses are described herein.

Accordingly in a first aspect, the invention features a composition including a first agent selected from the agents of Table 1, Table 2, and Table 3, or an analog thereof, and a second agent selected from the agents of Table 1, Table 2, Table 3, Table 4, and Table 5, or an analog thereof (e.g., Table 4 and Table 5, or excluding the combinations of Table 6). In certain embodiments, the first agent is sertraline, a sertraline analog, UK-416244, or a UK-4162244 analog (e.g., any of those described herein).

TABLE 1

| Compound | IC50* | Compound | IC50* |
|---|---|---|---|
| 1,2-Bis-(2-aminophenoxy)ethane N,N,N,N,- | 14.50 | Isosulfan Blue | 24.86 |
| tetreacetic acid | | | |
| 1,5-Isoquinolinediol | 25.88 | JSH-23 | 2.55 |
| 10-Deacetylbaccatine Iii | 10.34 | Levothyroxine (e.g., sodium) | 3.79 |
| 2′,2″-(Pentamethylenedioxy)diacetanilide | 3.14 | Loratadine | 8.16 |
| 2-Hydroxyflavanone | 2.48 | Manganese gluconate | 24.71 |
| 2-Methoxyestradiol | 7.91 | Maprotiline (e.g., hydrochloride) | 7.18 |
| 3,3′-(Pentamethylenedioxy)dianiline | 1.63 | Mebeverine (e.g., hydrochloride) | 14.88 |
| 6-Nitroquipazine | 16.41 | Mechlorethamine (e.g., hydrochloride) | 4.15 |
| AG-490 | 5.03 | Meclizine | 14.62 |
| AG-494 | 3.45 | Mecobalamin | 0.179 |
| Albendazole | 0.324 | Melphalan | 5.94 |
| Amitraz | 26.4* | Mequinol | 18.65 |
| Amitrole | 14.62 | Mesoridazine (e.g., Besylate) | 19.00 |
| Amorolfine (e.g., hydrochloride) | 1.62 | Mesterolone | 5.18 |
| Anisomycin | 0.608 | Methylglyoxal bis(guanylhydrazone) dihydrochloride | 10.80 |
| | | hydrate | |
| Auranofin | 1.07 | Methyltestosterone | 19.11 |
| Azelastine | 6.22 | Mianserin (e.g., hydrochloride) | 13.72 |
| Bay 11-7082 | 15.01 | Mitotane | 28.1* |
| Bay 41-2272 | 0.754 | ML 9 | 4.44 |
| Benoxinate (e.g., hydrochloride) | 3.02 | Mofebutazone | 14.60 |
| Benzamil (e.g., HCl) | 4.73 | Mometasone (e.g., furoate) | 11.35 |
| Benzocaine | 13.91 | Monobenzone | 1.59 |
| Benztropine (e.g., mesylate) | 5.70 | Mosapride (e.g., citrate) | 10.91 |
| Benzydamine (e.g., hydrochloride) | 9.00 | Narasin | 0.176 |
| Beta Escin | 4.27 | Noscapine | 15.83 |
| Beta-Carotene | 18.50 | NSC 663284 | 0.614 |
| Beta-Ionol | 21.00 | N-Tosyl-L-phenylalanine chloromethyl ketone | 16.67 |
| Betaxolol (e.g., hydrochloride) | 29.4* | Octyl Methoxycinnamate | 1.24 |
| BHQ | 23.28 | Oxeladin | 8.72 |
| Bifonazole | 6.15 | Oxfendazole | 7.30 |
| Bismuth subsalicylate | 18.09 | Oxibendazole | 0.300 |
| Bromhexine | 14.25 | Oxyphenbutazone (e.g., hydrate) | 4.17 |
| Bromocriptine (e.g., mesylate) | 3.38 | Paclitaxel | 0.0092 |
| Budesonide | 15.66 | Padimate O | 5.44 |
| Bufexamac | 8.29 | P-Aminosalicylic acid | 13.16 |
| Camptothecin | 0.026 | Parthenolide | 2.69 |
| Capsaicin | 11.72 | Perospirone | 3.60 |
| Carbaryl | 9.65 | Phenazopyridine (e.g., hydrochloride) | 7.85 |
| CAY10433 | 7.88 | Piceatannol | 5.47 |
| Celastrol | 0.449 | Picotamide | 28.7* |
| Cerulenin | 16.21 | PKR inhibitor | 1.75 |
| Chlorophyllin | 1.30 | Pramoxine (e.g., hydrochloride) | 5.17* |
| Chlorphenoxamine (e.g., hydrochloride) | 16.20 | Promazine (e.g., hydrochloride) | 16.12 |
| Citalopram (e.g., hydrobromide) | 27.30 | Propidium (e.g., iodide) | 9.38 |
| Cladribine | 0.112 | Quinacrine | 4.17 |
| Clomiphene (e.g., citrate) | 1.19 | Quinestrol | 5.43 |
| Cobamamide | 0.410 | R(+)-Verapamil (e.g., hydrochloride) | 15.67 |
| Cyclocytidine (e.g., hydrochloride) | 0.183 | Raloxifene (e.g., hydrochloride) | 3.74 |
| Cycloheximide | 0.184 | Repaglinide | 12.21 |
| Cyproheptadine (e.g., hydrochloride) | 17.97 | Rescinnamine | 7.88 |
| Dehydroepiandrosterone | 11.19 | Reserpine | 25.29 |
| Deptropine (e.g., citrate) | 11.14 | Rifabutin | 17.25 |
| Desloratadine | 6.07 | Rifaximin | 19.36 |
| Desoxycorticosterone (e.g., acetate) | 14.65 | Saponin | 361.62 |
| Dextrothyroxine (e.g., sodium) | 5.00 | Satraplatin | 4.80 |
| Dibucaine (e.g., hydrochloride) | 6.68 | SB-202190 | 5.18 |
| Dicyclomine (e.g., hydrochloride) | 25.01 | Sertraline (e.g., hydrochloride) | 5.39 |
| Dienestrol | 16.49 | Shikonin | 26.4* |
| Diethylstilbestrol | 12.18 | Siguazodan | 2.20 |
| Dihydroergotamine (e.g., mesylate) | 22.75 | Silver sulfadiazine | 2.20 |
| Dilazep (e.g., dihydrochloride) | 13.87 | Sirolimus | 0.005* |
| Diphenidol (e.g., hydrochloride) | 25.45 | Fusidic acid (e.g., sodium fusidate) | 7.72 |
| Disulfiram | 5.50 | Spiperone | 7.21 |
| DNA-PK inhibitor II | 6.52 | Stanozolol | 15.18 |
| Donepezil (e.g., hydrochloride) | 29.29 | Suberohydroxamic acid | 4.02 |
| Doxepin (e.g., hydrochloride) | 14.88 | Tamoxifen (e.g., citrate) | 3.13 |
| Dydrogesterone | 2.75 | Terconazole | 2.55 |
| Erbstatin | 7.63 | Testosterone | 8.11 |
| Ergoloid Mesylates | 15.25 | Thapsigargin | 0.0113 |
| Evans Blue | 1.94 | Thiostrepton | 3.84 |
| Exemestane | 29.04 | Thiram | 3.64 |
| Ezetimibe | 4.20 | Tioxolone | 16.24 |
| Fascaplysin | 0.444 | Tirapazamine | 1.83 |
| Fenbendazole | 0.419 | Tiratricol | 15.56 |
| Fenretinide | 2.26 | Tolterodine (e.g., tartrate) | 27.23 |
| Fenvalerate | 18.95 | Topotecan (e.g., hydrochloride) | 0.095 |
| Flubendazole | 0.173 | Toremifene | 15.86 |
| Fludarabine | 4.47 | Trequinsin (e.g., hydrochloride) | 2.93 |
| Fluorouracil | 18.66 | Trifluoperazine (e.g., hydrochloride) | 4.97 |
| Flupentixol (e.g., dihydrochloride) | 3.60 | Trifluperidol | 7.80 |
| Fluphenazine (e.g., hydrochloride) | 3.35 | Trimipramine (e.g., maleate) | 15.62 |
| Fluvoxamine (e.g., maleate) | 23.79 | Tyrphostin 23 | 14.61 |
| FR122047 | 23.01 | Tyrphostin 25 | 16.01 |
| Fulvestrant | 3.05 | Tyrphostin 46 | 21.22 |
| Gefitinib (Base) | 3.17 | Tyrphostin 47 | 18.3* |
| Gramicidin | 0.017 | Tyrphostin Ag 1478 | 3.41 |
| Griseofulvin (e.g., microcrystalline) | 11.53 | U18666A | 0.020 |
| GW 5074 | 2.36 | UCH-L1 inhibitor | 17.18 |
| Halcinonide | 17.40 | UCH-L3 inhibitor | 19.7* |
| Hydroquinone | 13.99 | Vanillin (e.g., acetate) | 3.73 |
| Hydroxocobalamin | 1.33 | Vinorelbine | 0.081 |
| Hydroxyzine (e.g., hydrochloride) | 10.93 | Vitamin B12 | 8.28 |
| Ifenprodil (e.g., tartrate) | 4.68 | Vitamin K5 | 19.59 |
| Imipramine (e.g., hydrochloride) | 16.93 | Wedelolactone | 4.66 |
| Indocyanine Green | 8.13 | Wortmannin | 3.16 |
| Iophenoxic acid | 10.63 | Zafirlukast | 18.49 |
| LY 294002 | 3.40 | Zimelidine (e.g., dihydrochloride) | 15.14 |
| (S,S)—N-Desmethyl sertraline (e.g., | 4.94 | 3′,3″-(Pentamethylenedioxy)diacetanilide | 9.35* |
| hydrochloride) | | | |
| 1,5-Bis(4-aminophenoxy)pentane | 1.70 | rac-cis-N-Desmethyl Sertraline, (e.g., hydrochloride) | 6.03 |
| Emetine (e.g., dihydrochloride hydrate) | 0.03 | 2,2′-(Pentamethylenedioxy)dianiline | 0.27 |
| Irinotecan (e.g., hydrochloride) | 1.56 | UK-416244 | 1.41 |

*Values noted with an asterisk (*) are IC25 values

TABLE 2

| Compound | IC50 | Compound | IC50 |
|---|---|---|---|
| Efavirenz | 15.45 | Cytarabine | 0.117 |
| Nelfinavir (e.g., mesylate) | 4.25 | Floxuridine | 0.0045 |
| Vidarabine | 26.71 | Edoxudine | 1.95 |
| Ritonavir | 14.91 | Cepharanthine | 19.48 |
| Aphidicolin | 1.71 | Tunicamycin | 0.107 |
| Andrographis | 8.39 | Triciribine | 2.14 |
| Saquinavir (e.g., mesylate) | 10.04 | Curcumin | 8.68 |
| Trifluridine | 0.380 | Vincristine (e.g., sulfate) | 0.02 |
| Arbidol | 12.20 | | |

TABLE 3

| Compound | IC50* | Compound | IC50* |
|---|---|---|---|
| Lovastatin | 1.41 | Artemisinin | 4.45 |
| Artemether | | Dihydroartemisinin | 3.87 |
| Artesunate | 3.73 | Nitazoxanide | 14.04 |
| Cyclosporine | 0.379 | Chloroquine | 4.78 |
| | | (e.g., phosphate) | |
| Ribavirin | 42.95 | Mevastatin | 3.45 |
| Simvastatin hydroxy acid, | 13.40 | TOFA | 5.53 |
| ammonium salt | | | |
| Mycophenolic Acid | 0.751 | 2′-C-Methylcytidine | 1.63 |
| Atorvastatin | 35.60 | Adefovir (e.g., dipivoxil) | 0.319 |
| Fluvastatin (e.g., sodium) | 22.20 | Telaprevir (VX-950) | 0.529 |
| Celgosivir | 6.25* | Valopicitabine (NM-283) | 11.2 |
| Merimepodib (VX-497) | 0.475 | HCV-796 | 0.0192 |
| Boceprevir (SCH 503034) | 0.259 | Gemcitabine | 0.06 |
| | | (e.g., hydrochloride) | |
| Interferon Alfa-2a | 2.35 | Simvastatin | 21.34 |

*Values noted with an asterisk (*) are IC25 values

In another aspect, the invention features a composition including (a) sertraline, a sertraline analog, UK-416244, or a UK-416244 analog; and (b) an HMG-CoA reductase inhibitor. The HMG-CoA reductase inhibitor may be fluvastatin, simvastatin, lovastatin, or rosuvastatin.

In another aspect, the invention features a composition including (a) sertraline, a sertraline analog, UK-416244, or a UK-416244 analog; and (b) an antihistamine. The antihistamine may be hydroxyzine.

In another aspect, the invention features structural analogs of sertraline and UK-416244 (e.g., those described herein). In certain embodiments, the invention features a compound having the formula:

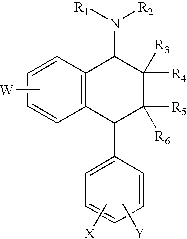

where R₁ and R₂ are independently selected from the group consisting of H, optionally substituted C₁₋₆ alkyl (CH₂)ₓCOOH, or CH₂CHOH(CH₂)ₓ, (CH₂)ₓN(CH₃)₂, where x is 1, 2, 3, 4, or 5, and optionally substituted C₁₋₇ heteroalkyl; R₃, R₄, R₅, and R₆ are independently H or optionally substituted C₁₋₆ alkyl; X and Y are each selected from the group consisting of H, F, Cl, Br, CF₃, C₁₋₆ alkoxy, and cyano; and W is NHCOPh, NHSO₂Ph, NHCOcyclopentyl, NHSO₂cyclopropyl, NHCOH, CONHPh,

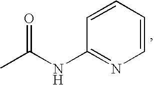

### CONHcyclopropyl,

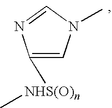

C(S)NH₂, NHC(S)CH₃, CH₂S(O)ₙR₁₁, where n is 0, 1, or 2 and R₁₁ is phenyl, C₂₋₆ heterocyclyl, C₄₋₈ unsubstituted alkyl, or C₃₋₈ substituted alkyl. The compound may have a structure selected from the group consisting of those listed in Table 9, or the compound may have the formula:

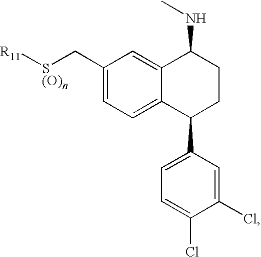

where n is 0, 1, or 2; and R₁₁ is phenyl, C₂₋₆ heterocyclyl, C₄₋₈ unsubstituted alkyl, or C₃₋₈ substituted alkyl. The compound may be part of a composition along with a pharmaceutically acceptable carrier.

In another aspect, the invention features a compound having the formula:

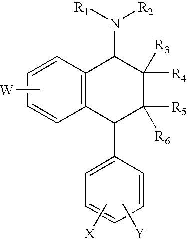

where R₁ is C₁₋₆ alkyl and R₂ is CH₂CH(OH)R₈, or CH₂CH(R₈)NR₉R₁₀, where R₈, R₉, and R₁₀ are independently H or C₁₋₆ alkyl; R₃, R₄, R₅, and R₆ are independently H or optionally substituted C₁₋₆ alkyl; X and Y are each selected from the group consisting of H, F, Cl, Br, CF₃, C₁₋₆ alkoxy, and cyano; and W is selected from the group consisting of H, F, Cl, Br, CF₃, C₁₋₃ alkoxy, COOH, CH₂CH₂OH, NHCOH, NHCOCH₃, CH₂NH₂, CH₂S(O)ₙCH₃, CONH₂, CH₂OH, NHCOPh, CH₂NHS(O)ₙCH₃, NHS(O)ₙPh, N(CH₃)₂, S(O)ₙNH₂, NHCOBu, NHS(O)ₙCH₃, NHCOcyclopropyl, NHCOcyclopentyl, CN, NHS(O)ₙcyclopropyl, NH₂, NO₂, I, SO₂N(CH₃)₂, SO₂NHMe, SO₂NHCH₂CH₂OH, CO₂Me, NHSO₂Bu, CONHCH₃, CH₂NHCOCH₃, CONHPh,

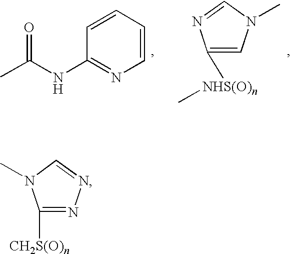

CONHcylopropyl, C(S)NH₂, NHC(S)CH₃, CONHCH₂COOCH₃, CONHCH₂COOH, CONHCH₂cyclopropyl, CON(CH₃)cyclopropyl, CONHcyclobutyl, N(CH₃)COCH₃, and CH₂S(O)ₙR₁₁, where n is 0, 1, or 2 and R₁₁ is phenyl, C₂₋₆ heterocyclyl, or optionally substituted C₁₋₈ alkyl (e.g., C₄₋₈ unsubstituted alkyl such as Bu or C₃₋₈ substituted alkyl), wherein said compound is not sertraline or an isomer thereof. In other embodiments, the compound has formula set forth herein (e.g., in the Examples).

The compound may have a formula selected from the group consisting of

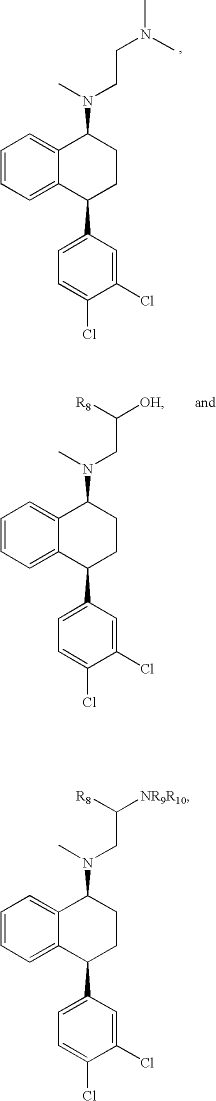

or have the formula:

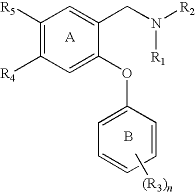

where R₁ and R₂ are independently H, C₁₋₆ alkyl, CH₂CH₃N(CH₃)₂, (CH₂)ₘ(C₃₋₆ cycloalkyl) where m is 0, 1, 2, or 3, or R₁ and R₂ together with the nitrogen to which they are attached form an azetidine ring; each R₃ is independently H, I, Br, F, Cl, C₁₋₆ alkyl, CF₃, CN, OCF₃, C₁₋₄ alkylthio, C₁₋₄ alkoxy, aryloxy, or CONR₆R₇; n is 1, 2, or 3; where one of R₄ and R₅ is A-X, where A is —CH═CH— or —(CH₂)ₚ— where p is 0, 1, or 2; X is H, F, Cl, Br, I, CONR₆R₇, SO₂NR₆R₇, SO₂NHC(═O)R₆, OH, C₁₋₄ alkoxy, NR₈SO₂R₉, NO₂, NR₆R₁₁, CN, CO₂R₁₀, CHO, SR₁₀, S(O)R₉ or SO₂R₁₀; R₆, R₇, R₈ and R₁₀ independently are H, C₁₋₆ alkyl, C₆₋₁₂ aryl optionally substituted independently by one or more R₁₂, or C₁₋₆ alkyl-aryl optionally substituted, and the other of R₄ and R₅ is SNHPh, SONHPh, or SO₂NHPh, where the phenyl is optionally substituted by one or more R₁₂; R₉ is C₁₋₆ alkyl optionally substituted independently by one or more R₁₂; R₁₁ is H, C₁₋₆ alkyl optionally substituted independently by one or more R₁₂, C(O)R₆, CO₂R₉, C(O)NHR₆, or SO₂NR₆R₇; R₁₂ is F (preferably up to 3), Br, OH, OCH₃, CO₂H, C₃₋₆ cycloalkyl, NH₂, CONH₂, C₁₋₆ alkoxy, C₁₋₆ alkoxycarbonyl, or a 5- or 6-membered heterocyclic ring containing 1, 2, or 3 heteroatoms selected from N, S, and O optionally substituted independently by one or more R₁₃; or R₆ and R₇, together with the nitrogen to which they are attached, form a 4-, 5-, or 6-membered heterocyclic ring optionally substituted independently by one or more R₁₃; or a 5- or 6-membered heterocyclic ring containing 1, 2, or 3 heteroatoms selected from N, S, and O optionally substituted independently by one or more R₁₃; where R₁₃ is hydroxy, C₁₋₄ alkoxy, F, C₁₋₆ alkyl, haloalkyl, haloalkoxy, —NH₂, —NH(C₁₋₆ alkyl), or —N(C₁₋₆ alkyl)₂—. The UK-416244 analog may have a structure listed in Table 10 or Table 11 or described in the Examples.

In another aspect, the invention features a compound having the structure:

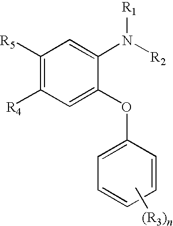

where R₁ is H or C₁₋₆ alkyl and R₂ is C₁₋₆ alkyl substituted with OH or is CH₂XR₁₄ or CH₂CH₂XR₁₄, where X is N, O, or S, and R₁₄ is H, C₁₋₆ alkyl, optionally substituted C₁₋₆ heteroalkyl, or (CH₂)_{q}(C₃₋₆ cycloalkyl) where q is 0, 1, 2, or 3, and R₃ is independently H, I, Br, F, Cl, C₁₋₆ alkyl, CF₃, CN, OCF₃, C₁₋₄ alkylthio, C₁₋₄ alkoxy, aryloxy, or CONR₆R₇; n is 1, 2, or 3; and R₄ and R₅ are independently A-X, where A is —CH═CH— or —(CH₂)ₚ— where p is 0, 1, or 2; X is H, F, Cl, Br, I, CONR₆R₇, SO₂NR₆R₇, SO₂NHC(═O)R₆, OH, C₁₋₄ alkoxy, NR₈SO₂R₉, NO₂, NR₆R₁₁, CN, CO₂R₁₀, CHO, SR₁₀, S(O)R₉, or SO₂R₁₀; R₆, R₇, R₈, and R₁₀ are independently H or C₁₋₆ alkyl, C₆₋₁₂ aryl optionally substituted independently by one or more R₁₂, or C₁₋₆ alkyl-aryl optionally substituted; R₉ is C₁₋₆ alkyl optionally substituted independently by one or more R₁₂; R₁₁ is H, C₁₋₆ alkyl optionally substituted independently by one or more R₁₂, C(O)R₆, CO₂R₉, C(O)NHR₆, or SO₂NR₆R₇; R₁₂ is F (preferably up to 3), OH, CO₂H, C₃₋₆ cycloalkyl, NH₂, CONH₂, C₁₋₆ alkoxy, C₁₋₆ alkoxycarbonyl or a 5- or 6-membered heterocyclic ring containing 1, 2, or 3 heteroatoms selected from N, S, and O optionally substituted independently by one or more R₁₃; or R₆ and R₇, together with the nitrogen to which they are attached, form a 4-, 5-, or 6-membered heterocyclic ring optionally substituted independently by one or more R₁₃; or a 5- or 6-membered heterocyclic ring containing 1, 2, or 3 heteroatoms selected from N, S, and O optionally substituted independently by one or more R₁₃; where R₁₃ is hydroxy, C₁₋₄ alkoxy, F, C₁₋₆ alkyl, haloalkyl, haloalkoxy, —NH₂, —NH(C₁₋₆ alkyl) or —N(C₁₋₆ alkyl)₂. In certain embodiments, R₁ is H, CH₃, or CH₂CH₃ and R₂ is CH₂CH₂OH, CH(OH)CH₃, CH₂CH₂CH₂OH, CH(CH₂)CH₂OH, and CH₂CH₂CH₂CH₂OH, CH(OH)CH₂CH₂CH₃, CH₂CH(OH)CH₂CH₃, and CH₂CH₂CH(OH)CH₃. In particular embodiments, the compound has the structure:

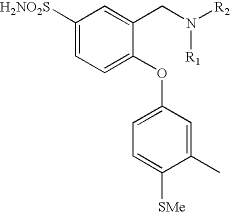

where R₁ is H or C₁₋₆ alkyl and R₂ is C₁₋₆ alkyl substituted with OH (e.g., where R₁ is H, CH₃, or CH₂CH₃ and R₂ is CH₂CH₂OH, CH(OH)CH₃, CH₂CH₂CH₂OH, CH(CH₂)CH₂OH, CH₂CH₂CH₂CH₂OH, CH(OH)CH₂CH₂CH₃, CH₂CH(OH)CH₂CH₃, or CH₂CH₂CH(OH)CH₃. The compound may have the structure

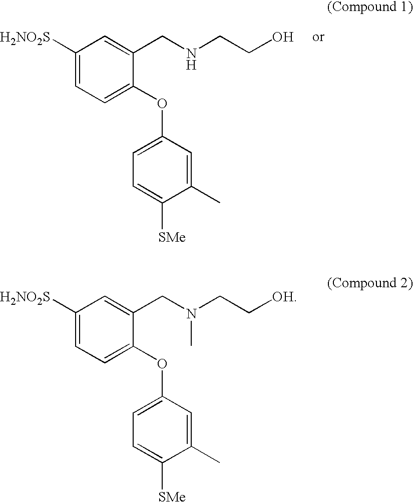

In other embodiments, the UK-416244 analog has the structure:

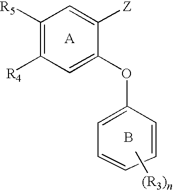

where R₃, R₄ and R₅ are as defined above and Z is CH₂NR₁R₂ where R₁ and R₂ are as defined above, NH₂, optionally substituted optional hetero C₁₋₈ alkyl (e.g., with hydroxyl, NH₂, NHC₁₋₆ alkyl), or is selected from the group consisting of:

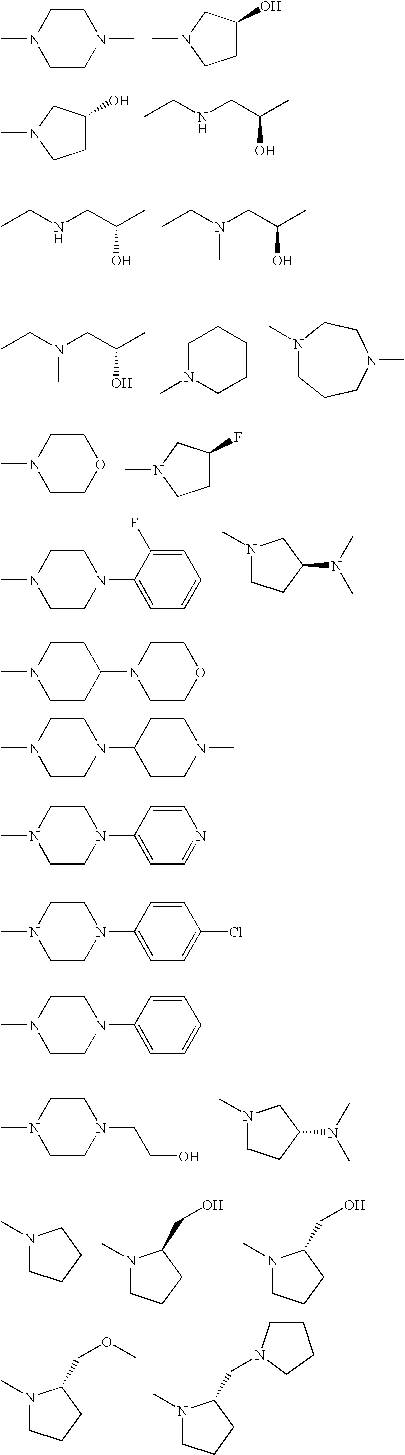

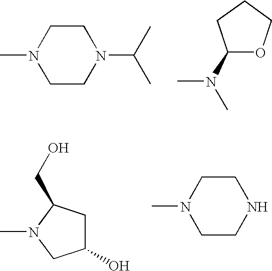

In certain embodiments, Z is CN, CH₂CH(CH₃)₂, CH₂OCH₃, CH₂N(CH₃)CH₂CH₂OH, N(CH₃)₂, CH₂N(CH₃)₂, COOH, CH₂NHCH₃, CH₂OH, CH₂NHCOCH₃, CONHCH₃, CH₂NH(CH₂)₂N(CH₃)₂, CH₂NH(CH₂)₃N(CH₃)₂, CHC(CH₃)₂, CH₂N(CH₃)(CH₂)₂N(CH₃)₂, CH₂N(CH₃)(CH₂)₃N(CH₃)₂, or CH₂CH(CH₃)₂.

In yet another aspect, the invention features a composition including a pair of agents selected from the group consisting of amorolfine and sertraline; fluvastatin and sertraline; rosuvastatin and sertraline; fulvestrant and satraplatin; amorolfine and mebeverine; amorolfine and satraplatin; ifenprodil and sertraline; amorolfine and tolterodine; atorvastatin and sertraline; amorolfine and irinotecan; lovastatin and sertraline; cytarabine and triciribine; artesunate and wortmannin; sertraline and simvastatin hydroxy acid, ammonium salt; amorolfine and cytarabine; sertraline and simvastatin; octyl methoxycinnamate and suberohydroxamic acid; 1,5-bis(4-aminophenoxy)pentane and amorolfine; (S,S)—N-desmethyl sertraline and simvastatin; artemisinin and SB-202190; interferon alfa-2a and sirolimus; amorolfine and indocyanine green; TOFA and triciribine; 3,3′-(pentamethylenedioxy)dianiline and artemisinin; artemisinin and wortmannin; 3,3″-(pentamethylenedioxy)diacetanilide and artemisinin; amorolfine and benzamil; artemisinin and triciribine; 2,2′-(pentamethylenedioxy)dianiline and amorolfine; (s,s)-n-desmethyl sertraline and simvastatin; levothyroxine and wedelolactone; 1,5-bis(4-aminophenoxy)pentane and artemisinin; benzamil and dextrothyroxine; amorolfine and trifluperidol; artemisinin and indocyanine green; dihydroartemisinin and wortmannin; flupentixol and sertraline; benzamil and levothyroxine; amorolfine and meclizine; pravastatin and sertraline; 1,5-bis(4-aminophenoxy)pentane and indocyanine green; 2-hydroxyflavanone and amorolfine; ritonavir and vinorelbine; benoxinate and dehydroepiandrosterone; ifenprodil and indocyanine green; amorolfine and arbidol; 3,3′-(pentamethylenedioxy)dianiline and indocyanine green; fulvestrant and vinorelbine; amorolfine and ezetimibe; amorolfine and Evans blue; amorolfine and gefitinib; amorolfine and topotecan; 2′,2″-(pentamethylenedioxy)diacetanilide and artemisinin; amorolfine and wedelolactone; 3,3′-(pentamethylenedioxy)dianiline and amorolfine; simvastatin and rac-cis-n-desmethyl sertraline; adefovir dipivoxil and triciribine; cytarabine and Evans blue; artemisinin and Evans blue; fluphenazine and sertraline; benzamil and SB-202190; artemisinin and rifabutin; fluphenazine and tolterodine; interferon alfa-2a and melphalan; amorolfine and melphalan; artemisinin and fulvestrant; ifenprodil and quinacrine; simvastatin and rac-cis-n-desmethyl sertraline; flupentixol and tolterodine; triciribine and wortmannin; loratadine and vinorelbine; meclizine and sertraline; budesonide and vinorelbine; 2-hydroxyflavanone and indocyanine green; hydroxyzine and sertraline; 2,2′-(pentamethylenedioxy)dianiline and artemisinin; amorolfine and flupentixol; artemisinin and chlorophyllin; ezetimibe and fluphenazine; benzamil and fluphenazine; artemisinin and wedelolactone; cytarabine and dydrogesterone; artemisinin and benzamil; 3,3′-(pentamethylenedioxy)dianiline and artemether; tolterodine and trifluperidol; artesunate and fluvastatin; artemisinin and trifluridine; adefovir dipivoxil and amorolfine; interferon alfa-2a and trifluridine; fulvestrant and triciribine; artesunate and dydrogesterone; artesunate and LY 294002; mosapride citrate and TOFA; bromocriptine and wedelolactone; artemisinin and sodium fusidate; celgosivir and interferon alfa-2a; amorolfine and dextrothyroxine; andrographis and fulvestrant; 2′-c-methylcytidine and artemisinin; amorolfine and gemcitabine; oxeladin and sertraline; artemisinin and parthenolide; artemisinin and ribavirin; dehydroepiandrosterone and tyrphostin AG 1478; sertraline and toremifene; dihydroartemisinin and fulvestrant; 2-hydroxyflavanone and TOFA; artesunate and repaglinide; mofebutazone and wedelolactone; artesunate and simvastatin; 2,2′-(pentamethylenedioxy)dianiline and artesunate; artemisinin and gemcitabine; dihydroartemisinin and ezetimibe; chlorophyllin and cytarabine; interferon alfa-2a and sirolimus; suberohydroxamic acid and VX-497; artemisinin and VX-497; artesunate and VX-497; tolterodine and VX-950; artemisinin and HCV-796; artemisinin and NM-283; NM-283 and wedelolactone; artemisinin and SCH 503034; cytarabine and SCH 503034; SCH 503034 and triciribine; interferon alfa-2a and melphalan; benoxinate and VX-950; HCV-796 and sirolimus; benoxinate and SCH 503034; melphalan and VX-950; ritonavir and VX-950; VX-950 and VX-497; artemisinin and VX-950; triciribine and VX-950; suberohydroxamic acid and VX-950; HCV-796 and suberohydroxamic acid; sirolimus and VX-950; melphalan and SCH 503034; SCH 503034 and wortmannin; SCH 503034 and tolterodine; ritonavir and SCH 503034; ezetimibe and VX-950; HCV-796 and VX-497; chlorophyllin and VX-497; HCV-796 and melphalan; capsaicin and NM-283; SCH 503034 and sirolimus; LY 294002 and SCH 503034; adefovir dipivoxil and SCH 503034; interferon alfa-2a and trifluridine; HCV-796 and trifluridine; GW 5074 and NM-283; mosapride and VX-950; interferon alfa-2a and VX-497; NM-283 and trequinsin; cytarabine and HCV-796; adefovir dipivoxil and VX-950; cytarabine and VX-950; SCH 503034 and saquinavir; VX-950 and wortmannin; capsaicin and VX-950; 2-hydroxyflavanone and NM-283; bromhexine and VX-950; HCV-796 and wortmannin; artemisinin and ribavirin; VX-950 and verapamil; SCH 503034 and verapamil; SCH 503034 and topotecan; HCV-796 and topotecan; trifluperidol and VX-950; irinotecan and SCH 503034; artesunate and SCH 503034; repaglinide and SCH 503034; topotecan and VX-950; tepaglinide and VX-950; arbidol and VX-950; chlorophyllin and HCV-796; benzydamine and VX-950; NM-283 and trifluperidol; capsaicin and HCV-796; NM-283 and phenazopyridine; NM-283 and trifluridine; and adefovir dipivoxil and HCV-796. In any of the pairs of agents above, the agent may be substituted with an analog of that agent (e.g., any analog described herein). In particular embodiments, sertraline is substituted with a sertraline analog, UK-416244, or a UK-416244 analog.

In certain embodiments, the combination is selected from group consisting of simvastatin and sertraline, a sertraline analog, UK-416244, or a UK-416244 analog; fluvastatin and sertraline, a sertraline analog, UK-416244, or a UK-416244 analog; fluphenazine and sertraline, a sertraline analog, UK-416244, or a UK-416244 analog; artesunate and simvastatin; artesunate and wortmannin; artemisinin and chlorophyllin; artemisinin and 3,3′-(pentamethylenedioxy)dianiline; amorolfine and meclizine; amorolfine and sertraline, a sertraline analog, UK-416244, or a UK-416244 analog; amorolfine and trifluridine; amorolfine and 2-hydroxyflavanone; amorolfine and ezetimibe; amorolfine and benzamil; amorolfine and trifluperidol; and octyl methoxycinnamate and suberohydroxamic acid.

In any of the above aspects, the two agents may be present in amounts that, when administered to a patient having a viral disease (e.g., any viral disease described herein), are effective to treat the patient. The composition may further include one or more (e.g., two, three, four, five, or six) additional agents selected from the agents of Table 1, Table 2, Table 3, Table 4, and Table 5 (e.g., where the agents are not a combination of agents selected from Table 7). The composition may be formulated, for example, for oral, systemic, parenteral, topical (e.g., ophthalmic, dermatologic), intravenous, or intramuscular administration.

In another aspect, the invention features a method for treating a patient having a viral disease. The method includes administering to the patient an agent selected from the agents of Table 1, or an analog thereof, in an amount effective to treat the patient. In certain embodiments, the agent is sertraline, a sertraline analog, UK-416244, or a UK-416244 analog (e.g., any of those described herein).

In another aspect, the invention features a method for treating a patient having hepatitis C. The method includes administering to the patient an agent selected from the agents of Table 1 and Table 2, or an analog thereof, in an amount effective to treat the patient. In certain embodiments, the agent is sertraline, a sertraline analog, UK-416244, or a UK-416244 analog (e.g., any of those described herein).

In another aspect, the invention features a method for treating a patient having a viral disease. The method includes administering to the patient a plurality of agents where the first agent is selected from the agents of Table 1, Table 2, and Table 3, or an analog thereof, and the second agent is selected from the agents of Table 1, Table 2, Table 3, Table 4, and Table 5 (e.g., Table 4 and Table 5), or an analog thereof, where the agents are administered within 28 days (e.g., within 21, 14, 10, 7, 5, 4, 3, 2, or 1 days) or within 24 hours (e.g., 12, 6, 3, 2, or 1 hours; or concomitantly) of each other in amounts that together are effective to treat the patient.

In another aspect, the invention features a method for treating a patient having a viral disease. The method includes administering to the patient sertraline, a sertraline analog, UK-416244, or a UK-416244 analog, and an HMG-CoA reductase inhibitor, where the two agents are administered within 28 days of each other in amounts that together are effective to treat the patient. The HMG-CoA reductase inhibitor may be fluvastatin, simvastatin, lovastatin, or rosuvastatin.

In another aspect, the invention features a method for treating a patient having a viral disease. The method includes administering to the patient sertraline, a sertraline analog, UK-416244, or a UK-416244 analog, and an antihistamine where the two agents are administered within 28 days of each other in amounts that together are effective to treat the patient. The antihistamine may be hydroxyzine.

In yet another aspect, the invention features a method for treating a patient having a viral disease. The method includes administering to the patient a pair of agents selected from the group consisting of amorolfine and sertraline, a sertraline analog, UK-416244, or a UK-416244 analog; fluvastatin and sertraline, a sertraline analog, UK-416244, or a UK-416244 analog; rosuvastatin and sertraline, a sertraline analog, UK-416244, or a UK-416244 analog; fulvestrant and satraplatin; amorolfine and mebeverine; amorolfine and satraplatin; ifenprodil and sertraline; amorolfine and tolterodine; atorvastatin and sertraline; amorolfine and irinotecan; lovastatin and sertraline, a sertraline analog, UK-416244, or a UK-416244 analog; cytarabine and triciribine; artesunate and wortmannin; sertraline, a sertraline analog, UK-416244, or a UK-416244 analog and simvastatin hydroxy acid, ammonium salt; amorolfine and cytarabine; sertraline, a sertraline analog, UK-416244, or a UK-416244 analog and simvastatin; octyl methoxycinnamate and suberohydroxamic acid; 1,5-bis(4-aminophenoxy)pentane and amorolfine; (S,S)—N-desmethyl sertraline and simvastatin; artemisinin and SB-202190; interferon alfa-2a and sirolimus; amorolfine and indocyanine green; TOFA and triciribine; 3,3′-(pentamethylenedioxy)dianiline and artemisinin; artemisinin and wortmannin; 3,3″-(pentamethylenedioxy)diacetanilide and artemisinin; amorolfine and benzamil; artemisinin and triciribine; 2,2′-(pentamethylenedioxy)dianiline and amorolfine; (s,s)-n-desmethyl sertraline and simvastatin; levothyroxine and wedelolactone; 1,5-bis(4-aminophenoxy)pentane and artemisinin; benzamil and dextrothyroxine; amorolfine and trifluperidol; artemisinin and indocyanine green; dihydroartemisinin and wortmannin; flupentixol and sertraline, a sertraline analog, UK-416244, or a UK-416244 analog; benzamil and levothyroxine; amorolfine and meclizine; pravastatin and sertraline, a sertraline analog, UK-416244, or a UK-416244 analog; 1,5-bis(4-aminophenoxy)pentane and indocyanine green; 2-hydroxyflavanone and amorolfine; ritonavir and vinorelbine; benoxinate and dehydroepiandrosterone; ifenprodil and indocyanine green; amorolfine and arbidol; 3,3′-(pentamethylenedioxy)dianiline and indocyanine green; fulvestrant and vinorelbine; amorolfine and ezetimibe; amorolfine and Evans blue; amorolfine and gefitinib; amorolfine and topotecan; 2′,2″-(pentamethylenedioxy)diacetanilide and artemisinin; amorolfine and wedelolactone; 3,3′-(pentamethylenedioxy)dianiline and amorolfine; simvastatin and rac-cis-n-desmethyl sertraline; adefovir dipivoxil and triciribine; cytarabine and Evans blue; artemisinin and Evans blue; fluphenazine and sertraline, a sertraline analog, UK-416244, or a UK-416244 analog; benzamil and SB-202190; artemisinin and rifabutin; fluphenazine and tolterodine; interferon alfa-2a and melphalan; amorolfine and melphalan; artemisinin and fulvestrant; ifenprodil and quinacrine; simvastatin and rac-cis-n-desmethyl sertraline; flupentixol and tolterodine; triciribine and wortmannin; loratadine and vinorelbine; meclizine and sertraline, a sertraline analog, UK-416244, or a UK-416244 analog; budesonide and vinorelbine; 2-hydroxyflavanone and indocyanine green; hydroxyzine and sertraline; 2,2′-(pentamethylenedioxy)dianiline and artemisinin; amorolfine and flupentixol; artemisinin and chlorophyllin; ezetimibe and fluphenazine; benzamil and fluphenazine; artemisinin and wedelolactone; cytarabine and dydrogesterone; artemisinin and benzamil; 3,3′-(pentamethylenedioxy)dianiline and artemether; tolterodine and trifluperidol; artesunate and fluvastatin; artemisinin and trifluridine; adefovir dipivoxil and amorolfine; interferon alfa-2a and trifluridine; fulvestrant and triciribine; artesunate and dydrogesterone; artesunate and LY 294002; mosapride citrate and TOFA; bromocriptine and wedelolactone; artemisinin and sodium fusidate; celgosivir and interferon alfa-2a; amorolfine and dextrothyroxine; andrographis and fulvestrant; 2′-c-methylcytidine and artemisinin; amorolfine and gemcitabine; oxeladin and sertraline, a sertraline analog, UK-416244, or a UK-416244 analog; artemisinin and parthenolide; artemisinin and ribavirin; dehydroepiandrosterone and tyrphostin AG 1478; sertraline, a sertraline analog, UK-416244, or a UK-416244 analog and toremifene; dihydroartemisinin and fulvestrant; 2-hydroxyflavanone and TOFA; artesunate and repaglinide; mofebutazone and wedelolactone; artesunate and simvastatin; 2,2′-(pentamethylenedioxy)dianiline and artesunate; artemisinin and gemcitabine; dihydroartemisinin and ezetimibe; chlorophyllin and cytarabine; interferon alfa-2a and sirolimus; suberohydroxamic acid and VX-497; artemisinin and VX-497; artesunate and VX-497; tolterodine and VX-950; artemisinin and HCV-796; artemisinin and NM-283; NM-283 and wedelolactone; artemisinin and SCH 503034; cytarabine and SCH 503034; SCH 503034 and triciribine; interferon alfa-2a and melphalan; benoxinate and VX-950; HCV-796 and sirolimus; benoxinate and SCH 503034; melphalan and VX-950; ritonavir and VX-950; VX-950 and VX-497; artemisinin and VX-950; triciribine and VX-950; suberohydroxamic acid and VX-950; HCV-796 and suberohydroxamic acid; sirolimus and VX-950; melphalan and SCH 503034; SCH 503034 and wortmannin; SCH 503034 and tolterodine; ritonavir and SCH 503034; ezetimibe and VX-950; HCV-796 and VX-497; chlorophyllin and VX-497; HCV-796 and melphalan; capsaicin and NM-283; SCH 503034 and sirolimus; LY 294002 and SCH 503034; adefovir dipivoxil and SCH 503034; interferon alfa-2a and trifluridine; HCV-796 and trifluridine; GW 5074 and NM-283; mosapride and VX-950; interferon alfa-2a and VX-497; NM-283 and trequinsin; cytarabine and HCV-796; adefovir dipivoxil and VX-950; cytarabine and VX-950; SCH 503034 and saquinavir; VX-950 and wortmannin; capsaicin and VX-950; 2-hydroxyflavanone and NM-283; bromhexine and VX-950; HCV-796 and wortmannin; artemisinin and ribavirin; VX-950 and verapamil; SCH 503034 and verapamil; SCH 503034 and topotecan; HCV-796 and topotecan; trifluperidol and VX-950; irinotecan and SCH 503034; artesunate and SCH 503034; repaglinide and SCH 503034; topotecan and VX-950; tepaglinide and VX-950; arbidol and VX-950; chlorophyllin and HCV-796; benzydamine and VX-950; NM-283 and trifluperidol; capsaicin and HCV-796; NM-283 and phenazopyridine; NM-283 and trifluridine; and adefovir dipivoxil and HCV-796, where the agents are administered within 28 days of each other in amounts that together are effective to treat the patient.

In another aspect, the invention features a method for treating a patient having a viral disease. The method includes administering to the patient a pair of agents selected from the group consisting of simvastatin and sertraline, a sertraline analog, UK-416244, or a UK-416244 analog; fluvastatin and sertraline, a sertraline analog, UK-416244, or a UK-416244 analog; fluphenazine and sertraline, a sertraline analog, UK-416244, or a UK-416244 analog; artesunate and simvastatin; artesunate and wortmannin; artemisinin and chlorophyllin; artemisinin and 3,3′-(pentamethylenedioxy)dianiline; amorolfine and meclizine; amorolfine and sertraline, a sertraline analog, UK-416244, or a UK-416244 analog; amorolfine and trifluridine; amorolfine and 2-hydroxyflavanone; amorolfine and ezetimibe; amorolfine and benzamil; amorolfine and trifluperidol; and octyl methoxycinnamate and suberohydroxamic acid, where the two agents are administered within 28 days of each other in amounts that together are effective to treat the patient.

The methods of any of the above aspects may be performed in conjunction with administering to the patient an additional treatment (e.g., an antiviral therapy such as those agents listed in Table 4 and Table 5, or an analog thereof) for a viral disease, where the method and the additional treatment (e.g., not a combination of agents selected from Table 6 and Table 7) are administered within 6 months (e.g., within 3, 2, or 1 months; within 28, 21, 14, 10, 7, 5, 4, 3, 2, or 1 days; within 24, 12, 6, 3, 2, or 1 hours; or concomitantly) of each other. The agents may be administered to the patient by intravenous, intramuscular, inhalation, topical (e.g., ophthalmic, determatologic), or oral administration.

In certain embodiments of any of the above methods (e.g., methods including administration of an antidepressant agent such as an SSRI or a tricyclic antidepressant), the patient being treated has not been diagnosed with or does not suffer from depression, major depressive disorder, obsessive-compulsive disorder, panic disorder, posttraumatic stress disorder, social anxiety disorder, generalized anxiety disorder, or premenstrual dysphoric disorder. In other embodiments, (e.g., methods including administration of an HMG-CoA reductase inhibitor), the patient being treated has not been diagnosed with or does not suffer from hypercholesteraolemia, primary familial hypercholesterolemia (heterozygous variant), mixed hyperlipidaemia (corresponding to type IIa and IIb of the Fredrickson classification), or coronary artery disease, or has not had a myocardial infarction, a cerebrovascular event, an coronary bypass surgery, or a translumen percutaneous coronary angioplasty.

In another aspect, the invention features a kit including an agent selected from any of the agents of Table 1, or an analog thereof; and instructions for administering the agent to a patient having a viral disease.

In another aspect, the invention features a kit including an agent selected from any of the agents of Table 1 and Table 2, or an analog thereof; and instructions for administering the agent to a patient having hepatitis C.

In another aspect, the invention features a kit including a composition including two or more (e.g., 3, 4, 5, 6, or 7) agents selected from any of the agents of Table 1, or an analog thereof, Table 2, and Table 3; and instructions for administering the composition to a patient having a viral disease.

In another aspect, the invention features a kit including a first agent selected from any of the agents of Table 1, Table 2, and Table 3, or an analog thereof; a second, different agent selected from any of the agents of Table 1, Table 2, and Table 3, or an analog thereof; and instructions for administering the first and second agents to a patient having a viral disease.

In another aspect, the invention features a kit including an agent selected from any one of the agents of Table 1, Table 2, and Table 3, or an analog thereof; and instructions for administering the agent with a second, different agent selected from any of the agents of Table 1, Table 2, and Table 3, or an analog thereof to a patient having a viral disease.

In another aspect, the invention features a kit including a composition including (i) a first agent selected from any one of the agents of Table 1, Table 2, and Table 3, or an analog thereof, and (ii) one or more agents of Table 4 and Table 5, or an analog thereof; and instructions for administering the composition to a patient having a viral disease.

In another aspect, the invention features a kit including (a) a first agent selected from any of the agents of Table 1, Table 2, and Table 3, or an analog thereof; (b) one or more agents of Table 4 and Table 5, or an analog thereof; and (c) instructions for administering (a) and (b) to a patient having a viral disease.

In another aspect, the invention features a kit including an agent selected from any of the agents of Table 1, or an analog thereof; and instructions for administering the agent and one or more agents of Table 4 or Table 5, or an analog thereof, to a patient having a viral disease.

In another aspect, the invention features a kit including an agent selected from any of the agents of Table 1 and Table 2, or an analog thereof; and instructions for administering the agent and one or more agents of Table 4 or Table 5, or an analog thereof, to a patient having hepatitis C.

In another aspect, the invention features a kit including (a) one or more agents of Table 4 and Table 5, or an analog thereof; and (b) instructions for administering the agent from (a) with any agent of Table 1, Table 2, and Table 3, or an analog thereof, to a patient having a viral disease.

In another aspect, the invention features a kit including a agent selected from the group consisting of sertraline, a sertraline analog, UK-416244, and a UK-416244 analog; an HMG-CoA reductase inhibitor (e.g., fluvastatin, simvastatin, lovastatin, or rosuvastatin); and instructions for administering the agent and the HMG-CoA reductase inhibitor to a patient having a viral disease.

In another aspect, the invention features a kit including a composition including sertraline, a sertraline analog, UK-416244, or a UK-416244 analog, and an HMG-CoA reductase inhibitor (e.g., fluvastatin, simvastatin, lovastatin, or rosuvastatin); and instructions for administering the composition to a patient having a viral disease.

In another aspect, the invention features a kit including sertraline, a sertraline analog, UK-416244, or a UK-416244 analog; an antihistamine (e.g., hydroxyzine); and instructions for administering the sertraline or sertraline analog and the antihistamine to a patient having a viral disease.

In another aspect, the invention features a kit including a composition including sertraline or UK-416244, and an antihistamine (e.g., hydroxyzine); and instructions for administering the composition to a patient having a viral disease.

In another aspect, the invention features a kit including (a) a pair of agents selected from the group consisting of amorolfine and sertraline; fluvastatin and sertraline; rosuvastatin and sertraline; fulvestrant and satraplatin; amorolfine and mebeverine; amorolfine and satraplatin; ifenprodil and sertraline; amorolfine and tolterodine; atorvastatin and sertraline; amorolfine and irinotecan; lovastatin and sertraline; cytarabine and triciribine; artesunate and wortmannin; sertraline and simvastatin hydroxy acid, ammonium salt; amorolfine and cytarabine; sertraline and simvastatin; octyl methoxycinnamate and suberohydroxamic acid; 1,5-bis(4-aminophenoxy)pentane and amorolfine; (S,S)—N-desmethyl sertraline and simvastatin; artemisinin and SB-202190; interferon alfa-2a and sirolimus; amorolfine and indocyanine green; TOFA and triciribine; 3,3′-(pentamethylenedioxy)dianiline and artemisinin; artemisinin and wortmannin; 3,3″-(pentamethylenedioxy)diacetanilide and artemisinin; amorolfine and benzamil; artemisinin and triciribine; 2,2′-(pentamethylenedioxy)dianiline and amorolfine; (S,S)-n-desmethyl sertraline and simvastatin; levothyroxine and wedelolactone; 1,5-bis(4-aminophenoxy)pentane and artemisinin; benzamil and dextrothyroxine; amorolfine and trifluperidol; artemisinin and indocyanine green; dihydroartemisinin and wortmannin; flupentixol and sertraline; benzamil and levothyroxine; amorolfine and meclizine; pravastatin and sertraline; 1,5-bis(4-aminophenoxy)pentane and indocyanine green; 2-hydroxyflavanone and amorolfine; ritonavir and vinorelbine; benoxinate and dehydroepiandrosterone; ifenprodil and indocyanine green; amorolfine and arbidol; 3,3′-(pentamethylenedioxy)dianiline and indocyanine green; fulvestrant and vinorelbine; amorolfine and ezetimibe; amorolfine and Evans blue; amorolfine and gefitinib; amorolfine and topotecan; 2′,2″-(pentamethylenedioxy)diacetanilide and artemisinin; amorolfine and wedelolactone; 3,3′-(pentamethylenedioxy)dianiline and amorolfine; simvastatin and rac-cis-n-desmethyl sertraline; adefovir dipivoxil and triciribine; cytarabine and Evans blue; artemisinin and Evans blue; fluphenazine and sertraline; benzamil and SB-202190; artemisinin and rifabutin; fluphenazine and tolterodine; interferon alfa-2a and melphalan; amorolfine and melphalan; artemisinin and fulvestrant; ifenprodil and quinacrine; simvastatin and rac-cis-n-desmethyl sertraline; flupentixol and tolterodine; triciribine and wortmannin; loratadine and vinorelbine; meclizine and sertraline; budesonide and vinorelbine; 2-hydroxyflavanone and indocyanine green; hydroxyzine and sertraline; 2,2′-(pentamethylenedioxy)dianiline and artemisinin; amorolfine and flupentixol; artemisinin and chlorophyllin; ezetimibe and fluphenazine; benzamil and fluphenazine; artemisinin and wedelolactone; cytarabine and dydrogesterone; artemisinin and benzamil; 3,3′-(pentamethylenedioxy)dianiline and artemether; tolterodine and trifluperidol; artesunate and fluvastatin; artemisinin and trifluridine; adefovir dipivoxil and amorolfine; interferon alfa-2a and trifluridine; fulvestrant and triciribine; artesunate and dydrogesterone; artesunate and LY 294002; mosapride citrate and TOFA; bromocriptine and wedelolactone; artemisinin and sodium fusidate; celgosivir and interferon alfa-2a; amorolfine and dextrothyroxine; andrographis and fulvestrant; 2′-c-methylcytidine and artemisinin; amorolfine and gemcitabine; oxeladin and sertraline; artemisinin and parthenolide; artemisinin and ribavirin; dehydroepiandrosterone and tyrphostin ag 1478; sertraline and toremifene; dihydroartemisinin and fulvestrant; 2-hydroxyflavanone and TOFA; artesunate and repaglinide; mofebutazone and wedelolactone; artesunate and simvastatin; 2,2′-(pentamethylenedioxy)dianiline and artesunate; artemisinin and gemcitabine; dihydroartemisinin and ezetimibe; chlorophyllin and cytarabine; interferon alfa-2a and sirolimus; suberohydroxamic acid and VX-497; artemisinin and VX-497; artesunate and VX-497; tolterodine and VX-950; artemisinin and HCV-796; artemisinin and NM-283; NM-283 and wedelolactone; artemisinin and SCH 503034; cytarabine and SCH 503034; SCH 503034 and triciribine; interferon alfa-2a and melphalan; benoxinate and VX-950; HCV-796 and sirolimus; benoxinate and SCH 503034; melphalan and VX-950; ritonavir and VX-950; VX-950 and VX-497; artemisinin and VX-950; triciribine and VX-950; suberohydroxamic acid and VX-950; HCV-796 and suberohydroxamic acid; sirolimus and VX-950; melphalan and SCH 503034; SCH 503034 and wortmannin; SCH 503034 and tolterodine; ritonavir and SCH 503034; ezetimibe and VX-950; HCV-796 and VX-497; chlorophyllin and VX-497; HCV-796 and melphalan; capsaicin and NM-283; SCH 503034 and sirolimus; LY 294002 and SCH 503034; adefovir dipivoxil and SCH 503034; interferon alfa-2a and trifluridine; HCV-796 and trifluridine; GW 5074 and NM-283; mosapride and VX-950; interferon alfa-2a and VX-497; NM-283 and trequinsin; cytarabine and HCV-796; adefovir dipivoxil and VX-950; cytarabine and VX-950; SCH 503034 and saquinavir; VX-950 and wortmannin; capsaicin and VX-950; 2-hydroxyflavanone and NM-283; bromhexine and VX-950; HCV-796 and wortmannin; artemisinin and ribavirin; VX-950 and verapamil; SCH 503034 and verapamil; SCH 503034 and topotecan; HCV-796 and topotecan; trifluperidol and VX-950; irinotecan and SCH 503034; artesunate and SCH 503034; repaglinide and SCH 503034; topotecan and VX-950; repaglinide and VX-950; arbidol and VX-950; chlorophyllin and HCV-796; benzydamine and VX-950; NM-283 and trifluperidol; capsaicin and HCV-796; NM-283 and phenazopyridine; NM-283 and trifluridine; and adefovir dipivoxil and HCV-796; and (b) instructions for administering the pair of agents to a patient having a viral disease. The kit may include a composition including the pair of agents. In certain embodiments of the kit, sertraline is substituted for a sertraline analog, UK-416244, or a UK-416244 analog.

In another aspect, the invention features a kit including (a) a pair of agents selected from the group consisting of simvastatin and sertraline; fluvastatin and sertraline; fluphenazine and sertraline; artesunate and simvastatin; artesunate and wortmannin; artemisinin and chlorophyllin; artemisinin and 3,3′-(pentamethylenedioxy)dianiline; amorolfine and meclizine; amorolfine and sertraline; amorolfine and trifluridine; amorolfine and 2-hydroxyflavanone; amorolfine and ezetimibe; amorolfine and benzamil; amorolfine and trifluperidol; and octyl methoxycinnamate and suberohydroxamic acid; and (b) instructions for administering the pair of agents to a patient having a viral disease. The kit may include a composition including the pair of agents. In certain embodiments of the kit, sertraline is substituted for a sertraline analog, UK-416244, or a UK-416244 analog.

In another aspect, the invention features a method of identifying a combination that may be useful for the treatment of a patient having a viral disease, or the prevention or reduction of the viral disease. The method includes the steps of contacting cells including at least a portion of the genome of a virus with an agent selected from any one the agents of Table 1, Table 2, and Table 3 and a candidate compound, wherein the portion of the genome (e.g., of any virus described herein) is capable of replication in the cells; and determining whether the combination of the agent and the candidate compound inhibits the replication of the portion of the genome relative to cells contacted with the agent but not contacted with the candidate compound, where a reduction in replication identifies the combination as a combination useful for the treatment of a patient having a viral disease, or the prevention or reduction of a viral disease. The reduction in replication may be the result of a decreased rate of DNA or RNA replication, a decreased rate of RNA translation, or inhibition of a protein required for viral replication (e.g., a protein coded for by the viral genome or the host organism). If the at least portion of a genome is from the hepatitis C genome, the reduction in replication may also be due to a decreased rate of polyprotein processing. The cells may be mammalian cells (e.g., hepatic cells, for example, any of those described herein) such as human cells.

The viral disease referred to in any of the above aspects of the invention, including the methods of treatment of the invention, the compositions and kits of the invention, and methods of the invention for identifying combinations may be caused by a single stranded RNA virus, a flaviviridae virus (e.g., a hepacivirus such as HCV, flavivirus, pestivirus, or hepatitis G virus), or a hepatic virus (e.g., any hepatic virus described herein such as hepatitis A, hepatitis B, hepatitis C, hepatitis D, hepatitis E, non-ABCDE hepatitis, or hepatitis G). In certain embodiments, the viral disease is caused by a flavivirus which include without limitation Absettarov, Alfuy, Apoi, Aroa, Bagaza, Banzi, Bouboui, Bussuquara, Cacipacore, Carey Island, Dakar bat, Dengue 1, Dengue 2, Dengue 3, Dengue 4, Edge Hill, Entebbe bat, Gadgets Gully, Hanzalova, Hypr, Ilheus, Israel turkey meningoencephalitis, Japanese encephalitis, Jugra, Jutiapa, Kadam, Karshi, Kedougou, Kokobera, Koutango, Kumlinge, Kunjin, Kyasanur Forest disease, Langat, Louping ill, Meaban, Modoc, Montana myotis leukoencephalitis, Murray valley encephalitis, Naranjal, Negishi, Ntaya, Omsk hemorrhagic fever, Phnom-Penh bat, Powassan, Rio Bravo, Rocio, royal farm, Russian spring-summer encephalitis, Saboya, St. Louis encephalitis, Sal Vieja, San Perlita, Saumarez Reef, Sepik, Sokuluk, Spondweni, Stratford, Tembusu, Tyuleniy, Uganda S, Usutu, Wesselsbron, west Nile, Yaounde, yellow fever, and Zika viruses, or any of the viruses described in Chapter 31 of Fields Virology, Fields, B. N., Knipe, D. M., and Howley, P. M., eds. Lippincott-Raven Publishers, Philadelphia, Pa., 1996. In other embodiments, the viral disease is caused by a pestivirus, which include bovine viral diarrhea virus (“BVDV”), classical swine fever virus (“CSFV,” also called hog cholera virus), border disease virus (“BDV”) and any of those discussed in Chapter 33 of Fields Virology, supra. In other embodiments, the viral disease is caused by a virus such as hepatitis A, hepatitis B. hepatitis C (e.g., genotype 1 such as 1a or 1b; genotype 2 such as 2a, 2b, or 2c; genotype 3; genotype 4; genotype 5; genotype 6); hepatitis D; or hepatitis E. The viral hepatitis may further be a non-ABCDE viral hepatitis (e.g., hepatitis G).

Additional viral therapies are described in Table 4 and Table 5.

TABLE 4

| | | | |
|---|---|---|---|
| (+)-Calanolide A | (+)-Dihydrocalanolide A | 145U87 | 2-Nor-cyclic GMP |
| 3,4-Dicaffeoylquinic acid | 3-Hydroxymethyl | 3-Hydroxyphthaloyl-beta- | 3-Nitrosobenzamide |
| | dicamphanoyl khellactone | lactoglobulin | |
| 4-Azidothymidine | 4-Methyl dicamphanoyl | 524C79 | 739W94 |
| | khellactone | | |
| A 160621 | A 315675 | A 315677 | A 5021 |
| A 74259 | A 74704 | A 77003 | A 80735 |
| A 80987 | A 91883A | A 98881 | Abacavir |
| AC 2 | Acemannan | Acetylcysteine-Zambon | ACH 126445 |
| ACH 126447 | Aciclovir (e.g., extended | Aciclovir-PMPA | ACP HIP |
| | release, controlled release, | | |
| | topical patch) | | |
| Actinohivin | AD 439 | AD 519 | Adamantylamide dipeptide |
| ADS J1 | Afovirsen | AG 1284 | AG 1350 |
| AG 1478 | AG 1859 | AG 555 | AG 6840 |
| AG 6863 | AGT-1 | AHA 008 | Aidfarel |
| AL 721 | Alamifovir | Albumin/interferon-alpha | ALN RSV01 |
| Alovudine | Alpha HGA | Alpha-1PDX | Alpha-antitrypsin |
| Alvircept sudotox | Alvocidib | ALX 0019 | ALX 404C |
| AM 285 | AM 365 | Amantadine | AMD 070 |
| AMD 3329 | AMD 3465 | AMD 8664 | Amdoxovir |
| Amidinomycin | Aminopeptidase | Amitivir | Ampligen |
| Amprenavir | AMZ 0026 | Ancriviroc | Anti-CCR5 monoclonal |
| | | | antibody |
| Anti-CCR5/CXCR4 sheep | Anti-CD3 monoclonal | Anti-CD4 monoclonal | Anti-CD7 monoclonal |
| monoclonal antibody | antibody CD4IgG conjugate | antibody | antibody |
| Anti-CD8 monoclonal antibody | Anti-CMV monoclonal | Anti-hepatitis B ribozyme | Anti-HIV catalytic antibody |
| | antibody | | |
| Anti-HIV immunotoxin (IVAX) | Anti-HIV-1 human | Anti-HIV-1 human | Anti-HIV-1 human |
| | monoclonal antibody 2F5 | monoclonal antibody 2G12 | monoclonal antibody 4E10 |
| Antineoplaston AS2 1 (e.g., oral) | Anti-RSV antibody (Intracel, | Antisense oligonucleotide PB2 | Aop-RANTES |
| | Corp.) | AUG | |
| Aplaviroc | Apricitabine | AQ 148 | AR 132 |
| AR 177 | ARB 95214 | ARB 97265 | ARB 97268 |
| | ARQ 323 | AS 101 | AT 61 |
| Atazanavir | Atevirdine | AV 1101 | AV 2921 |
| AV 2923 | AV 2925 | AV 2927 | Avarol |
| AXD 455 | Azidodideoxyguanosine | Azodicarbonamide | Bafilomycin A1 |
| Baicalin | BAY 414109 | BAY 439695 | BAY 504798 |
| BAY Z 4305 | BB 10010 | BB 2116 | BCH 10652 |
| BCH 371 | BCH 527 | BCTP | BCX 140 |
| BCX 1591 | BCX 1827 | BCX 1898 | BCX 1923 |
| BEA | BEA 005 | Bellenamine | Benanomicin A |
| Benzalkonium (e.g., chloride) | Benzalkonium | Beta-D-FDOC | Beta-L-ddC |
| | chloride/octoxynol 9 (e.g., | | |
| | vaginal gel) | | |
| Beta-L-FddC | Bevirimat | BG 777 | BGP 15 |
| BILA 2185 BS | BILR 355 | BIRM ECA 10-142 | BL 1743 |
| BM 510836 | BMS 181167-02 | BMS 181184 | BMS 182193 |
| BMS 186318 | BMS 187071 | BMS 488043 | BMS 806 |
| BMY 27709 | Brecanavir | Brefeldin A | Brequinar |
| Brivudine | BRL 47923DP | BSL 4 | BST 5001 |
| BTA 188 | BTA 798 | C 1605 | C 2507 |
| C31G | Calcium spirulan | Canventol | Capravirine |
| Carbendazim | Carbocyclic deazaadenosine | Carbopol polymer gel | Carbovir |
| CC 3052 | CD4 fusion toxin | CD4 IgG | CD4-ricin chain A |
| Cellulose sulfate | CF 1743 | CFY 196 | CGA 137053 |
| CGP 35269 | CGP 49689 | CGP 53437 | CGP 53820 |
| CGP 57813 | CGP 61783 | CGP 64222 | CGP 70726 |
| CGP 75136 | CGP 75176 | CGP 75355 | CI 1012 |
| CI 1013 | Cidofovir | Civamide | CL 190038 |
| CL 387626 | Clevudine | CMV 423 | CMX 001 |
| CNBA-Na | CNJ I02 | Cobra venom peptide | Conocurvone |
| Cosalane | Costatolide | CP 1018161 | CP 38 |
| CP 51 | CPFDD | CRL 1072 | Crofelemer |
| CS 8958 | CS 92 | CT 2576 | CTC 96 |
| Curdlan sulfate | Cyanovirin-N | CYT 99007 | Cytomegalovirus immune |
| | | | globulin |
| DAB486interleukin-2 | DABO 1220 | Dacopafant | DAP 30 |
| DAP 32 | Dapivirine | Darunavir | D-aspartic-beta-hydroxamate |
| DB 340 | DDCDP-DG | DDGA | Deazaadenosine |
| Deazaneplanocin A | DEB 025 | Delavirdine | Delmitide |
| Denileukin diftitox | Deoxyfluoroguanosine | DES 6 | Dexelvucitabine |
| Dextran sulfate | Dextrin 2-sulfate | DG 35 | Didanosine |
| Dideoxyadenosine | Dideoxyguanosine | Dideoxythymidine | Didox |
| Dihydrocostatolide | Dinitrochlorobenzene | DL 110 | DMP 323 |
| DMP 850 | DMP 851 | DmTr-ODN12 | Docosanol |
| DP 107 | DPC 082 | DPC 083 | DPC 681 |
| DPC 684 | DPC 961 | DPC 963 | Droxinavir |
| DUP 925 | DYE | E 913 | EB-Foscarnet |
| E-EPSEU | EGS 21 | EHT 899 | Elvucitabine |
| EM 1421 | EM 2487 | Emivirine | Emtricitabine |
| Emtricitabine/tenofovir | Enfuvirtide | Entecavir | Eosinophil-derived |
| disoproxil fumarate | | | neutralizing agent |
| Episiastatin B | ET 007 | Etanercept | Ether lipid analogue |
| Etoviram | Etravirine | F 105 | F 36 |
| F 50003 | Famciclovir | Fasudil | Fattiviracin A1 |
| FEAU | Feglymycin | Felvizumab | FGI 345 |
| Fiacitabine | Fialuridine | FLG | Flutimide |
| Fomivirsen | Fosalvudine tidoxil | Fosamprenavir | Foscarnet Sodium |
| Fozivudine | FP 21399 | F-PBT | FPMPA |
| FPMPDAP | FR 191512 | FR 198248 | Galactan sulfate |
| Ganciclovir | GAP 31 | GCA 186 | GCPK |
| GE 20372A | GE 20372B | GEM 122 | GEM 132 |
| GEM 144 | GEM 92 | GEM 93 | Glamolec |
| Glutathionarsenoxide | Glycovir | GMDP | GO 6976 |
| GO 7716 | GO 7775 | Gossypol | GPG-NH2 |
| GPI 1485 | GPI 2A | GPs 0193 | GR 137615 |
| GR 92938X | GS 2838 | GS 2992 | GS 3333 |
| GS 3435 | GS 4071 | GS 438 | GS 7340 |
| GS 9005 | GS 9160 | GS 930 | GW 275175 |
| GW 5950X | HB 19 | HBY 946 | HE 317 |
| Hepatitis B immune globulin | HEPT | HGS-H/A27 | HI 236 |
| HI 240 | HI 244 | HI 280 | HI 346 |
| HI 443 | HI 445 | HIV DNA vaccine (Antigen | Thiovir |
| | | Express, Inc.) | |
| HIV immune globulin | HIV immune plasma | HL 9 | HOE BAY 793 |
| HRG 214 | HS 058 | Hydroxycarbamide | Hydroxychloroquine |
| I 152 | IAZT | Idoxuridine | IM28 |
| ImmStat | ImmuDyn | Immunocal | Imreg 1 |
| Incadronic acid | INCB 9471 | Indinavir | Infliximab |
| Influenza matrix protein Zn2+ | Ingenol Triacetate | Inophyllum B | Inosine pranobex |
| finger peptide | | | |
| Interferon-tau | Interleukin-1 receptor type I | Interleukin-13 | Interleukin-15 |
| Interleukin-16 | Interleukin-2 agonist | Interleukin-4 | IPdR |
| Ipilimumab | ISIS 13312 | Iso ddA | ITI 002 |
| ITI 011 | JBP 485 | JCA 304 | JE 2147 |
| JM 1596 | JM 2763 | JTK 303 | K 12 |
| K 37 | K 42 | Kamizol | kethoxal |
| Kijimicin | Kistamicin | KKKI 538 | KM 043 |
| KNI 102 | KNI 241 | KNI 272 | KNI 413 |
| KNI 684 | Kootikuppala | KP 1461 | KPC 2 |
| KRH 1120 | L 689502 | L 693549 | L 696229 |
| L 696474 | L 696661 | L 697639 | L 697661 |
| L 708906 | L 731988 | L 732801 | L 734005 |
| L 735882 | L 738372 | L 738684 | L 738872 |
| L 739594 | L 748496 | L 754394 | L 756423 |
| L 870810 | L HSA ara AMP | Lamivudine/abacavir | Lamivudine/zidovudine |
| Lamivudine/zidovudine/abacavir | Lasinavir | LB 71116 | LB 71148 |
| LB 71262 | LB 71350 | LB 80380 | L-chicoric acid |
| Lecithinized superoxide | Leflunomide | Lentinan | Leukocyte interleukin injection |
| dismutase | | | (CEL-SCI Corp.) |
| Leukotriene B4-LTB4 | Levcycloserine | Levofloxacin | Lexithromycin |
| Liposomal ODG-PFA-OMe | Lithium succinate | Lobucavir | Lodenosine |
| Lopinavir | Loviride | Lufironil | LY 180299 |
| LY 214624 | LY 253963 | LY 289612 | LY 296242 |
| LY 296416 | LY 309391 | LY 309840 | LY 311912 |
| LY 314163 | LY 314177 | LY 316683 | LY 326188 |
| LY 326594 | LY 326620 | LY 338387 | LY 343814 |
| LY 354400 | LY 355455 | LY 366094 | LY 366405 |
| LY 368177 | LY 73497 | Lysozyme | M 40401 |
| M4N | Madu | Mannan sulfate | MAP 30 |
| Maraviroc | Maribavir | Masoprocol | MB-Foscarnet |
| MC 207044 | MC 207685 | MC 867 | mCDS71 |
| MDI-P | MDL 101028 | MDL 20610 | MDL 27393 |
| MDL 73669 | MDL 74428 | MDL 74695 | MDL 74968 |
| MDX 240 | ME 609 | MEDI 488 | MEN 10690 |
| MEN 10979 | MER N5075A | Met-enkephalin | Methisazone |
| MGN 3 | Michellamine B | Miglustat | MIV 150 |
| MIV 210 | Mivotilate | MK 0518 | MK 944A |
| MM 1 | MMS 1 | MOL 0275 | Monoclonal antibody 1F7 |
| Monoclonal antibody 2F5 | Monoclonal antibody 3F12 | Monoclonal antibody 447-52D | Monoclonal antibody 50-61A |
| Monoclonal antibody B4 | Monoclonal antibody HNK20 | Monoclonal antibody NM01 | Mopyridone |
| Moroxydine | Motavizumab | Motexafin gadolinium | Mozenavir |
| MPC 531 | MRK 1 | MS 1060 | MS 1126 |
| MS 8209 | MS 888 | MSC 127 | MSH 143 |
| MTCH 24 | MTP-PE | Murabutide | MV 026048 |
| MX 1313 | Mycophenolate mofetil | Navuridine | NB 001 |
| Neomycin B-arginine conjugate | Neotripterifordin | Nevirapine | Nitric oxide (e.g., ProStrakan) |
| Nitrodeazauridine | NM 01 | NM 49 | NM 55 |
| NNY-RANTES | Nonakine | NP 06 | NP 77A |
| NPC 15437 | NSC 158393 | NSC 20625 | NSC 287474 |
| NSC 4493 | NSC 615985 | NSC 620055 | NSC 624151 |
| NSC 624321 | NSC 627708 | NSC 651016 | NSC 667952 |
| NSC 708199 | NV 01 | Octoxynol 9 | OCX 0191 |
| OH 1 | OKU 40 | OKU 41 | Oltipraz |
| Omaciclovir | Opaviraline | OPT TL3 | Oragen |
| ORI 9020 | Oseltamivir | Oxetanocin | Oxothiazolidine carboxylate |
| PA 344/PA 344B | Palinavir | Palivizumab | PAMBAEEG |
| Papuamide A | PBS 119 | PC 1250 | PC 515 |
| PCL 016 | PD 0084430 | PD 144795 | PD 153103 |
| PD 157945 | PD 169277 | PD 171277 | PD 171791 |
| PD 173606 | PD 173638 | PD 177298 | PD 178390 |
| PD 178392 | PD 190497 | Pegaldesleukin | Peldesine |
| PEN 203 | Penciclovir | Pentosan polysulfate | Pentoxifylline |
| Peptide T | Peramivir | PETT 4 | PG 36 |
| Phellodendrine | Phosphatidyllamivudine | Phosphatidylzalcitabine | Phosphatidylzidovudine |
| Phosphazid | Phosphinic cyclocreatine | Pinosylvin | Pirodavir |
| PL 2500 | Pleconaril | Plerixafor | PM 104 |
| PM 19 | PM 523 | PM 92131 | PM 94116 |
| PMEDAP | PMS 601 | PMTG | PMTI |
| PN 355 | PNU 103657 | PNU 142721 | podophyllotoxin |
| Poly ICLC | Polyadenylic polyuridylic acid | Polysaccharide K | PP 29 |
| PPB 2 | PPL 100 | Pradefovir | Pradimicin A |
| Prasterone | PRO 140 | PRO 2000 | PRO 367 |
| PRO 542 | Probucol (Vyrex Corp.) | Propagermanium | Prostratin |
| Pseudohypericin | PSI 5004 | PTPR | PTX 111 |
| Pyriferone | Q 8045 | QM 96521 | QM 96639 |
| QR 435 | Quinobene | Quinoxapeptin A | Quinoxapeptin B |
| QYL 438 | QYL 609 | QYL 685 | QYL 769 |
| R 170591 | R 18893 | R 61837 | R 71762 |
| R 82150 | R 82913 | R 851 | R 87366 |
| R 91767 | R 944 | R 95288 | Raluridine |
| Ramatroban | Ranpirnase | RB 2121 | RBC CD4 |
| RD 30028 | RD 42024 | RD 42138 | RD 42217 |
| RD 42227 | RD 62198 | RD 65071 | RD6 Y664 |
| Regavirumab | Resobene | Respiratory syncytial virus | Retrogen |
| | | immune globulin | |
| REV 123 | RFI 641 | Rilpivirine | Rimantadine |
| RKS 1443 | RO 0334649 | RO 247429 | RO 250236 |
| RO 316840 | RO 53335 | Robustaflavone | Rolipram |
| RP 70034 | RP 71955 | RPI 312 | RPI 856 |
| RPR 103611 | RPR 106868 | RPR 111423 | RS 654 |
| RS 980 | RSV 604 | Rubitecan | Rupintrivir |
| S 1360 | S 2720 | S 9a | SA 1042 |
| SA 8443 | SB 180922 | SB 205700 | SB 206343 |
| SB 73 | SC 49483 | SC 55099 | SCH 350634 |
| SD 894 | S-DABO | SDF 1 | SDZ 282870 |
| SDZ 283053 | SDZ 283471 | SDZ 89104 | SDZ PRI 053 |
| SE 063 | Semapimod | Sevirumab | SF 950 |
| SF 953 | Siamycin 1 | Siamycin 2 | sICAM-1 |
| Sifuvirtide | SIGA 246 | Sizofiran | SJ 3366 |
| SK 034 | SKF 108922 | SKI 1695 | SO 324 |
| Sodium laurilsulfate | Solutein | Sorivudine (e.g., topical) | SP 10 |
| SP 1093V | Sparfosic acid | SPC 3 | SPD 756 |
| SpecifEx-Hep B | SPI 119 | SPL 2992 | SPL 7013 |
| SPV 30 | SR 10204 | SR 10208 | SR 11335 |
| SR 3745A | SR 3773 | SR 3775 | SR 3784 |
| SR 3785 | SR 41476 | SRL 172 | SRR SB3 |
| ST 135647 | Stachyflin | stallimycin | Stampidine |
| Statolon | Stavudine | Stepronin | Suksdorfin |
| Sulfated maltoheptaose | Superoxide dismutase | Suramin (e.g., sodium) | Sy 801 |
| T 1100 | T 118 | T 22 | T 30695 |
| T 611 | T 705 | T4GEN | Tacrine |
| TAK 220 | TAK 652 | TAK 779 | Talviraline |
| TAP 29 | TASP | Teceleukin | Tecogalan (e.g., sodium) |
| TEI 2306 | Telbivudine | Telinavir | Temacrazine |
| Tenidap | Tenofovir | Tenofovir disoproxil fumarate | TGG II 23A |
| TH 9407 | TH 9411 | Thalidomide | Thiophosphonoformic acid |
| Thymoctonan | Thymosin fraction 5 | Thymotrinan | tICAM-1 |
| Tifuvirtide | Tilarginine | Tipranavir | Tiviciclovir |
| Tivirapine | TJ 41 | TL 3024 | TMC 126 |
| TNF-alpha inhibitor | TNK 6123 | TNX 355 | Todoxin |
| Tomeglovir | Transforming growth factor- | TraT | Trecovirsen |
| | alpha | | |
| Tremacamra | Trichosanthin | Triconal | Trimidox |
| Trodusquemine | Tromantadine | Trovirdine | Tuvirumab |
| U 103017 | U 75875 | U 78036 | U 80493 |
| U 81749 | U 88204E | U 96988 | U 9843 |
| UA 926 | Ubenimex | UC 10 | UC 16 |
| UC 38 | UC 42 | UC 68 | UC 70 |
| UC 781 | UC 81 | UC 82 | UIC 94003 |
| Ukrain | UL36ANTI | UMJD 828 | Valaciclovir |
| Valganciclovir | Valomaciclovir | Valtorcitabine | Varicella zoster immune |
| | | | globulin |
| VB 19038 | Vesnarinone | VF 1634 | VGV 1 |
| Vicriviroc | VIR 101 | Viraprexin | Virodene |
| Viscum album extract | VRX 496 | VX 10166 | VX 10217 |
| VX 10493 | VX 11106 | WHI 05 | WHI 07 |
| WIN 49569 | WIN 49611 | WM 5 | WR 151327 |
| XK 216 | XK 234 | XN 482 | XP 951 |
| XQ 9302 | XR 835 | XU 348 | XU 430 |
| Y-ART-3 | YHI 1 | YK FH312 | Z 100 |
| Z 15 | Zalcitabine | Zanamivir | Zidovudine (e.g., phosphate- |
| | | | didanosine dimer) |
| Zidovudine triphosphate mimics | ZX 0610 | ZX 0620 | ZX 0791 |
| ZX 0792 | ZX 0793 | ZX 0851 | ZY II |

Additional hepatitis C therapies are described in Table 5.

TABLE 5

| | | | |
|---|---|---|---|
| Albuferon | | JTK 003 | R7128 |
| 2′-C-methyl-7-deaza-adenosine | HCV AB 68 | JTK 109 | Resiquimod |
| A-837093 | HCV-SM | KPE 00001113 | Rosiglitazone |
| AG-021541 | HE 2000 | KPE 02003002 | Sargramostim |
| Aldesleukin | Hepatitis C immune globulin | Lactoferrin | |
| ANA 971 | Hepex C | Lamivudine | SCH 6 |
| ANA 975 | Heptazyme | LB 84451 | Schisandra |
| AVI 4065 | Histamine | Licorice root | SCV 07 |
| AVR 118 | Histamine dihydrochloride | ME 3738 | SCY-635 |
| | (e.g., injection, oral) | | |
| Bavituximab | HuMax-HepC | Medusa Interferon | Silipide |
| BILN 303 SE | Hypericin | | Taribavirin |
| BIVN 401 | ICN 17261 | Milk thistle | |
| BLX 833 (e.g., controlled | IDN 6556 | Mitoquinone | Thymalfasin (e.g., Zadaxin) |
| release) | | | |
| | Imiquimod | NIM 811 | Thymus extract |
| CellCept | Interferon | N-nonyl-DNJ | TJ 9 |
| Ceplene | Interferon alfa-2b (e.g., | NOV 205 | Tucaresol |
| | inhalation) | | |
| Ciluprevir (BILN 2061) | Interferon alfacon-1 | NV-08 | Ursodeoxycholic acid |
| Civacir | Interferon alpha (e.g., | P 56 | UT 231B |
| | sustained release, intranasal, | | |
| | Omniferon) | | |
| Colloidal silver | | Peginterferon alfa-2a | Valopicitabine (NM 283) |
| CpG 10101 | Interferon alpha-2b (e.g., | Peginterferon alfa-2b | VGX 410 |
| | controlled release or | | |
| | transdermal) | | |
| DEBIO-025 | Interferon alpha-2b gene | PEGinterferon alfacon-1 | Virostat |
| | therapy | | |
| Edodekin alfa | Interferon alpha-n3 | PEGylated interferon | VP 50406 |
| EHC 18 | Interferon beta-1a | Pegylated thymalfasin | VRT 21493 |
| EMZ 702 | Interferon beta-1b | PF-03491390 | |
| Fas-ligand inhibitor | Interferon gamma-1b | PG 301029 | WF 10 |
| Ginseng | Interferon omega | PSI-6130 | XTL 2125 |
| Glycyrrhizin | Interleukin 10 (e.g., human | R 1518 | XTL 6865 |
| | recombinant) | | |
| GS 9132 | Isatoribine | R 1626 | |
| HCV 086 | ISIS 14803 | R 803 | |
| HCV 371 | ITMN-191 | R-1626 | |

TABLE 6

Interferon alpha-2b/ribavirin
Lopinavir/ritonavir
Peginterferon alfa-2b/ribavirin

TABLE 7

Peginterferon-alpha/ribavirin/EMZ 702
Efavirenz/emtricitabine/tenofovir disoproxil fumarate

Analogs of any of the compounds listed in Tables 1, 2, or 3 may be used in any of the compositions, methods, and kits of the invention. Such analogs include any agent from the same therapeutic class, having the same or related molecular targets, or from the same mechanistic class as those listed in Table 8. Exemplary analogs of these compounds are described throughout the specification.

TABLE 8

| Name | Therapeutic Classification | Molecular Target | Mechanism of Action | Misc Classification/Information |
|---|---|---|---|---|
| Mecobalamin | Vitamin (e.g., B12 analog) | Homocysteine | Coenzyme of methionine synthetase in the | Vitamin (hematopoietic) |
| | | Methionine synthetase | synthesis of methionine from | Vitamin B12 analog |
| | | | homocysteine; role in transmethylation | |
| Cobamamide | Vitamin | Methionine synthetase | Cofactor of Methionine synthetase | Vitamin B12 analog |
| | Liver extracts and combinations with | | | Coenzymic form of vitamin B12 |
| | B12 | | | |
| | Ophthalmological | | | |
| | Alimentary tract product | | | |
| | Systemic anabolics | | | |
| Curcumin | Alimentary tract product | | Transcription, activation | Antioxidant |
| | Anorectics | | Immunosuppressant | NSAID |
| | Antacids/antiflatulants carminative | | Platelet aggregation antagonist | Enzyme inhibitor |
| | Anti-atheroma preparation of natural | | Thromboxane synthase inhibitor | Dye |
| | origin | | NFκB inhibitor | |
| | Antidiarrheal | | Anti-inflammatory activity | |
| | Antiemetic | | Possible antineoplastic activity; | |
| | Antifungal | | antiproliferative effects; | |
| | Antiviral | | Induction of cell death in colon and | |
| | Antineoplastic | | melanoma tumor cells | |
| | Antihemorrhoidal | | Induces apoptosis independently of p53 | |
| | Antimigraine preparation | | status | |
| | Antirheumatic, non-steroidal | | | |
| | (NSAID) | | | |
| | Antiseptic and disinfectant | | | |
| | Appetite stimulant | | | |
| | Bile therapy and cholagogues | | | |
| | Cytostatic | | | |
| | Dermatological | | | |
| | Digestives | | | |
| | Hepatic Protector, Lipotropics | | | |
| | Laxative | | | |
| | Musculoskeletal product | | | |
| | Prostatic disease product | | | |
| | Stomach disorder prep | | | |
| | Topical vasoprotective | | | |
| | Wound healing agent | | | |
| Stanozolol | Systemic anabolic | | Anabolic | Commonly used as an ergogenic aid; |
| | Hematological agent | | Androgenic | banned substance in sports |
| | Anabolic steroid | | FSH antagonist | competition by International |
| | | | Protein catabolism inhibitor | Association of Athletics Federations |
| | | | ICSH antagonist | (IAAF). |
| | | | Testosterone release inhibitor | Used in treatment of hereditary |
| | | | | angioedema |
| Vitamin B12 | Cardiovascular product | Methionine synthase | Succinyl-CoA production | Hematinic |
| | Cerebral and peripheral | | Activates folate coenzymes | Vitamin (hematopoietic) |
| | vasotherapeutic | | Synthetic Adrenergic | Hematopoietic activity appears |
| | Anti-atheroma preparations of | | Participates in DNA-synthesis | identical to antianemia-factor in |
| | natural origin | | Participates in protein-synthesis | purified liver extract |
| | Cholesterol and triglyceride | | Hematopoiesis | |
| | reduction preparation | | Cell reproduction | |
| | Anti-anemic product | | Essential for growth | |
| | Non-narcotic analgesic | | Nucleoprotein synthesis | |
| | Anti-inflammatory enzyme | | Physiological role associated with | |
| | Musculoskeletal product | | Methylation | |
| | Systemic muscle relaxant | | Myelin synthesis | |
| | Antirheumatic | | | |
| | Systemic antihistamine | | | |
| | Neurotonic | | | |
| | Antidepressant | | | |
| | Stomatological | | | |
| | Blood coagulation | | | |
| | Antifibrinolytic | | | |
| | Digestive | | | |
| | Antidiarrheal micro-organisms | | | |
| | Appetite stimulant | | | |
| | Anorectic | | | |
| | Vitamin | | | |
| Vinorelbine | Cytostatics | Tubulin | Cytoskeleton | Vinca alkaloid |
| | Antineoplastic | | Tubulin destabilizer | Antineoplastic agent, phytogenic |
| | | | Mitotic inhibitor | Radiation-sensitizing agent |
| Sirolimus | Immunosuppressive agent | mTOR | mTOR inhibitor | May inhibit human T- and B- |
| (rapamycin) | Antifungal | Immunophilins | Blocks cytokine transcription | lymphocyte proliferation |
| | Antineoplastic | | | |
| Disulfiram | Alcohol deterrent | aldehyde dehydrogenase | Aldehyde dehydrogenase inhibitor | Acaricide |
| | Drugs used In alcohol dependence | | Metabolism, energy | Fungicide, bactericide, wood |
| | | | | preservative |
| | | | | Immunomodulator |
| | | | | Enzyme inhibitors |
| Hydroxocobalamin | Vitamin (e.g., B12) | | | Hematinics |
| | Anti-anemic product, including folic | | | Vitamin (hematopoietic) |
| | acid | | | Vitamin B12 analog |
| | Ophthalmological | | | |
| | Neurotonic | | | |
| | Non-narcotic analgesic | | | |
| | Musculoskeletal product | | | |
| | Antirheumatic | | | |
| Testosterone | Hormone | FSH | FSH antagonist | Androgen |
| | | ICSH | High dose: spermatogenesis-inhibitor | Hormone |
| | | | Gonadotropin antagonist | Activity in many tissues may depend |
| | | | ICSH antagonist | on reduction to dihydrotestosterone |
| | | | | which binds to cytosolic-receptor- |
| | | | | proteins |
| | | | | Exogenous administration inhibits |
| | | | | endogenous release via feedback |
| | | | | inhibition of pituitary ICSH |
| Paclitaxel | Cytostatic | Tubulin | Microtubule Inhibitor | Antineoplastic agents, phytogenic |
| | Antineoplastic | Microtubules | Tubulin stabilizer | Vinca Alkaloid |
| | | | Radiation sensitization | |
| Fludarabine | Antineoplastic | DNA polymerase alpha | Inhibition of DNA polymerase alpha by 2- | Nucleoside analog |
| | Cytostatic | | fluora-ara-ATP (metabolite of fludarabine) | |
| | Antimetabolite | | | |
| | Immunosuppressant | | | |
| Cycloheximide | | Ribosomal peptidyl transferase | Prostaglandin synthesis stimulant | |
| | | 23S rRNA | Ribosomal peptidyl transferase inhibitor | |
| | | | Translation, ribosome | |
| Wedelolactone | | IkB-α kinase | IKKα and IKKβ Kinase inhibitor | |
| | | IKKα Kinase | IkB-α kinase inhibitor | |
| | | IKKβ Kinase | | |
| Vidarabine | Antivirals (e.g., topical) | DNA polymerase | DNA polymerase inhibitor | Antimetabolite |
| | Ophthalmological (e.g., antiviral | | DNA synthesis inhibitor | Principal metabolite is hypoxanthine |
| | agent) | | DNA synthesis | arabinoside possesses virucidal |
| | Antineoplastic | | | activity |
| | | | | may interfere with early steps of viral |
| | | | | DNA synthesis |
| Wortmannin | Anti-inflammatory agents, steroidal | PI3K | Phosphodiesterase inhibitor | |
| | Immunosuppressive | phospholipase-d | Phosphatidylinositol 3-kinase inhibitor. | |
| | Antibiotic | phospholipase-c | Insulin antagonist | |
| | Antifungal | | Phospholipase d inhibitor | |
| | | | Phospholipase c inhibitor | |
| | | | Serotonin antagonist | |
| Aphidicolin | Antiviral | DNA polymerase | DNA polymerase inhibitor | May be of clinical use as an |
| | Antiherpetic | DNA polymerase II | DNA synthesis inhibitor | antiherpetic agent in AIDS patients |
| | Antiproliferative | Viral-induced DNA polymerase | | resistant to aciclovir. |
| | | DNA polymerase α | | |
| FR122047 | NSAID | COX-1 | Selective COX1 inhibitor | |
| | | | Metabolism, hormone, prostaglandin | |
| Fluorouracil | Cytostatic | Thymidylate synthase | DNA synthesis inhibitor | |
| | Antimetabolite | | Pyrimidine antagonist | |
| | Antineoplastic | | DNA metabolism, pyrimidine | |
| | Immunosuppressive | | Apparent deoxyuridylate methylation | |
| | | | inhibitor | |
| | | | Partial RNA synthesis inhibitor | |
| Evans Blue | | | | Dye |
| SB-202190 | | p38 MAPK | Eosinophil antagonist | Apoptosis inducer |
| | | p38α and β isoforms | MAP kinase inhibitor (e.g., p38) | |
| | | | TGF-beta stimulator | |
| JSH-23 | | | blocks nuclear translocation of NK-kB | |
| | | | NFκB translocation inhibitor | |
| | | | Transcription, activation | |
| N-Tosyl-L- | | NFκB | NFκB inhibitor | |
| phenylalanine | | | serine protease inhibitor | |
| chloromethyl ketone | | | | |
| GW 5074 | | cRAF1 | MAPK, cRAF1 inhibitor | |
| | | | Raf-1 kinase inhibitor | |
| ML 9 | | MAP kinase | MAP kinase inhibitor | Enzyme inhibitors |
| | | | Myosin light chain kinase inhibitor | Azepine |
| | | | Catecholamine secretion inhibitor | |
| | | | Protein kinase C (PKC) inhibitor | |
| | | | Protein cAMP-dependent protein kinase | |
| | | | (PKA) inhibitor | |
| Bay 11-7082 | Apoptosis promoter | IkB-alpha kinase | I-kappa B-alpha kinase inhibitor. | |
| | | | Kinase inhibitor | |
| | | | Inhibits NFκB | |
| PKR inhibitor | | RNA-dependent protein kinase | RNA-dependent protein kinase inhibitor | |
| Vitamin K5 | Antifungal | Coagulation factor II, VII, IX, and X | Required for conversion of prothrombin to | Insulin mimicking effect |
| | Coagulation factor | Protein C | thrombin | Antitumor activity |
| | | Protein S | Plays a role in coagulation factors II, VII, | |
| | | Protein Z | IX, and X, and Protein C, Protein S, and | |
| | | | Protein Z | |
| Saquinavir mesylate | Antiviral | HIV-1 Protease | HIV-1 and HIV-2 protease inhibitor | |
| | | HIV-2 Protease | Protein processing | |
| Nelfinavir mesylate | Antiviral | Proteases | HIV protease inhibitor | |
| | | | Peptide hydrolase inhibitor | |
| | | | Protein processing | |
| Fenbendazole | Anthelmintic | Tubulin | Binds to tubulin and prevents microtubule | |
| | Antinematodal | | formation | |
| Ritonavir | Antiviral | Proteases | HIV protease inhibitor | |
| | | | Protein processing | |
| Dextrothyroxine | Hypolipemics | | Thyroid hormone | |
| sodium | | | Stimulates hepatic-cholesterol catabolism | |
| | | | Reduces serum-cholesterol (e.g., LDL) | |
| | | | May reduce elevated lipoprotein-beta and | |
| | | | triglyceride fractions | |
| | | | Stimulates biliary excretion of cholesterol | |
| | | | and its degradation products | |
| Levothyroxine | Thyroid therapy | | Increases metabolic rate | Thyroid-hormone |
| Sodium | Muscle relaxant | | Protein, carbohydrate, and lipid | |
| | Stimulant | | metabolism stimulant | |
| Reserpine | Antihypertensive | | Adrenergic uptake inhibitor | Sympatholytics |
| | Beta blocker | | Dopamine antagonist | |
| | Antipsychotic | | | |
| Desloratadine | Antihistamine (e.g., systemic) | Histamine H1 | Histamine receptor antagonist (e.g., H1) | Anti-allergic agent |
| | Antioxidant | | Calcium antagonist | |
| | | | Eosinophil antagonist | |
| Tamoxifen citrate | Antiesterogen | Estrogen receptor | PKC inhibitor | Competes with estradiol and estrogen |
| | Antineoplastic | PKC | Estrogen receptor inhibitor, modulator | for receptor protein |
| | | | Estrogen agonist (e.g., in bone) | Selective estrogen receptor modulator |
| | | | Estrogen antagonist | |
| | | | Receptor, hormone | |
| Raloxifene | Antineoplastic | Estrogen receptor | Estrogen receptor modulator | Selective estrogen receptor modulator |
| hydrochloride | Anti-esterogenic | | Estrogen agonist (e.g., in bone) | |
| | | | Estrogen antagonist | |
| | | | Receptor, hormone | |
| Repaglinide | Antidiabetic | | Stimulates Insulin release | Hypoglycemic agent |
| Loratadine | Antihistamine (e.g., systemic) | | Histamine receptor antagonist (e.g., H1) | Antipruritic |
| Fluvoxamine | SSRI | Serotonin 5-HT transporter | Serotonin uptake inhibitor | Antiobsessional agent |
| maleate | | | Receptor, neural | little effect on noradrenaline uptake |
| Adefovir dipivoxil | Antiviral (e.g. HIV) | Reverse transcriptase | Reverse transcriptase inhibitor | |
| | | | Viral replication | |
| Efavirenz | Antiviral (e.g., HIV) | Reverse transcriptase | Reverse transcriptase inhibitor | Benzoxazinone |
| | | | Viral replication | Non-nucleoside reverse transcriptase |
| | | | | Inhibitor |
| Doxepin | Sedative | Norepinephrine transporter | Histamine receptor antagonist (H1, H2) | Tricyclic |
| hydrochloride | Antihistamine | Serotonin transporter | Inhibits noradrenaline and serotonin | Mild peripheral vasodilator |
| | | | reuptake at presynaptic neuron | Parasympatholytic |
| | | | Amine pump blocker | Antidepressant |
| | | | Adrenergic innervation | |
| Maprotiline | Sedative | Norepinephrine transporter | Alpha2-adrenergic receptor antagonist | Tetracylcic |
| hydrochloride | Antihistamine | | Amine pump blocker | Parasympatholytic |
| | Antidepressant | | Presynaptic serotonin and noradrenaline | Related structurally and functionally to |
| | | | uptake inhibitor | tricyclic antidepressants |
| | | | Mild peripheral vasodilator | |
| Ezetimibe | Antihyperlipoproteinemic | | Lipid transport inhibitor | |
| | | | Cholesterol absorption inhibitors | |
| Albendazole sulfone | Antiparasitic | Lanosterol 14-α-demethylase | Metabolism, sterol | |
| | Anthelmintic | Microtubules | Lanosterol 14-α-demethylase inhibitor | |
| | Non-steroidal respiratory | | Reported ATP-synthesis-inhibitor | |
| | antiinflammatory | | Reported to interact with microtubules | |
| | Amoebicide | | Activity against Giardia lamblia | |
| | Antiprotozoal | | | |
| | Anticestodal | | | |
| Hydroxyzine | Antihistamine | Histamine H1 | Possible subcortical CNS-depressant | Primary skeletal-muscle relaxant |
| (hydrochloride or | Antiemetic | | Mild gastric secretion inhibitor | Spasmolytic activity |
| pamoate) | | | Histamine (H1) blocker | Tranquilizer (minor) |
| Bromocriptine | Estrogens | Dopamine D2 receptor | Dopaminergic; dopamine agonist | Enzyme inhibitor (prolactin) |
| mesylate | Other sex hormones | Prolactin | Suppresses prolactin secretion | Ergot alkyloid |
| | Antiparkinson | | Stimulates dopamine receptors | Ergotamine |
| | | | Prolactin antagonist | |
| | | | Dopamine D2 receptor agonist | |
| Trifluoperazine | Antipsychotic | Calmodulin inhibitor | Calmodulin antagonist | Parasympatholytic |
| Hydrochloride | | Eg5 inhibitor | Sympatholytic-alpha | Phenothiazine |
| | | | Dopamine antagonist, release inhibitor | Increases neuronal firing-rate in the |
| | | | May depress reticular activating system | midbrain |
| | | | Dopamine turnover stimulant | Sedative hypnotic |
| Benzydamine | Analgesic | | Blocks action of cyclo-oxygenase | Analgesic |
| hydrochloride | Anti-inflammatory | | | Antipyretic |
| | NSAID | | | |
| Mebeverine | Digestive | | | Relaxant [smooth muscle] |
| | Antispasmodic and anticholinergic | | | Reported to be a direct-acting smooth |
| | Antispasmodic, Ataractic | | | muscle relaxant |
| | combinations | | | |
| Chlorophyllin | Stomatological | | May have antimutagenic and | Chlorophyll |
| | | | anticarcinogenic properties | |
| Mosapride citrate | Gastroprokinetic | 5-HT4 receptor antagonist | Serotonin 4 receptor agonist | |
| | Gastointestinal agent | | Enhances gastric emptying and colonic | |
| | | | motor activity | |
| | | | Dopamine antagonists | |
| Flupentixol | Neuroleptic | | Dopamine receptor antagonist | Parasympatholytic |
| | Antipsychotic | | Prolactin release stimulant | |
| | | | Dopamine turnover stimulant | |
| | | | Ganglionplegic | |
| | | | Heat regulating center inhibitor | |
| | | | Membrane stabilizer | |
| | | | Benzodiazepine agonist | |
| | | | Sympatholytic-alpha | |
| | | | Dopamine antagonist (e.g., D2) | |
| Rescinnamine | Antihypertensive | | Probable mechanism: peripheral | Related structurally to reserpine and |
| | | | adrenaline-depletor | yohimbine |
| | | | peripheral noradrenaline-depletor | |
| | | | angiotensin-converting enzyme inhibitor | |
| Dydrogesterone | Hormonal contraceptive | | Progestogen | Hormone |
| | Estrogen, progestogen combination | | Tocolytic | Progestational hormones, synthetic |
| | Progestogen | | | Progestin |
| Rifabutin | Antibiotic | | RNA polymerase inhibitor | |
| | Antitubercular, tuberculostatic | | Interferes with bacterial DNA-synthesis | |
| | Rifampicin/Rifamycin | | | |
| P-Aminosalicylic acid | Antitubercular | | Inhibits bacterial resistance to | Active only against mycobacteria (e.g., |
| (e.g., sodium salt) | Bacteriostatic | | streptomycin and isoniazid. | Mycobacterium tuberculosis). |
| | Antibiotic | | May inhibit folic acid synthesis without | |
| | | | potentiation with antifolic compounds | |
| | | | May inhibit synthesis of mycobactin, thus | |
| | | | reducing iron uptake by M. tuberculosis. | |
| Sertraline | SSRI | | Inhibition of seratonin re-uptake | Antidepressant |
| hydrochloride | | | | |
| Benztropine | Antihistamine | | Muscarinic antagonist | Parasympatholytic |
| mesylate | Antiparkinsonian | | Dopamine uptake inhibitor | Synthetic compound containing |
| | | | Anticholinergic | structural features of atropine and |
| | | | | diphenhydramine. |
| Fluphenazine | Antipsychotic | Dopamine (D1, D2) receptor | Dopamine receptor antagonist | Parasympatholytic |
| hydrochloride | | | (postsynaptic) | Similar to chlorpromazine |
| | | | Dopamine release inhibitor | |
| | | | Sympatholytic alpha | |
| | | | Dopamine antagonist | |
| | | | Dopamine turnover stimulant | |
| | | | Calmodulin antagonist | |
| Andrographis | Hepatic protectors, lipotropics | | Arrest of cell growth caused by viruses | Contains analgesic, antithrombotic, |
| | Antineoplastic | | Anticancer activity | thrombolytic, hypoglycemic, and |
| | Antiviral (e.g., HIV) | | | antipyretic compounds. |
| | Antipyretic | | | Andrographolide is major labdane |
| | | | | diterpenoidal constituent of |
| | | | | Andrographis paniculata |
| Perospirone | Antipsychotic | | | |
| Meclizine | Antiemetic, antinauseant | | Histamine (H1) agonist | Benzhydryl compounds |
| | Antihistamine | | | Piperazines |
| Bufexamac | Antihemorrhoidal | | Prostaglandin antagonist | Benzeneacetamides |
| | Antipruritic | | | Analgesic |
| | Anti-inflammatory (e.g., non- | | | antipyretic |
| | steriodal) | | | Anti-inflammatory agents, topical |
| | Antirheumatic (e.g., topical, non- | | | |
| | steroidal) | | | |
| | Antipsoriasis | | | |
| | Antifungal | | | |
| Mesterolone | Steroid | | Anabolic | |
| | Androgen | | Androgen | |
| Trifluperidol | Antipsychotic | | Benzodiazepine agonist | Parasympatholytic |
| | | | Dopamine antagonist | Butyrophenone |
| | | | Ganglionplegic | Similar properties to haloperidol |
| | | | Membrane stabilizer | |
| | | | Dopamine turnover stimulant | |
| | | | Sympatholytic-alpha | |
| | | | Heat regulating center inhibitor | |
| | | | Prolactin release stimulant | |
| | | | Dopamine-2 antagonist | |
| Clomiphene citrate | Estrogen agonist | | Metabolism, sterol | Gonad-stimulating principle |
| | Estrogen antagonist | | Ovary stimulant | Hormone |
| | | | Squalene epoxidase inhibitor | |
| Trimipramine | Antidepressant | Serotonin 5-HT transporter | Presynaptic serotonin reuptake inhibitor | Parasympatholytic |
| Maleate | SSRI | | Presynaptic noradrenaline reuptake | Dibenzazepines |
| | Sedative | | inhibitor | Tricyclic |
| | Antihistamine | | Amine pump blocker | |
| | | | Mild peripheral vasodilator | |
| Fenretinide | Retinoic acid receptor agonist | PPAR agonist | PPAR agonist | Retinoid |
| | Antineoplastic | | Transcription, activation | Inhibits the growth of prostate cancer |
| | Retention of cyctotoxicity under | | | in rats |
| | hypoxia. | | | Decreases plasma retinol and retinol- |
| | | | | binding protein levels in breast cancer |
| | | | | patients |
| | | | | Increases levels of ceramide. |
| Budesonide | Antiinflammatory (e.g., intestinal, | GC receptor | GC receptor activator | Glucocorticoids, topical |
| | steroidal) | | Transcription, activation | Hormone |
| | Corticosteroid (e.g., topical, | | | |
| | systemic) | | | |
| | Antiasthmatic (e.g., B2-stimulant, | | | |
| | corticoid, xanthines) | | | |
| | Bronchodilator | | | |
| Toremifene citrate | Cytostatic | Estrogen receptor | Estrogen antagonist | Hormone |
| | Antineoplastic | | Estrogen agonist | Anti-estrogen |
| | | | Estrogen receptor inhibitor | |
| Cladribine | Antimetabolite | DNA polymerase | Arrests cell division | May disrupt later stages of cell division |
| | Cytostatic | Adenosine receptor | Incorporates into DNA | Activity against low-grade lymphocytic |
| | Antineoplastic | DNA | DNA polymerase inhibitor | malignancies; Inhibits T and B cell |
| | Immunosuppressant | | Adenosine receptor agonist | proliferation |
| | | | Immunosuppressive activity possibly | Prolongs the survival of skin and small |
| | | | mediated by triggering apoptosis in | bowel allografts in animals; |
| | | | monocytes and lymphocytes | Reduces hypodense lesions in patients |
| | | | Cytotoxic in lymphoid and myeloid | with multiple sclerosis. |
| | | | neoplasms | |
| Cytarabine | Antimetabolite | DNA polymerase | Blocks progression from G₁-phase to S- | |
| | Antineoplastic | DNA polymerase-α | phase | |
| | Antiviral | DNA | Virucidal activity | |
| | Cytostatic | | Primarily active in S-phase | |
| | Immunosuppressive agent | | DNA polymerase inhibitor | |
| | | | Damages DNA/chromosomes | |
| | | | Incorporated into DNA and RNA | |
| Melphalan | Antineoplastic | DNA | Bifunctional alkylating-agent | |
| | Cytostatic | | reported DNA-crosslinker | |
| | Alkylating agent | | DNA alkylator | |
| | Immunosuppressant | | DNA damage | |
| Mechlorethamine | Alkylating agent | DNA | DNA damage | Destructive to mucous membranes |
| hydrochloride | Antineoplastic | | DNA alkylator | |
| Trequinsin | | Phosphodiesterase | Phosphodiesterase inhibitor | Platelet aggregation inhibitor |
| hydrochloride | | | | |
| Auranofin | Antirheumatic | | | |
| Ergoloid mesylates | Antihypertensive (e.g., herbal) | | Decreases vascular tone and slows the | Mixture of the mesylates (methane |
| | Peripheral vasodilator | | heart rate | sulfonates) of dihydroergocornine, |
| | | | Blocks alpha-receptors. | dihydroergocristine, and the α- and β- |
| | | | May increase oxygen uptake and cerebral | isomers of dihydroergocryptine. |
| | | | metabolism, thereby normalizing | Used to treat decreasing mental |
| | | | depressed neurotransmitter levels. | capacity with age |
| Bismuth | Antibacterial | | Inhibits growth of Helicobacter pylori in | Fungicide, bactericide, wood |
| subsalicylate | Antidiarrheal | | peptic ulcer | preservative |
| | | | Influences capsular polysaccharide | |
| | | | production | |
| | | | Possible prostaglandin synthesis inducer | |
| | | | Possible enhancer of aminoglycoside | |
| | | | production | |
| Bromhexine | Antiasthmatic, B2 stimulant | Mucus glands | Acts on mucus formation | Mucolytic |
| | Cough sedative | Acid mucopolysaccharide fibers | Disrupts structure of acid | Expectorant |
| | Expectorant | | mucopolysaccharide fibers | |
| | | | Produces less viscous mucus | |
| Phenazopyridine | Anesthetic | | Mechanism of action unknown | Exerts a topical analgesic effect on the |
| hydrochloride | Analgesic | | Produces prompt and effective local | urinary-tract mucosa during excretion |
| | | | analgesia and relief of urinary symptoms | |
| | | | by its rapid excretion in the urinary tract. | |
| | | | Effects are confined to the genitourinary | |
| | | | system and are not accompanied by | |
| | | | generalized sedation or narcosis. | |
| Diethylstilbestrol | Estrogens (nonsteroidal) | | inhibits luteinizing hormone secretion by | Hormone |
| | Antineoplastic | | the pituitary, thereby inhibiting testosterone | Contraceptives, postcoital, synthetic |
| | | | secretion. | |
| Dicyclomine | Antispasmodic | | Gastric secretion inhibitor | Anticholinergic |
| hydrochloride | Anesthetic | | | Parasympatholytic |
| Indocyanine Green | Ophthalmological diagnostic agent | | | Diagnostic aid (cardiac output and |
| | Imaging agent | | | hepatic function) |
| | Diagnostic | | | Dyes |
| Dibucaine | Anesthetic (e.g., local) | | Calcium antagonist | primary site of action may be sodium |
| hydrochloride | | | Nerve sodium permeability inhibitor | transport proteins |
| | | | Sensory nerve impulse inhibitor | |
| | | | Calmodulin antagonist | |
| Vanillin acetate | | | | Scent |
| Flubendazole | Anthelmintic | | | Antiprotozoal |
| | Antinematodal | | | |
| Oxfendazole | Anthelmintic | | | Antinematodal agents |
| Griseofulvin, | Antirheumatic nonsteroidal | Phosphodiesterase | Phosphodiesterase inhibitor | Fungicide, bactericide, wood |
| microcrystalline | Antifungal | Tubulin | Tubulin inhibitor | preservative |
| Citalopram | SSRI | Serotonin 5-HT transporter | Serotonin-reuptake-inhibitor | |
| hydrobromide | Antidepressant | | Serotonin 5-HT transporter | |
| Imipramine | Antihistamine | Serotonin 5-HT transporter | Serotoninergic | |
| hydrochloride | Sedative | | Mild peripheral vasodilator | |
| | Tricyclic antidepressant | | Presynaptic serotonin-reuptake-inhibitor | |
| | Antidepressant | | Amine pump blocker | |
| | | | Presynaptic noradrenaline reuptake | |
| | | | inhibitor | |
| Azelastine | Antihistamine | Histamine H1 | Platelet aggregation inhibitor | May interfere with calcium-dependent |
| | Preparations for non-specific | | Histamine Receptor Antagonist (H1) | translocation |
| | conjuctivitis | | May interfere with leukotriene-B4 synthesis | |
| | Non-Steroidal respiratory | | and release | |
| | antiinflammatory | | May interfere with HETE-5-synthesis and | |
| | Rhinologicals (topical, systemic) | | release | |
| | Bronchodilators and antiasthmatics | | Interferes with activation/mobilization of | |
| | NSAID | | Lipoxygenase-5 | |
| | | | Lipoxygenase inhibitor | |
| | | | May stabilize pulmonary epithelium | |
| | | | May interfere with leukotriene-C4- | |
| | | | synthesis/release | |
| | | | May inhibit leukocyte migration | |
| | | | Mast cell stabilizer | |
| Cyproheptadine | Antihistamine | Histamine H1 | Histamine receptor antagonist (H1) | |
| hydrochloride | | | Serotonin antagonist | |
| Mometasone furoate | Corticosteroid (topical) | GC receptor | ACTH secretion inhibitor | |
| | Topical rhinological | Progesterone receptor | Causes protein catabolism | |
| | Antiasthmatic, corticoid | | Glycogen deposition inhibitor | |
| | Steriodal anti-inflammatory | | Calcium mobilizer | |
| | Glucocorticoids, topical | | GC receptor activator | |
| | Anti-allergic | | Transcription activator | |
| | | | Immunomodulator | |
| | | | Gluconeogenesis promoter | |
| | | | Phosphorus mobilizer | |
| | | | Inhibits production of reactive protein by | |
| | | | inflammatory cells | |
| | | | Inhibits migration of inflammatory cells | |
| Fulvestrant | Cytostatic hormone antagonist | Estrogen receptor | Estrogen antagonist | |
| | Antineoplastic | | Estrogen receptor inhibitor | |
| Topotecan | Antineoplastic | DNA topoisomerase I | DNA topoisomerase I inhibitor | |
| hydrochloride | | | DNA damage | |
| Irinotecan | Antineoplastic | DNA topoisomerase I | DNA topoisomerase I inhibitor | |
| hydrochloride | | | DNA damage | |
| Amorolfine | Antifungal | C-14 sterol reductase | Metabolism, sterol | Antimycotic |
| hydrochloride | | | C-14 sterol reductase inhibitor | |
| Exemestane | Cytostatic | Aromatase | Estrogen antagonist | |
| | Hormone antagonist | | aromatase inhibitor | |
| | | | Metabolism, hormone, estrogen | |
| Benzocaine | Anesthetic (e.g., local) | | May block sodium channels | |
| | Stomatological | | Nerve sodium permeability inhibitor | |
| | Ophthalmological, otological | | Sensory nerve impulse inhibitor | |
| | Antipruritic | | | |
| | Wound healing agent | | | |
| | Topical vasoprotective | | | |
| | Antihemorrhoidal | | | |
| | Anorectic | | | |
| | Scabicides and ectoparasiticide | | | |
| | Non-narcotic analgesic | | | |
| | Antiemetic | | | |
| | Antirheumatic | | | |
| Padimate O | Dermatological | | Absorbs UVB, which forms excited species | |
| | Emollients and protectives | | that inflict DNA damage | |
| | Sunscreen | | | |
| R(+)-Verapamil | Antihypertensive | Calcium channel | Calcium channel blocker | |
| hydrochloride | Antiarrhythmic | | Class IV anti-arrhythmia agent | |
| Terconazole | Antifungal | | Possible fungal-cell-membrane- | |
| | Trichomonacide | | permeabilizer | |
| Halcinonide | Antiinflammatory | | ACTH antagonist | Glucocorticoids, topical |
| | Corticosteroid (e.g., topical) | | Glycogen deposition inhibitor | |
| | | | Calcium mobilizer | |
| | | | ACTH secretion inhibitor | |
| | | | Gluconeogenesis promoter | |
| | | | Phosphorus mobilizer | |
| | | | Immunosuppressive | |
| Rifaximin | Antidiarrheal and oral electrolyte | β-subunit of DNA-dependent RNA | Acts on the β-subunit DNA-dependent | |
| | replacer | polymerase | RNA polymerase of microorganisms to | |
| | Rifampicin/rifamycin | | inhibit RNA synthesis. | |
| | Antibiotic | | | |
| Quinestrol | Antineoplastic | Estrogen receptor | Estrogen receptor agonist | |
| | Estrogen | | | |
| Zafirlukast | Antileukotriene | | Leukotriene D4 and E4 antagonist | IC50 in our hands of 18.5 uM |
| | Antiasthmatic | | | |
| Tolterodine tartrate | Antispasmodic | | Muscarinic receptor antagonist | |
| | Anti-Incontinence | | | |
| | Genitourinary smooth muscle | | | |
| | relaxant | | | |
| Diphenidol | Antiemetic | | | |
| hydrochloride | Antivertigo agent | | | |
| Benoxinate | Local anesthetic | | Na⁺ channel binder | |
| hydrochloride | | | Blocks sensory nerve endings near the | |
| | | | site of application. | |
| Mesoridazine | Tranquilizer | | Dopamine antagonist | |
| besylate | Antipsychotic | | Sympatholytic alpha | |
| | Phenothiazine | | Benzodiazepine agonist | |
| | Antihistamine | | Heat regulating center inhibitor | |
| | | | Membrane stabilizer | |
| | | | Dopamine turnover stimulant | |
| | | | Prolactin release stimulant | |
| | | | Ganglionplegic | |
| | | | Parasympatholytic | |
| | | | Dopamine-2 antagonist | |
| Desoxycorticosterone | Diuretic | | Binds mineralocorticoid receptor | Adrenocortical steroid (salt-regulating) |
| acetate | Anti-Addison agent | | | |
| Oxeladin | Cough suppressant | | | |
| Manganese | Mineral supplement | | | |
| gluconate | Antioxidant | | | |
| Oxibendazole | Antihelmintic | | Reported ATP-synthesis-inhibitor | |
| Sodium fusidate | Antibiotic | | Protein synthesis inhibitor | |
| | | | Chloramphenicol acetyltransferase | |
| | | | inhibitor | |
| Noscapine | Non-narcotic analgesic | | | |
| | Cough sedatives (antitussive) | | | |
| | Antiasthmatic (e.g., xanthines) | | | |
| | Expectorant cough preparation | | | |
| Narasin | Antibiotic | membranes | Increases ion transport through | |
| | Coccidiostat | | membranes | |
| | Growth stimulant | | | |
| Promazine | Antipsychotic | | Neuron receptor blocker | |
| hydrochloride | Antiemetic | | Dopamine receptor antagonist | |
| | Neuroleptic | | | |
| | Phenothiazine | | | |
| Zimelidine | Antidepressant | | Inhibition of serotonin uptake | |
| dihydrochloride | SSRI | | | |
| Benzamil HCL | | Sodium, proton channel | Ion transport | |
| | | | Sodium, proton channel inhibitor | |
| Thiostrepton | Antibiotic | Ribosome | Inhibits ribosome function | Cyclic peptide from Streptomyces |
| | | | Translation, ribosome | active against gram-positive bacteria |
| Mianserin | Antihistamine | α-adrenergic receptor, Histamine H1 | Antihistamine H1 | Tetracylic compound |
| hydrochloride | Antidepressant | receptor Serotonin receptor | Norepinephrine transporter | |
| | | Norepinephrine transporter | Antiserotonin | |
| Quinacrine | Antiparasitic | Monoamine oxygenase | DNA replication inhibitor | Probably active against |
| | Antihelmintic | DNA | Binds DNA | Diphyllobothrium latum |
| | Antiprotozoal (e.g., antimalarial) | | Transcription inhibitor | Giardia lamblia |
| | Antineoplastic | | Protein synthesis inhibitor | Hymenolepsis nana |
| | Antinematodal | | Destroys ribosomes | activity against: Taenia |
| | Anticestodal | | Monoamine oxygenase inhibitor | phospholipase inhibitor |
| | | | Inhibits succinate oxidation | DNA incorporation |
| | | | Interferes with electron transport | |
| | | | Destroys gametocytes of quartan malaria | |
| | | | and vivax malaria | |
| | | | Destroys trophozoites of quarta malaria, | |
| | | | falciparum malaria, and vivax malaria | |
| Bifonazole | Antifungal | Lanosterol 14-alpha-demethylase | Reported carnitine acetyltransferase | Appears to increase permeability of |
| | | | stimulator | fungal-cell-membrane, causing |
| | | | Interferes with sterol biosynthesis | leakage of intracellular components |
| | | | Lanosterol 14-alpha-demethylase inhibitor | |
| | | | May enhance peroxisomal-β-oxidation | |
| | | | system | |
| | | | Reported carnitine-palmitoyl transferase- | |
| | | | stimulator | |
| Bay 41-2272 | | Guanylate cyclase | NO-sensitive guanylate cyclase activator | |
| Erbstatin | Cytostatic | EGFR | EGFR tyrosine kinase inhibitor | Isolated from Actinomyces MH435-hF |
| | Antineoplastic agent | | Receptor, growth factor | Enzyme inhibitor |
| | | | | Growth inhibitor |
| Gefitinib (base) | Antineoplastic | EGFR | Receptor, growth factor | |
| | Protein kinase inhibitor | | EGFR tyrosine kinase inhibitor | |
| Tyrphostin Ag 1478 | Antineoplastic | EGFR | Receptor, growth factor | Tyrphostin |
| | | | EGFR tyrosine kinase inhibitor | |
| Floxuridine | Antimetabolite | DHFR | DNA polymerase inhibitor | |
| | Antineoplastic | DNA polymerase | DHFR inhibitor | |
| | Cytostatic | | DNA metabolism, pyrimidine | |
| | Analgesic | | Apparent deoxyuridylate-methylation- | |
| | Antiviral | | inhibitor | |
| | | | Inhibits thymydilate synthase | |
| | | | Partial RNA-synthesis-inhibitor | |
| | | | DNA-synthesis-inhibitor | |
| Spiperone | Antipsychotic | Aldosterone receptor | Dopamine receptor antagonist | Butyrophenone |
| | | Dopamine receptor | Receptor, renin-angiotensin | |
| | | | Aldosterone receptor antagonist | |
| | | | Dopamine antagonist | |
| Donepezil | Nootropic | Acetylcholinesterase | Acetylcholinesterase inhibitor | Cholinesterase inhibitors |
| hydrochloride | Parasympathomimetic | | | |
| Capsaicin | Stimulant | Vanilloid | Reported gastric-motility-inhibitor | |
| | Analgesic (e.g., narcotic) | Nociceptin | Probable mechanism: substance-P- | |
| | Musculoskeletal product | | depletor | |
| | Antigout preparation | | Nociceptin antagonist | |
| | Topical antirheumatic | | Vanilloid receptor agonist | |
| | Antipruritic | | Prevents reaccumulation of substance-P in | |
| | | | peripheral sensory neurons | |
| Isosulfan Blue | | | Selectively picked up by lymphatic vessels | Rosaniline dye |
| | | | delineating them from surrounding tissue | Imaging agent |
| | | | possibly due to a protein-binding | |
| | | | phenomenon | |
| | | | May weakly bind serum-albumin | |
| Dienestrol | Estrogens (e.g., nonsteroidal) | Estrogen receptor | Estrogen receptor agonist | Hormone |
| Octyl | Antiacne | | Esterogenic | Sunscreen ingredient |
| methoxycinnamate | Emollients and Protectives | | Absorber of ultraviolet light | |
| Hydroquinone | Vitamin | | Desceases formation of melanin | Depigmentor |
| | Topical nonsteroidal products for | | Melanin antagonist | Reduces Skin Pigmentation By |
| | inflammatory skin disorders | | Tyrosine oxidation inhibitor | Inhibiting Enzymatic Oxidation Of |
| | including psoriasis | | | Tyrosine |
| | Antiacne | | | Radiation-protective agents |
| | Vitamin A and D | | | |
| Monobenzone | Depigmentor | | Depigmenting agent; unknown mechanism | Depigmentor |
| Mitotane | Cytostatic | Adrenal cortex | Antiadrenal cortex; adrenal-suppressant | Can cause adrenal inhibition without |
| | Antineoplastic | | Reduces measurable 17- | cellular destruction |
| | | | hydroxycorticosteroids | Insecticide |
| | | | Increases formation of hydroxycortisol-6-β | Dichlorodiphenyldichloroethane |
| | | | Corticosteroid-antagonist | derivative |
| | | | Alters peripheral hydrocortisone | |
| | | | metabolism | |
| Trifluridine | Antiviral (e.g., ophthalmological) | Thymidine kinase (e.g., HSV, VSV) | Antimetabolite (pyrimidine) | In-vitro activity against adenovirus |
| | Ophthalmological | Viral DNA polymerase | herpes-simplex-virus type-2 | Interferes with DNA synthesis in |
| | Antimetabolite | | Thymidine phosphorylase inhibitor | cultured mammalian-cells |
| | | | Activity against herpes simplex virus type-1 | |
| | | | vaccinia-virus | |
| Gramicidin | Anti-infective | Membranes | Bacterial membrane disruptor | |
| | Antibiotic (e.g., topical, peptide) | | | |
| 2-Hydroxyflavanone | Antioxidant | | | Flavonoid |
| | | | | Isolated from Collinsonia canadensis |
| 10- | Antineoplastic | | | extracted from the needles of the Yew |
| Deacetylbaccatine III | | | | tree, Taxus baccata L. |
| | | | | Precursor to taxol drugs |
| Ifenprodil tartrate | Vascular dilator | NMDA receptor | 5-HT3 receptor antagonist | Traxoprodil, an analog of ifenprodil, is |
| | | | alpha1-adrenoceptor antagonist | highly selective for the NR2B subunit |
| | | | NMDA receptor antagonist | of the NMDA receptor. |
| | | | Possible glutamate antagonist | |
| 3,3′- | | | | |
| (Pentamethylenedioxy)dianiline | | | | |
| Tiratricol | Anorectic | | Antioxidant | |
| | Thyroid therapy | | Thyroid-hormone activity (metabolite of T3) | |
| | | | Inhibits of TSH production and secretion | |
| | | | by the pituitary gland. | |
| Oxyphenbutazone | Antiinflammatory | | | |
| hydrate | NSAID | | | |
| | Antirheumatic | | | |
| Siguazodan | Vasodilator | Cyclic nucleotide phosphodiesterase type | Phosphodiesterase inhibition | |
| | | III | selective inhibition of cyclic nucleotide | |
| | | | phosphodiesterase type III. | |
| Chlorphenoxamine | Antihistamine | | Sedative | Parasympatholytic |
| hydrochloride | | | Anticholinergic | |
| Edoxudine | Antiviral (e.g., topical) | Thymidine kinase | Thymidine kinase inhibitor | |
| Thiram | Antifungal | | Aldehyde dehydrogenase inhibitor | Insect attractant, repellent and |
| | Antiseptic | | Glutathione reductase inactivator | chemost |
| | | | | Pesticide |
| | | | | Fungicide, bactericide, wood |
| | | | | preservative |
| Beta Escin | Systemic vasoprotective | | Inhibits edema formation | |
| | Systemic muscle relaxant | | Decreases vascular fragility | |
| Carbaryl | Insecticide (e.g., carbamate) | | Inhibits cholinesterase | Acaricide |
| | Scabicide | | | Growth regulator/Fertilizer |
| | Ectoparasiticide | | | Cholinesterase inhibitors |
| | Antiparasitic | | | |
| Iophenoxic Acid | Contrast agent | Bilirubin | Increases fluorescence of bilirubin bound | Contrast media |
| | | Human serum albumin | to human serum albumin at drug/albumin | |
| | | | molar ratios lower then 1. The increase | |
| | | | may result from a conformational change | |
| | | | in the albumin, which in turn causes | |
| | | | displacement of bilrubin | |
| Piceatannol | Antineoplastic agent | Syk | Tyrosine kinase inhibitor | Platelet aggregation inhibitor |
| | | Lck | Protein kinase inhibitor | |
| | | Mitochondrial F1 ATPase | Syk inhibitor | |
| | | | Lck inhibitor | |
| | | | mitochondrial F1 ATPase inhibitor | |
| U18666A | | Seladin-1 | 2,3 oxidosqualene-lanosterol cyclase | |
| | | D⁸-sterol isomerase | inhibitor | |
| | | | D⁸-sterol isomerase inhibitor | |
| | | | Seladin-1 inhibitor | |
| | | | Cholesterol synthesis inhibitor | |
| Methylglyoxal | | S-adenosyl-L-methionine decarboxylase | S-adenosyl-L-methionine decarboxylase | Flavoring agent |
| | | Lactoylglutathione lyase | inhibitor | |
| | | | Lactoylglutathione lyase inhibitor | |
| Anisomycin | Antibiotic | Ribosomal peptidyl transferase | Ribosomal peptidyl transferase inhibitor | |
| | Antifungal | p38 | p38 activator | |
| | | JNK | JNK activator | |
| | | | p54 activator | |
| | | | MAP kinase activator | |
| | | | Stress-activated protein kinases activator | |
| Celastrol | Antioxidant | HSF1 | Suppresses LPS-induced pro- | triterpenoid isolated from the root of a |
| | Anti-inflammatory | DNA topoisomerase I | inflammatory cytokines release | Chinese medicinal herb, Tripterygium |
| | | Tyrosine kinase | Suppresses LPS-induced NF-kB activation | regeli, is a DNA topoisomerase |
| | | 20S proteasome | and NO production | inhibitor |
| | | | HSF1 inhibitor | |
| | | | Transcription activator | |
| | | | DNA topoisomerase I inhibitor | |
| | | | Tyrosine kinase inhibitor | |
| | | | Inhibits chymotrypsin-like activity of 20S | |
| | | | proteasome | |
| Cerulenin | | HMG-CoA synthetase | Irreversible inhibitor of fatty acid synthase | |
| | | | Metabolism, sterol | |
| | | | HMG-CoA synthetase inhibitor | |
| Camptothecin | Antineoplastic | DNA topoisomerase I | DNA topoisomerase I inhibitor | |
| Tirapazamine | Antineoplastic | DNA strand breaker | DNA damage | |
| | Radiation-sensitizing agent | | DNA strand breaker | |
| | | | Kills hypoxic cells | |
| Fascaplysin | Antiangiogenic | Cdk4/Cyclin D1 | Cdk4/Cyclin D1 inhibitor | |
| | | Cdk6/D1 | Cdk6/D1 inhibitor | |
| | | | ATP competitive inhibitor | |
| Triciribine | Antineoplastic | AKT1/2/3 | Metabolite triciribine phosphate inhibits | |
| | Antiviral (e.g., HIV) | | amidophosphoribosyl transferase and | |
| | | | IMP-dehydrogenase | |
| | | | Signaling, kinase, PKB | |
| | | | AKT1/2/3 inhibitor | |
| | | | Inhibits nuclear import of HIV | |
| Deptropine citrate | Antihistamine (H1) | | Antiserotonin | |
| | | | Anticholinergic | |
| Mequinol | Antineoplastic | | | Antioxidant |
| | Hypopigmenting agent | | | |
| Pramoxine | Anesthetic (e.g., topical) | | Reduces sodium permeability of nerves | Inhibits generation and conduction of |
| hydrochloride | | | | nerve impulses from sensory nerves |
| Betaxolol | Antihypertensive | | Cardioselective beta-1-adrenergic | Anti-adrenergic |
| hydrochloride | Sympatholytic | | antagonist | |
| Dihydroergotamine | Cardiac sympathomimetic | | Antiserotonin | Anti-adrenergic |
| mesylate | Antimigraine preparation | | Sympatholytic | |
| | Peripheral vasodilator | | Dopamine agonist | |
| | Systemic vasoprotective | | | |
| | Vasoconstrictor | | | |
| Beta-lonol | Antioxidant | | Prevents toxic effect of thiophenol on rats. | |
| | | | Increase o-demethylase activity of | |
| | | | cytochrome P-450 | |
| | | | Activates cytosol and microsomal | |
| | | | glutathione-dependent enzymes. | |
| | | | Protects erythrocytes from peroxide | |
| | | | damage by thiophenol and simultaneously | |
| | | | enhanced its prooxidant effect in the liver. | |
| Thapsigargin | | Endoplasmic reticulum Ca²⁺-ATPase | Histaminergic | Tumor promoter |
| | | | Ca²⁺ pump inhibitor | |
| | | | Calcium ATPase pump inhibitor | |
| | | | Calcium channel antagonist | |
| Dilazep | Vasodilator | | Calcium antagonist | Antiarrhythmic activity |
| dihydrochloride | Antithrombotic | | Adenosine uptake inhibitor | Antiplatelet |
| | | | coronary and cerebral vasodilator | |
| Cyclocytidine | Antimetabolite | | DNA synthesis inhibitor | Specific for S-phase of the cell-cycle |
| hydrochloride | Antineoplastic | | Cell proliferation inhibitor | |
| Saponin | | | Permeabilizes cell membranes | Saponin is any glucosides that occur in |
| | | | hemolytic activity | plants and are characterized by the |
| | | | | property of producing a soapy lather. |
| | | | | A moisture absolving amorphous |
| | | | | saponin mixture can be used as a |
| | | | | foaming and emulsifying agent and |
| | | | | detergent When it is digested, it yields |
| | | | | a sugar and a sapogenin aglycone. |
| Mofebutazone | Anti-inflammatory agent | | | |
| | Antirheumatic, non-steroidal | | | |
| | NSAID | | | |
| Dehydroepiandrosterone | Anabolic | | Androgen | Adjuvants, immunologic |
| | Androgen | | | Hormone |
| Amitrole (4) | | Catalase | Catalase inhibitor | Herbicide |
| | | | | Pesticide |
| Tioxolone | Antiacne | | | |
| 6-Nitroquipazine | SSRI | 5-HT transporter complex | Inhibits serotonin reuptake | Serotonin antagonists |
| Shikonin | Antibacterial | Caspase 3/8 | Signaling, apoptosis, inducer | |
| | Anti-inflammatory | | Caspase 3/8 activator | |
| | Antitumor | | Angiogenesis inhibitor | |
| | | | Blocks expression of integrin αᵥβ₃ | |
| Picotamide | Anticoagulants and platelet | Thromboxane A2/prostaglandin | Antiaggregant | |
| | aggregation inhibitor | endoperoxide H2 (TXA2/PGH2) receptor | TXA2/PGH2 receptor inhibitor | |
| | | Thromboxane A2 (TXA2) synthase | TXA2 synthase inhibitor | |
| Amitraz | Insecticide | | Alpha-adrenergic receptor agonist | Scabicide |
| | Antiparasitic | | Monoamine oxidase inhibitor | Insect repellent |
| | | | | Acaricide |
| Cepharanthine | Antiallergic | PKC | Interferes with release of histamine from | Antineoplastic agents, phytogenic |
| | Antineoplastic | ODC | mast cells | |
| | NSAID | | May inhibit linkage of H1-histone with | |
| | Antiviral (e.g., Anti-HIV) | | phospholipid vesicles | |
| | Antiinflammatory | | Blocks IL-1 release | |
| | Antiallergenic | | PKC inhibitor | |
| | | | Reported protein-kinase-C-inhibitor | |
| | | | Suppresses NO production | |
| | | | ODC inhibitor | |
| UCH-L3 inhibitor | | UCH-L3 | UCH-L3 inhibitor | |
| (4,5,6,7- | | | Proteasome | |
| Tetrachloroindan- | | | | |
| 1,3-dione) | | | | |
| UCH-L1 inhibitor | | UCH-L1 | Protein processing | |
| (LDN-57444) | | | UCH-L1 inhibitor | |
| 2-Methoxyestradiol | Anti-angiogenic | PARP | Proliferation inhibitor | Steroid |
| | | Tubulin | Angiogenesis inhibitor | Estrogen |
| | | HIF-1 | Signaling, apoptosis | |
| | | | PARP inhibitor | |
| | | | Tubulin binder | |
| | | | HIF-1 antagonist | |
| 1,5-Isoquinolinediol | | PARP | PARP inhibitor | neuroprotective agent |
| | | | Potent inhibitor of Poly(ADP-ribose) | |
| | | | synthetase | |
| | | | Blocks nitric oxide-induced neuronal | |
| | | | toxicity | |
| AG-490 | | JAK-2 | Kinase inhibitor | Tyrphostin |
| | | JAK-3 | JAK-2 tyrosine kinase inhibitor | possible antineoplastic |
| | | STAT-3 | Inhibits constitutive activation of STAT-3 | |
| | | | DNA binding | |
| | | | Inhibits IL-2-induced growth of MF tumor | |
| | | | cells | |
| | | | JAK-3 tyrosine kinase inhibitor | |
| 1,2-bis-(2 | | Ca²⁺ | Calcium chelator | |
| aminophenoxy)ethane | | | | |
| N,N,N,N,- | | | | |
| tetreacetic acid | | | | |
| CAY10433 | | Histone deacetylase | Transcription, chromatin | |
| | | | HDAC inhibitor | |
| Suberohydroxamic | | Histone deacetylase | Transcription, chromatin | |
| Acid | | | HDAC inhibitor | |
| Tyrphostin 23 | Antineoplastic | EGFR/PDGFR kinase | Tyrosine kinase inhibitor | Tyrphostin |
| | | | Aldosterone secretion inhibitor | Growth inhibitor |
| | | | Suppresses MAPK kinase activation | Enzyme inhibitors |
| | | | Receptor, growth factor | |
| | | | EGFR/PDGFR kinase inhibitor | |
| Tyrphostin 47 | Antineoplastic | EGFR/PDGFR kinase | Receptor, growth factor | Tyrphostin |
| | | | EGFR/PDGFR kinase inhibitor | Blocks HT-29 colon cancer cell |
| | | | | proliferation |
| AG-494 | Antineoplastic | EGFR | JAK-2 tyrosine kinase inhibitor | Tyrphostin |
| | | JAK-2 tyrosine kinase | EGFR inhibitor | |
| | | HER1 | Selective HER1 inhibitor (vs. HER1-2; | |
| | | | IC50: HER1 1.1 μM; HER1-2 45 μM2.) | |
| | | | Receptor, growth factor | |
| | | | Blocks Cdk2 activation | |
| Tyrphostin 25 | Antineoplastic | EGFR | Inhibits substrate binding on protein | Tyrphostin |
| | | Transducin | tyrosine kinases | Enzyme inhibitors |
| | | | Inhibits EGFR tyrosine kinase | |
| | | | Inhibits GTPase activity of transducin | |
| | | | Inhibits neuromedin B-induced | |
| | | | phosphorylation of p125FAK | |
| | | | Blocks induction of inducible nitric oxide | |
| | | | synthase in glial cells. | |
| | | | Induces apoptosis in human leukemic cell | |
| | | | lines. | |
| Tyrphostin 46 | Antineoplastic | EGFR | Inhibits EGFR tyrosine kinase and EGFR | Tyrphostin |
| | | ERK1 | phosphorylation | |
| | | ERK2 | Inhibits EGF-dependent cell proliferation | |
| | | | Inhibits ERK1 and ERK2 | |
| DNA-PK inhibitor II | | DNA-PK | DNA-PK inhibitor | |
| NSC 663284 | | CDC25 phosphatase | CDC25 phosphatase inhibitor | |
| | | | Arrests cell cycle progression | |
| | | | Inhibits Cdk dephosphorylation | |
| | | | Delays tumor growth | |
| BHQ | | Calcium ATPase | Mobilizes Ca²⁺ specifically from | |
| | | Prostaglandin E₂ | Ins(1,4,5)P₃-sensitive Ca²⁺ stores by | |
| | | | inhibiting microsomal and sarcoplasmic | |
| | | | reticulum Ca²⁺-ATPase activity. Does not | |
| | | | affect mitochondrial Ca2+ fluxes or plasma | |
| | | | membrane Ca2+/Mg2+ ATPase activity | |
| | | | Inhibits prostaglandin E₂ | |
| | | | Calcium ATPase inhibitor | |
| Fenvalerate | | Calcineurin | Calcineurin inhibitor | Insecticide |
| | | | Induces depolarization by keeping Na⁺ | |
| | | | channels open. | |
| Satraplatin | Antineoplastic | | | Platinum agent |
| Parthenolide | | NFκB | Interleukin-1 antagonist | |
| | | | NFκB inhibitor | |
| | | | Prostaglandin E2 antagonist | |
| | | | Prostaglandin antagonist | |
| | | | Interleukin antagonist | |
| | | | Nitric oxide antagonist | |
| | | | TNF-alpha antagonist | |
| | | | MAP kinase activation inhibitor | |
| Silver sulfadiazine | Wound healing agent | DHFS | DHFS inhibitor | Anti-infective agents, local |
| | Antipruritic | | DNA metabolism, pyrimidine | Acts on cell-membrane and cell wall |
| | Antibiotic and/or sulphonamide | | | Silver is released slowly in |
| | (e.g., topical) | | | concentrations toxic to bacteria |
| | Antiseptic and disinfectant | | | |
| Beta-carotene | Vitamin | | Vitamin A | Antioxidant |
| | Neurotonic | | | Food coloring agent |
| | Emollient and protective | | | Ultraviolet screen |
| | Anti-atheroma preparation (e.g., of | | | |
| | natural origin) | | | |
| | Preparations to prevent cataract, | | | |
| | anticataractogenic | | | |
| | Vitamin A | | | |
| Methyltestosterone | Androgen, female hormone | | ICSH antagonist | increased pharmacologic activity |
| | combination | | Gonadotropin antagonist | compared with testosterone |
| | Estrogen, progestogen | | Testosterone release inhibitor | |
| | combinations | | Spermatogenesis inhibitor | |
| | Androgen | | Protein catabolism inhibitor | |
| | | | Predominant anabolic activity | |
| | | | Anabolic | |
| | | | High-dose: FSH antagonist | |
| | | | Minor androgenic activity | |
| Propidium iodide | | DNA | Reported to intercalate DNA | |
| | | Cholinesterase | Cholinesterase inhibitor | |
| Tunicamycin | Antibiotic | P-MurNAc penapeptide synthase; | Protein modification | Nucleoside |
| | Antifungal | Glycosyltransferase | P-MurNAc penapeptide synthase; | |
| | Antiviral | | Glycosyltransferase inhibitor | |
| | | | Inhibits expression of thrombin receptors | |
| 2′,2″- | | | | |
| (Pentamethylenedioxy)diacetanilide | | | | |
| 3′,3″- | | | | |
| (Pentamethylenedioxy)diacetanilide | | | | |
| Lovastatin | Cardiovascular agent | HMG-CoA reductase | HMG-CoA reductase Inhibitor | |
| | Hypolipemics/antiatheroma | | Metabolism, sterol | |
| | Cholesterol and triglyceride | | | |
| | reduction | | | |
| Cyclosporine | Immunosuppressive | Calcineurin | Inhibits lymphokine production | Prolongs survival of allogeneic |
| | Cytostatic | | Suppresses humoral immunity | transplanted tissue |
| | Immunosuppressant | | Inhibits helper-T-cells preferentially | Action may be due to specific and |
| | Immunomodulator | | T-suppressor-cells may be suppressed | reversible inhibition of |
| | Antirheumatic | | interleukin-2-release-inhibitor | immunocompetent lymphocytes in the |
| | Antifungal | | Calcineurin inhibitor | G₀-phase or G₁-phase of the cell-cycle |
| | | | Suppresses cell-mediated reactions | |
| | | | including: allograft-rejection | |
| Ribavirin | Antivirals (e.g., HIV, topical) | RNA polymerase | RNA polymerase inhibitor | In-vitro activity against respiratory |
| | Antimetabolite | Inosine phosphate dehydrogenase | Inosine phosphate dehydrogenase | syncytial virus, influenza virus, herpes |
| | | | inhibitor | simplex virus |
| | | | Transcription, machinery | |
| Simvastatin | Hypolipemic | HMG-CoA reductase | cholesterol-synthesis-inhibitor | Anticholesteremic agent |
| | Angiotensin II antagonist | | decreases LDL-cholesterol-levels, VLDL- | Antihyperlipidemic |
| | Cholesterol and triglyceride | | cholesterol-levels and plasma-triglycerides | Antilipemic agents |
| | reduction | | increases HDL-cholesterol-levels | |
| | Cardiovascular product | | HMG-CoA reductase inhibitor | |
| | Cardiac glycoside | | | |
| Mycophenolic acid | Antibiotic | INPDH (inosine phosphate | INPDH inhibitor | Antibiotics, antineoplastic |
| | Immunosuppressant | dehydrogenase) | Inhibits T- and B-lymphocyte proliferation | Enzyme inhibitor |
| | | | | Antineoplastic |
| Atorvastatin | Antilipemic/hypolipemic | HMG-CoA reductase | Metabolism, sterol | |
| | Cholesterol and triglyceride | | HMG-CoA reductase inhibitor | |
| | reduction | | | |
| | Antidiabetic | | | |
| | Anti-atheroma preparation (e.g., of | | | |
| | natural origin) | | | |
| Fluvastatin Sodium | Hypolipemic | HMG-CoA reductase inhibitor | HMG-CoA reductase inhibitor | |
| | Cardiac glycoside | | Metabolism, sterol | |
| | Cholesterol and triglyceride | | | |
| | reduction | | | |
| Artemisinin | Antimalarial | Iron | Interacts with iron to generate free | Toxicity specific to cells with high iron |
| | Antiparasitic | | radicals, toxicity to parasites | content |
| | Antiprotozoal | | | |
| | Antineoplastic | | | |
| Nitazoxanide | Antiprotozoal | pyruvate:ferredoxin oxidoreductase | Interferes with the PFOR enzyme- | |
| | Antiparasitic | (PFOR) | dependent electron transfer reaction | |
| | Anti-infective | | | |
| | Anticestodal | | | |
| | Antiviral | | | |
| Chloroquine | Antiprotozoal | Heme polymerase | Inhibits heme polymerase | |
| | Antimalarial | | Inhibits biosynthesis of nucleic acids | |
| Mevastatin | Antibiotic | HMG-CoA reductase | Inhibits protein geranylgeranylation | |
| | | | HMG-CoA reductase inhibitor | |
| | | | May induce bone morphogenic protein-2 | |
| | | | (BMP-2) | |
| | | | Causes cell cycle arrest in late G₁ phase | |
| TOFA | | Acetyl-CoA carboxylase | Inhibitor of acetyl-CoA carboxylase (ACC), | |
| | | | key enzyme involved in fatty acid | |
| | | | biosynthesis | |
| 2′-C-Methylcytidine | Antiviral | | Ribonucleoside analog | |
| | Antimetabolite | | | |
| LY 294002 | | Phosphoinositide 3-kinases | Inhibitor of phosphoinositide 3-kinase | |
| Telaprevir (VX-950) | Antiviral | NS3-4A serine protease | Inhibitor of NS3-4A serine protease | |
| | Anti-HCV | | | |
| Merimepodib (VX- | Antiviral | Inosine monophosphate dehydrogenase | Inhibitor of IMPDH | |
| 497) | Anti-HCV | (IMPDH) | | |
| Valopicitabine (NM- | Antiviral | HCV RNA polymerase | Inhibitor of RNA polymerase | |
| 283) | Anti-HCV | | Inhibitor of HCV RNA polymerase | |
| Boceprevir | Antiviral | NS3 protease | Inhibitor of NS3 protease | |
| (SCH 503034) | Anti-HCV | | | |
| Celgosivir | Antiviral | α-Glucosidase I | Inhibitor of α-glucosidase I | |
| | Anti-HCV | | | |
| HCV-796 | Antiviral | HCV RNA polymerase | Inhibitor of RNA polymerase | Benzofuran |
| | Anti-HCV | | Inhibitor HCV RNA polymerase | |
| Emetine | Antiamoebic | 40S ribosome | Inhibitor of eukaryotic protein synthesis | Can cause vomiting or diarrhea |
| | Antiprotozoal | | Binds 40S ribosome | |
| | Antiparasitic | | Inhibits translocation | |
| Arbidol | Antiviral | | Induces interferon production | |
| | | | Inhibition of membrane fusion | |
| Gemcitabine | Pyrimadine analog | DNA | Inhibits DNA replication | |
| | Antineoplastic | DNA polymerase | | |
| | Nucleoside analog | | | |
| Vincristine | Antiviral | Tubulin | Inhibits mitosis by binding tubulin | Isolated from Vinca Rosea |
| | Antineoplastic | Tubilin dimers | | |
| | | Microtubules | | |
| Dihydroergotamine | Antimigraine | Serotonin receptor | Partial agonist of α-adrenergic receptors | |
| mesylate | Vasoconstrictor | 5-HT1_{Da} receptor | Partial agonist of dopamine D2 and D3 | |
| | | 5-HT1_{Db} receptor | receptors | |
| | | 5-HT_{1A} receptor | Binds to 5-HT1_{Da}, 5-HT1_{Db}, 5-HT_{1A}, 5-HT_{2A}, | |
| | | 5-HT_{2A} receptor | and 5-HT_{2C} receptors | |
| | | 5-HT_{2C} receptor | Inhibits release of proinflammatory | |
| | | α-Adrenergic receptor | neuropeptides | |
| | | Dopamine D2L receptor | | |
| | | Dopamine D3 receptor | | |
| Interferon alfa-2a | Antiviral | IFN-α receptor | Inhibits viral replication | |
| | Antineoplastic | | Upregulation of MHC I protein expression | |
| | Anti-HIV | | | |

Compounds useful in the invention include those described herein in any of their pharmaceutically acceptable forms, including isomers such as diastereomers and enantiomers, salts, solvates, and polymorphs thereof, as well as racemic mixtures. Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

By “patient” is meant any animal (e.g., a mammal such as a human). Any animal can be treated using the methods, compositions, and kits of the invention.

To “treat” is meant to administer one or more agents to measurably slow or stop the replication of a virus in vitro or in vivo, to measurably decrease the load of a virus (e.g., any virus described herein including a hepatitis virus such as hepatitis A, B, C, D, or E) in a cell in vitro or in vivo, or to reduce at least one symptom (e.g., those described herein) associated with having a viral disease in a patient. Desirably, the slowing in replication or the decrease in viral load is at least 20%, 30%, 50%, 70%, 80%, 90%, 95%, or 99%, as determined using a suitable assay (e.g., a replication assay described herein). Typically, a decrease in viral replication is accomplished by reducing the rate of DNA or RNA polymerization, RNA translation, polyprotein processing, or by reducing the activity of a protein involved in any step of viral replication (e.g., proteins coded by the genome of the virus or host protein important for viral replication).

By “an effective amount” is meant the amount of a compound, alone or in combination with another therapeutic regimen, required to treat a patient with a viral disease (e.g., any virus described herein including a hepatitis virus such as hepatitis A, B, C, D, or E) in a clinically relevant manner. A sufficient amount of an active compound used to practice the present invention for therapeutic treatment of conditions caused by a virus varies depending upon the manner of administration, the age, body weight, and general health of the patient. Ultimately, the prescribers will decide the appropriate amount and dosage regimen. Additionally, an effective amount may be an amount of compound in the combination of the invention that is safe and efficacious in the treatment of a patient having a viral disease over each agent alone as determined and approved by a regulatory authority (such as the U.S. Food and Drug Administration).

By “more effective” is meant that a treatment exhibits greater efficacy, or is less toxic, safer, more convenient, or less expensive than another treatment with which it is being compared. Efficacy may be measured by a skilled practitioner using any standard method that is appropriate for a given indication.

By “hepatic virus” is meant a virus that can cause hepatitis. Such viruses include hepatitis A, hepatitis B, hepatitis C, hepatitis D, hepatitis E, non-ABCDE hepatitis, and hepatitis G.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that inhibits viral replication and that is formulated for administration by intravenous injection will differ from a low dosage of the same agent formulated for oral administration.

By a “high dosage” is meant at least 5% (e.g., at least 10%, 20%, 50%, 100%, 200%, or even 300%) more than the highest standard recommended dosage of a particular compound for treatment of any human disease or condition.

By “hypercholesterolemia” is meant an total cholesterol level of at least 200 mg/dl. High risk groups include those with at least 240 mg/dl. Normal cholesterol levels are below 200 mg/dl. Hypercholesterolemia may also be defined by low density lipoprotein (LDL) levels. Less than 100 mg/dl is considered optimal; 100 to 129 mg/dl is considered near optimal/above optimal; 130 to 159 mg/dl borderline high; 160 to 189 mg/dl high; and 190 mg/dl and above is considered very high.

By a “candidate compound” is meant a chemical, be it naturally-occurring or artificially-derived. Candidate compounds may include, for example, peptides, polypeptides, synthetic organic molecules, naturally occurring organic molecules, nucleic acid molecules, peptide nucleic acid molecules, and components or derivatives thereof.

In the generic descriptions of compounds of this invention, the number of atoms of a particular type in a substituent group is generally given as a range, e.g., an alkyl group containing from 1 to 4 carbon atoms or C₁₋₄ alkyl. Reference to such a range is intended to include specific references to groups having each of the integer number of atoms within the specified range. For example, an alkyl group from 1 to 4 carbon atoms includes each of C₁, C₂, C₃, and C₄. A C₁₋₁₂ heteroalkyl, for example, includes from 1 to 12 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 12 ring carbon atoms, inclusive. Exemplary cyclic groups include cyclopropyl, cyclobutyl, cyclopentyl, and cyclohexyl groups.

By “C₁₋₄ alkyl” is meant a branched or unbranched hydrocarbon group having from 1 to 4 carbon atoms. A C₁₋₄ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₄ alkyls include, without limitation, methyl, ethyl, n-propyl, isopropyl, cyclopropyl, cyclopropylmethyl, n-butyl, iso-butyl, sec-butyl, tert-butyl, and cyclobutyl.

By “C₂₋₄ alkenyl” is meant a branched or unbranched hydrocarbon group containing one or more double bonds and having from 2 to 4 carbon atoms. A C₂₋₄ alkenyl may optionally include monocyclic or polycyclic rings, in which each ring desirably has from three to six members. The C₂₋₄ alkenyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₄ alkenyls include, without limitation, vinyl, allyl, 2-cyclopropyl-1-ethenyl, 1-propenyl, 1-butenyl, 2-butenyl, 3-butenyl, 2-methyl-1-propenyl, and 2-methyl-2-propenyl.

By “C₂₋₄ alkynyl” is meant a branched or unbranched hydrocarbon group containing one or more triple bonds and having from 2 to 4 carbon atoms. A C₂₋₄ alkynyl may optionally include monocyclic, bicyclic, or tricyclic rings, in which each ring desirably has five or six members. The C₂₋₄ alkynyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₄ alkynyls include, without limitation, ethynyl, 1-propynyl, 2-propynyl, 1-butynyl, 2-butynyl, and 3-butynyl.

By “C₂₋₆ heterocyclyl” is meant a stable 5- to 7-membered monocyclic or 7- to 14-membered bicyclic heterocyclic ring which is saturated, partially unsaturated, or unsaturated (aromatic), and which consists of 2 to 6 carbon atoms and 1, 2, 3, or 4 heteroatoms independently selected from N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3, or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups. Examples of C₁₋₇ heteroalkyls include, without limitation, methoxymethyl and ethoxyethyl.

By “halide” or “halogen” is meant bromine, chlorine, iodine, or fluorine.

By “fluoroalkyl” is meant an alkyl group that is substituted with a fluorine atom.

By “perfluoroalkyl” is meant an alkyl group consisting of only carbon and fluorine atoms.

By “carboxyalkyl” is meant a chemical moiety with the formula —(R) —COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “hydroxyalkyl” is meant a chemical moiety with the formula —(R) —OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substituent of the formula —OR, wherein R is a C₆₋₁₂ aryl group.

By “alkylthio” is meant a chemical substituent of the formula —SR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl, heteroalkyl, heteroaryl, and/or aryl groups, resulting in a positive charge at the nitrogen atom.

Other features and advantages of the invention will be apparent from the following Detailed Description and the claims.

### BRIEF DESCRIPTION OF THE DRAWINGS

FIG. 1 is a graph showing survival data for sertraline and oseltamivir in the lethal infection of influenza A/PR/8/34 induced in C57/BL6 mice.

FIG. 2 is a graph showing dose dependant increase in survival rate of sertraline-treated groups as compared to vehicle-treated groups.

### DETAILED DESCRIPTION

We have identified compounds that decrease replication of a hepatitis C(HCV) replicon in mammalian cells. Accordingly, the present invention provides compositions, methods, and kits useful in the treatment of viral diseases, which may be caused by a single stranded RNA virus, a flaviviridae virus, or a hepatic virus (e.g., described herein). In certain embodiments, the viral disease is viral hepatitis (e.g., hepatitis A, hepatitis B, hepatitis C, hepatitis D, and hepatitis E). The invention also features screening methods useful for the identification of novel compounds for the treatment of viral diseases. Compositions of the invention can include one or more agents selected from the agents of Table 1, Table 2, Table 3, Table 4, and Table 5. Treatment methods of the invention include administration of one or more agents selected from the agents of Table 1, Table 2, and Table 3, optionally along with an additional antiviral therapy (e.g., administration of one or more agents of Table 4 or Table 5) to a patient (e.g., a mammal such as a human). Optionally, functional or structural analogs (e.g., those described herein) of these agents or agents of the same therapeutic or mechanistic class as those described herein (see, e.g., Table 8) may be employed in the compositions, methods, and kits of the invention. The ability of a composition to reduce replication of a virus may be due to a decrease in RNA or DNA polymerization, RNA translation, RNA or DNA transcription, a decrease in posttranslational protein processing (e.g., polyprotein processing in hepatitis C), or a decrease in activity of a protein involved in viral replication (e.g., a protein coded for by the viral genome or a host protein required for viral replication). The compounds or combinations of compounds may also enhance the efficacy of the other therapeutic regimens such that the dosage, frequency, or duration of the other therapeutic regimen is lowered to achieve the same therapeutic benefit, thereby moderating any unwanted side effects.

In one particular example, the patient being treated is administered two agents listed in Table 1, Table 2 and/or Table 3 within 28 days of each other in amounts that together are sufficient to treat a patient having a viral disease. The two agents can be administered within 14 days of each other, within seven days of each other, within twenty-four hours of each other, or even simultaneously (i.e., concomitantly). If desired, either one of the two agents may be administered in low dosage.

### Viral Diseases

The invention relates to the treatment of viral disease, which can be caused by any virus. Viruses include single stranded RNA viruses, flaviviridae viruses, and hepatic viruses. In particular, the flaviviridae family of viruses include hepacivirus (e.g., HCV); flaviviruses; pestiviruses, and hepatitis G virus.

Flaviviruses generally are discussed in Chapter 31 of Fields Virology, supra. Exemplary flaviviruses include Absettarov, Alfuy, Apoi, Aroa, Bagaza, Banzi, Bouboui, Bussuquara, Cacipacore, Carey Island, Dakar bat, Dengue 1, Dengue 2, Dengue 3, Dengue 4, Edge Hill, Entebbe bat, Gadgets Gully, Hanzalova, Hypr, Ilheus, Israel turkey meningoencephalitis, Japanese encephalitis, Jugra, Jutiapa, Kadam, Karshi, Kedougou, Kokobera, Koutango, Kumlinge, Kunjin, Kyasanur Forest disease, Langat, Louping ill, Meaban, Modoc, Montana myotis leukoencephalitis, Murray valley encephalitis, Naranjal, Negishi, Ntaya, Omsk hemorrhagic fever, Phnom-Penh bat, Powassan, Rio Bravo, Rocio, royal farm, Russian spring-summer encephalitis, Saboya, St. Louis encephalitis, Sal Vieja, San Perlita, Saumarez Reef, Sepik, Sokuluk, Spondweni, Stratford, Tembusu, Tyuleniy, Uganda S, Usutu, Wesselsbron, west Nile, Yaounde, yellow fever, and Zika viruses.

Pestiviruses generally are discussed in Chapter 33 of Fields Virology, supra. Specific pestiviruses include, without limitation: bovine viral diarrhea virus, classical swine fever virus (also called hog cholera virus), and border disease virus.

### Hepatitis Viruses

Viruses that can cause viral hepatitis include hepatitis A, hepatitis B, hepatitis C, hepatitis D, and hepatitis E. In addition, non-ABCDE cases of viral hepatitis have also been reported (see, for example, Rochling et al., Hepatology 25:478-483, 1997). Within each type of viral hepatitis, several subgroupings have been identified. Hepatitis C, for example, has at least six distinct genotypes (1, 2, 3, 4, 5, and 6), which have been further categorized into subtypes (e.g., 1a, 1b, 2a, 2b, 2c, 3a, 4a) (Simmonds, J. Gen. Virol. 85:3173-3188, 2004).

In the case of hepatitis C, acute symptoms can include jaundice, abdominal pain, fatigue, loss of appetite, nausea, vomiting, low-grade fever, pale or clay-colored stools, dark urine, generalized itching, ascites, and bleeding varices (dilated veins in the esophagus). Hepatitis C can become a chronic infection, which can lead to liver infection and scarring of the liver, which can, in turn, require the patient to undergo a liver transplant.

Hepatitis C is an RNA virus taken up specifically by hepatic cells. Once inside the cells, the RNA is translated into a polyprotein of about 3,000 amino acids. The protein is then processed into three structural and several non-structural proteins necessary for viral replication. Accordingly, HCV may be treated by reducing the rate any of the steps required for its replication or inhibiting any molecule involved in replication, including but not limited to, entry into a target cell, viral genome replication, translation of viral RNA, protolytic processing, and assembly and release from the target cell (e.g., using the agents described herein).

### Compounds

Certain compounds that may be employed in the methods, compositions, and kits of the present invention are discussed in greater detail below. It will be understood that analogs of any compound of Table 1, Table 2, or Table 3 can be used instead of the compound of Table 1, Table 2, or Table 3 in the methods, compositions, and kits of the present invention.

### HMG-CoA Reductase Inhibitors

In certain embodiments, an HMG-CoA reductase inhibitor can be used in the compositions, methods, and kits of the invention. By an “HMG-CoA reductase inhibitor” is a compound that inhibits the enzymatic activity of 3-hydroxy-3-methylglutaryl-coenzyme A (HMG-CoA) reductase by at least about 10%. HMG-CoA reductase inhibitors include but are not limited to simvastatin, lovastatin, mevastatin, pravastatin, monacolin M, monacolin X, fluvastatin, atorvastatin, cerivastatin, rosuvastatin, fluindostatin, velostatin, compactin, dihydrocompactin, rivastatin, dalvastatin, pitavastatin, BAY102987, BAY X 2678, BB476, bervastatin, BMY21950, BMY22089, colestolone, CP83101, crilvastatin, DMP565, glenvastatin, L659699, L669262, P882222, P882284, PD134965, PD135022, RP61969, S2468, SC37111, SC45355, SQ33600, SR12813, SR45023A, U20685, and U88156, as well as pharmaceutically acceptable salts thereof (e.g., simvastatin sodium, lovastatin sodium, fluvastatin sodium, etc.). Additional HMG-CoA reductase inhibitors and analogs thereof useful in the methods and compositions of the present invention are described in U.S. Pat. Nos. 3,983,140; 4,231,938; 4,282,155; 4,293,496; 4,294,926; 4,319,039; 4,343,814; 4,346,227; 4,351,844; 4,361,515; 4,376,863; 4,444,784; 4,448,784; 4,448,979; 4,450,171; 4,503,072; 4,517,373; 4,661,483; 4,668,699; 4,681,893; 4,719,229; 4,738,982; 4,739,073; 4,766,145; 4,782,084; 4,804,770; 4,841,074; 4,847,306; 4,857,546; 4,857,547; 4,940,727; 4,946,864; 5,001,148; 5,006,530; 5,075,311; 5,112,857; 5,116,870; 5,120,848; 5,166,364; 5,173,487; 5,177,080; 5,273,995; 5,276,021; 5,369,123; 5,385,932; 5,502,199; 5,763,414; 5,877,208; and 6,541,511; and U.S. Pat. Application Publication Nos. 2002/0013334 A1; 2002/0028826 A1; 2002/0061901 A1; and 2002/0094977 A1.

### Clozapine

In certain embodiments, clozapine or a clozapine analog can be used in the compositions, methods, and kits of the invention. Suitable clozapine analogs include acetophenazine maleate, alentemol hydrobromide, alpertine, azaperone, batelapine maleate, benperidol, benzindopyrine hydrochloride, brofoxine, bromperidol, bromperidol decanoate, butaclamol hydrochloride, butaperazine, butaperazine maleate, carphenazine maleate, carvotroline hydrochloride, chlorpromazine, chlorpromazine hydrochloride, chlorprothixene, cinperene: cintriamide, clomacran phosphate, clopenthixol, clopimozide, clopipazan mesylate, cloroperone hydrochloride, clothiapine, clothixamide maleate, cyclophenazine hydrochloride, droperidol, etazolate hydrochloride, fenimide, flucindole, flumezapine, fluphenazine decanoate, fluphenazine enanthate, fluphenazine hydrochloride, fluspiperone, fluspirilene, flutroline, gevotroline hydrochloride, halopemide, haloperidol, haloperidol decanoate, iloperidone, imidoline hydrochloride, lenperone, mazapertine succinate, mesoridazine, mesoridazine besylate, metiapine, milenperone, milipertine, molindone hydrochloride, naranol hydrochloride, neflumozide hydrochloride, ocaperidone, olanzapine, oxiperomide, penfluridol, pentiapine maleate, perphenazine, pimozide, pinoxepin hydrochloride, pipamperone, piperacetazine, pipotiazine palmitate, piquindone hydrochloride, prochlorperazine edisylate, prochlorperazine maleate, promazine hydrochloride, remoxipride, remoxipride hydrochloride, rimcazole hydrochloride, seperidol hydrochloride, sertindole, setoperone, spiperone, thioridazine, thioridazine hydrochloride, thiothixene, thiothixene hydrochloride, tioperidone hydrochloride, tiospirone hydrochloride, trifluoperazine hydrochloride, trifluperidol, triflupromazine, triflupromazine hydrochloride, and ziprasidone hydrochloride. Additional clozapine analogs are described in U.S. Pat. Nos. 2,519,886; 2,921,069, 3,084,161, 3,155,669, 3,155,670, 3,438,991, 3,161,644, 4,045,445, 4,308,207, 4,459,232, 4,460,508, 4,460,587, 4,507,311, 4,595,535, 4,192,803, 5,955,459, and 6,197,764.

Trifluperidol

In certain embodiments, trifluperidol or an analog thereof can be used in the compositions, methods, and kits of the invention. The structure of trifluperidol is:

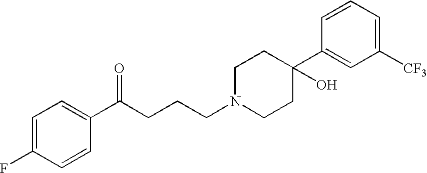

Analogs of trifluperidol are described for example in U.S. Pat. No. 3,438,991 and have the general structure:

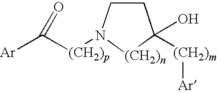

where Ar and Ar′ are monocyclic aryl rings, is 2 to 4, n is 1 or 2, m is 0, 1, or 2, and X is a hydrogen or a methyl group. Ar and Ar′ can represent halophenyls such as fluorophenyl, chlorophenyl, bromophenyl, and iodophenyl; alkoxyphenyls such as methoxyphenyl, ethoxyphenyl, dimethoxyphenyl, and trimethoxyphenyl; monocyclic aromatic hydrocarbon radicals such as phenyl, tolyl, xylyl, isopropylphenyl, and tertiary butyl phenyl; and a trifluoromethylphenyl radical. (CH₂)ₚ can represent a lower alkylene group, e.g., 2 to 4 carbon atoms such as ethylene, trimethylene, propylene, butylene, methylpropylene, and tetramethylene.

### Paclitaxel

In certain embodiments, paclitaxel or a paclitaxel analog can be used in the compositions, methods, and kits of the invention. Paclitaxel is described in U.S. Pat. No. 4,814,470. Paclitaxel analogs include isoserine, taxol, taxotere, cephalomannine, 10-deacetylbaccatine III and those compounds described in U.S. Pat. Nos. 4,814,470, 4,857,653, 4,876,399, 4,924,011, 4,924,012, 4,942,184, 4,960,790, 5,015,744, 5,059,699, 5,136,060, 5,157,049, 5,192,796, 5,227,400, 5,243,045, 5,248,796, 5,250,683, 5,254,580, 5,271,268, 5,272,171, 5,283,253, 5,284,864, 5,290,957, 5,292,921, 5,294,637, 5,319,112, 5,336,684, 5,338,872, 5,350,866, 5,380,751, 5,380,916, 5,399,726, 5,430,160, 5,438,072, 5,470,866, 5,489,601, 5,508,447, 5,539,103, 5,547,981, 5,556,878, 5,574,156, 5,580,899, 5,580,998, 5,587,489, 5,587,493, 5,606,083, 5,622,986, 5,635,531, 5,646,176, 5,654,447, 5,677,470, 5,688,977, 5,693,666, 5,703,117, 5,710,287, 5,714,512, 5,714,513, 5,717,115, 5,721,268, 5,728,725, 5,728,850, 5,739,362, 5,750,562, 5,760,219, 5,773,464, 5,807,888, 5,821,363, 5,840,748, 5,840,929, 5,840,930, 5,854,278, 5,912,264, 5,919,815, 5,902,822, 5,965,739, 5,977,386, 5,990,325, 5,994,576, 5,998,656, 6,011,056, 6,017,935, 6,018,073, 6,028,205, 6,051,724, 6,066,747, 6,080,877, 6,107,332, 6,118,011, 6,124,481, 6,136,961, 6,147,234, 6,177,456, 6,307,064, 6,310,201, 6,350,886, 6,362,217, 6,455,575, 6,462,208, 6,482,963, 6,495,704, 6,515,151, 6,545,168, 6,710,191, 6,762,309, 6,794,523, 6,797,833, 6,878,834, 6,911,549, and 7,019,150.

### Estrogenic Compounds

In certain embodiments, an estrogenic compound can be used in the compositions, methods, and kits of the invention. Estrogenic compounds include estradiol (e.g., estradiol valerate, estradiol cypionate), colpormon, 2-methyoxyestradiol, conjugated estrogenic hormones, equilenin, equilin, dienestrol, ethinyl estradiol, estriol, mestranol, moxestrol, quinestradiol, quinestrol, estrone, estrone sulfate, equilin, diethylstilbestrol, broparoestrol, chlorotrianisine, fosfestrol, hexestrol, methestrol, and genistein. Estrogenic compounds are also described in U.S. Pat. Nos. 2,096,744, 2,465,505, 2,464,203, and 3,159,543.

### Aminopyridines

In certain embodiments, an aminopyridine can be used in the composition, methods, and kits of the invention. By “aminopyridine” is meant any pyridine ring-containing compound in which the pyridine has one, two, or three amino group substituents. Other substituents may optionally be present. Exemplary aminopyridines include phenazopyridine, 4-aminopyridine, 3,4-diaminopyridine, 2,5-diamino-4-methylpyridine, 2,3,6-triaminopyridine, 2,4,6-triaminopyridine, and 2,6-diaminopyridine, the structures of which are depicted below. Phenazopyridine and derivatives thereof have been disclosed in U.S. Pat. Nos. 1,680,108 through 1,680,111. Modifications of di-amino(phenylazo)pyridines have been performed to improve solubility in water by reacting these compounds with alkylating agents (e.g., alkyl halides and alkyl sulphates) to produce quaternary pyridinium bases (see, e.g., U.S. Pat. No. 2,135,293). Heterocyclic azo derivatives and N-substituted diaminopyridines have also been described (U.S. Pat. Nos. 2,145,579 and 3,647,808).

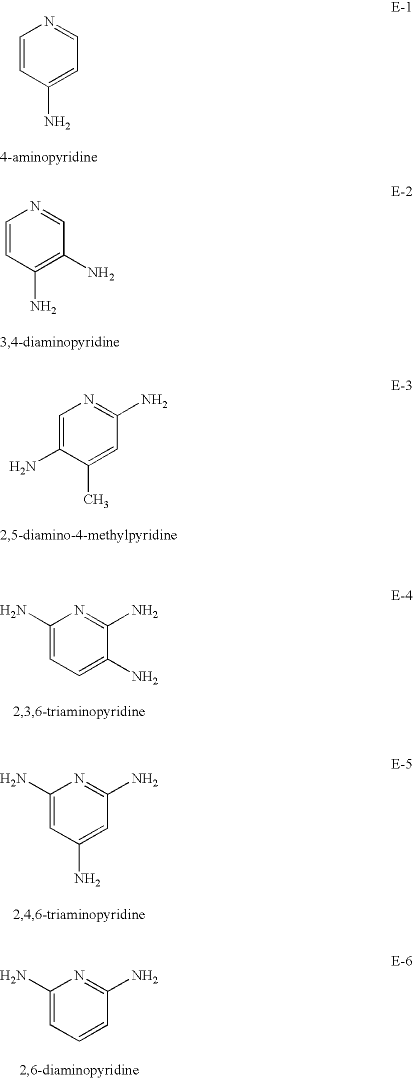

### Antiestrogens

In certain embodiments, an antiestrogen can be used in the methods, compositions, and kits of the invention. Antiestrogens include tamoxifen, 4-hydroxy tamoxifen, clomifene, raloxifene, faslodex, nafoxidine, fulvestrant, CI-680, CI-628, CN-55,956-27, MER-25, U-11,555A, U-11,100A, ICI-46,669, ICI-46,474, diphenolhydrochrysene, erythro-MEA, Parke Davis CN-35,945, allenolic acid, cyclofenil, ethamoxytriphetol, and triparanol and those compounds described in U.S. Pat. Nos. 5,384,332, 4,894,373, 4,536,516, 4,418,068, and 2,914,563.

### Calcium Channel Inhibitors

In certain embodiments, a calcium channel inhibitor can be used in the compositions, methods, and kits of the invention. Calcium channel inhibitors include thapsigargin, verapamil, anipamil, bepridil, gallopamil, devapamil, falipamil, tiapamil, nifedipine, amlodipine, dazodipine, felodipine, isradipine, lanicardipine, nicardipine, nimodipine, nisoldipine, nitrendipine, ryosidie, diltiazem, cinnarizine, flunarizine, BAY-m 4786, and diperdipine.

Verapamil

In certain embodiments, verapamil or an analog thereof can be used in the compositions, methods, and kits of the invention. The structure of verapamil is:

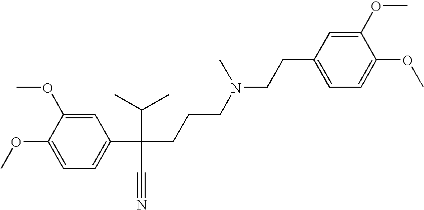

Verapamil analogs are described, for example, in U.S. Pat. No. 3,261,859 and have the general formula:

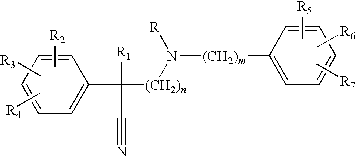

where R is a lower aliphatic hydrocarbon radical; R₁ is hydrogen, a lower alkyl radical, a saturated or unsaturated cyclic or bicyclic hydrocarbon radical, the benzyl radical, or the phenyl radical; R₂, R₃, R₄, R₅, R₆, and R₇ are hydrogen, halogen, lower alkyl radicals, lower alkoxy groups, or two of said substituents together forming the methylene dioxy group; n is an integer between 2 and 4; and m is an integer between 1 and 3.

### Tricyclic Compounds

In certain embodiments, a tricyclic compound can be used in the compositions, methods, and kits of the invention. By “tricyclic compound” is meant a compound having one the formulas (I), (II), (III), or (IV):

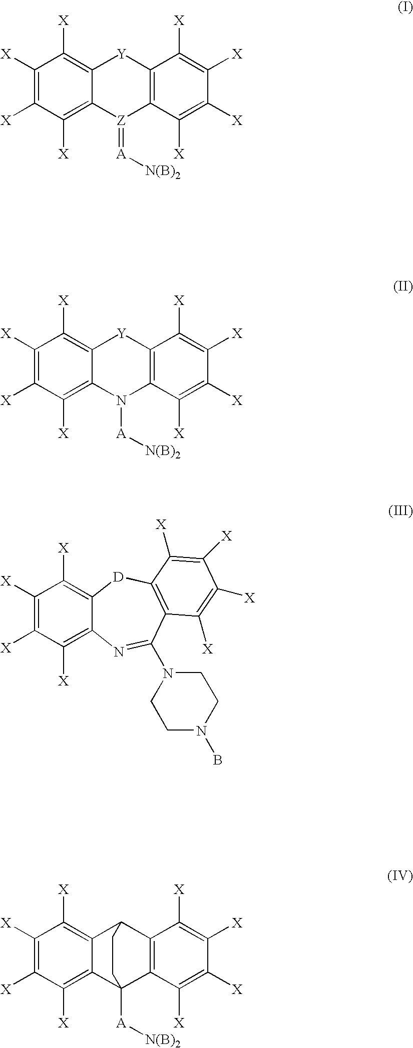

wherein each X is, independently, H, Cl, F, Br, I, CH₃, CF₃, OH, OCH₃, CH₂CH₃, or OCH₂CH₃; Y is CH₂, O, NH, S(O)₀₋₂, (CH₂)₃, (CH)₂, CH₂O, CH₂NH, CHN, or CH₂S; Z is C or S; A is a branched or unbranched, saturated or monounsaturated hydrocarbon chain having between 3 and 6 carbons, inclusive; each B is, independently, H, Cl, F, Br, I, CX₃, CH₂CH₃, OCX₃, or OCX₂CX₃; and D is CH₂, O, NH, or S(O)₀₋₂. In preferred embodiments, each X is, independently, H, Cl, or F; Y is (CH₂)₂, Z is C; A is (CH₂)₃; and each B is, independently, H, Cl, or F. Other tricyclic compounds are described below. Tricyclic compounds include tricyclic antidepressants such as amoxapine, 8-hydroxyamoxapine, 7-hydroxyamoxapine, loxapine (e.g., loxapine succinate, loxapine hydrochloride), 8-hydroxyloxapine, amitriptyline, clomipramine, doxepin, imipramine, trimipramine, desipramine, nortriptyline, and protriptyline, although compounds need not have antidepressant activities to be considered tricyclic compounds of the invention.

Tricyclic compounds that can be used in connection with the invention include amitriptyline, amoxapine, clomipramine, desipramine, dothiepin, doxepin, imipramine, lofepramine, maprotiline, mianserin, mirtazapine, nortriptyline, octriptyline, oxaprotiline, protriptyline, trimipramine, 10-(4-methylpiperazin-1-yl)pyrido(4,3-b)(1,4)benzothiazepine; 11-(4-methyl-1-piperazinyl)-5H-dibenzo(b,e)(1,4)diazepine; 5,10-dihydro-7-chloro-10-(2-(morpholino)ethyl)-11H-dibenzo(b,e)(1,4)diazepin-11-one; 2-(2-(7-hydroxy-4-dibenzo(b,f)(1,4)thiazepine-11-yl-1-piperazinyl)ethoxy)ethanol; 2-chloro-11-(4-methyl-1-piperazinyl)-5H-dibenzo(b,e)(1,4)diazepine; 4-(11H-dibenz(b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; (Z)-2-butenedioate 5H-dibenzo(b,e)(1,4)diazepine; adinazolam; amineptine; amitriptylinoxide; butriptyline; clothiapine; clozapine; demexiptiline; 11-(4-methyl-1-piperazinyl)-dibenz(b,f)(1,4)oxazepine; 11-(4-methyl-1-piperazinyl)-2-nitro-dibenz(b,f)(1,4)oxazepine; 2-chloro-11-(4-methyl-1-piperazinyl)-dibenz(b,f)(1,4)oxazepine monohydrochloride; dibenzepin; 11-(4-methyl-1-piperazinyl)-dibenzo(b,f)(1,4)thiazepine; dimetacrine; fluacizine; fluperlapine; imipramine N-oxide; iprindole; lofepramine; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; noxiptilin; opipramol; oxaprotiline; perlapine; pizotyline; propizepine; quetiapine; quinupramine; tianeptine; tomoxetine; flupenthixol; clopenthixol; piflutixol; chlorprothixene; and thiothixene. Other tricyclic compounds are described in U.S. Pat. Nos. 2,554,736, 3,046,283, 3,058,979, 3,310,553, 3,177,209, 3,194,733, 3,205,264, 3,244,748, 3,271,451, 3,272,826, 3,282,930, 3,282,942, 3,299,139, 3,312,689, 3,389,139, 3,399,201, 3,409,640, 3,419,547, 3,438,981, 3,454,554, 3,467,650, 3,505,321, 3,527,766, 3,534,041, 3,539,573, 3,574,852, 3,622,565, 3,637,660, 3,663,696, 3,758,528, 3,922,305, 3,963,778, 3,978,121, 3,981,917, 4,017,542, 4,017,621, 4,020,096, 4,045,560, 4,045,580, 4,048,223, 4,062,848, 4,088,647, 4,128,641, 4,148,919, 4,153,629, 4,224,321, 4,224,344, 4,250,094, 4,284,559, 4,333,935, 4,358,620, 4,548,933, 4,691,040, 4,879,288, 5,238,959, 5,266,570, 5,399,568, 5,464,840, 5,455,246, 5,512,575, 5,550,136, 5,574,173, 5,681,840, 5,688,805, 5,916,889, 6,545,057, and 6,600,065, and phenothiazine compounds that fit Formula (I) of U.S. patent application Ser. Nos. 10/617,424 (published as U.S. 2004/0116407) or 60/504,310.

Sertraline and Analogs thereof.

In certain embodiments, sertraline or an analog thereof can be used in the compositions, methods, and kits of the invention. Sertraline has the structure:

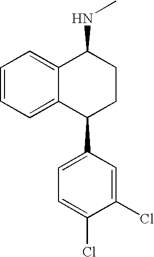

Structural analogs of sertraline are those having the formula:

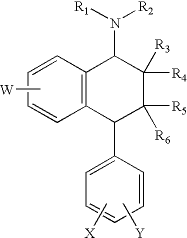

where R₁ and R₂ are independently selected from the group consisting of H, optionally substituted C₁₋₆ alkyl (e.g., CH₃, (CH₂)ₓOH, cyclopropyl, (CH₂)ₓCOOH, or CH₂CHOH(CH₂)ₓ, (CH₂)ₓN(CfH₃)₂, where x is 1, 2, 3, 4, or 5), and optionally substituted C₁₋₇ heteroalkyl (e.g., CH₂CH₂N(CH₃)₂) or R₁ and R₂ together form a C₃₋₈ cycloalkyl optionally heterocyclic, optionally substituted (e.g., forming a morpholine ring), R₃, R₄, R₅, and R₆ are independently H, Cl, F, Br, OH, or optionally substituted C₁₋₆ alkyl; X and Y are each selected from the group consisting of H, F, Cl, Br, CF₃, C₁₋₆ alkoxy (e.g., OPh and OCH₃), and cyano; and W is selected from the group consisting of H, F, Cl, Br, CF₃, C₁₋₃ alkoxy, COOH, CH₂CH₂OH, NHCOH, NHCOCH₃, CH₂S(O)ₙCH₃, CH₂NH₂, CONH₂, CH₂OH, NHCOPh, CH₂NHS(O)ₙCH₃, NHS(O)ₙPh, N(CH₃)₂, S(O)ₙNH₂, NHCOBu, NHS(O)ₙCH₃, NHCOcyclopentyl, CN, NHS(O)ₙcyclopropyl, NH₂, NO₂, I, SO₂N(CH₃)₂, SO₂NHMe, SO₂NHCH₂CH₂OH, CO₂Me, NHSO₂Bu, CONHCH₃, CH₂NHCOCH₃, CONHPh,

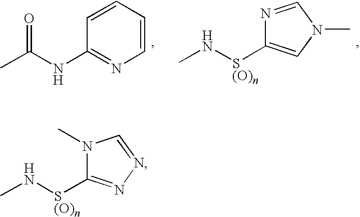

CONHcylopropyl, C(S)NH₂, NHC(S)CH₃, CONHCH₂COOCH₃, CONHCH₂COOH, CONHCH₂cyclopropyl, CON(CH₃)cyclopropyl, CONHcyclobutyl, NHCOcyclopropyl, NH(CH₃)COCH₃, N(CH₃)COCH₃, and CH₂S(O)ₙR₁₁, where n is 0, 1, or 2 and R₁₁ is phenyl, C₂₋₆ heterocyclyl, optionally substituted C₁₋₈ alkyl (e.g., C₄₋₈ unsubstituted alkyl such as Bu or C₃₋₈ substituted alkyl). In certain embodiments, R₁ is CH₃ and R₂ is CH₃, CH₂CH₂OH, cyclopropyl, CH₂COOH, CH₂CH₂NH₂, CH₂CH(OH)R₈, or CH₂CH(R₈)NR₉R₁₀, where n is 0, 1, or 2 and R₈, R₉, and R₁₀ are independently H or C₁₋₆ alkyl. In certain embodiments, X is H and Y is p-OPh, p-OCF₃, o-OCH₃ m-OCH₃, or p-OCH₃. In certain embodiments of the above structure, the sertraline analog has the formula:

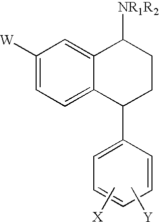

Other sertraline analogs have the formula:

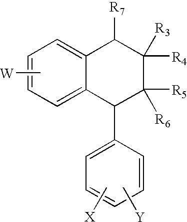

where R₃, R₄, R₅, R₆, W, X, and Y are as defined above, and R₇ is independently H, NH(CH₂)ₘCH₃, O(CH₂)ₘCH₃, OH, O(CH₂)ₘCH₃, ═O, C₁₋₆ alkyl (e.g., isopropyl), or C₁₋₆ alkyoxy, where m is 0, 1, 2, 3, 4, 5, or 6. In certain embodiments, R₃, R₄, R₅, and R₆ are H; X and Y are each Cl at the 3 and 4 positions of the benzyl ring. Exemplary analogs include:

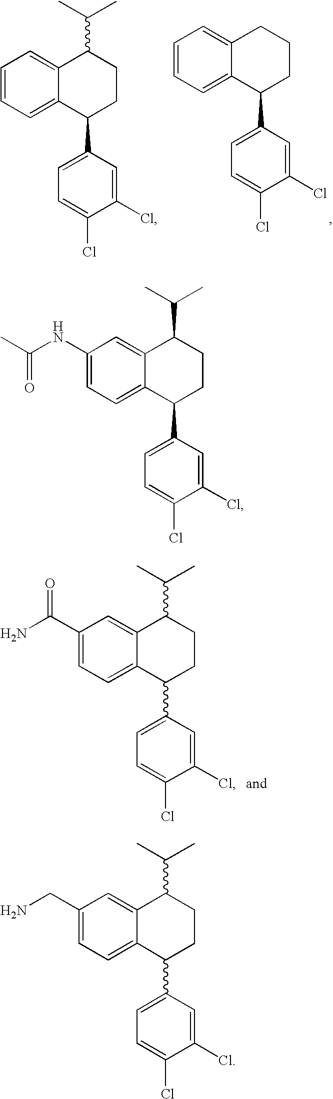

Other sertraline analogs have the formula:

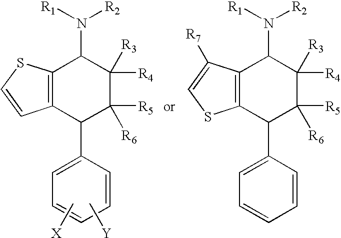

where R₁, R₂, R₃, R₄, R₅, R₆, X and Y are as defined above, and R₇ is H or C₁₋₆ optionally substituted alkyl.
Other sertraline analogs are described by the formula:

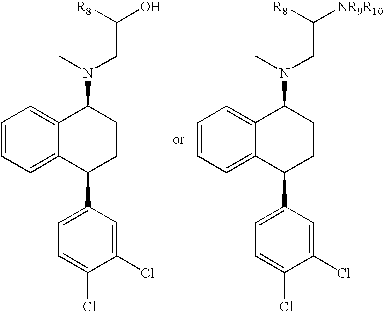

wherein R₈, R₉, and R₁₀ are independently H, optionally substituted C₁₋₆ alkyl (e.g., CH₃, (CH₂)ₓOH, cyclopropyl, (CH₂)ₓCOOH, or CH₂CHOH(CH₂)ₓ, (CH₂)ₓN(CfH₃)₂, where x is 1, 2, 3, 4, or 5), and optionally substituted C₁₋₇ heteroalkyl (e.g., CH₂CH₂N(CH₃)₂)

In certain embodiments, sertraline analogs are in the cis-isomeric configuration. The term “cis-isomeric” refers to the relative orientation of the NR₁R₂ and phenyl moieties on the cyclohexene ring (i.e., they are both oriented on the same side of the ring). Because both the 1- and 4-carbons are asymmetrically substituted, each cis-compound has two optically active enantiomeric forms denoted (with reference to the 1-carbon) as the cis-(1R) and cis-(1S) enantiomers. Sertraline analogs are also described in U.S. Pat. No. 4,536,518. Other related compounds include (S,S)—N-desmethylsertraline, rac-cis-N-desmethylsertraline, (1S,4S)-desmethyl sertraline, 1-des (methylamine)-1-oxo-2-(R,S)-hydroxy sertraline, (1R,4R)-desmethyl sertraline, sertraline sulfonamide, sertraline (reverse) methanesulfonamide, 1R,4R sertraline enantiomer, N,N-dimethyl sertraline, nitro sertraline, sertraline aniline, sertraline iodide, sertraline sulfonamide NH₂, sertraline sulfonamide ethanol, sertraline nitrile, sertraline-CME, dimethyl sertraline reverse sulfonamide, sertraline reverse sulfonamide (CH₂ linker), sertraline B-ring ortho methoxy, sertraline A-ring methyl ester, sertraline A-ring ethanol, sertraline N,N-dimethylsulfonamide, sertraline A-ring carboxylic acid, sertraline B-ring para-phenoxy, sertraline B-ring para-trifluoromethane, N,N-dimethyl sertraline B-Ring para-trifluoromethane, sertraline A-ring methyl sulfoxide (CH₂ linker), sertraline A-ring carboxamide, sertraline A-ring reverse carboxamide, Sertraline A-ring methanamine, sertraline A-ring sulfonylmethane (CH₂ linker), sertraline (reverse) methanesulfonamide, sertraline A-ring thiophene, reduced sulfur sertraline A-ring methyl sulfoxide (CH₂ linker), and hetrocyclic substituted stertraline (reverse) methanesulfonamide. Structures of these analogs and others are shown in Table 9 below. Analogs are also described in Tables 19-24 below.

TABLE 9

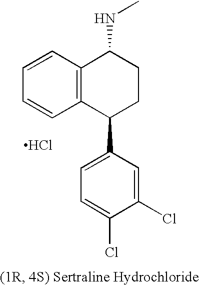

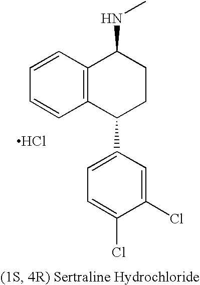

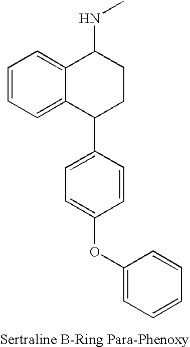

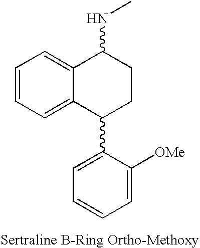

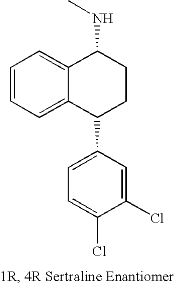

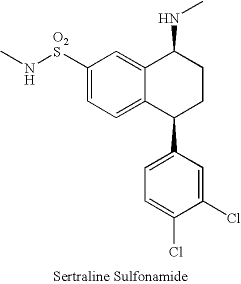

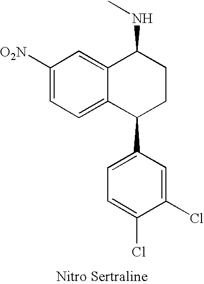

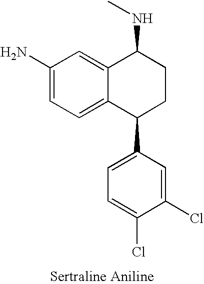

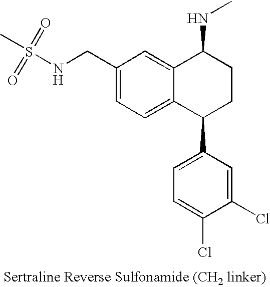

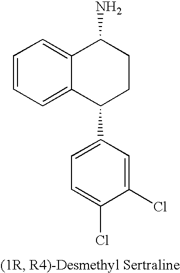

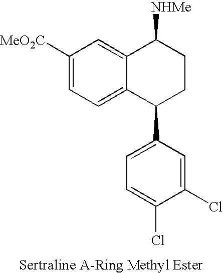

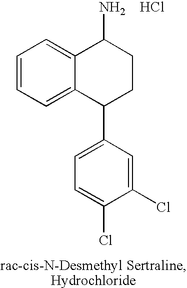

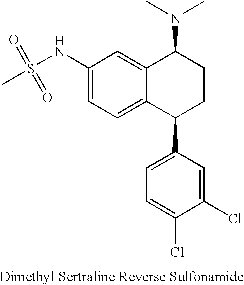

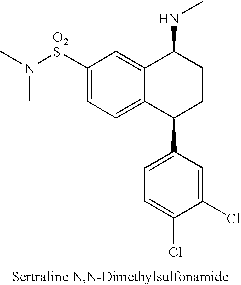

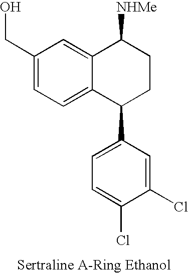

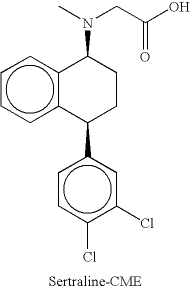

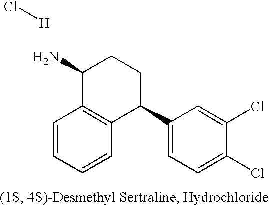

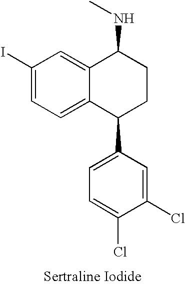

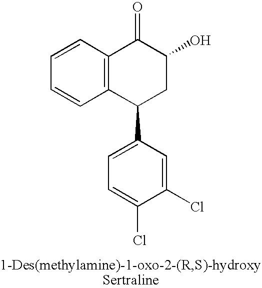

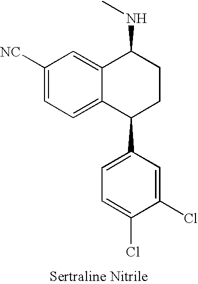

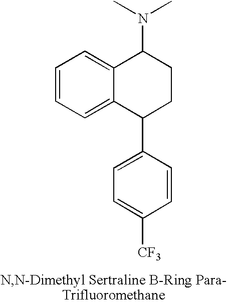

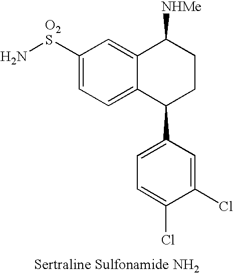

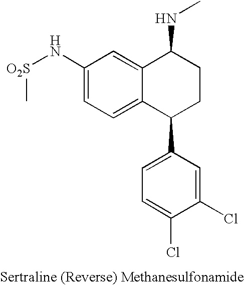

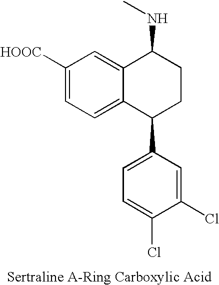

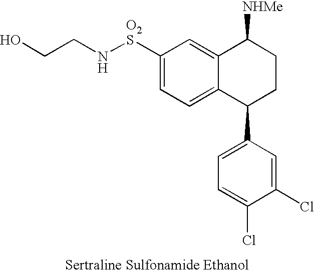

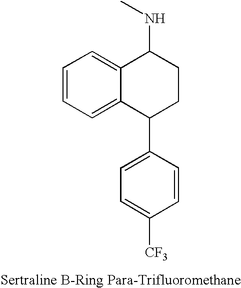

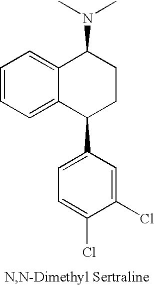

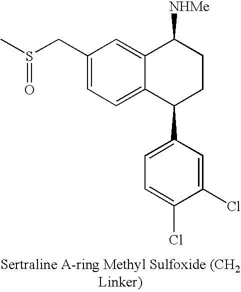

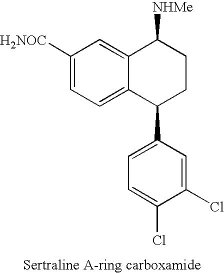

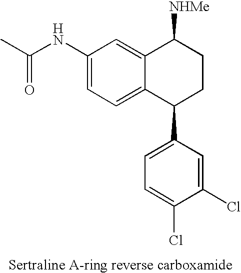

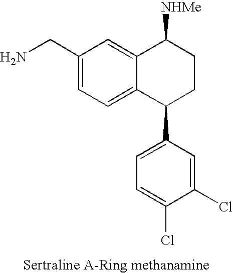

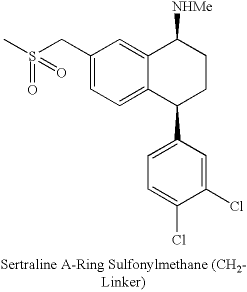

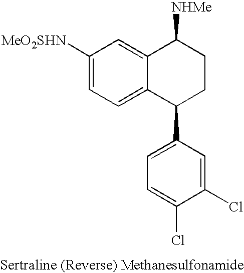

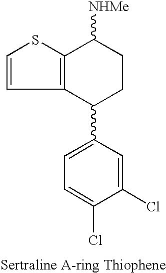

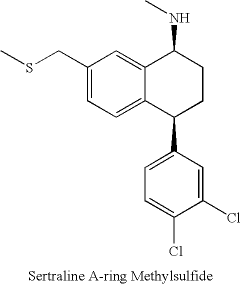

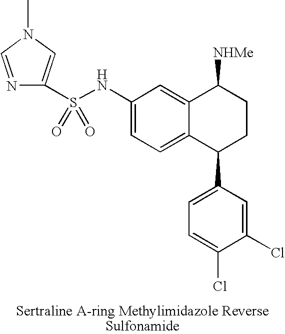

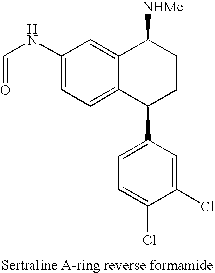

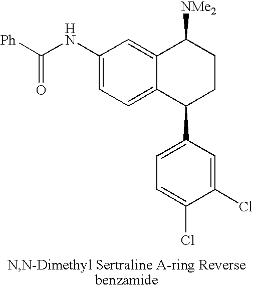

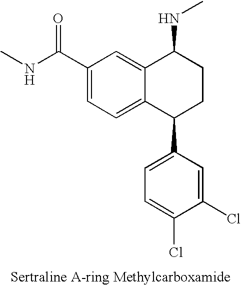

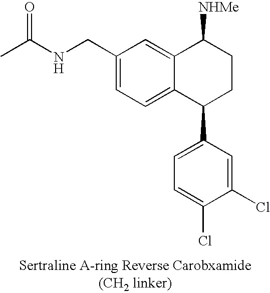

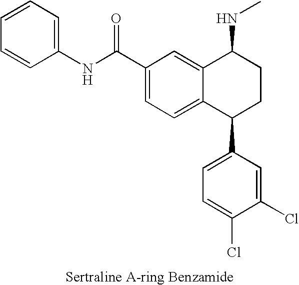

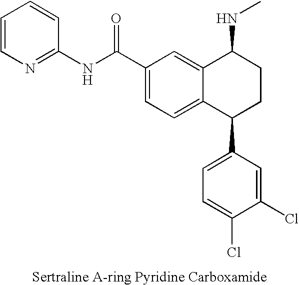

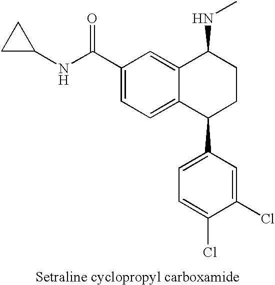

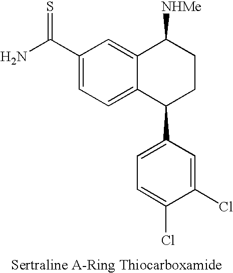

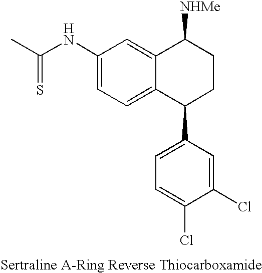

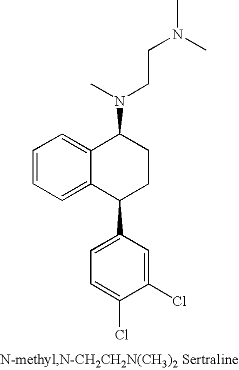

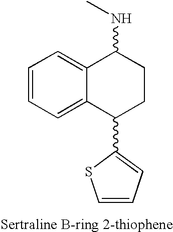

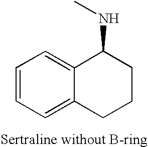

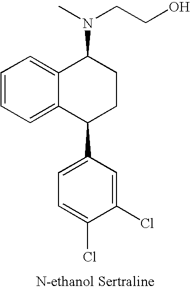

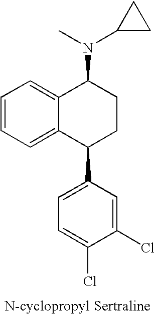

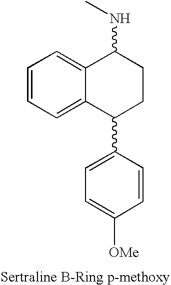

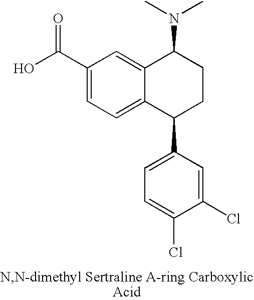

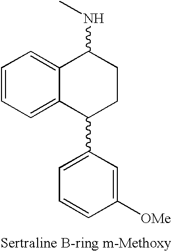

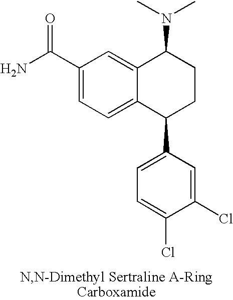

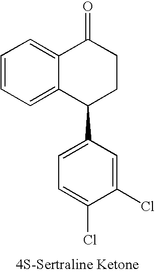

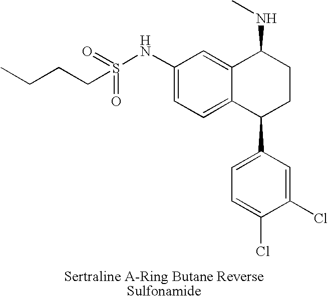

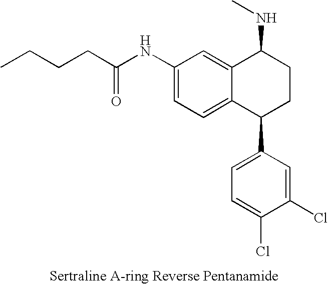

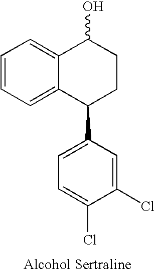

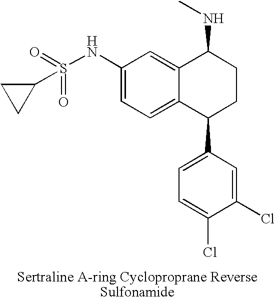

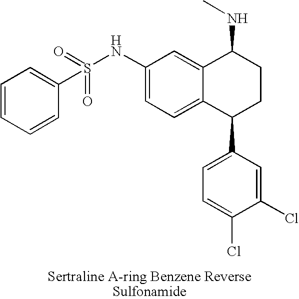

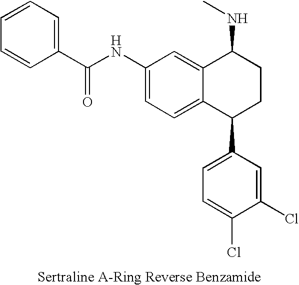

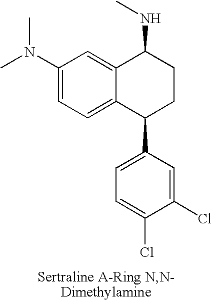

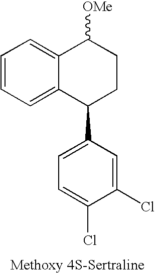

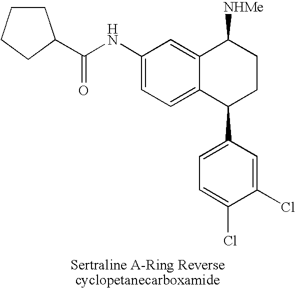

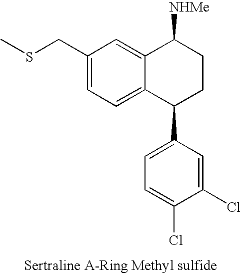

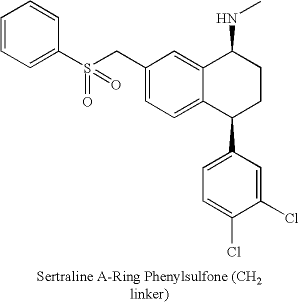

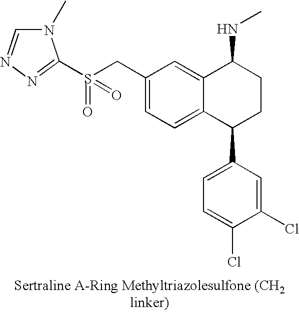

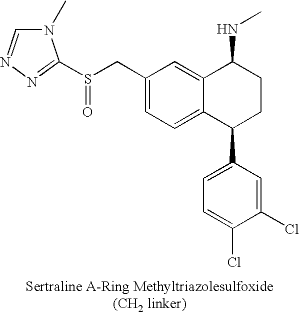

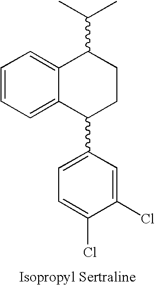

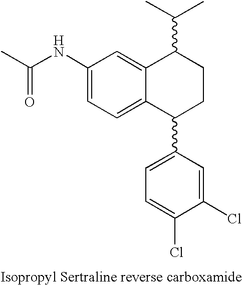

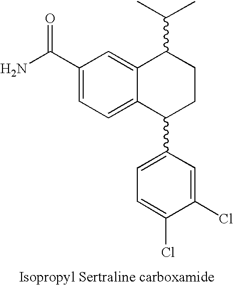

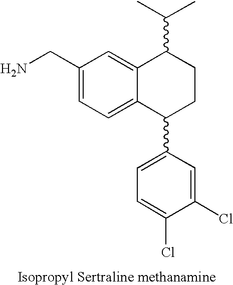

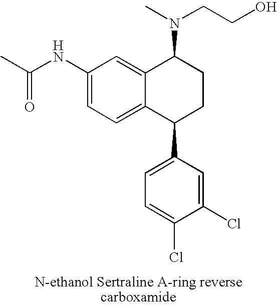

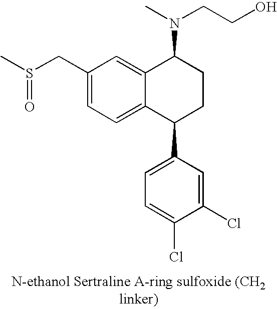

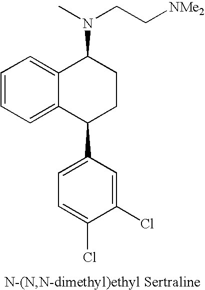

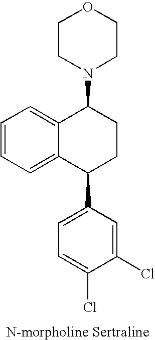

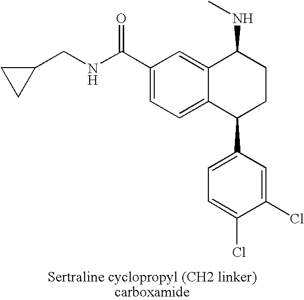

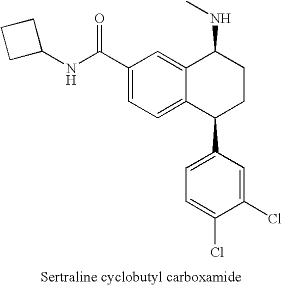

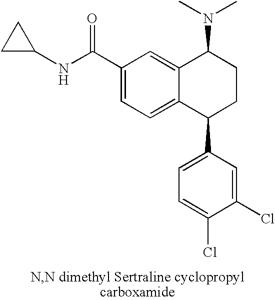

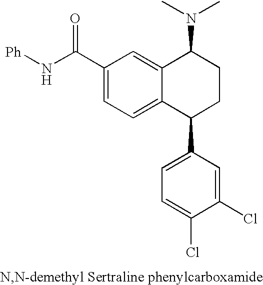

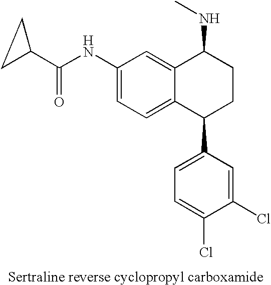

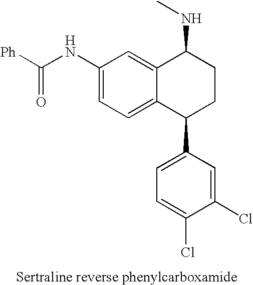

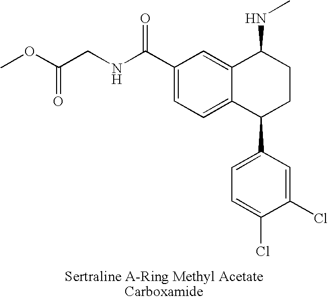

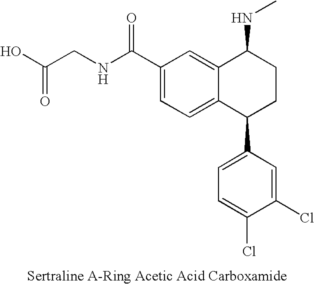

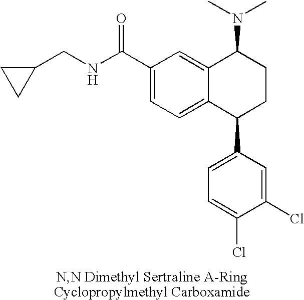

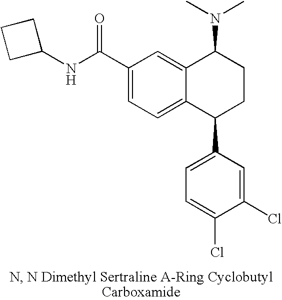

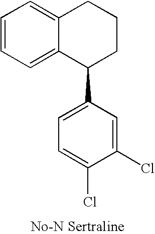

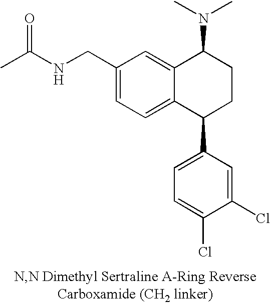

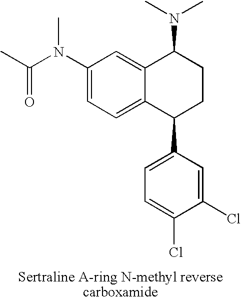

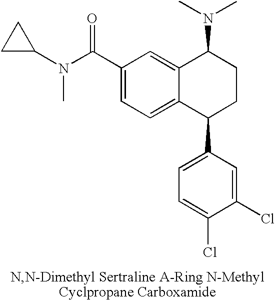

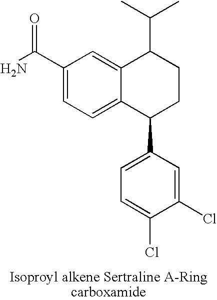

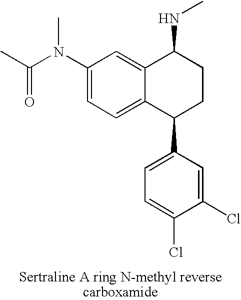

Particularly useful are the following compounds, in either the (1S)-enantiomeric or (1S)(1R) racemic forms, and their pharmaceutically acceptable salts: cis-N-methyl-4-(3,4-dichlorophenyl)-1,2,3,4-tetrahydro-1-naphthalenamine; cis-N-methyl-4-(4-bromophenyl)-1,2,3,4-tetrahydro-1-naphthalenamine; cis-N-methyl-4-(4-chlorophenyl)-1,2,3,4-tetrahydro-1-naphthalenamine; cis-N-methyl-4-(3-trifluoromethyl-phenyl)-1,2,3,4-tetrahydro-1-naphthalenamine; cis-N-methyl-4-(3-trifluoromethyl-4-chlorophenyl)-1,2,3,4-tetrahydro-1-naphthalenamine; cis-N,N-dimethyl-4-(4-chlorophenyl)-1,2,3,4-tetrahydro-1-naphthalenamine; cis-N,N-dimethyl-4-(3-trifluoromethyl-phenyl)-1,2,3,4-tetrahydro-1-naphthalenamine; and cis-N-methyl-4-(4-chlorophenyl)-7-chloro-1,2,3,4-tetrahydro-1-naphthalenamine. Of interest also is the (1R)-enantiomer of cis-N-methyl-4-(3,4-dichlorophenyl)-1,2,3,4-tetrahydro-1-naphthalenamine.

### UK-416244

UK-416244 is an SSRI that is phenoxybenzylamine derivative. UK-416244 has the structure:

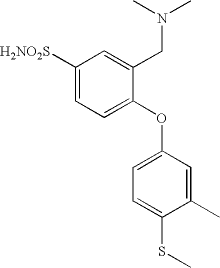

Structural analogs of UK-416244 are compounds having the formula:

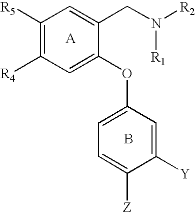

where R₁ and R₂, independently, are H, C₁₋₆ alkyl (e.g., CH₃) or substituted heteroalkyl (e.g., CH₂CH₂N(CH₃)₂ and CH₂OCH₃), or (CH₂)_{d}(C₃₋₆ cycloalkyl) where d is 0, 1, 2, or 3; or R₁ and R₂ together with the nitrogen to which they are attached form an azetidine ring; Z or Y is —S(O)ₙR₃ and the other Z or Y is halogen or —R₃; where R₃ is independently C₁₋₄ alkyl optionally substituted with fluorine (e.g., where R₃ is or is not CF₃) and n is 0, 1, or 2; or Z and Y are linked so that, together with the interconnecting atoms, Z and Y form a fused 5 to 7-membered carbocyclic or heterocyclic ring which may be saturated, unsaturated, or aromatic, and where when Z and Y form a heterocyclic ring, in addition to carbon atoms, the linkage contains one or two heteroatoms independently selected from O, S, and N; (e.g., with the proviso that when R₅ is F and R₂ is methyl then the fused ring is not 1,3-dioxolane and Z and Y together do not form a fused phenyl ring); R₄ and R₅ are, independently, A-X, where A is —CH═CH— or —(CH₂)ₚ— where p is 0, 1, or 2; X is H, F, Cl, Br, I, NH₂, OH, CONR₆R₇, SO₂NR₆R₇, SO₂NHC(═O)R₆, C₁₋₄ alkoxy, NR₈SO₂R₉, NO₂, NR₆R₁₁ (e.g., N(CH₃)₂, CN, CO₂R₁₀ (e.g., COOH), CHO, SR₁₀, S(O)R₉ or SO₂R₁₀; R₆, R₇, R₈ and R₁₀ independently are H, C₁₋₆ alkyl (e.g., CH₃, (CH₂)₃CH₃ or cyclopropyl), C₆₋₁₂ aryl (e.g., phenyl) optionally substituted independently by one or more R₁₂, or C₁₋₆ alkyl-aryl optionally substituted (e.g., CH₂Ph); R₉ is C₁₋₆ alkyl optionally substituted independently by one or more R₁₂; R₁₁ is H, C₁₋₆ alkyl optionally substituted independently by one or more R₁₂, C(O)R₆, CO₂R₉, C(O)NHR₆, or SO₂NR₆R₇; R₁₂ is F (preferably up to 3), Br, OCH₃, OH, CO₂H, C₃₋₆ cycloalkyl, NH₂, CONH₂, C₁₋₆ alkoxy, C₁₋₆ alkoxycarbonyl, or a 5- or 6-membered heterocyclic ring containing 1, 2, or 3 heteroatoms selected from N, S, and O optionally substituted independently by one or more R₁₃; or R₆ and R₇, together with the nitrogen to which they are attached, form a 4-, 5-, or 6-membered heterocyclic ring optionally substituted independently by one or more R₁₃; or a 5- or 6-membered heterocyclic ring containing 1, 2, or 3 heteroatoms selected from N, S, and O optionally substituted independently by one or more R₁₃; where R₁₃ is hydroxy, C₁₋₄ alkoxy, F, C₁₋₆ alkyl, haloalkyl, haloalkoxy, —NH₂, —NH(C₁₋₆ alkyl), or —N(C₁₋₆ alkyl)₂—;

or compounds having the formula:

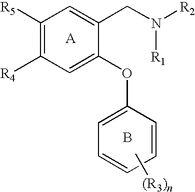

where R₁ and R₂ are independently H, C₁₋₆ alkyl (e.g., CH₃) or substituted heteroalkyl, (CH₂)ₘ(C₃₋₆ cycloalkyl) where m is 0, 1, 2, or 3, or R₁ and R₂ together with the nitrogen to which they are attached form an azetidine ring; each R₃ is independently H, I, Br, F, Cl, C₁₋₆ alkyl (e.g., CH₃), CF₃, CN, OCF₃, C₁₋₄ alkylthio (e.g., SCH₃), C₁₋₄ alkoxy (e.g., OCH₃), aryloxy (e.g., OPh), or CONR₆R₇; n is 1, 2, or 3; and R₄ and R₅ are independently A-X, where A is —CH═CH— or —(CH₂)ₚ— where p is 0, 1, or 2; X is H, F, Cl, Br, I, CONR₆R₇, SO₂NR₆R₇, SO₂NHC(═O)R₆, OH, C₁₋₄ alkoxy, NR₈SO₂R₉, NO₂, NR₆R₁₁, CN, CO₂R₁₀ (e.g., COOH), CHO, SR₁₀, S(O)R₉, or SO₂R₁₀; R₆, R₇, R₈, and R₁₀ are independently H or C₁₋₆ alkyl (e.g., (CH₂)₃CH₃ or cyclopropyl), C₆₋₁₂ aryl (e.g., phenyl) optionally substituted independently by one or more R₁₂, or C₁₋₆ alkyl-aryl optionally substituted; R₉ is C₁₋₆ alkyl optionally substituted independently by one or more R₁₂; R₁₁ is H, C₁₋₆ alkyl optionally substituted independently by one or more R₁₂, C(O)R₆, CO₂R₉, C(O)NHR₆, or SO₂NR₆R₇; R₁₂ is F (preferably up to 3), OH, CO₂H, C₃₋₆ cycloalkyl, NH₂, CONH₂, C₁₋₆ alkoxy, C₁₋₆ alkoxycarbonyl or a 5- or 6-membered heterocyclic ring containing 1, 2, or 3 heteroatoms selected from N, S, and O optionally substituted independently by one or more R₁₃; or R₆ and R₇, together with the nitrogen to which they are attached, form a 4-, 5-, or 6-membered heterocyclic ring optionally substituted independently by one or more R₁₃; or a 5- or 6-membered heterocyclic ring containing 1, 2, or 3 heteroatoms selected from N, S, and O optionally substituted independently by one or more R₁₃; where R₁₃ is hydroxy, C₁₋₄ alkoxy, F, C₁₋₆ alkyl, haloalkyl, haloalkoxy, —NH₂, —NH(C₁₋₆ alkyl) or —N(C₁₋₆ alkyl)₂ (e.g., where when R₁ and R₂ are methyl, R₄ and R₅ are hydrogen and n is 1, R₃ is not a —SMe group para to the ether linkage linking rings A and B). In certain embodiments, n is 1 or 2, and the R₃ group(s) is/are at positions 3 and/or 4 of the B ring, for example, are CH₃, SCH₃, OCH₃, Br, or CF₃. For either of the above structures, R₄ or R₅ can be SO₂NHPh, SO₂NHCH₃, CN, H, Br, CONH₂, COOH, SO₂NHCH₂Ph, SO₂NHCOCH₃, CH₂NHSO₂CH₃ NH₂, ORNO₂, benzyl amide, acylsulfonamide, reverse sulfonamide, NHCH₃, N(CH₃)₂, SO₂NH₂, CH₂OH, NHSO₂CH₃, SO₂NHCH₂CCH₂, CH₂NH₂, SO₂NHBu, and SO₂NHcyclopropyl. UK-416244 structural analogs are described in U.S. Pat. Nos. 6,448,293 and 6,610,747. UK-416244 analogs are described below.

Other analogs of UK-416244 can be described by the formula:

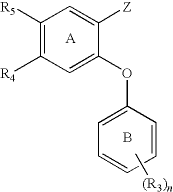

where are R₃, R₄, and R₅ are as defined above and Z is CH₂NR₁R₂ where R₁ and R₂ are as defined above, NH₂, optionally substituted optionally hetero C₁₋₈ alkyl (e.g., substituted with hydroxyl, NH₂, NHC₁₋₆ alkyl), or is selected from the group consisting of:

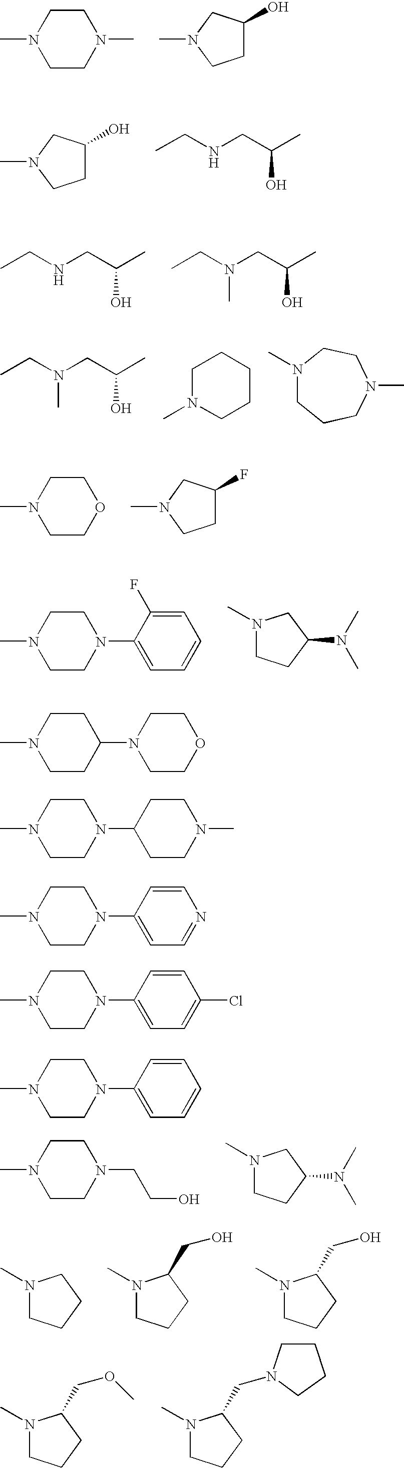

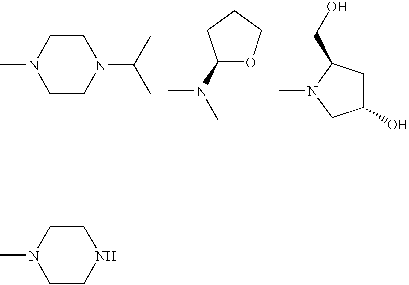

In certain embodiments, Z is CN, CH₂CH(CH₃)₂, CH₂OCH₃, CH₂N(CH₃)CH₂CH₂OH, N(CH₃)₂, CH₂N(CH₃)₂, COOH, CH₂NHCH₃, CH₂OH, CH₂NHCOCH₃, CONHCH₃, CH₂NH(CH₂)₂N(CH₃)₂, CH₂NH(CH₂)₃N(CH₃)₂, CHC(CH₃)₂, CH₂N(CH₃)(CH₂)₂N(CH₃)₂, CH₂N(CH₃)(CH₂)₃N(CH₃)₂, or CH₂CH(CH₃)₂.

Other UK-416244 analogs are described by the formula.

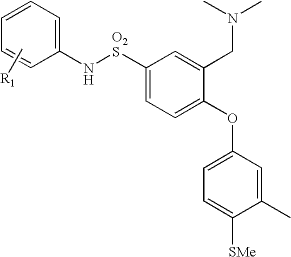

where R₁ is H, I, Br, F, Cl, C₁₋₆ alkyl (e.g., CH₃), CF₃, CN, OCF₃, C₁₋₄ alkylthio (e.g., SCH₃), C₁₋₄ alkoxy (e.g., OCH₃), aryloxy, or CONR₂R₃; n is 1, 2, or 3; R₂ and R₃ are independently H or C₁₋₆ alkyl (e.g., (CH₂)₃CH₃ or cyclopropyl), C₆₋₁₂ aryl (e.g., phenyl) optionally substituted independently by one or more R₄, or C₁₋₆ alkyl-aryl optionally substituted; R₄ is F (preferably up to 3), OH, CO₂H, C₃₋₆ cycloalkyl, NH₂, CONH₂, C₁₋₆ alkoxy, C₁₋₆ alkoxycarbonyl or a 5- or 6-membered heterocyclic ring containing 1, 2, or 3 heteroatoms selected from N, S, and O optionally substituted independently by one or more R₅; or R₂ and R₃, together with the nitrogen to which they are attached, form a 4-, 5-, or 6-membered heterocyclic ring optionally substituted independently by one or more R₅; or a 5- or 6-membered heterocyclic ring containing 1, 2, or 3 heteroatoms selected from N, S, and O optionally substituted independently by one or more R₅; where R₅ is hydroxy, C₁₋₄ alkoxy, F, C₁₋₆ alkyl, haloalkyl, haloalkoxy, —NH₂, —NH(C₁₋₆ alkyl) or —N(C₁₋₆ alkyl)₂. In certain embodiments, where R₁ is Br, OMe, NO₂, CO₂Me, or CN. R₁ may be at the ortho, meta, or para position)

Still other UK-416244 analogs are described by the formula:

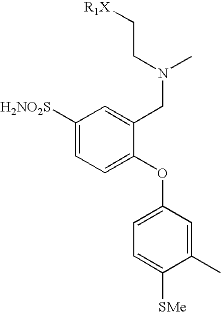

where X is N, O, or S, and R₁ is H, C₁₋₆ alkyl or substituted heteroalkyl, (CH₂)ₘ(C₃₋₆ cycloalkyl) where m is 0, 1, 2, or 3.

Additional compounds have the structure:

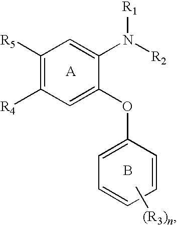

where R₁ is H or C₁₋₆ alkyl (e.g., CH₃, CH₂CH₃) and R₂ is C₁₋₆ alkyl substituted with OH, such as CH₂OH, CH₂CH₂OH, CH(OH)CH₃, CH₂CH₂CH₂OH, CH(CH₂)CH₂OH, and CH₂CH₂CH₂CH₂OH, CH(OH)CH₂CH₂CH₃, CH₂CH(OH)CH₂CH₃, and CH₂CH₂CH(OH)CH₃) or is CH₂XR₁₄ or CH₂CH₂XR₁₄, where X is N, O, or S, and R₁₄ is H, C₁₋₆ alkyl or substituted heteroalkyl, (CH₂)_{q}(C₃₋₆ cycloalkyl) where q is 0, 1, 2, or 3, and where R₃, R₄, and R₅ are as defined above. In certain embodiments, the compound has the structure,

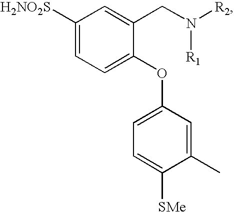

where R₁ is H or C₁₋₆ alkyl (e.g., CH₃, CH₂CH₃) and R₂ is C₁₋₆ alkyl substituted with OH, e.g., CH₂OH, CH₂CH₂OH, CH(OH)CH₃, CH₂CH(OH)CH₃, CH₂CH₂CH₂OH, CH(CH₂)CH₂OH, and CH₂CH₂CH₂CH₂OH, CH(OH)CH₂CH₂CH₃, CH₂CH(OH)CH₂CH₃, and CH₂CH₂CH(OH)CH₃). In particular embodiments, the compound is:

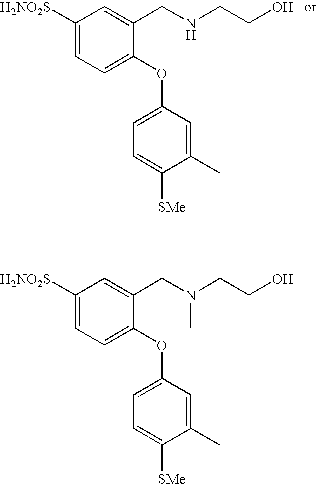

In any of the UK-416244 analogs, the bridge between the A and B rings may be replaced with an —NH— bridge (e.g., Compound 108).

Particular UK-416244 analogs include those of Table 10:

TABLE 10

| | |
|---|---|
| Compound 1 | 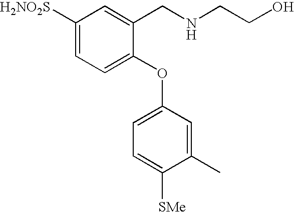 |
| Compound 2 | 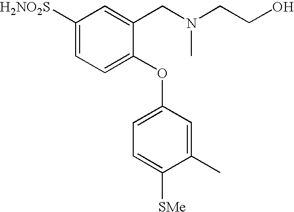 |
| Compound 3 | 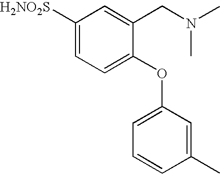 |
| Compound 4 | 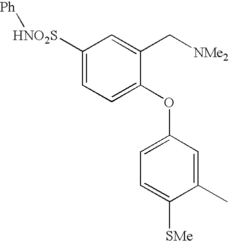 |
| Compound 5 | 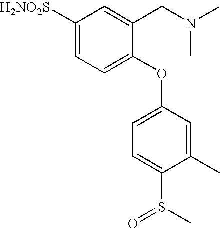 |

Other UK-416244 analogs include those of Table 11.

TABLE 11

| | |
|---|---|
| | Compound 6 |
| 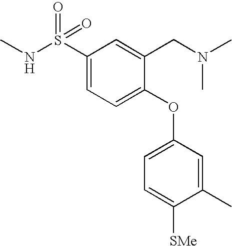 | |
| | Compound 7 |
| 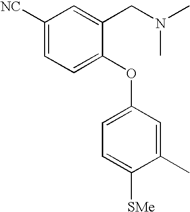 | |
| | Compound 8 |
| 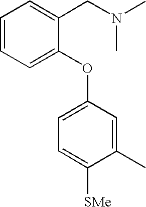 | |
| | Compound 9 |
| 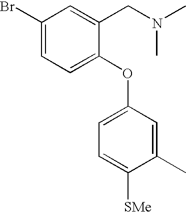 | |

| | Compound 10 |
|---|---|
| 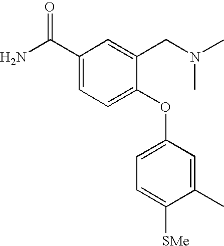 | |
| | Compound 11 |
| 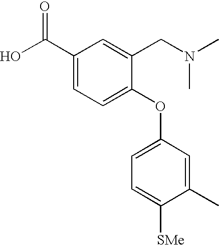 | |
| | Compound 12 |
| 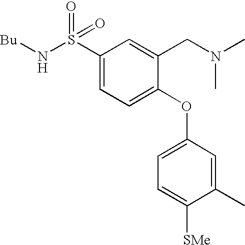 | |
| | Compound 13 |
| 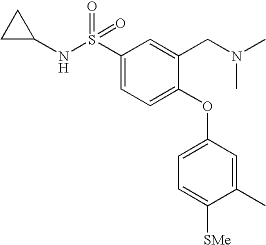 | |
| | Compound 14 |
| 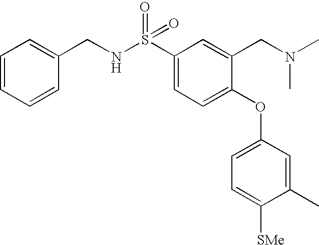 | |
| | Compound 15 |
| 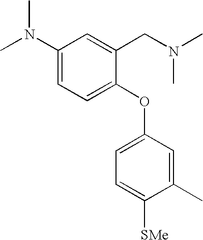 | |
| | Compound 16 |
| 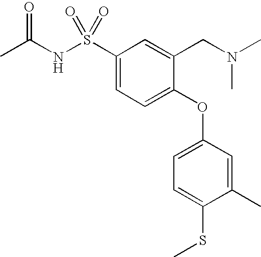 | |
| | Compound 17 |
| 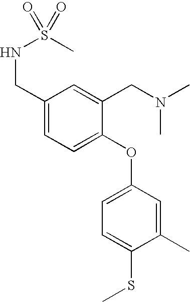 | |
| | Compound 18 |
| 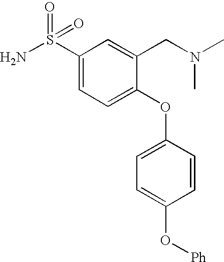 | |
| | Compound 19 |
| 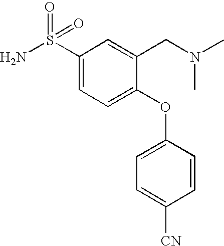 | |
| | Compound 20 |
| 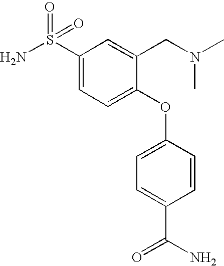 | |
| | Compound 21 |
| 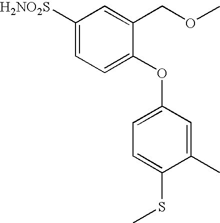 | |
| | Compound 22 |
| 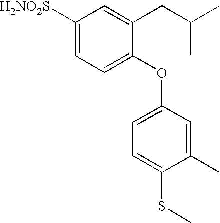 | |
| | Compound 23 |
| 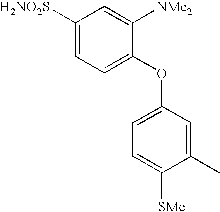 | |
| | Compound 24 |
| 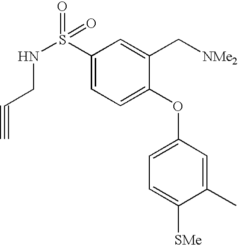 | |
| | Compound 25 |
| 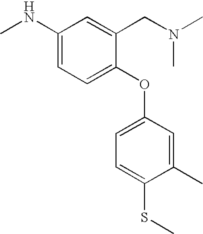 | |
| | Compound 26 |
| 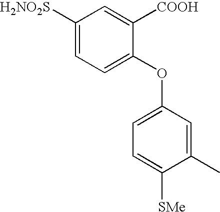 | |
| | Compound 27 |
| 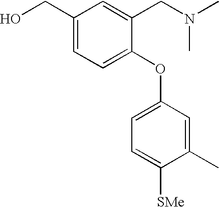 | |
| | Compound 28 |
| 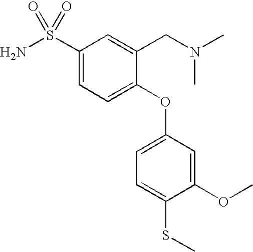 | |
| | Compound 29 |
| 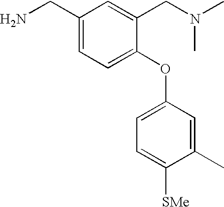 | |
| | Compound 30 |
| 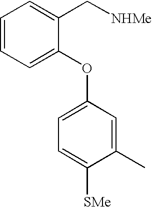 | |
| | Compound 31 |
| 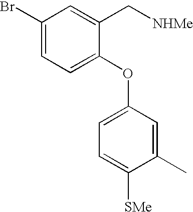 | |
| | Compound 32 |
| 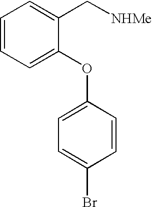 | |
| | Compound 33 |
| 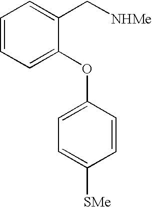 | |
| | Compound 34 |
| 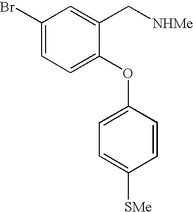 | |
| | Compound 35 |
| 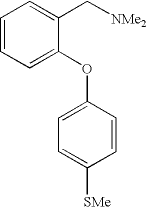 | |
| | Compound 36 |
| 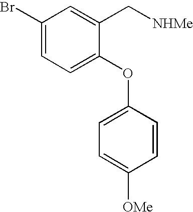 | |
| | Compound 37 |
| 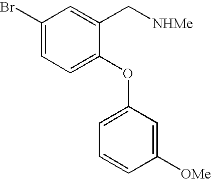 | |
| | Compound 38 |
| 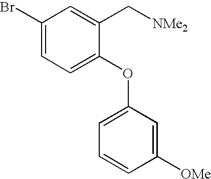 | |
| | Compound 39 |
| 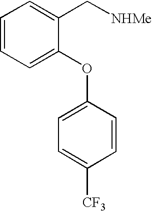 | |
| | Compound 40 |
| 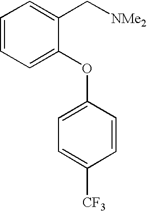 | |
| | Compound 41 |
| 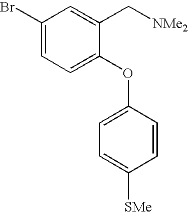 | |
| | Compound 42 |
| 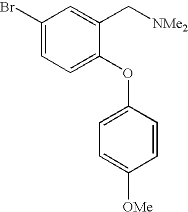 | |
| | Compound 43 |
| 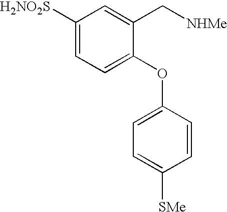 | |
| | Compound 44 |
| 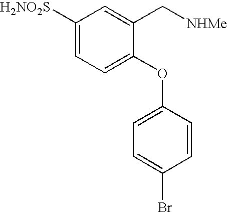 | |
| | Compound 45 |
| 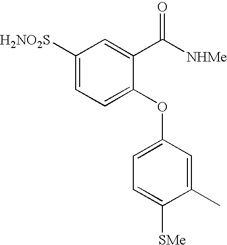 | |
| | Compound 46 |
| 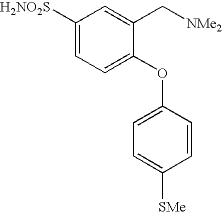 | |
| | Compound 47 |
| 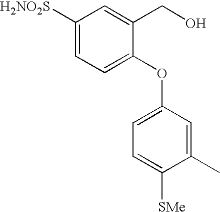 | |
| | Compound 48 |
| 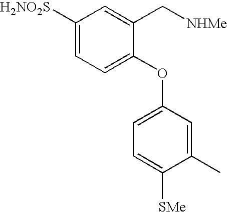 | |
| | Compound 49 |
| 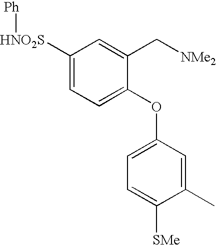 | |
| | Compound 50 |
| 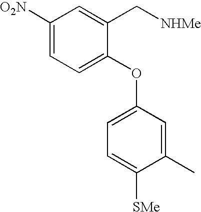 | |
| | Compound 51 |
| 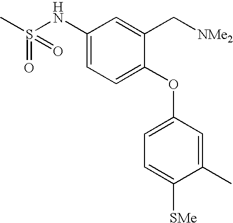 | |
| | Compound 52 |
| 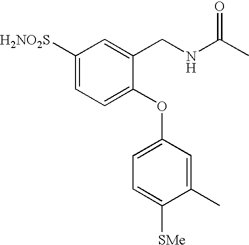 | |
| | Compound 53 |
| 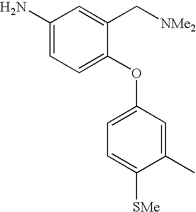 | |
| | Compound 54 |
| 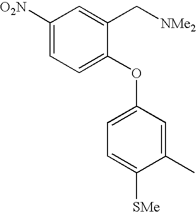 | |
| | Compound 55 |
| 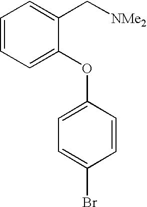 | |
| | Compound 56 |
| 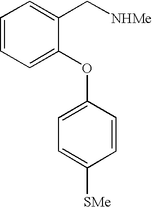 | |
| | Compound 57 |
| 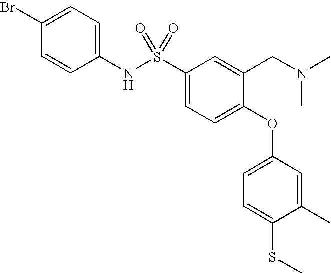 | |
| | Compound 58 |
| 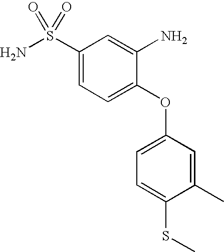 | |
| | Compound 59 |
| 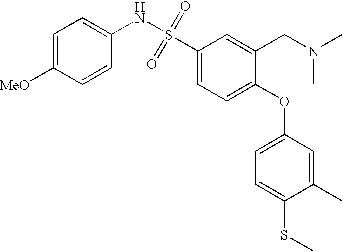 | |
| | Compound 60 |
| 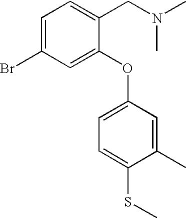 | |
| | Compound 61 |
| 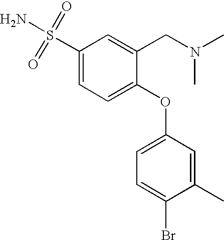 | |
| | Compound 62 |
| 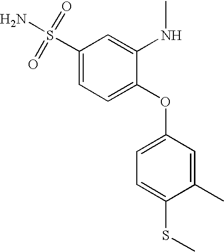 | |
| | Compound 63 |
| 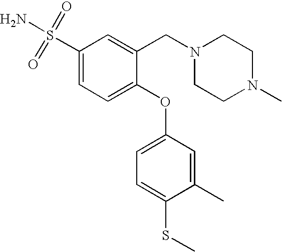 | |
| | Compound 64 |
| 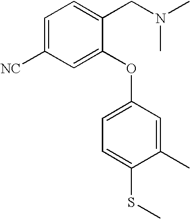 | |
| | Compound 65 |
| 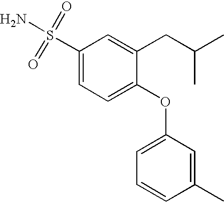 | |
| | Compound 66 |
| 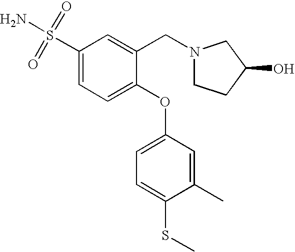 | |
| | Compound 67 |
| 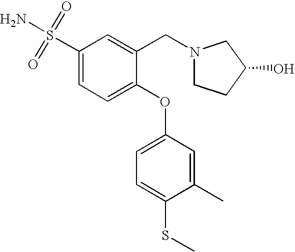 | |
| | Compound 68 |
| 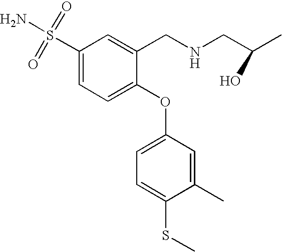 | |
| | Compound 69 |
| 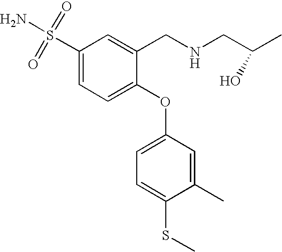 | |
| | Compound 70 |
| 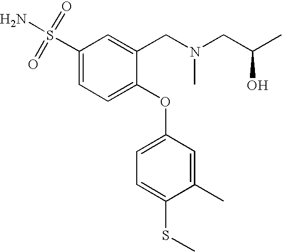 | |
| | Compound 71 |
| 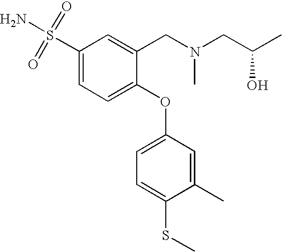 | |
| | Compound 72 |
| 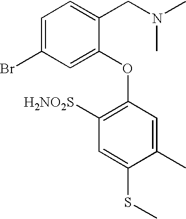 | |
| | Compound 73 |
| 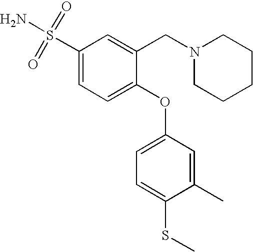 | |
| | Compound 74 |
| 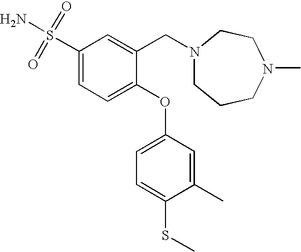 | |
| | Compound 75 |
| 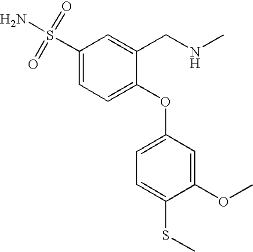 | |
| | Compound 76 |
| 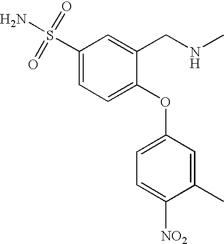 | |
| | Compound 77 |
| 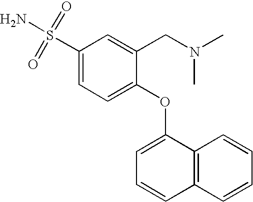 | |
| | Compound 78 |
| 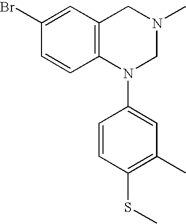 | |
| | Compound 79 |
| 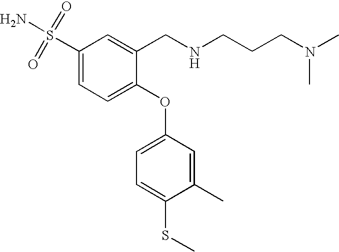 | |
| | Compound 80 |
| 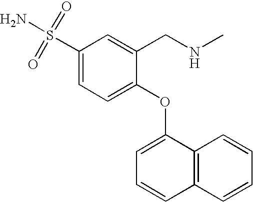 | |
| | Compound 81 |
| 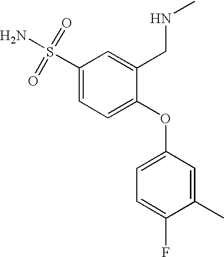 | |
| | Compound 82 |
| 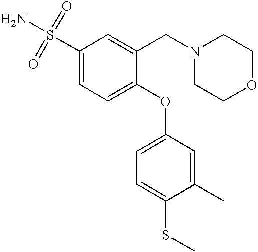 | |
| | Compound 83 |
| 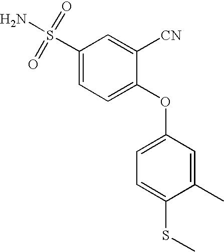 | |
| | Compound 84 |
| 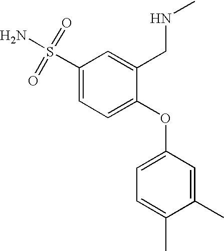 | |
| | Compound 85 |
| 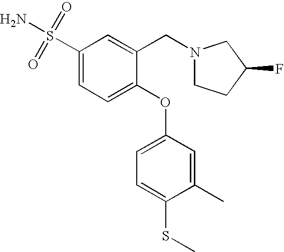 | |
| | Compound 86 |
| 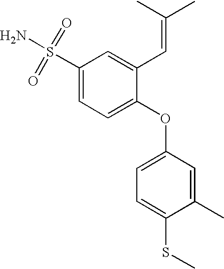 | |
| | Compound 87 |
| 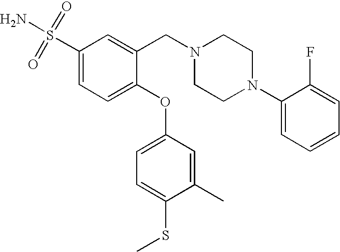 | |
| | Compound 88 |
| 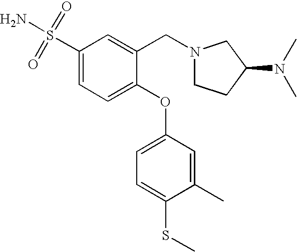 | |
| | Compound 89 |
| 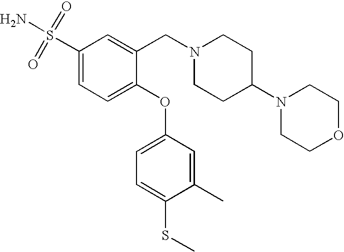 | |
| | Compound 90 |

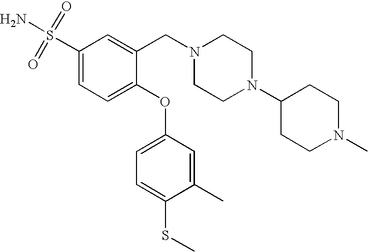

| | Compound 91 |
|---|---|
| 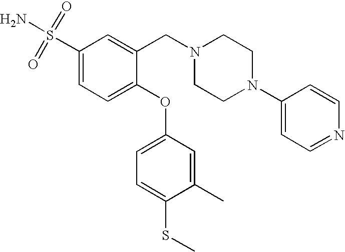 | |
| | Compound 92 |

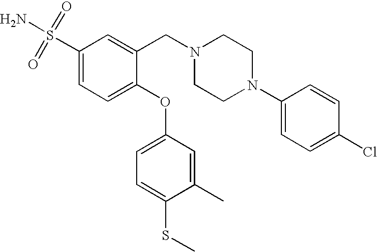

| | Compound 93 |
|---|---|
| 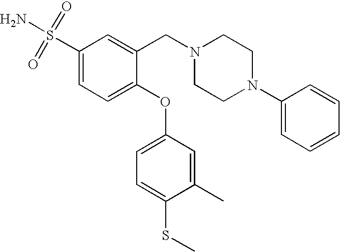 | |
| | Compound 94 |
| 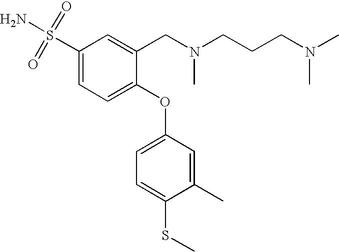 | |
| | Compound 95 |

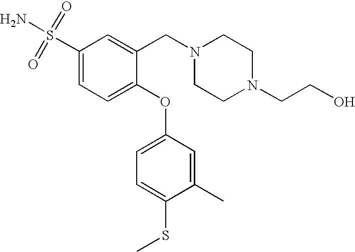

| | |
|---|---|
| | Compound 96 |
| 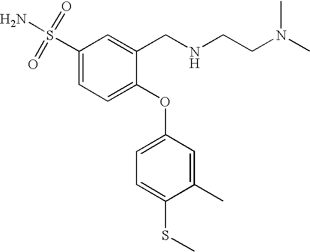 | |
| | Compound 97 |
| 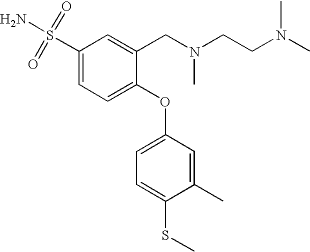 | |
| | Compound 98 |
| 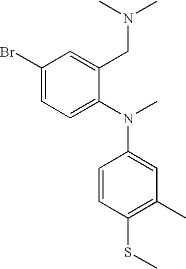 | |
| | Compound 99 |
| 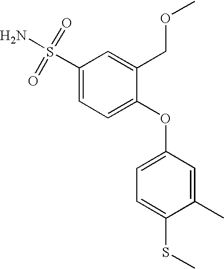 | |
| | Compound 100 |
| 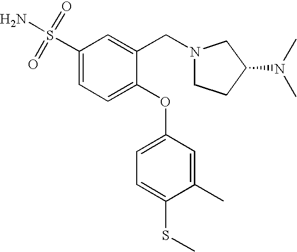 | |
| | Compound 101 |
| 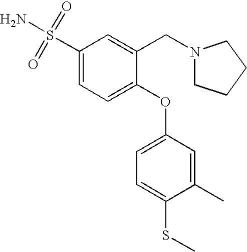 | |
| | Compound 102 |
| 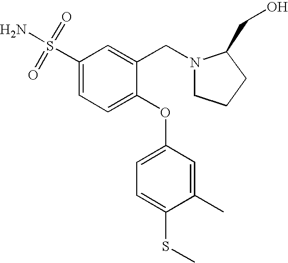 | |
| | Compound 103 |
| 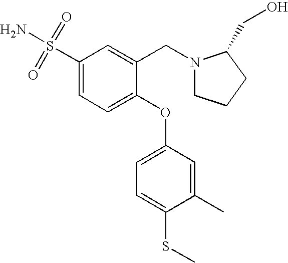 | |
| | Compound 104 |
| 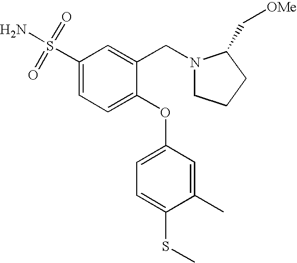 | |
| | Compound 105 |
| 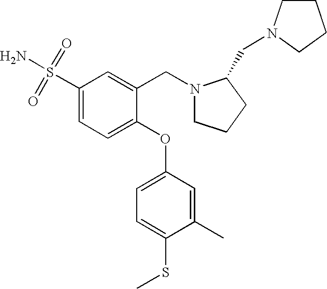 | |
| | Compound 106 |
| 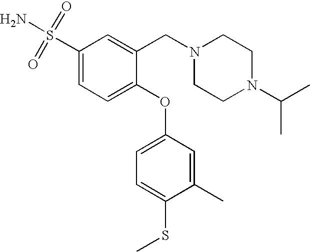 | |
| | Compound 107 |
| 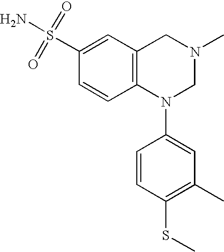 | |
| | Compound 108 |
| 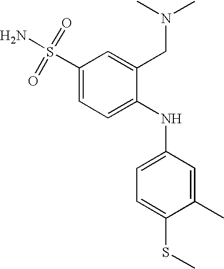 | |
| | Compound 109 |
| 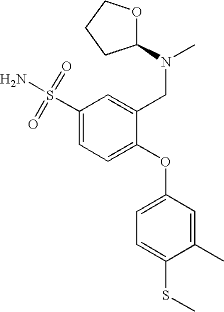 | |
| | Compound 110 |
| 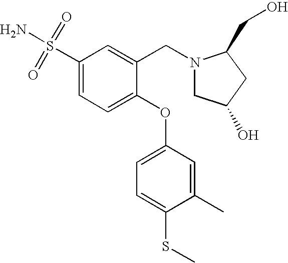 | |
| | Compound 111 |
| 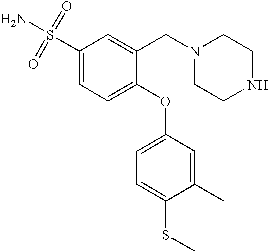 | |

### Corticosteroids

In certain embodiments, a corticosteroid can be used in the compositions, methods, and kits of the invention. If desired, one or more corticosteroid may be administered in a method of the invention or may be formulated with a tricyclic compound in a composition of the invention. Suitable corticosteroids include 11-alpha,17-alpha,21-trihydroxypregn-4-ene-3,20-dione; 11-beta,16-alpha,17,21-tetrahydroxypregn-4-ene-3,20-dione; 11-beta,16-alpha,17,21-tetrahydroxypregn-1,4-diene-3,20-dione; 11-beta,17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta,21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha,9-alpha-difluoroprednisolone 21′-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide (analogs described in U.S. Pat. No. 3,929,768); calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone (analogs described in 4,472,393); mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin or derivatives thereof (see, e.g., U.S. Pat. No. 7,081,475).

Steroid Receptor Modulators

Steroid receptor modulators (e.g., antagonists and agonists) may be used as a substitute for or in addition to a corticosteroid in the compositions, methods, and kits of the invention.

Glucocorticoid receptor modulators that may used in the compositions, methods, and kits of the invention include compounds described in U.S. Pat. Nos. 6,380,207, 6,380,223, 6,448,405, 6,506,766, and 6,570,020, U.S. Pat. Application Publication Nos. 2003/0176478, 2003/0171585, 2003/0120081, 2003/0073703, 2002/015631, 2002/0147336, 2002/0107235, 2002/0103217, and 2001/0041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

### Bufexamac

In certain embodiments, bufexamac or a bufexamac analog can be used in the compositions, methods, and kits of the invention. By “bufexamac analog” is meant a compound having the formula (VI):

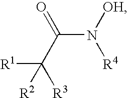

wherein R¹ is

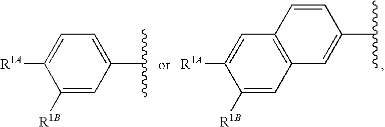

wherein R^{1A} is and R^{1B} is H, halo, CF₃, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₁₋₆ alkoxy, or optionally substituted C₁₋₆ thioalkoxy; each of R² and R³ is, independently, H, C₁₋₄ alkyl, or CF₃; and R⁴ is optionally substituted C₁₋₆ alkyl or optionally substituted C₃₋₈ cycloalkyl.

### Antiviral Agents

In certain embodiments, an antiviral agent can be used in the compositions, methods, and kits of the invention. Suitable antiviral agents include, without limitation, abacavir, acemannan, acyclovir, adefovir, amantadine, amidinomycin, ampligen, amprenavir, aphidicolin, atevirdine, capravirine cidofovir, cytarabine, delavirdine, didanosine, dideoxyadenosine, n-docosanol, edoxudine, efavirenz, emtricitabine, famciclovir, floxuridine, fomivirsen, foscamet sodium, ganciclovir, idoxuridine, imiquimod, indinavir, inosine pranobex, interferon-α, interferon-β, kethoxal, lamivudine, lopinavir, lysozyme, madu, methisazone, moroxydine, nelfinavir, nevirapine, nitazoxanide, oseltamivir, palivizumab, penciclovir, enfuvirtide, pleconaril, podophyllotoxin, ribavirin, rimantadine, ritonavir, saquinavir, sorivudine, stallimycin, statolon, stavudine, tenofovir, tremacamra, triciribine, trifluridine, tromantadine, tunicamycin, valacyclovir, valganciclovir, vidarabine, zalcitabine, zanamivir, zidovudine, resiquimod, atazanavir, tipranavir, entecavir, fosamprenavir, merimepodib, docosanol, vx-950, and peg interferon. Additional antiviral agents are listed in Table 4 and Table 5.

Structural analogs of antiviral agents which may be used in the combinations of the invention include 9-((2-aminoethoxy)methyl)guanine, 8-hydroxyacyclovir, 2′-O-glycyl acyclovir, ganciclovir, PD 116124, valacyclovir, omaciclovir, valganciclovir, buciclovir, penciclovir, valmaciclovir, carbovir, theophylline, xanthine, 3-methylguanine, enprofylline, cafaminol, 7-methylxanthine, L 653180, BMS 181164, valomaciclovir stearate, deriphyllin, acyclovir monophosphate, acyclovir diphosphate dimyristoylglycerol, and etofylline.

Edoxudine analogs are described in U.S. Pat. No. 3,553,192. Efavirenz analogs are described in European Patent 582,455 and U.S. Pat. No. 5,519,021. Floxuridine analogs are described in U.S. Pat. Nos. 2,970,139 and 2,949,451. Nelfinavir analogs are described in U.S. Pat. No. 5,484,926. Aphidicolin analogs are described in U.S. Pat. No. 3,761,512. Trifluridine analogs are described in U.S. Pat. No. 3,201,387. Cytarabine analogs are described in U.S. Pat. No. 3,116,282. Triciribine analogs, including triciribine 5′-phosphate and triciribine-dimethylformamide, are described in U.S. Pat. No. 5,633,235. Nitazoxanide analogs are described in U.S. Pat. No. 3,950,391.

Ritonavir

Ritonavir is an antiviral used in treatment of HIV and has the structure:

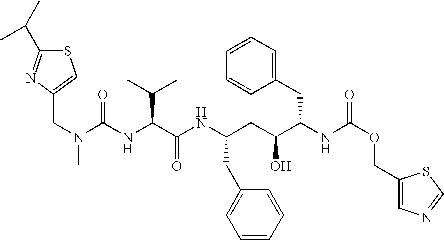

Ritonavir analogs are described, for example, in U.S. Pat. No. 5,541,206 and have the general structure:

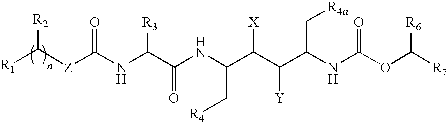

where R₁ is monosubstituted thiazolyl, monosubstituted oxazolyl, monosubstituted isoxazolyl or monosubstituted isothiazolyl wherein the substituent is selected from (i) loweralkyl, (ii) loweralkenyl, (iii) cycloalkyl, (iv) cycloalkylalkyl, (v) cycloalkenyl, (vi) cycloalkenylalkyl, (vii) heterocyclic wherein the heterocyclic is selected from aziridinyl, azetidinyl, pyrrolidinyl, piperidinyl, piperazinyl, morpholinyl, thiomorpholinyl, thiazolyl, oxazolyl, isoxazolyl, isothiazolyl, pyridinyl, pyrimidinyl, pyridazinyl and pyrazinyl and wherein the heterocyclic is unsubstituted or substituted with a substituent selected from halo, loweralkyl, hydroxy, alkoxy and thioalkoxy, (viii) (heterocyclic)alkyl wherein heterocyclic is defined as above, (ix) alkoxyalkyl, (x) thioalkoxyalkyl, (xi) alkylamino, (xii) dialkylamino, (xiii) phenyl wherein the phenyl ring is unsubstituted or substituted with a substituent selected from halo, loweralkyl, hydroxy, alkoxy and thioalkoxy, (xiv) phenylalkyl wherein the phenyl ring is unsubstituted or substituted as defined above, (xv) dialkylaminoalkyl, (xvi) alkoxy and (xvii) thioalkoxy; n is 1, 2 or 3; R₂ is hydrogen or loweralkyl; R₃ is loweralkyl; R₄ and R₄ₐ are independently selected from phenyl, thiazolyl and oxazolyl wherein the phenyl, thiazolyl or oxazolyl ring is unsubstituted or substituted with a substituent selected from (i) halo, (ii) loweralkyl, (iii) hydroxy, (iv) alkoxy and (v) thioalkoxy; R₆ is hydrogen or loweralkyl; R₇ is thiazolyl, oxazolyl, isoxazolyl or isothiazolyl wherein the thiazolyl, oxazolyl, isoxazolyl or isothiazolyl ring is unsubstituted or substituted with loweralkyl; X is hydrogen and Y is —OH or X is —OH and Y is hydrogen, with the proviso that X is hydrogen and Y is —OH when Z is —N(R₈)— and R₇ is unsubstituted and with the proviso that X is hydrogen and Y is —OH when R₃ is methyl and R₇ is unsubstituted; and Z is absent, —O—, —S—, —CH₂— or —N(R₈)— wherein R₈ is loweralkyl, cycloalkyl, —OH or —NHR₈ₐ wherein R₈ₐ is hydrogen, loweralkyl or an N-protecting group.

Saquinavir

In certain embodiments, saquinavir or its analogs can be used in the compositions, methods, and kits of the invention. Saquinavir is a protease inhibitor that is highly specific for the HIV-1 and HIV-2 proteases. The structure of saquinavir is:

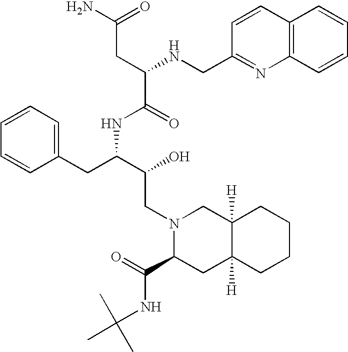

Saquinavir analogs are described, for example, in U.S. Pat. No. 5,196,438 and have the general structure:

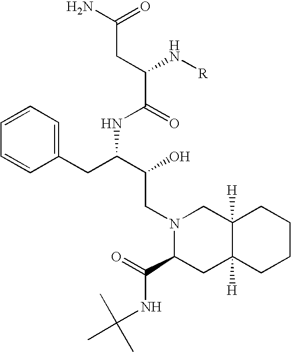

where R is benzyloxycarbonyl or 2-quinolylcarbonyl, and pharmaceutically acceptable acid addition salts thereof.

Adefovir Dipivoxil

In certain embodiments, adefovir dipivoxil or its analogs can be used in the compositions, methods, and kits of the invention. Analogs of adefovir dipivoxil are described, for example, in U.S. Pat. No. 4,808,716 and include compounds with the general structure:

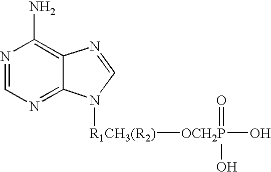

wherein R₁ is a hydrogen atom, an alkyl group containing one to three carbon atoms, or a hydroxymethyl group, and R₂ is a methylene, ethylene, propylene, ethylidene, methoxyethylene, benzyloxyethylene, tetrahydropyran-2-yloxyethylene, (1-ethoxyethoxy)ethylene, or 1,2-O-isopropylidene-1,2-dihydroxypropylene group.

Celgosivir

In certain embodiments, celgosivir or an analog thereof can be used in the compositions, methods, and kits of the invention. Celgosivir is a prodrug of castanospermine, a natural product derived from the Australian Black Bean chestnut tree. It has antiviral (e.g., anti-HCV) activity, and acts as an inhibitor of α- and β-glucosidase. The structure of celgosivir is:

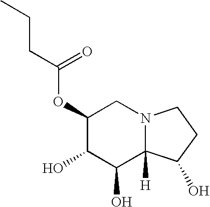

Analogs of celgosivir are described, for example, in PCT Publication No. WO 2006/096285 and have the general structure:

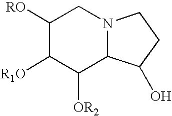

where R, R₁ and R₂ are independently hydrogen, C₁₋₄ alkanoyl, C₂₋₁₄ alkenoyl, cyclohexanecarbonyl, C₁₋₈ alkoxyacetyl,

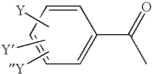

naphthalenecarbonyl optionally substituted by methyl or halogen; phenyl(C₂₋₆ alkanoyl) wherein the phenyl is optionally substituted by methyl or halogen; cinnamoyl; pyridinecarbonyl optionally substituted by methyl or halogen; dihydropyridine carbonyl optionally substituted by C₁₋₁₀ alkyl; thiophenecarbonyl optionally substituted by methyl or halogen; or furancarbonyl optionally substituted by methyl or halogen; Y is hydrogen, C₁₋₄ alkyl, C₁₋₄ alkoxy, halogen, trifluoromethyl, C₁₋₄ alkylsulphonyl, C₁₋₄ alkylmercapto, cyano or dimethylamino; Y′ is hydrogen, C₁₋₄ alkyl, C₁₋₄ alkoxy, halogen or it is combined with Y to give 3,4-methylenedioxy; Y″ is hydrogen, C₁₋₄ alkyl, C₁₋₄ alkoxy or halogen; and pharmaceutically acceptable salts thereof.

### Nonsteroidal Immunophilin-Dependent Immunosuppressants

In certain embodiments, a nonsteroidal immunophilin-dependent immunosuppressant can be used in the compositions, methods, and kits of the invention. Suitable NsIDIs include cyclosporine, tacrolimus, rapamycin (sirolimus), everolimus, and pimecrolimus.

Cyclosporines

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T cell-dependent immune response by inhibiting antigen-triggered signal transduction. This inhibition decreases the expression of proinflammatory cytokines, such as IL-2.

Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Pat. Application Publication No. 2002/0132763 A1). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH—Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂—OH)-8-Cs, and D-Ser-8-Cs, which are described in Cruz et al. (Antimicrob. Agents Chemother. 44:143-149, 2000).

Tacrolimus

Tacrolimus and tacrolimus analogs are described by Tanaka et al., (J. Am. Chem. Soc., 109:5031, 1987) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus

Pimecrolimus is the 33-epi-chloro derivative of the macrolactam ascomyin. Pimecrolimus structural and functional analogs are described in U.S. Pat. No. 6,384,073.

Rapamycin

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

### Peptide Moieties

Peptides, peptide mimetics, peptide fragments, either natural, synthetic or chemically modified, that impair the calcineurin-mediated dephosphorylation and nuclear translocation of NFAT are suitable for use in practicing the invention. Examples of peptides that act as calcineurin inhibitors by inhibiting the NFAT activation and the NFAT transcription factor are described, e.g., by Aramburu et al., Science 285:2129-2133, 1999) and Aramburu et al., Mol. Cell. 1:627-637, 1998). As a class of calcineurin inhibitors, these agents are useful in the methods of the invention.

### Antihistamines

In certain embodiments, an antihistamine or an antihistamine analog can be used in the compositions, methods, and kits of the invention. Antihistamines are compounds that block the action of histamine. Classes of antihistamines include:

(1) Ethanolamines (e.g., bromodiphenhydramine, carbinoxamine, clemastine, dimenhydrinate, diphenhydramine, diphenylpyraline, and doxylamine);

(2) Ethylenediamines (e.g., pheniramine, pyrilamine, tripelennamine, and triprolidine);

(3) Phenothiazines (e.g., diethazine, ethopropazine, methdilazine, promethazine, thiethylperazine, and trimeprazine);

(4) Alkylamines (e.g., acrivastine, brompheniramine, chlorpheniramine, desbrompheniramine, dexchlorpheniramine, pyrrobutamine, and triprolidine);

(5) piperazines (e.g., buclizine, cetirizine, chlorcyclizine, cyclizine, meclizine, hydroxyzine);

(6) Piperidines (e.g., astemizole, azatadine, cyproheptadine, desloratadine, fexofenadine, loratadine, ketotifen, olopatadine, phenindamine, and terfenadine);

(7) Atypical antihistamines (e.g., azelastine, levocabastine, methapyrilene, and phenyltoxamine).

In the compositions, methods, and kits of the invention, both non-sedating and sedating antihistamines may be employed. Non-sedating antihistamines include loratadine and desloratadine. Sedating antihistamines include azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other antihistamines suitable for use in the compositions, methods, and kits of the invention are acrivastine; ahistan; antazoline; astemizole; azelastine (e.g., azelsatine hydrochloride); bamipine; bepotastine; benztropine, bietanautine; brompheniramine (e.g., brompheniramine maleate); carbinoxamine (e.g., carbinoxamine maleate); cetirizine (e.g., cetirizine hydrochloride); cetoxime; chlorocyclizine; chloropyramine; chlorothen; chlorphenoxamine; cinnarizine; clemastine (e.g., clemastine fumarate); clobenzepam; clobenztropine; clocinizine; cyclizine (e.g., cyclizine hydrochloride; cyclizine lactate); deptropine; dexchlorpheniramine; dexchlorpheniramine maleate; diphenylpyraline; doxepin; ebastine; embramine; emedastine (e.g., emedastine difumarate); epinastine; etymemazine hydrochloride; fexofenadine (e.g., fexofenadine hydrochloride); histapyrrodine; hydroxyzine (e.g., hydroxyzine hydrochloride; hydroxyzine pamoate); isopromethazine; isothipendyl; levocabastine (e.g., levocabastine hydrochloride); mebhydroline; mequitazine; methafurylene; methapyrilene; metron; mizolastine; olapatadine (e.g., olopatadine hydrochloride); orphenadrine; phenindamine (e.g., phenindamine tartrate); pheniramine; phenyltoloxamine; p-methyldiphenhydramine; pyrrobutamine; setastine; talastine; terfenadine; thenyldiamine; thiazinamium (e.g., thiazinamium methylsulfate); thonzylamine hydrochloride; tolpropamine; triprolidine; and tritoqualine.

Antihistamine analogs may also be used in according to the invention. Antihistamine analogs include 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; SCH 37370; SCH 434; tecastemizole; thiazinamium; thiopropazate; thioridazine (e.g., thioridazine hydrochloride); and 3-(10,11-dihydro-5H-dibenzo(a,d)cyclohepten-5-ylidene)-tropane.

Other compounds that are suitable for use in the invention are AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that are suitable for use in the invention are described in U.S. Pat. Nos. 2,595,405, 2,709,169, 2,785,202, 2,899,436, 3,014,911, 3,813,384, 3,956,296, 4,254,129, 4,254,130, 4,282,833, 4,283,408, 4,362,736, 4,394,508, 4,285,957, 4,285,958, 4,440,933, 4,510,309, 4,550,116, 4,659,716, 4,692,456, 4,742,175, 4,833,138, 4,908,372, 5,204,249, 5,375,693, 5,578,610, 5,581,011, 5,589,487, 5,663,412, 5,994,549, 6,201,124, and 6,458,958.

Hydroxyzine

In certain embodiments, hydroxyzine or an analog thereof can be used in the compositions, methods, and kits of the invention. The structure of hydroxyzine is:

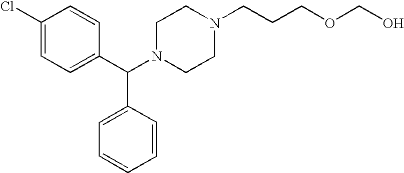

Analogs of hydroxyzine are described, for example, in U.S. Pat. No. 2,899,436 and have the general structure:

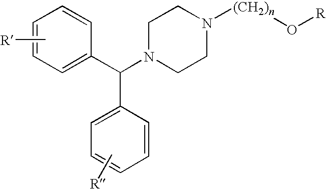

wherein R′ and R″ are a hydrogen atom, a halogen atom, an alkyl group, or an alkoxy group, R′ and R″ being in ortho, meta, or para positions; R contains 2 to 11 carbon atoms and is alkyl, phenyl, alkyl substituted phenyl, aralkyl, cycloalkyl, hydroxyalkyl, hydroxycycloalkyl or —CH₂—CH₂—O—CH₂—CH₂—OH, and n is an integer from 1 to 6, inclusive. The compound may be in the form of a mineral acid salt or an organic acid salt.

### Irinotecan

In certain embodiments, irinotecan, topotecan, or their analogs can be used in the compositions, methods, and kits of the invention. Analogs of irinotecan are described, for example, in U.S. Pat. No. 4,604,463 and have the general structure:

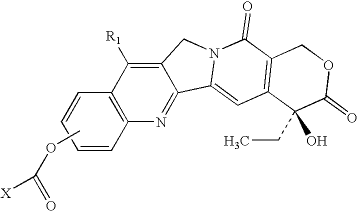

where R₁ is a hydrogen atom, a halogen atom, or a C₁₋₄ alkyl, and X is a chlorine or —NR₂R₃, wherein R₂ and R₃ are the same or different and each represents a hydrogen atom, a C₁₋₄ alkyl, or a substituted or unsubstituted carbocyclic or heterocyclic group, with the proviso that when both R₂ and R₃ are the substituted or unsubstituted alkyl groups, they may be combined together with the nitrogen atom, to which they are bonded, to form a heterocyclic ring which may be interrupted with —O—, —S—, and/or >N—R₄ in which R₄ is a hydrogen atom, a substituted or unsubstituted C₁₋₄ alkyl, or a substituted or unsubstituted phenyl group and where the grouping —O—CO—X is bonded to a carbon atom located in any of the 9-, 10-, and 11-positions in the ring A of camptothecin.
Analogs of topotecan are described, for example, in European Patent No. 321122 and include compounds with the general formula:

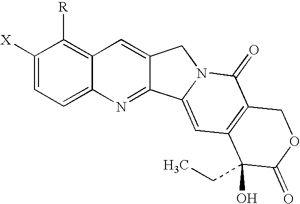

where X is hydroxy, hydrogen, cyano, —CH₂NH₂, or formyl; R is hydrogen when X is cyano, CH₂NH₂ or formyl or R is —CHO or —CH₂R₁ when X is hydrogen or hydroxy; R₁ is —O—R₂, —S—R₂, —N—R₂(R₃); or —N⁺—R₂—(R₃)(R₄), R₂, R₃, and R₄ are the same or different and are selected from H, C₁₋₆ alkyl, C₂₋₆ hydroxyalkyl, C₁₋₆ dialkyamino, C₁₋₆-dialkylaminoC₂₋₆alkyl, C₁₋₆ alkyamino-C₂₋₆ alkyl, C₂₋₆ aminoalkyl, or a 3-7 member unsubstituted or substituted carbocyclic ring; and when R₁ is —N—R₂(R₃), the R₂ and R₃ groups may be combined together to form a ring.

### Camptothecins

In certain embodiments, the anti-infective therapeutic agent is camptothecin, or an analogue or derivative thereof. Camptothecins have the following general structure.

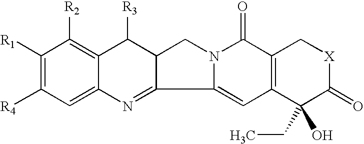

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

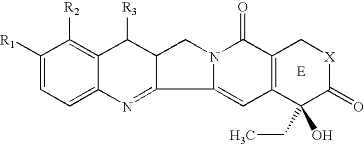

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity.

Camptothecins are believed to function as topoisomerase I inhibitors and/or DNA cleavage agents.

### Disulfuram

Disulfuram is used in the treatment of alcoholism; its mechanism of action is inhibition of alcohol dehydrogenase. The structure of disulfuram is:

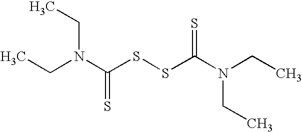

Analogs of disulfuram are described in, for example, U.S. Pat. No. 1,796,977 and have the general structure:

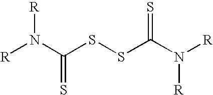

wherein the R groups represent same of dissimilar organic groups (e.g., C₁₋₄ alkyls).

Analogs include thiram. Disulfuram is a crystal, barely soluble in water, and is soluble in solvents such as alcohol, ether, acetone, and benzene. Disulfuram is available in tablet form, and is typically administered orally.

### Auranofin

Auranofin is an anti-inflammatory agent and an antirheumatic. The structure of auranofin is:

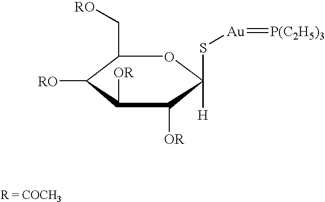

Analogs of auranofin are described, for example, in U.S. Pat. No. 3,635,945, and can be represented by the general formulas:

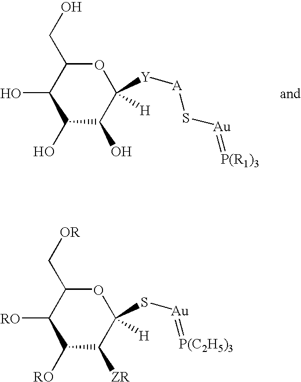

where R represents acetyl or, when Z is oxygen, hydrogen; R₁ represents a C₁₋₄ alkyl; A represents a C₂₋₅ alkylene chain, straight or branched; Y represents oxygen or sulfur; and Z represents oxygen or —NH—.

Auronfin is a white, odorless, crystalline powder and is insoluble in water. It is administered orally in tablet form.

### NSAIDs

In certain embodiments, an NSAID can be used in the compositions, methods, and kits of the invention. Suitable NSAIDs include A183827, ABT963, aceclofenac, acemetacin, acetyl salicylic acid, AHR10037, alclofenac, alminoprofen, ampiroxicam, amtolmetin guacil, apazone, aspirin, atliprofen methyl ester, AU8001, azelastine, benoxaprofen, benzydamine, benzydamine flufenamate, benzydamine hydrochloride, bermoprofen, bezpiperylon, BF388, BF389, BIRL790, BMS347070, bromfenac, bucloxic acid, butibufen, BW755C, C53, C73, C85, carprofen, CBS1108, celecoxib, CHF2003, chlorobiphenyl, choline magnesium trisalicylate, CHX108, cimicoxib, cinnoxicam, clidanac, CLX1205, CP331, CS502, CS706, D1367, curcumin, darbufelone, deracoxib, dexibuprofen, dexibuprofen lysine, dexketoprofen, DFP, DFU, diclofenac (e.g., diclofenac potassium, diclofenac sodium), diflunisal, DPI 55, DRF4367, E5110, E6087, eltenac, ER34122, esflurbiprofen, etoricoxib, F025, felbinac ethyl, fenbufen, fenclofenac, fenclozic acid, fenclozine, fenoprofen, fentiazac, feprazone, filenadol, flobufen, florifenine, flosulide, flubichin methanesulfonate, flufenamic acid, fluprofen, flurbiprofen, FPL62064, FR122047, FR123826, FR140423, FR188582, FS205397, furofenac, GR253035, GW406381, HAI105, HAI106, HCT2035, HCT6015, HGP12, HN3392, HP977, HX0835. HYAL AT2101, ibufenac, ibuprofen, ibuproxam-beta-cyclodextrin, icodulinum, IDEA070, iguratimod, imrecoxib, indomethacin, indoprofen, IP751, isoxepac, isoxicam, KC764, ketoprofen, L652343, L745337, L748731, L752860, L761066, L768277, L776967, L783003, L784520, L791456, L804600, L818571, LAS33815, LAS34475, licofelone, LM 4108, lobuprofen, lomoxicam, lumiracoxib, mabuprofen, meclofenamic acid, meclofenamate sodium, mefenamic acid, meloxicam, mercaptoethylguanidine, mesoporphyrin, metoxibutropate, miroprofen, mofebutazone, mofezolac, MX1094, nabumetone, naproxen sodium, naproxen-sodium/metoclopramide, NCX1101, NCX284, NCX285, NCX4016, NCX4215, NCX530, niflumic acid, nitric oxide-based COX-2 inhibitors and NSAIDs (NitroMed), nitrofenac, nitroflurbiprofen, nitronaproxen, NS398, ocimum sanctum oil, ONO3144, orpanoxin, oxaprozin, oxindanac, oxpinac, oxycodone/ibuprofen, oxyphenbutazone, P10294, P54, P8892, pamicogrel, parcetasal, parecoxib, PD138387, PD145246, PD164387, pelubiprofen, pemedolac, phenylbutazone, pirazolac, piroxicam, piroxicam beta-cyclodextrin, piroxicam pivalate, pirprofen, pranoprofen, resveratrol, R-ketoprofen, R-ketorolac, rofecoxib, RP66364, RU43526, RU54808, RWJ63556, S19812, S2474, S33516, salicylsalicylic acid, satigrel, SC236, SC57666, SC58125, SC58451, SFPP, SKF105809, SKF86002, sodium salicylate, sudoxicam, sulfasalazine, sulindac, suprofen, SVT2016, T3788, TA60, talmetacin, talniflumate, tazofelone, tebufelone, tenidap, tenoxican, tepoxalin, tiaprofenic acid, tilmacoxib, tilnoprofen arbamel, tinoridine, tiopinac, tioxaprofen, tolfenamic acid, tolmetin, triflusal, tropesin, TY10222, TY10246, TY10474, UR8962, ursolic acid, valdecoxib, WAY120739, WY28342, WY41770, ximoprofen, YS134, zaltoprofen, zidometacin, and zomepirac.

Other NSAIDs are described in U.S. Pat. Nos. 2,745,783, 3,318,905, 5,344,991, 5,380,738, 5,393,790, 5,401,765, 5,418,254, 5,420,287, 5,434,178, 5,466,823, 5,475,018, 5,474,995, 5,486,534, 5,504,215, 5,508,426, 5,510,368, 5,510,496, 5,516,907, 5,521,193, 5,521,207, 5,534,521, 5,565,482, 5,596,008, 5,616,601, 5,633,272, 5,639,777, 5,663,180, 5,668,161, 5,670,510, 5,672,626, 5,672,627 5,736,579, 5,739,166, 5,760,068, 5,756,529, 5,859,257, 5,886,016, 5,908,852, 5,916,905, 6,294,558, 6,476,042, 6,486,203, 6,492,411, 6,608,095, 6,649,645, 6,673,818, 6,689,805, 6,696,477, 6,727,268, 6,699,884, 6,727,238, 6,777,434, 6,846,818, 6,849,652, 6,949,536, 6,967,213, 7,019,144, and 7,041,694, PCT Publication Nos. WO94/13635, WO94/15932, WO94/20480, WO94/26731, WO96/03387, WO96/03388, WO96/09293, WO97/16435, WO98/03484, WO98/47890, WO96/06840, WO96/25405, WO95/15316, WO94/15932, WO94/27980, WO95/00501, and WO94/2673, and GB 839,057, GB 2,294,879, and EP 0745596.

Benzydamine

In certain embodiments, an NSAID such as benzydamine or an analog thereof can be used in the compositions, methods, and kits of the invention. The structure of benzydamine is:

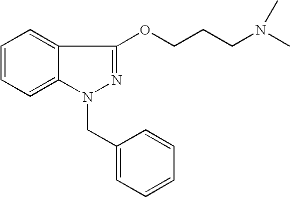

Analogs of benzydamine are described, for example, in U.S. Pat. No. 3,318,905 and have the general structure:

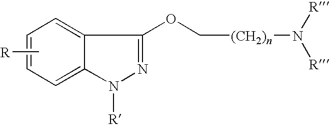

wherein R is selected from the class consisting of hydrogen and chlorine; R′ is selected from the class consisting of lower alkyl and phenyl groups which latter may be substituted or not in their phenyl nucleus by halogen atoms or lower alkyl or lower alkoxy groups; R″ is a member selected from the class consisting of hydrogen and lower alkyl groups; R′″, which may be like or unlike, are lower alkyl residues; n is selected from the group consisting of 1 and 2.

### Androgens

In certain embodiments, an androgen such as testerone or a testosterone analog can be used in the compositions, methods, and kits of the invention. Androgens such as androstenols include 14-hydroxyandrost-4-ene-3,6,17-trione, 16-acetoxy-17-acetoxymethyl-11,17-dihydroxy-D-homoandrosta-1,4-diene-3,17-dione, 17beta-((1R)-1-hydroxy-2-propynyl)androst-4-en-3-one, 17beta-amino-3beta-methoxy-5-androstene, 17beta-hydroxy-17-(2-methylallyl)-9beta,10alpha-androst-4-en-3-one, 17-(cyclopropylamino)androst-5-en-3-ol, 17-acetamido-5-androsten-3-ol-4-bis(2-chloroethyl)aminophenylacetate, 17-beta-hydroxy-7alpha-methyl-androst-5-en-3-one, 17-ethynyl-(5a)-androst-2-ene-17-ol-17-nicotinate, 17-ethynylandrost-2-ene-17-ol-17-acetate, 17-hydroxy-17-methyl-3-oxospiro(androst-5-ene-4,1′-cyclopropane)-2-carbonitrile, 17-methyl-17-hydroxyandrosta-1,4,6-trien-3-one, 19-ethynyl-19-hydroxyandrost-4-en-17-one, 2,3,17,19-tetrahydroxyandrost-4-ene, 2-beta-hydroxy-19-oxo-4-androstene-3,17-dione, 3beta-methoxy-5-androsten-17-one, 3′-azido-3′-deoxy-5′-O-((11-hydroxy-3-oxo-17-androst-4-enyl)carbonyl)thymidine, 3,15,17-trihydroxy-5-androstene, 3,16,19-trihydroxy-5-androsten-17-one, 3,17-dihydroxy-7-(4-methoxyphenyl)-androst-5-ene 3,17-diacetate, 3-hydroxy-17-methyl-18-norandrost-13(17)-ene-16-one, 3-methoxy-17-aza-homoandrost-5-ene-17-one, 5alpha-androst-16-en-3beta-ol, 5-androstene-3,16,17-triol, 9-fluoro-11,16,17-trihydroxy-17-hydroxymethyl-D-homoandrosta-1,4-diene-3,17-dione, 9-fluoro-16-methyl-6,11,16-trihydroxy-1,4-androstadiene-3,17-dione, abiraterone, androst-16-en-3-ol, androst-16-en-3-ol sulfoconjugate, androst-5-en-3-ol, androst-5-ene-3,16,17-triol-3-sulfate, androsta-2,4-diene-17beta-ol, androsta-5,16-dien-3beta-ol, Androstenediols (e.g., 17-cyano-9,17-dihydroxyandrost-4-ene-3-one, 2-carbamoyl-4,5-epoxyandrost-2-ene-3,17-diol, 3beta,17 beta-dihydroxyandrost-5-en-16-one, 3,16-dihydroxyandrost-5-ene-17,19-dione, 4-androstene-3,17-diol, 4a,17-dimethyl-A-homo-B,19-dinor-3,4-secoandrost-9-ene-3,17-diol, androst-4-ene-3beta,17beta-diol dicyclopentylpropionate, androst-4-ene-3beta,17 beta-diol dienanthate, androstenediol, cortienic acid, delta (2,16)-5alpha-androstadiene-3,17-diol-3,17-diacetate, Fluoxymesterone, formyldienolone, Methandriol, and viridiol), azastene, cyanoketone (e.g., Win 19578), Dehydroepiandrosterone (e.g., 1-hydroxydehydroepiandrosterone, 15beta-carboxyethylmercaptodehydroepiandrosterone, 15-hydroxydehydroisoandrosterone, 16-hydroxydehydroepiandrosterone, 16-hydroxydehydroepiandrosterone sulfate, 7-hydroxydehydroepiandrosterone, 7-oxodehydroepiandrosterone, androst-5-en-17-one, dehydroepiandrosterone acetate, dehydroepiandrosterone enanthate, dehydroepiandrosterone sulfate, dehydroepiandrosterone-3-O-methylthiophosphonate, fluasterone, gonasterone, gynodian, OH 8356, and testosterone mustard), epostane, etiocholenic acid, methyl 14-hydroxy-1,7,17-trioxoandrost-8-ene-19-oate, mexrenoate potassium, nordinone, ratibol, RS 21314, RS 85095, stenbolone, stenbolone acetate, testosterone, and thiomesterone.

Testosterone derivatives include 11-ketotestosterone, 11-oxatestosterone, 15beta-carboxyethylmercaptotestosterone, 15-carboxymethyltestosterone, 17beta-aminocarbonyloxy-4-androsten-3-one, 17-bromoacetoxy-4-androsten-3-one, 17-ethinyl-11-oxa-testosterone, 19-O-carboxymethoxytestosterone, 4-(carboxymethylmercapto)testosterone, 6-dehydrotestosterone, 6-methylenetestosterone acetate, ablacton, androsta-3,5-diene-3,17-diol diacetate, bolasterone, boldenone undecylenate, climacterone, clostebol, D-4-chloro-17beta-hydroxy-3-oxo-17alpha-methylandrosta-1,4-diene, dehydrotestosterone, deladumone, dimeric testosterone, epitestosterone, estandron prolongatum, ethynodiol testosterone ester, gonasterone, hydroxytestosterones, metharmon F, methenolone, methyltestosterone, nichlotest, synovex-H, testosterone 17beta-carboxylic acid, testosterone 17beta-cypionate, testosterone 17-cyclohexanecarboxylate, testosterone 17-enanthate 3-benzilic acid hydrazone, testosterone 3-(O-dimethylaminopropyl)oxime, testosterone 4-n-butylcyclohexylcarboxylic acid, testosterone acetate, testosterone decanoate, testosterone enanthate, testosterone formate, testosterone glucuronate, testosterone isobutyrate, testosterone isocaproate, testosterone palmitate, testosterone pivalate, testosterone propionate, testosterone undecanoate, testosterone-17-succinate, testosterone-17-sulfate, testosterone-19-hemisuccinate, testosterone-3-(n-hexyl)cyclobutane carboxylate, testosterone-3-oxime, testosterone-4-n-pentylcyclohexyl carboxylate, testosterone-cysteamine-DANS, testosterone-DAH-fluorescein, testosterone-DAP-fluorescein, testosteronyl 4-dimethylaminobutyrate, testoviron-depot, topterone, trofodermin, and turinabol.

Androstanols include 1,2-seco-A-bis(norandrostan-17-ol)acetate, 1,3,5,6-tetrahydroxyandrostan-17-one, 1,3-trimethylene-2′,5-epoxyandrostane-3,17-diol 17-propionate, 11,17-dihydroxy-6-methyl-17-(1-propynyl)androsta-1,4,6-triene-3-one, 16,17-epoxyandrostan-3-ol, 17beta-(3-furyl)-5beta,14beta-androstane-3beta,14beta-diol, 17-(3′-thiophenyl)androstane-3,14-diol 3-glucopyranoside, 17-acetamido-5-androstan-3-ol-4-bis(2-chloroethyl)aminophenylacetate, 17-ethyl-17-hydroxyandrostane, 17-hydroxy-2,3-cyclopropanoandrostane, 17-methyl-17a-chloro-D-homoandrostan-3-ol, 2-(2-(3-hydroxy-12-(2-methyl-1-oxobutoxy)-5-androstan-17-yl)ethyl)tetrahydro-4-hydroxy-2H-pyran-6-one, 3beta-acetoxy-5,6beta-dichloromethylene-5beta-androstan-17-one, 3,3-difluoroandrostane-17-ol acetate, 3-acetoxy-7,15-oxido-16-oxaandrostan-17-one, 3-hydroxy-17-(1H-1,2,3-triazol-1-yl)androsta-5,16-diene, 3-hydroxy-5-androstane-17-carbonitrile, 3-hydroxyetianic acid, 3-keto-5,10-epoxy-nor-19-methylandrostane-17-acetate, 4,5-epoxy-17-hydroxy-2-methylsulfonyl-3-androstanone, 5-bromo-3,6-dihydroxyandrostan-17-one-3-acetate, amafolone, androsol acetate, androstan-17-ol, androstan-3-ol, androstane-3,17-diol or derivatives thereof (e.g., 17-hydroxyandrostane-3-glucuronide, 17-methyl-D-homoandrostane-3,17-diol, 2,4-cycloandrostane-3,17-diol diacetate, 3-desacetylpipecuronium, 4-ethenylideneandrostane-3,17-diol, 4-ethenylideneandrostane-3,17-dione, androstane-2,3,17-triol, androstane-3,14-diol, androstane-3,16,17-triol, androstane-3,17-diol 17-sulfate, androstane-3,17-diol dipropionate, androstane-3,17-diol glucuronide, androstane-3,6,17-triol, androstane-3,7,17-triol, androstane-3,7-diol disulfate), androsterone or its derivatives (e.g., 11beta-hydroxyandrosterone, 11-ketoandrosterone, 16beta-hydroxyandrosterone, 16-bromoepiandrosterone, 17-hydroxy-6,6-ethylene-4-androsten-3-one, 19-hydroxy-4-androsten-17-one, 3-bromoacetoxyandrostan-17-one, 3-hydroxy-4-androsten-17-one, androsterone 3-benzoate, androsterone 3-palmitate, androsterone glucuronide, and androsterone sulfate), BOMT, CCI 22277, dihydrotestosterone or its derivatives (e.g., 11-fluoro-19-nor-dihydro-testosterone, 11-fluoro-dihydro-testosterone, 16-iodostanolone, 17-(2-iodoethenyl)androsta-4,6-dien-17-ol-3-one, 17-(2-iodoethynyl)androsta-4,6-dien-17-ol-3-one, 17-(2-iodovinyl)dihydrotestosterone, 17-hydroxyandrostan-19-ol-3-one, 17-hydroxyandrostan-3-one 17-sulfate, 17-ketotrilostane, 17-N,N-diethylcarbamoyl-4-methyl-4-azaandrostane-3-one, 17-N,N-diisopropylcarbamoyl-4-azaandrostan-3-one, 18-hydroxy-18-methyl-16,17-methylene-D-homoandrostane-3-one, 2,17-dimethyldihydrotestosterone, 2-bromo-5-dihydrotestosterone, 2-chloroethylnitrosocarbamoylalanine 17-dihydrotestosterone ester, 3-hydroxyandrostan-16-one, 4,17-dimethyltrilostane, 4,5-secodihydrotestosterone, 5-dihydrotestosterone 3,17-bromoacetate, androstan-3,17-diol-11-one, androstan-3-one, demalon, dihydrotestosterone 17-bromoacetate, dihydrotestosterone glucuronide, dihydrotestosterone heptanoate, dihydrotestosterone propionate, dihydrotestosterone-17-N-bis(2-chloroethyl)carbamate, mestanolone, mesterolone, nitrostanolone, stanolone benzoate, testiphenon, and trilostane), dromostanolone, dromostanolone propionate, epitiostanol, etiocholanolone or its derivatives (e.g., 11-ketoetiocholanolone, 3,7-dihydroxyandrostan-17-one, 3-hydroxyandrostane-7,17-dione, and androstane-3,17-dione), furazabol, mebolazine, mepitiostane, N-cyano-2-aza-A-norandrostan-17-ol acetate, nisterime acetate, ORG 9943, ORG 9991, Org NA 13, oxandrolone, oxymetholone or its derivatives (e.g., 17-hydroxy-2-(hydroxymethylene)androstan-3-one), Pancuronium or its derivative (e.g., (dideacetoxy)pancuronium, 2,16-dipiperidinoandrostane-3,17-diol dipivalate, 3alpha,17beta-dibutyryloxy-2beta,16beta-dipiperidino-5alpha-androstane dimethobromide, 3-(deacetoxy)pancuronium, 3-desacetylpancuronium, dacuronium, and Org 6368), RU 26988, rubrosterone, samanine, spiro-3-oxiranylandrostan-17-ol, stanozolol or its derivatives (e.g., 16-hydroxystanozolol and 4,16-dihydroxystanozolol), vecuronium bromide or its derivatives (e.g., (dideacetoxy)vecuronium, 17-deacetylvecuronium, 3,17-bis-deacetylvecuronium, 3-(deacetoxy)vecuronium, 3-deacetylvecuronium, Org 7617, Org 7678, Org 7684, Org 9273, and Org 9616).

Stanozolol analogs are described in U.S. Pat. No. 3,030,358. Mesterolone analogs are described in U.S. Pat. No. 3,361,773. Methyltestosterone analogs are described in U.S. Pat. No. 2,374,370.

### Tyrphostins

In certain embodiments, a tyrophostin can be used in the compositions, methods, and kits of the invention. The tyrphostins are family of synthetic kinase inhibitors. Exemplary tyrphostins include 6,7-dimethoxy-2-phenylquinoxaline, AG 127, AG 183, AG 30, AG 494, AG 556, AG 879, RG 13022, RG 14620, RG 50810, RG 50864, tyrphostin 11, tyrphostin 23, tyrphostin 25, tyrphostin 8, tyrophostin 47, tyrphostin A46, tyrphostin A51, tyrphostin A9, tyrphostin AG 1024, tyrphostin AG 1112, tyrphostin AG 1296, tyrphostin AG 1478, tyrphostin AG 555, tyrphostin AG 568, tyrphostin AG-490, tyrphostin AG17, tyrphostin AG879, and tyrphostin AG957. Tyrphostins are described in U.S. Pat. Nos. 5,728,868 and 5,854,285.

### Vitamin B₁₂

Vitamin B₁₂ and B₁₂ analogs can be used in the compositions, methods, and kits of the invention. Vitamin B₁₂, its derivatives, and its analogs are cofactors in folate enzymes and methionine synthase. 5-Deoxyadenosyl cobalamin is a cofactor required by the enzyme that converts L-methylmalonyl-CoA to succinyl-CoA. Other vitamin B₁₂ analogs include 1,N(6)-ethenoadenosylcobalamin, 2′,5′-dideoxyadenosylcobalamin, 2-methyl-2-aminopropanol-B₁₂, adeninylethylcobalamin, ambene, aminopropylcobalamin, aquacobalamin, biofer, Co-(carboxymethyl)cobalamin, cob(II) alamin, cobamides (e.g., (2-amino-5,6-dimethylbenzimidazolyl)cobamide, (2-hydroxy-5,6-dimethylbenzimidazolyl)cobamide, 2-methylsulfinyladenylcobamide, 2-methylsulfonyladenylcobamide, 4-cresolylcobamide, adenosylcobinamide methyl phosphate, coalpha-(alpha-5,6-dimethylbenzimidazolyl)-cobeta-cyanocobamide, cobamamide, cobamamide 5′-phosphate, cobinamide, phenolyl cobamide, thiobanzyme), cobyric acid, cobyrinic acid, cobyrinic acid hexamethyl ester f-nitrile, compound 102804, cyanocobalamin-b-monocarboxylic acid, cyanocobalamin-e-monocarboxylic acid, cysteinylcobalamin, factor A, factor III, ferribalamin, formylmethylcobalamin, FV 82, glutathionylcobalamin, hepavis, hydroxocobalamin (e.g., nitrosocobalamin and acetatocobalamin), Jectofer compound, mecobalamin, methylcobalamine chlorpalladate, nitritocobalamin, nitrosylcobalamin, proheparum, pseudovitamin B₁₂, sulfitocobalamin, Transcobalamins, triredisol, and vitamin B₁₂ factor B. Cobamamide analogs are described in U.S. Pat. No. 3,461,114.

### Histone Deacetylase (HDAC) Inhibitors

Histone deacetylase inhibitors and their analogs may be used in the compositions, methods, and kits of the invention. Exemplary HDACs include CAY10433 and suberohydroxamic acid. Histone deacetylase inhibitors are used, for example, in cancer therapy, and in the treatment of inflammation and are a group of compounds that include, for example, cyclic peptides (e.g., depsipeptides such as FK228), short chain fatty acids (e.g., phenylbutyrate and valproic acid), benzamides (e.g., CI-994 and MS-27-275), and hydroxamic acids (e.g., suberoylanilide hydroxamic acid (SAHA)) as described in Richon and O'Brien ((2002) Clin. Canc. Res. 8, 662-664). Cyclic peptides and analogs useful in the invention are described, for example, in U.S. Pat. No. 6,403,555. Short chain fatty acid HDAC inhibitors are described in, for example, U.S. Pat. Nos. 6,888,027 and 5,369,108. Benzamides analogs are described, for example, in U.S. Pat. No. 5,137,918. Analogs of SAHA are described, for example, in U.S. Pat. No. 6,511,990. Other HDACs include anacardic acid, apicidin, histone deacetylase inhibitor I, histone deacetylase inhibitor II, histone deacetylase inhibitor III, ITSA1, oxamflatin, SBHA, scriptaid, sirtinol, splitomicin, trichostatin A, and valproic acid (e.g., sodium salt). Any of these compounds or other HDAC inhibitors may be used in the compositions, methods, or kits of the invention.

### Platinum Complexes

In certain embodiments, a platinum compound can be used in the compositions, methods, and kits of the invention. In general, suitable platinum complexes may be of Pt(II) or Pt(IV) and have this basic structure:

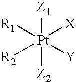

wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

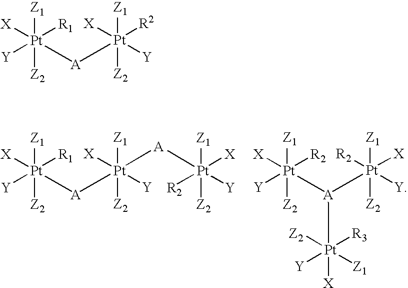

Exemplary platinum compounds are cisplatin, carboplatin, oxaliplatin, and miboplatin having the structures:

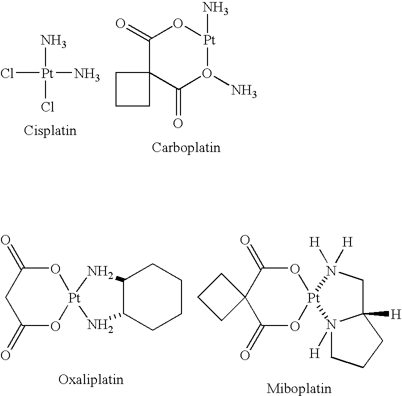

Other representative platinum compounds include (CPA)₂Pt(DOLYM) and (DACH)Pt(DOLYM) cisplatin (Choi et al., Arch. Pharmacal Res. 22(2):151-156, 1999), Cis-(PtCl₂(4,7-H-5-methyl-7-oxo)1,2,4(triazolo(1,5-a)pyrimidine)₂) (Navarro et al., J. Med. Chem. 41(3):332-338, 1998), (Pt(cis-1,4-DACH)(trans-Cl₂)(CBDCA)).½MeOH cisplatin (Shamsuddin et al., Inorg. Chem. 36(25):5969-5971, 1997), 4-pyridoxate diammine hydroxy platinum (Tokunaga et al., Pharm. Sci. 3(7):353-356, 1997), Pt(II) . . . Pt(II) (Pt₂(NHCHN(C(CH₂)(CH₃)))₄) (Navarro et al., Inorg. Chem. 35(26):7829-7835, 1996), 254-S cisplatin analogue (Koga et al., Neurol. Res. 18(3):244-247, 1996), o-phenylenediamine ligand bearing cisplatin analogues (Koeckerbauer & Bednarski, J. Inorg. Biochem. 62(4):281-298, 1996), trans, cis-(Pt(OAc)₂I₂(en)) (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1):75, 1996), cis-1,4-diaminocyclohexane cisplatin analogues (Shamsuddin et al., J. Inorg. Biochem. 61(4):291-301, 1996), 5′ orientational isomer of cis-(Pt(NH₃)(4-aminoTEMP-O){d(GpG)}) (Dunham & Lippard, J. Am. Chem. Soc. 117(43):10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Otto et al., J. Cancer Res. Clin. Oncol. 121(1):31-8, 1995), (ethylenediamine)platinum(II) complexes (Pasini et al., J. Chem. Soc., Dalton Trans. 4:579-85, 1995), CI-973 cisplatin analogue (Yang et al., Int. J. Oncol. 5(3):597-602, 1994), cis-diaminedichloroplatinum(II) and its analogues cis-1,1-cyclobutanedicarbosylato(2R)-2-methyl-1,4-butanediamineplatinum(II) and cis-diammine(glycolato)platinum (Claycamp & Zimbrick, J. Inorg. Biochem. 26(4):257-67, 1986; Fan et al., Cancer Res. 48(11):3135-9, 1988; Heiger-Bernays et al., Biochemistry 29(36):8461-6, 1990; Kikkawa et al., J. Exp. Clin. Cancer Res. 12(4):233-40, 1993; Murray et al., Biochemistry 31(47):11812-17, 1992; Takahashi et al., Cancer Chemother. Pharmacol. 33(1):31-5, 1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2):311-15, 1991), trans-diamminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum(II) and 3H-cis-1,2-diaminocyclohexane-malonatoplatinum (II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57, 1988), aminoalkylaminoanthraquinone-derived cisplatin analogues (Kitov et al., Eur. J. Med. Chem. 23(4):381-3, 1988), spiroplatin, carboplatin, iproplatin and JM40 platinum analogues (Schroyen et al., Eur. J. Cancer Clin. Oncol. 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2):157-65, 1987), JM8 and JM9 cisplatin analogues (Harstrick et al., Int. J. Androl. 10(1); 139-45, 1987), (NPrⁿ₄)₂((PtCL₄).cis-(PtCl₂—(NH₂Me)₂)) (Brammer et al., J. Chem. Soc., Chem. Commun. 6:443-5, 1987), aliphatic tricarboxylic acid platinum complexes (EPA 185225), and cis-dichloro(amino acid) (tert-butylamine)platinum(II) complexes (Pasini & Bersanetti, Inorg. Chim. Acta 107(4):259-67, 1985). Oxaliplatin analogs are described in U.S. Pat. Nos. 4,169,846, 5,290,961, 5,298,642, and 6,153,646. Satraplatin is described in Choy, Expert Rev. Anticancer Ther. 6(7):973-982, 2006). These compounds are thought to function by binding to DNA, i.e., acting as alkylating agents of DNA.

### Flavanones

In certain embodiments, a flavanone can be used in the compositions, methods, and kits of the invention. Exemplary flavanones include 2-hydroxyflavanone, 137 L, 2′,3,5,7-tetrahydroxyflavanone, 3′-prenylnaringenin, 6-(1,1-dimethylallyl)naringenin, 7-hydroxyflavanone, 7-O-methyleriodictyol, 8-prenylnaringenin, baicalein, BE 14348D, carthamidin, desmal, eriodictyol, eriodictyol 7-glucuronide, flavanone, flemiphilippinin D, Hesperidin (e.g., Cirkan N. D., dehydro-sanol-tri, essaven, fleboplex, hesperetin, hesperetin 5-O-glucoside, hesperetin 7-O-lauryl ether, hesperidin methylchalcone, methyl hesperidin, neohesperidin dihydrochalcone, and S 5682), liquiritigenin, naringenin, naringenin-6-C-glucoside, naringin, pinobanksin, pinocembrin, plantagoside, scuteamoenin, scuteamoenoside, shinflavanone, uralenin, vexibinol, wogonin, and WS 7528.

### Amorolfine

In certain embodiments, amorolfine or an amorolfine derivative such as benzamil can be used in the compositions, methods, and kits of the invention. Amorolfine is an antifungal agent that is typically administered topically. The structure of amorolfine is:

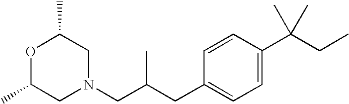

Analogs of amorolfine are described, for example, in U.S. Pat. No. 4,202,894 and have the general structure:

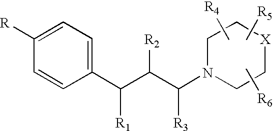

wherein R is alkyl of 4 to 12 carbon atoms, cycloalkyl of 3 to 7 carbon atoms, mono(lower alkyl)-substituted cycloalkyl of 4 to 7 carbon atoms, cycloalkylalkyl of 4 to 12 carbon atoms, phenyl or aryl-(lower alkyl) of 7 to 12 carbon atoms; R₁, R₂, and R₃, independently, are hydrogen or alkyl of 1 to 8 carbon atoms; R₄, R₅, and R₆, independently, are hydrogen or alkyl of 1 to 8 carbon atoms, and two of R₄, R₅, and R₆ can each be bonded to the same carbon atom or together can form a fused alicyclic or aromatic 6-membered ring; provided that when R is tert.-butyl, at least one of R₁ and R₃ is alkyl of 2 to 8 carbon atoms or R₂ is hydrogen or alkyl of 2 to 8 carbon atoms or at least one of R₄, R₅, and R₆ is alkyl of 5 to 8 carbon atoms; X is methylene or an oxygen atom; z is zero or 1 and the dotted bonds can be hydrogenated, and acid addition salts of those compounds of formula I which are basic, where the term “lower alkyl” denotes a straight-chain or branched-chain hydrocarbon group of 1 to 4 carbon atoms, such as, methyl, ethyl, propyl, isopropyl, butyl, isobutyl and tert.-butyl. Alkyl groups of 4 to 12 carbon atoms are straight-chain or branched-chain hydrocarbon groups, for example, butyl, isobutyl, tert.-butyl, neopentyl, 1,1-dimethylpropyl, 1,1-dimethylpentyl, 1,1-diethylpropyl, 1,1-dimethylbutyl, 1-isopropyl-3-methyl-but-1-yl, 1-ethyl-1-methylbutyl, dodecyl, and the like. Cycloalkylalkyls include, in particular, those groups in which the alkyl moiety is branched. The term “aryl-(lower alkyl)” includes not only groups which are mono- or di(lower alkyl)-substituted in the aryl ring but also groups which are mono- or di(lower alkyl)-substituted in the lower alkyl moiety. Exemplary of aryl(lower alkyl) groups are benzyl, phenylethyl, (lower alkyl)-benzyl, for example, methylbenzyl and dimethylbenzyl, naphthylmethyl, 2-phenyl-propan-2-yl, 1-phenyl-1-ethyl, or the like.

Amorolfine is a member of the morpholines, which include ((2-azido-4-benzyl)phenoxy)-N-ethylmorpholine, (+)-(S)-5,5-dimethylmorpholinyl-2-acetic acid, (morpholinyl-2-methoxy)-8-tetrahydro-1,2,3,4-quinoline, 1,1′-hexamethylenebis(3-cyclohexyl-3-((cyclohexylimino)(4-morpholinyl)methyl)urea), 1,4-bis(3′-morpholinopropyl-1′-yl-1′)benzene, 1,4-thiomorpholine-3,5-dicarboxylic acid, 1,4-thiomorpholine-3-carboxylic acid, 1-(morpholinomethyl)-4-phthalimidopiperidine-2,6-dione, 1-deoxy-1-morpholino-psicose, 1-deoxy-1-morpholinofructose, 1-phenyl-2,3-dimethyl-4-naphthalanmorpholinomethylpyrazolin-5-one, 1-phenyl-2-palmitoylamino-3-morpholino-1-propanol, 2,6-bis(carboxymethyl)-4,4-dimethylmorpholinium, 2,6-dimethylmorpholine, 2,6-dioxo-N-(carboxymethyl)morpholine, 2-(((3-(morpholinylmethyl)-2H-chromen-8-yl)oxy)methyl)morpholine, 2-(3-trifluoromethyl)phenyltetrahydro-1,4-oxazine, 2-(4-morpholino)ethyl-1-phenylcyclohexane-1-carboxylate, 2-(4-morpholino-6-propyl-1,3,5-triazin-2-yl)aminoethanol, 2-(4-morpholinyl)-4H-1-benzopyran-4-one, 2-(4-morpholinyl)-8-phenyl-4H-1-benzopyran-4-one, 2-(4-nitrophenyl)-4-isopropylmorpholine, 2-(morpholin-4-yl)benzo[h]chromen-4-one, 2-(N-methylmorpholinium)ethyl acetate, 2-(N-morpholino)ethanesulfonic acid, 2-benzylmorpholine, 2-hydroxy-4,4-dimethyl-2-(4-tolyl)morpholinium, 2-methyl-3-(2-methyl-2,3-diphenyl-4-morpholinyl)-1-phenyl-1-propanone, 2-morpholinomethyl-2′,3′,4′-trimethoxyacrylophenone, 2-n-pentyloxy-2-phenyl-4-methylmorpholine, 2-phenyl-5,5-dimethyltetrahydro-1,4-oxazine, 2-thiomorpholinoethylacrylamide, 3,5,5-trimethyl-2-morpholinon-3-yl radical dimer, 3-((benzyloxy)methyl)morpholine, 3-(beta-morpholinoethoxy)-1H-indazole, 3-cyano-2-morpholino-5-(pyrid-4-yl)pyridine, 3-thiomorpholinopropylacrylamide, 4,4′-dithiodimorpholine, 4,4-methylenedimorpholine, 4-(2-morpholinoethoxy)benzophenone, 4-(3,7,11,15-tetramethyl-6,10,14-hexadecatrienoyl)morpholine, 4-amino-5-chloro-2-ethoxy-N-((2-morpholinyl)methyl)benzamide, 4-amino-N-((4-benzyl-2-morpholinyl)methyl)-5-chloro-2-ethoxybenzamide, 4-amino-N-((4-benzyl-2-morpholinyl)methyl)-5-chloro-2-methoxybenzamide, 4-benzylphenoxy-N-ethylmorpholine, 4-cyclododecyl-2,6-dimethylmorpholine acetate, 4-methoxyphenyl-(5-methyl-6-(2-(4-morpholinyl)ethyl)-6H-thieno(2,3-b)pyrrol-4-yl)phenylmethanone, 4-methylmorpholine, 4-methylmorpholine N-oxide, 4-morpholinedithiocarbamate, 4-morpholinocarbonitrile, 5-pentyl-N-nitrosomorpholine, A 74273, AH 19437, aprepitant, AWD 140076, befol, BIBW 22, bis(3,5-dimethyl-5-hydroxymethyl-2-oxomorpholin-3-yl), BW 175, cetethyl morpholinium, CGP 53437, C11033, ciclosidomine, CNK 6001, CNK 6004, CP 80794, CP 84364, CS 722, delmopinol, detensitral, Dextromoramide, di-beta-(morpholinoethyl)selenide, dimethomorph, dimethyl morpholinophosphoramidate, dimorpholamine, ES 6864, ES 8891, fenpropimorph, filenadol, FK 906, fominoben, FR 76830, Go 8288, GYKI 11679, indeloxazine, L 689502, L 742694, L 760735, landiolol, lateritin, M&B 16573, MDL 101146, MF 268, mofarotene, Molsidomine, morfolep, Moricizine, morlincain, moroxybrate, moroxydine, morpholine, morpholineoethylamino-3-benzocyclohepta(5,6-c)pyridazine, morpholinoamidine, morpholinophosphordichloridite, morpholinopropane sulfonic acid, morpholinosulfonic acid, morpholinylethoxy-3-methyl-4-(2′-naphthyl)-6-pyridazine, mosapride, N,N′-dicyclohexyl-4-morpholinecarboxamidine, N-((4-benzyl-2-morpholinyl)methyl)-5-chloro-4-(dimethylamino)-2-methoxybenzamide, N-(3,N′-morpholinopropyl)-2-(3-nitropyrrolo-(2,3-b)pyridine-1-yl)ethanoic acid amide, N-(3-nitro-4-quinoline)morpholino-4-carboxamidine, N-dodecylmorpholine, N-ethylmorpholine, N-hexylmorpholine-2′,5′-oligoadenylate, N-nitromorpholine, N-oxydiethylene-2-benzothiazole sulfenamide, O—(N-morpholinocarbonyl)-3-phenyllacetic acid, oxaflozane, oxymorphindole, P 1487, P 34081, PD 132002, phendimetrazine, Phenmetrazine, phenyl 2-(2-N-morpholinoethoxy)phenyl ether, pholcodine, phosphorodiamidate morpholino oligomer, pinaverium, pramoxine, proctofoam-HC, promolate, RE 102, reboxetine, Ro 12-5637, Ro 12-8095, RS 1893, RV 538, S 12024, S 14001, S-anisylformamidino-4-(N-methylisothioamide)morpholine, S-phenethylformamidino-4-(N-ethylisothioamide)morpholine, SC 46944, Seda-Miroton, silatiemonium iodide, SIN 1C, SR 121463A, Stymulen, sufoxazine, teomorfolin, theniloxazine, thiamorpholine, tiemonium iodide, tiemonium methylsulfate, tridemorph, trifenmorph, trimetozine, trimorfamid, trithiozine, TVX 2656, U 37883A, U 84569, U 86983, UP 614-04, Viloxazine, Win 55212-2, and YM 21095.

### Andrographis

In certain embodiments, andrographis, or an extract or component thereof, can be used in the compositions, methods, and kits of the invention. Andographis paniculata is medicinal herb, which has been used as an antipyretic, an anti-inflammatory agent, and a liver protectant. It also is reported to have anticancer and antiviral (e.g., anti-HCV and anti-HIV) properties. The primary active agent in andrographis is andrographolide. The structure of andrographolide is:

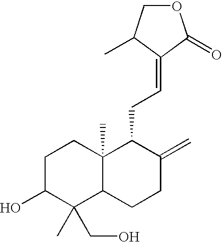

Andrographolide analogs are described, for example, in U.S. Pat. Application Publication No. 2006/0223785 and have the general structure:

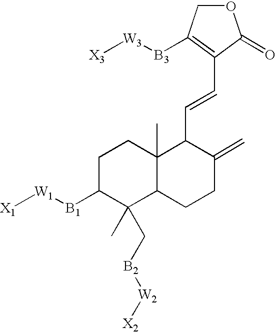

or its cis isomer, or its pharmaceutically acceptable salt, ester, salt of an ester or prodrug, wherein: B₁, B₂ and B₃ are independently CR₁R₂, C(Y₁), O, NR₄, PR₅, P(═Y₂)R₆, P(═Y₃)₂, S(═Y₄)ₖ, a spacer group or a covalent bond; and k can be 0, 1 or 2; and W₁, W₂ and W₃ are independently CR₇R₈, CR₉, C, C(Y₅), O, NR₁₀, PR₁₁, P(═Y₆)R₁₂, P(═Y₇)₂, S(═Y₈)_{f} or a covalent bond; and f can be 0, 1 or 2; or B₁—W₁, B₂—W₂, and/or B₃—W₃ are independently CR₃═CR₉ or C≡C; and X₁, X₂ and X₃ are independently hydrogen, CR₁₈R₁₉R₂₀, C═R₂₁R₂₂, C≡R₂₃, C≡N, C(═Y₉)R₂₄, OR₂₅, NR₂₆R₂₇, N═NR₂₈, P(═Y₁₀)_{d}(R₂₉)V, S(═Y₁₁)_{d}(R₃₀)ᵢ or NO₂; and d can be 0, 1 or 2; and v can be 0, 1 or 2; and i can be independently 0 or 1; and Y₁, Y₂, Y₃, Y₄, Y₅, Y₆, Y₇, Y₈, Y₉, Y₁₀, and Y₁₁ are independently O, S, or NZ; and Z can be independently hydrogen, R₁₃, OR₁₄, SR₁₅ or NR₁₆R₁₇; and R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₈, R₁₉, R₂₀, R₂₁, R₂₂, R₂₃, R₂₄, R₂₅, R₂₆, R₂₇, R₂₈, R₂₉, R₃₀, R₃₁ and R₃₂ are independently hydrogen, alkyl, alkenyl, alkynyl, cycloalkyl, cycloalkenyl, cycloalkynyl, aryl, alkaryl, arylalkyl, heterocyclic, heteroaromatic, acyl, aldehyde, carbamide, alkoxy, amino, halogen, silyl, thiol, sulfoxy, sulfinyl, sulfamoyl, hydroxyl, ester, carboxylic acid, amide, nitro, cyano, phosphonyl, phosphinyl, phosphoryl, imide, thioester, ether, acid halide, oxime, carbamate, thioether, residue of a natural or synthetic amino acid or a carbohydrate, any of which can be optionally attached to the targeting moiety or oxygen radical through a spacer group; or alternatively, R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₈, R₁₉, R₂₀, R₂₁, R₂₂, R₂₃, R₂₄, R₂₅, R₂₆, R₂₇, R₂₈, R₂₉, R₃₀, R₃₁ and R₃₂ can individually come together to form a bridged compound comprising of alkyl, alkenyl, alkynyl, cycloalkyl, cycloalkenyl, cycloalkynyl, aryl, alkaryl, aryl alkyl, heterocyclic, heteroaromatic, acyl, carbamide, alkoxy, amino, halogen, silyl, thiol, sulfinyl, sulfamoyl, ester, amide, phosphonyl, phosphinyl, phosphoryl, imide, thioester, ether, oxime, carbamate, thioether, residue of a natural or synthetic amino acid or a carbohydrate, any of which can be optionally attached to the targeting moiety or oxygen radical through a spacer group; and each carbon atom cannot be covalently bound to more than two heteroatoms; and wherein each B, W and X cannot be all heteroatom moieties unless B, W and X are all nitrogen based or B and X are independently O or N and W is PR₁₁, POR₁₂, PO₂, S(Y₄)ₘ and m is 1 or 2; and wherein each B and W or W and X cannot both be of the general formula C(Y), POR₁₂, PO₂, S(═Y₄)ₜ and t is 1 or 2.

In one subembodiment of formula I, B₁, B₂, and B₃ are independently CR₁R₂, C(Y₁), O, or a covalent bond; W₁, W₂ and W₃ are independently CR₇R₈, CR₉, C, C(Y₅), O, or a covalent bond; and X₁, X₂ and X₃ are independently hydrogen, CR₁₈R₁₉R₂₀, C═R₂₁R₂₂, C≡R₂₃. In one subembodiment of formula I, at least one of B₁, B₂, and B₃ and at least one W₁, W₂, and W₃ is a covalent bond and at least one X₁, X₂, and X₃ is hydrogen.

In another embodiment of the above formula, at least one R₁, R₂, R₃, R₄, R₅, R₆, R₇, R₈, R₉, R₁₀, R₁₁, R₁₂R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, and R₃₂ is selected from an aliphatic, saturated or unsaturated alkyl, alkenyl or alkynyl. In one subembodiment, the alkyl, alkenyl or alkynyl groups are substituted, and can be halogen substituted.

In one embodiment of the above formula, at least one R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁ and R₃₂ is selected from a carbonyl containing groups, including, but not limited to, aldehyde, ketone, carboxylic acid, ester, amide, enone, acyl chloride or anhydride.

In one embodiment of the above formula, at least one R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁ and R₃₂ is selected from an alkyl, aryl, heteroaryl or heteroaromatic ring.

In one embodiment of the above formula, at least one R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁ and R₃₂ is independently selected from alkyl, nitro, a phosphate, a sulfate, a thiol, and an amine.

### Arbidol

In certain embodiments, arbidol or an analog thereof can be used in the compositions, methods, and kits of the invention. Arbidol is an antiviral that has anti-influenza activity and functions by inhibition of the fusion of influenza A and B viruses within endosomes. The structure of arbidol is:

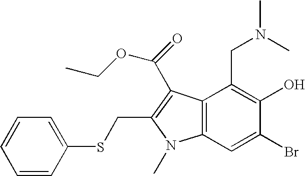

Arbidol is typically administered orally.

### Artemisinins

In certain embodiments, artemisinin or an analog thereof can be used in the compositions, methods, and kits of the invention. The artemeisins are a family of compounds that include antimalarials such as artemisinin and artemether, a semi-synthetic derivative of artemisinin. The structure of artemisinin is:

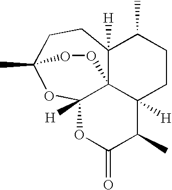

The structure of artemether is:

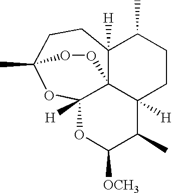

The structure of artesunate is:

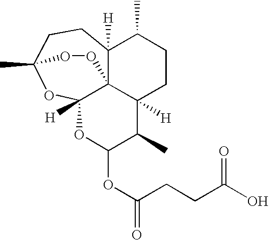

Other artemisinins include 3-hydroxydeoxyartemisinin, α-propoxycarbonyldihydroartemisine, arteannuin B, arteether, arteflene, artelinic acid, artemether, artemisic acid, artemisin, artemisinin B, artemisinine, artemisitene, artesunate, artesunic acid, deoxoartemisinin, deoxyartemisinin, and dihydroquinghaosu. The active metabolite of artemisinins is dihydroartemisinin.

### Benoxinate

In certain embodiments, procaine or a derivative thereof such as benoxiante can be used in the compositions, methods, and kits of the invention. Benoxinate is an anesthetic agent. The structure of benoxinate is:

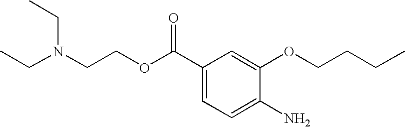

Benoxinate is a procaine derivative. Other procaine derivatives include 4-bromoacetamidoprocaine, analgesin, aslavital, benoxinate, bivelin, Cardioplegin, celnovocaine, chloroprocaine, efatin, Fluress, Impletol, impletol depot Bayer, N,N-diethylaminoethyl(2-N-methyl)benzoate, N-acetylprocaine, nicotinoylprocaine, novdimal, Penicillin G, Procaine, procaine acryloyl polymer, procaine azide, procaine isothiocyanate, Renovaine, sulfocamphocaine, Tardomyocel compound, and turigeran.

### Amiloride

In certain embodiments, amiloride or an analog thereof such as benzamil can be used in the compositions, methods, and kits of the invention. Amiloride is a diuretic agent. The structure of amiloride is:

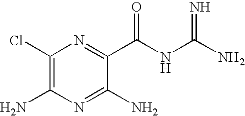

The structure of benzamil is:

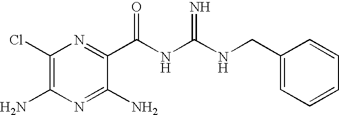

Amiloride derivatives are described, for example, in U.S. Pat. No. 3,313,813 and can be represented by the following formula:

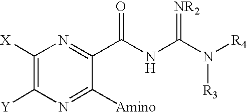

where X represents hydrogen, a halogen or halogen-like radical, such as, chloro, bromo, iodo or trifluoromethyl, or a lower-alkyl, lower-cycloalkyl, mononuclear aryl, either unsubstituted or substituted, advantageously with a halogen especially a chloro or bromo substituent, amino, Z-thio or Z-sulfonyl wherein Z is lower alkyl or phenyl-lower alkyl; Y represents hydrogen, hydroxyl or mercapto, lower alkoxy or lower alkyl-thio, halogen, especially chlorine, lower-alkyl, lower-cycloalkyl, mononuclear aryl, especially phenyl or amino, advantageously having the structure NRR₁, wherein R and R1 can be similar or dissimilar radicals and respectively represent hydrogen, amino or mono- or di-lower-alkylamino, (advantageously forming a hydrazino group at the 5-position carbon), lower alkoxy, Y represents substituted amino, —NRR₁, where R and R₁ represent lower alkyl either straight or branched chain or cyclic (3- to 6-membered rings) and either unsubstituted or containing one or more substituents such as hydroxyl, halogen (chlorine, bromine, fluorine and the like), a cycloalkyl substituent having 3 to 6 carbons in the cycloalkyl structure, an aryl substituent preferably phenyl or substituted phenyl such as lower-alkyl-phenyl and halophenyl as chlorophenyl, bromophenyl, fluorophenyl, and the like, or a heterocyclic substituent especially furyl, pyridyl, and (CH₂)ₙN— wherein n is one of the numerals 4 through 6, or an amino substituent as the unsubstituted amino, or mono- or di4ower-alkyl amino, and when R and R₁ each represents a lower alkyl, the lower alkyl groups can be linked together to form a cyclic structure with the nitrogen atom to which they are attached, particularly a 5- to 8-membered ring, advantageously forming with the nitrogen atom a 1-pyrrolidinyl, piperidino, hexahydro-1-azepinyl, or octahydro-1-azocinyl radical and the like, Y represents substituted amino, —NRR₁, where R and R₁ represent lower alkenyl, aryl, advantageously an unsubstituted or substituted phenyl, wherein the substituent(s) are preferably halogen (chlorine, bromine, fluorine) or lower alkyl (methyl, ethyl, propyl, iso-propyl) and the like, amidino or substituted amidino, especially an N,N-di-lower alkyl-imidino, such as N,N-dimethylamidino; X and Y, in addition, can be linked together to form a 4-membered carbon chain that can be either unsaturated or saturated and that can be unsubstituted or substituted, and if substituted the substituent advantageously is a halogen, especially a chloro-atom. R₂ represents hydrogen and lower alkyl; R₃ represents hydrogen, lower alkyl, either saturated or unsaturated and substituted or unsubstituted, the substituent group(s) preferably being hydroxyl, aryl, either mono- or di-nuclear aryl, as phenyl or naphthyl, and either unsubstituted or containing one or more substituents, especially selected from lower alkyl, definition of substituents, continued substituents on aryl moiety of aryl-alkyl group halogen, lower alkyl, lower alkoxy, or any combination of these substituent groups, mono- or di-lower-alkylamino, wherein the alkyl groups may be linked to form a hetero structure with the aminonitrogen to which they are attached such as to form an azacycloalkyl group, heterocyclic, and especially the pyridyl group, halogen, aryl or substituted aryl, the substituent group(s) preferably being halogen, and lower alkyl, heterocyclic, advantageously a pyridyl radical, alkylideneamino, and acyl; R₄ represents hydrogen, lower alkyl, either saturated or unsaturated and substituted or unsubstituted as described above for R₃ or R₃ and R₄ can be lower alkyl groups linked directly together or through a hetero atom, especially through oxygen or nitrogen to produce a 5 to 8 membered cyclic structure, thus forming with the nitrogen atom to which they are attached a 1-pyrrolidinyl, piperidino, 1-piperazinyl, especially a 4-lower alkyl-1-piperazinyl or morpholino, and the like radicals; and when R₂ and R₃ (or R₄) each represents a lower alkyl, they can be linked together to form a cyclic structure with the nitrogen atoms to which they are attached, particularly to form a 2-(2-imidazolinyl) radical. The 3-position amino group can be an unsubstituted amino as well as mono- or di-substituted amino groups, the substituent(s) advantageously being lower alkyl and lower alkanoyl and also where the substituents are linked to form a heterocyclic structure with the amino nitrogen to which they are attached.

Amiloride derivatives include 2′,4′-dichlorobenzamil amiloride, 2′,4′-dimethylbenzamil, 2′-methoxy-5′-nitrobenzamil, 2-chlorobenzylamiloride, 3′,4′-dichlorobenzamil, 3,5-diamino-6-fluoro-2-pyrazinoylguanidine, 3,5-diamino-N-(aminoiminomethyl)-6-bromopyrazine-N-methylcarboxamide, 4-(((((3,5-diamino-6-chloropyrazinyl)carbonyl)amino)iminomethyl)amino)-2,2,6,6-tetramethyl-1-piperidinyloxy, 5,6-dichloroamiloride, 5-(ethylpropyl)amiloride, 5-(N,N-hexamethylene)amiloride, 5-(N-2′-(4″-azidosalicylamidino)ethyl-N′-isopropyl)amiloride, 5-(N-2′-aminoethyl-N′-isopropyl)amiloride-N-(4″-azidosalicylamide), 5-(N-4-chlorobenzyl)-N-(2′,4′-dimethyl)benzamil, 5-(N-butyl-N-methyl)amiloride, 5-(N-ethyl-(2′-methoxy-5′-nitrobenzyl))amiloride, 5-(N-methyl-N-isobutyl)amiloride, 5-(N-methyl-N-propyl)amiloride, 5-(N-propyl-N-butyl)-2′,4′-dichlorobenzamil amiloride, 5-(N-tert-butyl)amiloride, 5-diethylamiloride, 5-dimethylamiloride, 5-N-(3-aminophenyl)amiloride, 5H-amiloride, 6-bromoamiloride, 6-bromobenzamil, 6-chloro-3,5-diaminopyrazine-3-carboxamide, 6-iodoamiloride, alpha′,2′-benzobenzamil, amiloride caproate, benzamil, co-amilozide, Esmalorid, ethylisopropylamiloride, frumil, kalten, methylisopropylamiloride, moducrin, N(5)-piperazine-amiloride, N(5)-piperidine-amiloride, phenylamil, and uranidil A.

### Ergotamine Alkaloids

In certain embodiments, ergotamine alkaloids such as bromocriptine, can be used in the compositions, methods, and kits of the invention. Bromocriptin analogs are described, for example, in U.S. Pat. No. 4,145,549. Ergotamine alkaliods include 1-methylergotamine, 9,10-dihydroergosine, bellataminal, Bellergal, beta-ergoptine, Bromocriptine, dihydroergocornine, dihydroergocristine, dihydroergocryptine, dihydroergotamine, dihydroergotoxine, ergosine, ergotamine, ergovaline, and neo-secatropin.

### Chlorophyllin

In certain embodiments, a chlorophyllide or an analog thereof can be used in the compositions, methods, and kits of the invention. Chlorophyllin is a derivative of chlorophyl, and a member of the chlorophyllides. Other chlorophyllides include chlorophyllide a, chlorophyllide b, methylchlorophyllide A, and methylchlorophyllide B.

### Cytarabine

In certain embodiments, cytarabine or an analog thereof can be used in the compositions, methods, and kits of the invention. Cytarabine is an antimetabolic and an antiviral agent. Cytarabine analogs are described in U.S. Pat. No. 3,116,282.

### Thyroxines

In certain embodiments, a thyroxine or derivative thereof can be used in the compositions, methods, and kits of the invention. Thyroxines are thyroid hormones and include levo thyroxine and dextrothyroxine, which has been used as antihyperlipidemic. The formula for dextrathyroxine is:

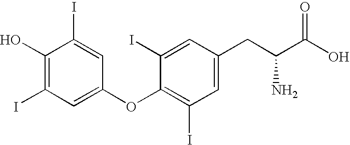

Dextrathyroxine can be administered orally and is typically provided in 2 mg or 4 mg tablets. Levothyroxine is used to increase the metabolic rate of cells.

### Pregnadienes

In certain embodiments, a pregnadiene or an analog or derivative thereof such as dydrogesterone can be used in the compositions, methods, and kits of the invention. Dydrogesterone is a progesterone and used thus to treat progesterone deficiency. Pregnadienes include 12-hydroxy-3-oxo-1,4-pregnadiene-20-carboxylic acid, 17-benzoyloxy-11-hydroxy-3,20-dioxo-1,4-pregnadien-21-al hemiacetal, 20-carboxy-1,4-pregnadien-3-one, 20-succinamylpregna-1,4-dien-3-one, 21-hydroxypregna-1,4-diene-3,11,20-trione, 3alpha-hydroxy-5alpha-pregna-9(11),16-diene-20-one, 3-hydroxy-5,7-pregnadien-20-one, canrenoate potassium, canrenone, chlormadinone acetate, cymegesolate, cyproterone, danazol, domoprednate, fluocinolone acetonide, GR 2-1159, icometasone enbutate, medrogestone, megestrol, melengestrol acetate, nivazol, oxyma, pregnadienediols, pregnadienetriols, rimexolone, Ro 12-2503, Ro 14-9012, Ro 6-1963, and triamcinolone.

### Evans Blue

In certain embodiments, a azo dye such as Evans blue can be used in the compositions, methods, and kits of the invention. Evans blue is used in blood volume and cardiac output measurement by the dye dilution method. It is very soluble, strongly bound to plasma albumin. The structure of Evans blue is:

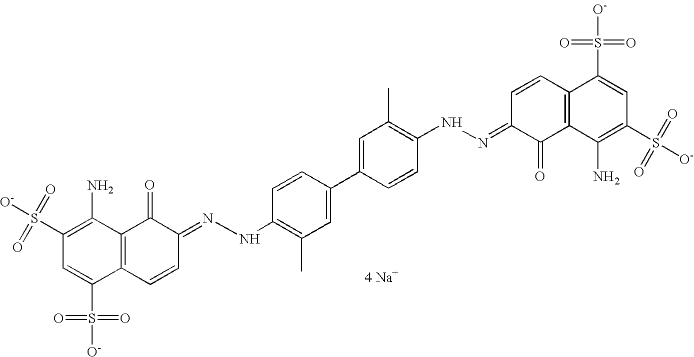

### Azetidines

In certain embodiments, an azetidine or derivative thereof such as ezitamibe can be used in the compositions, methods, and kits of the invention. The structure of ezitamibe is:

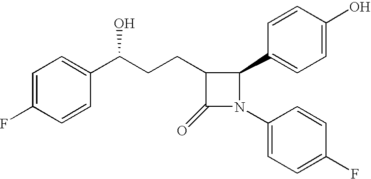

Analogs of ezitamibe are described, for example, in U.S. Pat. No. 5,767,115 and are described by the formula:

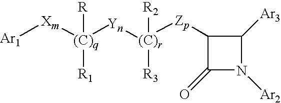

where Ar₁ and Ar₂ are independently selected from the group consisting of aryl and R₄-substituted aryl; Ar₃ is aryl or R₅-substituted aryl; X, Y and Z are independently selected from the group consisting of —CH₂—, —CH(lower alkyl)- and —C(dilower alkyl)-; R and R₂ are independently selected from the group consisting of —OR₆, —O(CO)R₆, —O(CO)OR₉ and —O(CO)NR₆R₇; R₁ and R₃ are independently selected from the group consisting of hydrogen, lower alkyl and aryl; q is 0 or 1; r is 0 or 1; m, n and p are independently 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5; R₄ is 1-5 substituents independently selected from the group consisting of lower alkyl, —OR₆, —O(CO)R₆, —O(CO)OR₉, —O(CH₂)₁₋₅OR₆, —O(CO)NR₆R₇, —NR₆R₇, —NR₆(CO)R₇, —NR₆(CO)OR₉, —NR₆ (CO)NR₇R₈, —NR₆ SO₂R₉, —COOR₆, —CONR₆R₇, —COR₆, —SO₂NR₆R₇, S(O)₀₋₂R₉, —O(CH₂)₁₋₁₀—COOR₆, —O(CH₂)₁₋₁₀CONR₆R₇, -(lower alkylene)COOR₆, —CH═CH—COOR₆, —CF₃, —CN, —NO₂ and halogen; R₅ is 1-5 substituents independently selected from the group consisting of —OR₆, —O(CO)R₆, —O(CO)OR₉, —O(CH₂)₁₋₅OR₆, —O(CO)NR₆R₇, —NR₆R₇, —NR₆(CO)R₇, —NR₆(CO)OR₉, —NR₆(CO)NR₇R₈, —NR₆SO₂R₉, —COOR₆, —CONR₆R₇, —COR₆, —SO₂NR₆R₇, S(O)₀₋₂R₉, —O(CH₂)₁₋₀—COOR₆, —O(CH₂)₁₋₁₀CONR₆R₇, -(lower alkylene)COOR₆ and —CH═CH—COOR₆; R₆, R₇ and R₈ are independently selected from the group consisting of hydrogen, lower alkyl, aryl and aryl-substituted lower alkyl; and R₉ is lower alkyl, aryl or aryl-substituted lower alkyl. R₄ is preferably 1-3 independently selected substituents, and R₅ is preferably 1-3 independently selected substituents. Preferred are compounds of formula I wherein Ar₁ is phenyl or R₄-substituted phenyl, especially (4-R₄)-substituted phenyl. Ar₂ is preferably phenyl or R₄-substituted phenyl, especially (4-R₄)-substituted phenyl. Ar₃ is preferably R₅-substituted phenyl, especially (4-R₅)-substituted phenyl. When Ar₁ is (4-R₄)-substituted phenyl, P4 is preferably a halogen. When Ar₂ and Ar₃ are R₄- and R₅-substituted phenyl, respectively, R₄ is preferably halogen or —OR₆ and R₅ is preferably —OR₆, wherein R₆ is lower alkyl or hydrogen. Especially preferred are compounds wherein each of Ar₁ and Ar₂ is 4-fluorophenyl and Ar₃ is 4-hydroxyphenyl or 4-methoxyphenyl.

Other azetidines include 1,4-bis(4-methoxyphenyl)-3-(3-phenylpropyl)-2-azetidinone, 1-(N-(3-ammoniopropyl)-N-(n-propyl)amino)diazen-1-ium-1,2-diolate, 1-methyl-2-(3-pyridyl)azetidine, 2-oxo-3-phenyl-1,3-oxazetidine, 2-tetradecylglycidyl-coenzyme A, 3-(2-oxopropylidene)azetidin-2-one, 3-aminonocardicinic acid, 3-phenyl-2-methylazetidine-3-ol, 4-((4-carboxyphenyl)oxy)-3,3-diethyl-1-(((phenylmethyl)amino)carbonyl)-2-azetidinone, 4-(3-amino-2-oxoazetidinonyl-1)methylbenzoic acid, 4-(3-amino-2-oxoazetidinonyl-1)methylcyclohexanecarboxylic acid, AHR 11748, azetidine, azetidine platinum(II), azetidinecarboxylic acid, azetidyl-2-carboxylic acid, azetirelin, BDF 9148, BMS-262084, E 4695, fluzinamide, L 652117, L 684248, N-(2-chloromethylphenyl)-3,3-difluoroazetidin-2-one, SCH 60663, SF 2185, tabtoxinine beta-lactam, tazadolene succinate, and ximelagatran.

### Thioxanthanes

In certain embodiments, thioxanthanes such as flupentixol can be used in the compositions, methods, and kits of the invention. Flupentixol is a antipsychotic that acts as a dopamine (D2 receptor) antagonist. Thioxanthane analogs are described, for example, in U.S. Pat. No. 3,951,961. Thioxanthane analogs include 2-(beta-diethylaminoethylamino)-3,4-cyclohexenothia-xanthone, 2-chlorothioxanthen-9-one, 2-thioxanthene, 3-carboxy-thioxanthone-10,10-dioxide, 4-(beta-diethylaminoethylamino)-1,2-cyclohexenothiaxanthone, 4-(bis(2′-chloroethyl)amino)propylamino-1,2-cyclohexenothioxanthone, 7-oxo-7-thiomethoxyxanthone-2-carboxylic acid, BW 616U76, chlorprothixene, clopenthixol, doxantrazole, flupenthixol, hycanthone, lucanthone, methixene, piflutixol, pimethixene, prothixene, quantacure QTX, spasmocanulase, teflutixol, thiothixene, and WIN 33377.

### Gemcitabine

In certain embodiments, gemcitabine or an analog thereof can be used in the compositions, methods, and kits of the invention. Gemcitabine is a nucleoside with antineoplastic activity.

Analogs of gemcitabine are described, for example, in U.S. Pat. No. 4,808,614 and have the general structure:

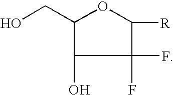

wherein R is a base of one of the formulae:

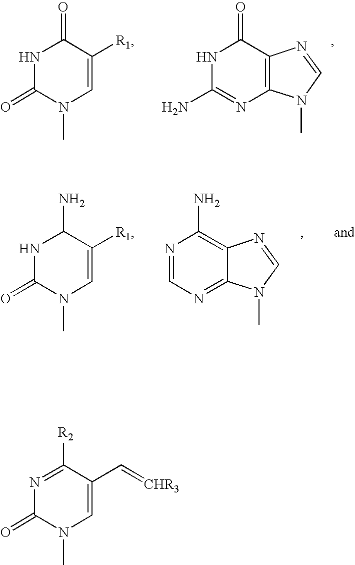

wherein R₁ is hydrogen, methyl, bromo, fluoro, chloro, or iodo; R₂ is hydroxy or amino; R₃ is hydrogen, bromo, chloro, or iodo.

### GW 5074

In certain embodiments, GW 5074 or an analog thereof can be used in the compositions, methods, and kits of the invention. GW 5074 is a benzylidene-1,3-dihydro-indol-2-one derivative which acts as a receptor tyrosine kinase inhibitor (e.g., raf such as cRaf1). The structure of GW 5074 is:

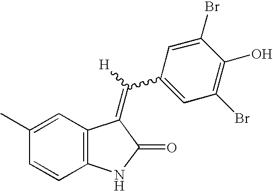

Analogs of GW 5074 are described, for example, in U.S. Pat. No. 6,268,391 and have the general structure:

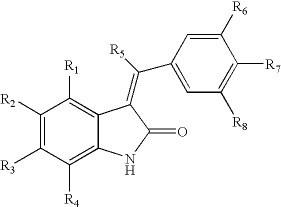

wherein R₁ is H or optionally joined with R₂ to form a fused ring selected from the group consisting of five to ten membered aryl, heteroaryl or heterocyclyl rings, said heteroaryl or said heterocyclyl rings having one to three heteroatoms where zero to three of said heteroatoms are N and zero to 1 of said heteroatoms are O or S and where said fused ring is optionally substituted by one to three of R₉, where R₂ and R₉ are as defined below; R₂ and R₃ are independently H, HET, aryl, C₁₋₁₂ aliphatic, CN, NO₂, halogen, R₁₀, —OR₁₀, —SR₁₀, —S(O)R₁₀, —SO₂R₁₀, —NR₁₀R₁₁, —NR₁₁R₁₂, —NR₁₂COR₁₁, —NR₁₂CO₂R₁₁, —NR₁₂CONR₁₁R₁₂, —NR₁₂SO₂R₁₁, —NR₁₂C(NR₁₂)NHR₁₁, —COR₁₁, —CO₂R₁₁, —CONR₁₂R₁₁, —SO₂NR₁₂R₁₁, —OCONR₁₂R₁₁, C(NR₁₂)NR₁₂R₁₁ where said C₁₋₁₂ aliphatic optionally bears one or two insertions of one to two groups selected from C(O), O, S, S(O), SO₂ or NR₁₂; with said HET, aryl or C₁₋₁₂ aliphatic being optionally substituted by one to three of R₁₀; and where R₂ is optionally joined with R₃ to form a fused ring selected from the group consisting of five to ten membered aryl, heteroaryl or heterocyclyl rings, said heteroaryl or said heterocyclyl rings having zero to three heteroatoms where zero to three of said heteroatoms are N and zero to one of said heteroatoms are O or S and where said fused ring is optionally substituted by one to three of R₉, where HET, R₉, R₁₀, R₁₁ and R₁₂ are as defined below; R₄ is H, halogen, NO₂ or CN; R₅ is H or C₁₋₁₂ aliphatic optionally substituted by one to three of halo, hydroxyl, heteroaryl, or aryl; R₆ and R₇ are independently halogen, CN, NO₂, —CONR₁₀R₁₁, —SO₂NR₁₀R₁₁, —NR₁₀R₁₁, or —OR₁₁, where R₁₀ and R₁₁ are as defined below; R₈ is OH, NHSO₂R₁₂ or NHCOCF₃; R₉ is each independently halogen, C₁₋₁₂ aliphatic, CN, —NO₂, R₁₀, —OR₁₁, —SR₁₁, —S(O)R₁₀, —SO₂R₁₀, —NR₁₀R₁₁, —N₁₁R₁₂, —NR₁₂COR₁₁, —NR₁₂CO₂R₁₁, —NR₁₂CONR₁₁R₁₂, —NR₁₂SO₂R₁₁, —NR₁₂C(NR₁₂)NHR₁₁, —CO₂R₁₁, —CONR₁₂R₁₁, —SO₂NR₁₂R₁₁, —OCONR₁₂R₁₁ or C(NR₁₂)NR₁₂R₁₁, where R₁₀, R₁₁ and R₁₂ are as defined below; R₁₀ is each independently H, halogen, C₁₋₁₂ aliphatic, aryl or HET, where said C₁₋₁₂ aliphatic optionally bears an inserted one to two groups selected from O, S, S(O), SO₂ or NR₁₂, where said C₁₋₁₂ aliphatic, aryl or HET is optionally substituted by one to three of halo, another HET, aryl, CN, —SR₁₂, —OR₁₂, —N(R₁₂)₂, —S(O)R₁₂, —SO₂R₁₂, —SO₂N(R₁₂)₂, —NR₁₂COR₁₂, —NR₁₂CO₂R₁₂, —NR₁₂CON(R₁₂)₂, —NR₁₂(NR₁₂)NHR₁₂, —CO₂R₁₂, —CON(R₁₂)₂, —NR₁₂SO₂R₁₂, —OCON(R₁₂)₂, where HET and R₁₂ are as defined below; R₁₁ is H or R₁₀; R₁₂ is H, C₁₋₁₂ aliphatic or HET, said C₁₋₁₂ aliphatic optionally substituted by one to three of halogen or OH where HET is as defined below; and HET is a five to ten-membered saturated or unsaturated heterocyclic ring selected from the group consisting of benzofuran, benzoxazole, dioxin, dioxane, dioxolane, dithiane, dithiazine, dithiazole, dithiolane, furan, imidazole, indole, indazole, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, quinoline, quinazoline, tetrahydrofuran, tetrazine, tetrazole, thiophene, thiadiazine, thiadiazole, thiatriazole, thiazine, thiazole, thiomorpholine, thianaphthalene, thiopyran, triazine, and triazole; and the pharmaceutically acceptable salts, biohydrolyzable esters, biohydrolyzable amides, biohydrolyzable carbamates, biohydrolyzable carbonates, biohydrolyzable ureides, solvates, hydrates, or prodrugs of the as defined above.

### Melphalan

In certain embodiments, melphalan or an analog thereof can be used in the compositions, methods, and kits of the invention. Melphalan is an alkylating nitrogen mustard used as an antineoplastic in the form of the levo isomer, melphalan. The racemic mixture is merphalan, and the dextro isomer is medphalan. Melphalan analogs are described, for example, in U.S. Pat. No. 3,032,584.

### Mosapride

In certain embodiments, mosapride or an analog thereof can be used in the compositions, methods, and kits of the invention. Mosapride is a benzamide that acts as a selective 5-HT₄ receptor agonist and is used as a gastroprokinetic. The structure of mosparide is:

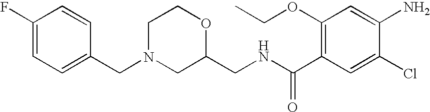

Analogs of mosparide are described, for example, in U.S. Pat. No. 4,870,074 and have the general structure:

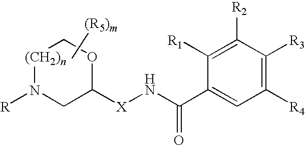

wherein R is hydrogen, a C₂-C₅ alkoxycarbonyl, benzyloxycarbonyl, a heteroaryl(C₁-C₃)alkyl in which the heteroaryl is furyl, thienyl, pyridyl, or 1,2-benzisoxazolyl, a phenyl(C₃-C₅)alkenyl, or -T-(Y)ₚ—R₆ (wherein T is a single bond or a C₁-C₆ alkylene, Y is oxygen, sulfur or carbonyl, R₆ is phenyl, a phenyl substituted by one to five members each independently selected from the group consisting of a halogen, a C₁-C₄ alkyl, trifluoromethyl, a C₁-C₄ alkoxy, nitro, cyano and amino, naphthyl, or diphenylmethyl, and p is 0 or 1, provided that when T is a single bond, p is 0), R₁ is a halogen, hydroxy, a C₁-C₁₂ alkoxy, a C₃-C₆ cycloalkyloxy, a C₃-C₈ alkenyloxy, a C₃-C₈ alkynyloxy, a C₂-C₆ alkoxy interrupted by one or two oxygens or carbonyls, a C₁-C₄ alkylthio, amino, a monosubstituted amino in which the substituted is a C₁-C₈ alkyl, a phenyl(C₁-C₃)alkyl or a C₃-C₆ cycloalkyl, a C₂-C₆ alkoxy in which the carbon atom at any position other than the 1-position is substituted by one hydroxy or amino, or a substituted C₁-C₆ alkoxy in which the substituent is a halogen, cyano, a C₂-C₅ alkoxycarbonyl, phthalimido, a C₃-C₆ cycloalkyl, a phenyl optionally substituted by one halogen, a phenoxy optionally substituted by one halogen, or a benzoyl optionally substituted by one halogen, R₂ is hydrogen, R₃ is hydrogen, a halogen, amino, a C₁-C₄ alkylamino, a di(C₁-C₄ alkyl)amino, a C₂-C₅ alkanoylamino, or nitro, R₄ is hydrogen, a halogen, nitro, sulfamoyl, a C₁-C₄ alkylsulfamoyl, or a di(C₁-C₄ alkyl)sulfamoyl, or any two adjacent groups of the R₁, R₂, R₃ and R₄ combine to form a C₁-C₃ alkylenedioxy, and the remaining two groups are each hydrogen, R₅ is hydrogen or a C₁-C₄ alkyl, X is a C₁-C₃ alkylene, and m and n are each 1 or 2, provided that at least one of the groups R₂, R₃ and R₄ is not hydrogen.

Mosapride is a benzamide. Other benzamides include 1-((4-fluorobenzoylamino)ethyl)-4-(7-methoxy-1-naphthyl)piperazine hydrochloride, 1-(3,4-dihydroxyphenyl)-2-(3-(4-carbamylphenyl)-1-methylpropylamino)ethanol, 1-nitrohydroxyphenyl-N-benzoylalanine, 2,2′-dithiobis(N-2-hydroxypropylbenzamide), 2,3-dimethoxy-5-iodo-N-((1-(4′-fluorobenzyl)-2-pyrrolidinyl)methyl)benzamide, 2,3-dimethoxy-N-(1-(4-fluorobenzyl)piperidin-4-yl)benzamide, 2,3-dimethoxy-N-(9-(4-fluorobenzyl)-9-azabicyclo(3.3.1)nonan-3-yl)benzamide, 2,4-dichloro-6-nitrophenolamide, 2,6-dichlorobenzamide, 2,6-difluorobenzamide, 2-(2-chloro-4-iodophenylamino)-N-cyclopropylmethoxy-3,4-difluoro-5-bromobenzamide, 2-chlorobenzamide, 2-hexyloxybenzamide, 2-methoxy-4-fluoro-3-amino-N-((2-methylcyclopropylamino)ethyl)benzamide, 264 CP, 3,4,5-trimethoxybenzamide, 3,4-dichloro-N,N-di-sec-butylbenzamide, 3-(3-(dimethylamino)propyl)-4-hydroxy-N-(4-(4-pyridinyl)phenyl)benzamide, 3-(cyclopentyloxy)-N-(3,5-dichloro-4-pyridyl)-4-methoxybenzamide, 3-(N-butyrylamino)benzamide, 3-acetamidobenzamide, 3-aminobenzamide, 3-carbamyl-(3′-picolyl)-4-methoxy-1-benzamide, 3-chloro-N-(4,6-dimethyl-2-pyridiny) benzamide, 3-iodo-2-hydroxy-6-methoxy-N-((1-ethyl-2-pyrrolidinyl)methyl)benzamide, 3-methoxybenzamide, 3-nitrosobenzamide, 4-((methylsulfonyl)amino)-N-((4-phenylpiperazin-2-yl)methyl)benzamide, 4-(1H-tetrazol-5-yl)-N-(4-(1H-tetrazol-5-yl)phenyl)benzamide, 4-(3-(2-hydroxy-2-phenyl)ethylamino-3-methylbutyl)benzamide, 4-(5-benzo(1,3)dioxol-5-yl-4-pyridin-2-yl-1H-imidazol-2-yl)benzamide, 4-(alpha-(4-allyl-2,5-dimethyl-1-piperazinyl)-3-methoxybenzyl)-N,N-diethylbenzamide, 4-(trifluoromethyl)benzamide, 4-amino-5-chloro-2-ethoxy-N-((2-morpholinyl)methyl)benzamide, 4-amino-N-((4-benzyl-2-morpholinyl)-methyl)-5-chloro-2-ethoxybenzamide, 4-amino-N-((4-benzyl-2-morpholinyl)methyl)-5-chloro-2-methoxybenzamide, 4-aminobenzamidopyridine, 4-azido-5-iodoclebopride, 4-chloro-N-(hydroxymethyl)benzamide, 4-diethoxyphosphorylmethyl-N-(4-bromo-2-cyanophenyl)benzamide, 4-dimethylamino-N-(4-(2-hydroxycarbamoylvinyl)benzyl)benzamide, 4-fluorobenzamide, 4-fluorobenzylamine, 4-hydroxybenzamide, 4-iodo-N-(2-(4-morpholinyl)ethyl)benzamide, 4-iodo-N-piperidinoethylbenzamide, 5-(aziridin-1-yl)-2-nitro-4-nitrosobenzamide, 5-bromo-2,3-dimethoxy-N-((1-(4-fluorobenzyl)-2-pyrrolidinyl)methyl)benzamide, 5-bromo-2-ethoxybenzamide, 5-fluoropropylepidepride, 7-(3-(2-(cyclopropylmethyl)-3-methoxy-4-((methylamino)carbonyl)phenoxy)propoxy)-3,4-dihydro-8-propyl-2H-1-benzopyran-2-propanoic acid, A 22700, AH 7921, aklomide, alloclamide, ameltolide, azapride, BA 74, befol, benodanil, benzamide, benzamide adenine nucleotide, benzcoprine, benzotripte, bis(2-(N-phenylcarboxamido)phenyl)diselenide, BRL 24682, BRL 32872, BRL 34778, bromadoline, bromtianide, brovanexine, BW 373U86, BWA 466C, BWA 728C, Card-Instenon, cinitapride, Cisapride, clebopride, cloxacepride, dazopride, DEET, dehydroxymethylepoxyquinomicin, desbenzylclebopride, Diethyltoluamide-20, dimetpramid, Dinitolmide, dobupride, ecabapide, EL 494, epidepride, ethamivan, ethyl 2-(4′-carboxybenzamido)-4-aminobenzoate, ethyl 2-(4′-carboxybenzamido)-4-propionamidobenzoate, FLA 981, flatoril, FLB 524, fluoroclebopride, fluphenacur, flurfamide, fomesafen, gentisamide, GGTI 297, GGTI 298, GR11665, GW 300, GW 532, GW 575, hexafluoron, Hippurates, HMR 1098, Indoramin, Instenon, iodopride, iofratol, isoxaben, itopride, L 1215, L 7063, LY 135114, LY 188544, LY 201409, meglitinide, Metoclopramide, Moclobemide, N(1)-(4-chlorobenzoyl)-N(2)-(1-(1-naphthyl)ethyl)-1,2-diaminocyclohexane, N,N-dimethylbenzamide, N-((4-benzyl-2-morpholinyl)methyl)-5-chloro-4-(dimethylamino)-2-methoxybenzamide, N-((4-methylphenyl)sulfonyl)-3-(2-quinolinylmethoxy)benzamide, N-(1′-benzyl-4′-piperidyl-N-oxide)-4-amino-5-chloro-2-methoxybenzamide, N-(2,6-dimethylphenyl)-4-(((diethylamino)acetyl)amino)benzamide, N-(2-(diethylamino)ethyl)-4-iodobenzamide, N-(2-(diethylamino)ethyl)benzamide, N-(2-aminocyclohexyl)-3,4-dichlorobenzamide, N-(2-aminoethyl)-2-anisamide, N-(2-aminophenyl)-4-(N-(pyridin-3-ylmethoxycarbonyl)aminomethyl)benzamide, N-(2-dimethylaminoethyl)-2-anisamide, N-(2-methylaminocyclohexyl)-3,4-dichlorobenzamide, N-(2-picolyl)-3,5-dimethylbenzamide, N-(3,4,5-trimethoxybenzoyloxy)-3,4,5-trimethoxybenzamide, N-(3-picolyl)-3,5-dimethylbenzamide, N-(4′-(delta-1′-piperidyl-N-oxide))-4-amino-5-chloro-2-methoxybenzamide, N-(4′-(N-hydroxypiperidyl))-4-amino-5-chloro-2-methoxybenzamide, N-(4,6-dimethyl-2-pyridinyl)benzamide, N-(4-(2-(dimethylamino)ethoxy)benzyl)-3,4-dimethoxybenzmide, N-(4-(5-bromo-2-pyrimidinyloxy)-3-chlorophenyl)-N′-(2-nitrobenzoyl)urea, N-(4-acetyl-1-piperazinyl)-4-fluorobenzamide monohydrate, N-(4-amino-1-butyl)-N-nitrosobenzamide, N-(4-chlorobenzoyl)-N-methyl-4-(4-dimethylaminomethylphenyl)cyclohexylamine, N-(acetoxymethyl)-4-chlorobenzamide, N-(exo-(hexahydro-1H-pyrrolizine-1-yl)methyl)-2-methoxy-4-amino-5-chlorobenzamide, N—(N-benzylpiperidin-4-yl)-4-iodobenzamide, N-2-fluorenylbenzamide, N-acetylbenzamide, N-butyrylbenzamide, N-demethylbromadoline, N-didemethylbromadoline, N-ethylbenzamide, N-formylbenzamide, N-hydroxymethyl-N-methylbenzamide, N-hydroxymethylbenzamide, N-isopropyl-4-hydroxymethylbenzamide, N-methyl-2,3-dihydroxybenzamide, N-methylbenzamide, N-octyl-3-nitro-2,4,6-trihydroxybenzamide, N-propionylbenzamide, N-pyrimidinobenzamide-2-carboxylic acid, nemonapride, nitromide, norcisapride, NP 101A, pancopride, parsalmide, Pellit, penfluoron, picobenzide, picobenzide N-oxide, Procainamide, Procarbazine, pronamide, Raclopride, rebemide, Remoxipride, renzapride, RG-4, RG-7, riparin, Ro 12-5637, Ro 12-8095, Ro 16-3177, Ro 16-6491, roflumilast, S 1688, SC 53116, sirtinol, SNC 121, spectramide, SR 48968, Sulpiride, T 0070907, teflubenzuron, tegalide, Tiapride, tonabersat, triflumuron, trimethobenzamide, WAY 100289, YM-08050, Z 338, and zacopride.

### Octyl Methoxycinnamate

In certain embodiments, telaprevir or an analog thereof can be used in the compositions, methods, and kits of the invention. Octyl methoxycinnamate absorbs ultraviolet (UV) light and is used in sunscreens and other topical applications where UV protection is desired. The structure of octyl methoxycinnamte is:

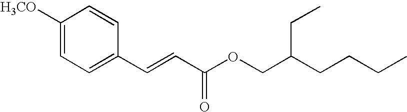

Cinnamic acid derivatives are described, for example, in U.S. Pat. No. 5,457,226 and have the general structure:

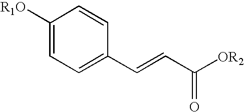

wherein R₁ signifies hydrogen or C₁₋₈-alkyl and R₂ signifies hydrogen, C₁₋₁₀-alkyl, C₁₋₁₀-hydroxyalkyl or C₁₋₄-alkoxy-C₁₋₁₀-alkyl. Cinnamic acid derivative include Other cinnmates include (4-(dimethylamino)cinnamoyl)imidazole, (N-(3,5-dimethoxy-4-n-octyloxycinnamoyl)-N′-(3,4-dimethylphenyl)piperazine), 1,1-dimethylallyl-3′,4′-dihydroxycinnamic acid ester, 2,3-dihydroxycinnamic acid, 2-(4-amylcinnamoyl)amino-4-chlorobenzoic acid, 2-chlorocinnamic acid, 2-ethylhexyl-4-methoxycinnamate, 2-fluoro-p-hydroxycinnamate, 2-fluorocinnamic acid, 3,4,5-trimethoxycinnamic acid, 3,4-di(OH)-cinnamate, 3,4-dihydroxyhydrocinammic acid (1-aspartic acid dibenzyl ester) amide, 3,5-dihydroxycinnamic acid, 3,5-dimethoxycinnamic acid, 3,7-dimethyl-1,6-octadien-3-yl cinnamtae, 3-(3,4-dimethoxyphenyl)propenoic acid, 3-(4′-hydroxy-3′-adamantylbiphenyl-4-yl)acrylic acid, 3-(4-(1,2-diphenylbut-1-enyl)phenyl)acrylic acid, 3-(4-methoxyphenyl)-2-propenoic acid 3-methylbutyl ester, 3-(trifluoromethyl)cinnamide, 3-bromocinnamamide, 3-bromocinnamic acid, 3-fluorocinnamic acid, 4-(3,3-dimethyl-1-triazeno)cinnamic acid, 4-(3-(1-adamantyl)-4-hydroxyphenyl)-3-chlorocinnamic acid, 4-amidinophenyl 2-methylcinnamate, 4-amidinophenyl cinnamate, 4-amylcinnamoylanthranilic acid, 4-dimethylaminocinnamaldehyde, 4-fluorocinnamic acid, 4-hydroxy-3-methoxycinnamylpiperidine, 4-hydroxycinnamic acid (1-phenylalanine methyl ester) amide, 4-methoxycinnamate methyl ester, 4-methoxycinnamic acid, 5-(2-(methyl(2-phenethyl)amino)-2-oxoethyl)-2-(benzyloxy)cinnamic acid, A 25794, adamon, alpha-cyanocinnamate, alpha-methyl-2-hydroxy-4-diethylaminocinnamic acid, alpha-phenylcinnamate, aminocinnamonitrile, antithiamine factor, asarumin C, BM 42304, caffeic acids (e.g., 1,1-dimethylallyl caffeic acid ester, 2-S-glutathionylcaffeic acid, 3,4-dihydroxyphenylpropionic acid, 7-caffeoylloganin, caffeic acid, caffeic acid phenethyl ester, calceolarioside A, chicoric acid, crenatoside, dehydrodicaffeic acid dilactone, ethyl caffeate, ethyl ferulate, eugenol, fukinolic acid, methyl caffeate, myriceron caffeoyl ester, N-(3,4-diacetoxycinnamoyl)-2-pyrrolidone, N-caffeoyl-4-aminobutyric acid, octyl caffeate, petasiphenol, phenylethyl 3-methylcaffeate, salvianolic acid A, suspensaside, and swertiamacroside), caracasanamide, chlorogenic acid, cinametic acid, cinanserin or derivatives thereof (e.g., SQ 10631 and SQ 11447), cinnamic acid, cinnamic anhydride, cinnamoyl chloride, cinnamyl isobutyrate, cinromide, CKA 1303, clocinnamox, coniferin, coumaric acids (e.g., (3,4-disinapoyl)fructofuranosyl-(6-sinapoyl)glucopyranoside, (3-sinapoyl)fructofuranosyl-(6-sinapoyl)glucopyranoside, 1-(4-coumaroyl)alpha-rhamnopyranose, 2-hydroxycinnamic acid, 3-coumaric acid, 4-coumaric acid, 4-coumaric acid methyl ester, 4-hydroxycinnamoylmethane, 5-hydroxyferulic acid, 5-O-feruloylarabinose, alpha-cyano-3-hydroxycinnamate, alpha-cyano-4-hydroxycinnamate, angoroside C, asprellic acid A, coniferyl ferulate, cycloartenol ferulic acid ester, dihydro-3-coumaric acid, ferulic acid, feruloylputrescine, feruloyltyramine, karenin, methyl 5-O-feruloylarabinofuranoside, and sinapinic acid), cyclamen aldehyde, cyclamen aldehyde methyl anthranilate, diacetylcymarol, dimethylaminoethyl-alpha-phenylcinnamate, Dolo-Adamon, ethyl 2,5-dihydroxycinnamate, ethyl cinnamate, fagaramide, gagaminine, hordatine M, hygromycin A, igmesine, isoferulic acid, kutkin, linusitamarin, maxafil, methyl 2,5-dihydroxycinnamate, methyl 3-phenyl-2,3-epoxypropanoate, methyl 4-(dimethylamino)cinnamate, methyl cinnamate, N,N-dimethylhydrocinnamide, N-hydroxy-N-methyl-3-(2-(methylthio)phenyl)-2-propenamide, O-(alpha-(benzoylamino)-4-(phenylazo)cinnamoyl)-beta-phenyllactate, O-(alpha-(benzoylamino)cinnamoyl)-beta phenyllactate, octylmethoxycinnamate, ONO 8713, penupogenin, picroside I, picroside II, puromycin or derivative thereof (e.g., 2′-deoxypuromycin, 4-azidopuromycin, carbocyclic puromycin, cyclohexylpuromycin, cytidine-2′(3′)-P-5′-puromycin, methionylpuromycin, N-(2-nitro-4-azidobenzoyl)puromycin, N-acetylphenylalanylpuromycin, N-iodoacetylpuromycin, O-demethylpuromycin, puromycin aminonucleoside, and sparsopuromycin), Ro 03-6037, rosmarinic acid, S 8932, SC 1001A, sibirate, SQ 10624, ST 638, SU 1498, tolibut, trans-3-(2′-methylphenyl)-2-propene-1-carboxamide, vanicoside A, and vanicoside B.

### Oxeladin

In certain embodiments, oxeladin or an analog thereof can be used in the compositions, methods, and kits of the invention. Oxeladin is a used as an antitussive agent. The structure of oxeladin is:

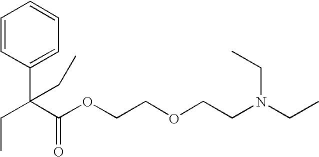

Oxeladin derivates are described, for example, in U.S. Pat. No. 2,885,404 and have the general structure:

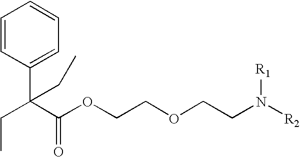

in which R₁ and R₂ are alkyl groups containing together not more than 12 carbon atoms, or together form a cyclic structure wherein —NR₁R₂ represents pyrrolidino, piperideino or piperidino. The groups R₁ and R₂ may be the same or different. Particular derivatives include 2-(β-diethylaminoethoxy)ethyl diethylphenylacetate, 2-(β-N-pyrrolidinoethoxy)ethyl diethylphenylacetate, 2-(β-N-piperidinoethoxy)ethyl diethylphenylacetate, 2-(β-N-Δ³-piperideinoethoxy)ethyl diethylphenylacetate, 2-(β-N-ethylmethylaminoethoxy)ethyl diethylphenylacetate, 2-(β-N-ethylpropylaminoethoxy)ethyl diethylphenylacetate, 2-(β-N-di-n-butylaminoethoxy)ethyl diethylphenylacetate and 2-(β-di-n-hexylaminoethoxy)ethyl diethylphenylacetate.

### Parthenolide

In certain embodiments, parthenolide or an analog thereof can be used in the compositions, methods, and kits of the invention. Parthenolide is a sesquiterpene lactone found in plants such as feverfew and Chrysanthemum parthenium. It has anti NFκB activity. The structure of parthenolide is:

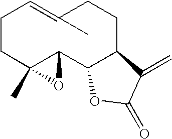

Analogs of parthenolide are described, for example, in U.S. Pat. Application Publication No. 2005/0032886 and have the following structure.

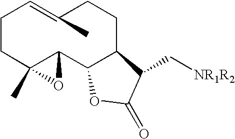

wherein R₁ and R₂ may be the same or different; R₁ is selected from hydrogen, alkyl, substituted alkyl, cycloalkyl, substituted cycloalkyl, hydroxyalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, substituted heterocyclic, trifluoromethyl, perfluoroalkyl, cyano, cyanomethyl, carboxyl, carbamate, sulfonyl, sulfonamide and aryloxyalkyl, or OR₁, wherein, O is an oxygen; R₂ is selected from hydrogen, alkyl, substituted alkyl, cycloalkyl, substituted cycloalkyl, hydroxyalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, substituted heterocyclic, trifluoromethyl, perfluoroalkyl, cyano, cyanomethyl, carboxyl, carbamate, sulfonyl, sulfonamide and aryloxyalkyl. In certain embodiments, R₁ is hydrogen or optionally substituted lower alkyl; and R₂ is optionally substituted lower alkyl. R₁ and R₂ can be each —CH₃, or each —CH₂CH₃. R₁ can be —CH₂CH₃ and R₂ can be —CH₃. R₁ can be —CH₂CH₂CH₃ and R₂ can be —CH₃. R₁ can be —CH(CH₃)₂, and R₂ can be —CH₃. R₁ and R₂ also can combine with N to form a ring system. Examples of such combination include —CH₂(CH₂)ₙCH₂—; where n is selected from 0 to 5. These ring systems can also have one or more substituents selected from alkyl, substituted alkyl, cycloalkyl, substituted cycloalkyl, hydroxyalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, substituted heterocyclic, trifluoromethyl, perfluoroalkyl, cyano, cyanomethyl, carboxyl, carbamate, sulfonyl, sulfonamide, aryloxyalkyl and halogen as set forth above. This ring system can also be —CH₂(CH₂)ₙCH₂Z-; where Z is O, S, Se, Si, P, —CO—, —SO—, —SO₂—, —PO—; and —CH₂(CH₂)ₙCH₂— are the groups as set forth above. Alternatively, this ring system can be —(CH₂)ₐ-Z-(CH₂)_{b}—; where a and b are the same or different and are from 1 to 4; and Z is O, N, S, Se, Si, P, —CO—, —SO—, —SO₂— or —PO—. This ring system can also be a uracil ring and its derivatives with one or more substituents. These ring systems can also have one or more substituents connected to the carbon atom(s) and/or Z. The substituent is selected from alkyl, substituted alkyl, cycloalkyl, substituted cycloalkyl, hydroxyalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, substituted heterocyclic, trifluoromethyl, perfluoroalkyl, cyano, cyanomethyl, carboxyl, carbamate, sulfonyl, sulfonamide, aryloxyalkyl and halogen as set forth above. These ring systems can also be aromatic, such as pyrrole, imidazole, purine, and pyrazole and substituted derivative of these heterocyclics listed above with one or more substituents selected from alkyl, substituted alkyl, cycloalkyl, substituted cycloalkyl, hydroxyalkyl, alkenyl, substituted alkenyl, alkynyl, substituted alkynyl, aryl, substituted aryl, alkylaryl, substituted alkylaryl, arylalkyl, substituted arylalkyl, arylalkenyl, substituted arylalkenyl, arylalkynyl, substituted arylalkynyl, heterocyclic, substituted heterocyclic, trifluoromethyl, perfluoroalkyl, cyano, cyanomethyl, carboxyl, carboxylate, carboxaldehyde, carboxamide, carbamate, hydroxy, alkoxy, isocyanate, isothiocyanate, nitro, nitroso, nitrate, sulfate, sulfonyl, sulfonamide, thiol, thioalkyl, aryloxyalkyl and halogen as set forth above. Any of the above ring systems comprising NR₁R₂ may optionally be fused with another ring to form an optionally substituted bicyclic or tricyclic ring system, each of the rings optionally comprising one or more heteroatoms. Preferred ring systems include aziridin-1-yl, azetidin-1-yl, pyrrolidin-1-yl, piperidin-1-yl, homopiperidyn-1-yl and heptamethyleneimin-1-yl, each being optionally substituted with one or more substituents as set forth above. Exemplary parthenolide derivatives include 11βH,13-Dimethylaminoparthenolide; 11βH,13-Diethylaminoparthenolide; 11βH,13-(tert-Butylamino)parthenolide; 11βH,13-(Pyrrolidin-1-yl)parthenolide; 11βH,13-(Piperidin-1-yl)parthenolide; 11βH,13-(Morpholin-1-yl)parthenolide; 11βH,13-(4-Methylpiperidin-1-yl)parthenolide; 11βH,13-(4-Methylpiperazin-1-yl)parthenolide; 11βH,13-(Homopiperidin-1-yl)parthenolide; 11βH,13-(Heptamethyleneimin-1-yl)parthenolide; 11βH,13-(Azetidin-1-yl)parthenolide; and 11βH,13-Diallylaminoparthenolide.

### Quinacrine

In certain embodiments, quinacrine or an analog thereof can be used in the compositions, methods, and kits of the invention. Quinacrine is an antiparasitic and an antiprotozoal (e.g., antimalarial) agent. The structure of quinacrine is:

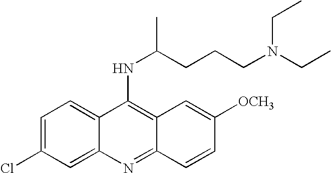

Analogs of quincrine are described, for example, in U.S. Pat. No. 1,782,272 and have the following structure:

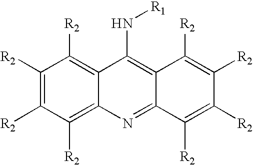

wherein R₁ stands for hydrogen or alkyl, at least one R₂ for the nitro group and another R₂ for a basic residue, the remaining R₂ representing hydrogen, halogen, or a nitro-, alkyl- or alkoxy group, where a “basic residue” is By the term “basic residue” is to be understood in the sense of the foregoing formula such groups contain at least one aliphatically bound N-atom and which may be linked to the acridine ring for instance through the medium of oxygen (in the manner of an ether), of nitrogen (in the manner of an amine), or of carbon (in the manner of a C—C linkage). Derivatives of quinacrine include acrisuxine, collagenan, dimethylquinacrine, Preparation ABP, quinacrine half mustard, and quinacrine mustard.

Quinacrine is an aminoacridine. Other aminoacridines include (((amino-2-ethyl)-2-aminomethyl)-2-pyridine-6-carboxylhistidyl-gamma-(2-amino-2-deoxyglucosyl)glutamylglycylamino)-4-phenyl-1-aminoacridine, (N-(2-((4-((2-((4-(9-acridinylamino)phenyl)amino)-2-oxoethyl)amino)-4-oxobutyl)amino)-1-(1H-imidazol-4-ylmethyl)-1-oxoethyl)-6-(((−2-aminoethyl)amino)methyl)-2-pyridinecarboxamidato) iron(1+), 1,2,3,4-tetrahydro-N-(3-iodophenyl-methyl)-9-acridinamine, 1,2,3,4-tetrahydro-N-(phenyl-methyl)-9-acridinamine, 1-nitro-9-(dimethylamino)acridine, 10-N-nonylacridinium orange, 2-(3,6-bis(dimethylamino)-10-acridinyl)ethyl-(2,3-di-O-palmitoylglycero)phosphate, 2-aminoacridone, 3,6-diamino-10-methylacridinium, 3,6-diamino-9-(4-(methylsulfonyl)aminophenyl)aminoacridine, 3-amino-6-methoxy-9-(2-hydroxyethylamino)acridine, 3-amino-6-methoxyacridine, 3-amino-7-methoxyacridine, 3-amino-9-(diethylaminoethylthio)acridine, 3-aminothioacridone, 3-dimethylamino-6-methoxyacridine, 4-(9-acridinylamino)-N-(4-(((4-amino-1-methylpyrrol-2-yl)carbonyl)amino)-1-methylpyrrol-2-carbonyl)glycylaniline, 4-(9-acridinylamino)-N-(glycyl-histidyl-lysyl-glycyl)aniline, 9-((6-(4-nitrobenzoyloxy)hexyl)amino)acridine, 9-(2-(2-nitro-1-imidazolyl)ethylamino)acridine, 9-(5-carboxypentylamino)acridine, 9-(6-(2-diazocyclopentadienylcarbonyloxy)hexylamino)acridine, 9-(6-(4-azidobenzamido)hexylamino)acridine, 9-amino-2-hydroxyacridine, 9-amino-3-azido-7-methoxyacridine, 9-amino-6-chloro-2-methoxyacridine, 9-amino-6-chloroacridine-2-phosphate, 9-aminoacridine-4-carboxamide, acridine mustard, acridine orange, acridine yellow, acriflavine, aminacrine, Amsacrine, C 1310, C 1311, C 325, C 829, coriphosphine, ethacridine, euchrysine, fluoroquinacrine, N-((2-dimethylamino)ethyl)-9-aminoacridine-4-carboxamide, N-((4-dimethylamino)butyl)-9-aminoacridine-4-carboxamide, N-(6-azido-2-methoxy-9-acridinyl)-N′-(9-acridinyl)octane-1,8-diamine, N-(9-acridinyl)bromoacetamide, Nitracrine, NLA 1, NSC 210733, proflavine, pyracrine phosphate, SDM, suronacrine, and tacrine.

### Repaglinide

In certain embodiments, repaglinide or an analog thereof can be used in the compositions, methods, and kits of the invention. Repaglinide is an antidiabetic agent which lowers glucose levels by closing potassium channels in the b-cell membrane. The structure of repaglinide is:

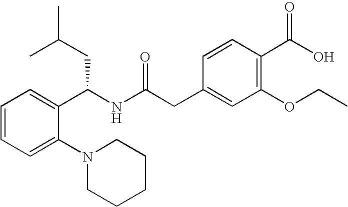

Analogs of repaglinide are described, for example, in U.S. Pat. No. 5,312,924 and can be represented as follows:

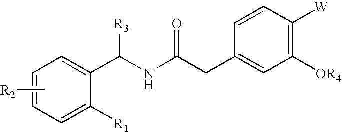

wherein R₁ represents an unbranched alkyleneimino group with 4 to 6 carbon atoms optionally mono- or di-(alkyl of 1 to 3 carbon atoms)-substituted; R₂ represents a hydrogen or halogen atom or a methyl or methoxy group; R₃ represents a hydrogen atom, an alkyl group with 1 to 7 carbon atoms, a phenyl group optionally substituted by a halogen atom or a methyl or methoxy group, an alkyl group with 1 or 2 carbon atoms substituted by a hydroxy, alkoxy, alkanoyloxy, tetrahydrofuranyl, tetrahydropyranyl, cycloalkyl or phenyl group, in which the alkoxy part can contain from 1 to 3 carbon atoms, the alkanoyloxy part can contain 2 to 3 carbon atoms and the cycloalkyl part can contain 3 to 7 carbon atoms, an alkenyl group with 3 to 6 carbon atoms, an alkynyl group with 3 to 5 carbon atoms, a carboxy group or an alkoxycarbonyl group with a total of 2 to 5 carbon atoms; R₄ represents a hydrogen atom, a methyl, ethyl or allyl group; and W represents a methyl, hydroxymethyl, formyl, carboxyl, alkoxycarbonyl, cyanomethyl, 2-cyano-ethyl, 2-cyano-ethenyl, carboxymethyl, 2-carboxyethyl, 2-carboxyethenyl, alkoxycarbonylmethyl, 2-alkoxycarbonyl-ethyl or 2-alkoxycarbonylethenyl group, in which each alkoxy part can contain from 1 to 4 carbon atoms and can be substituted by a phenyl group; and when R₃ is other then hydrogen and/or the radical R₁ contains an optically active carbon atom, the enantiomeres and the diastereomeres thereof or their mixtures; when W is carboxyl, a non-toxic salt thereof formed with an inorganic or organic base; or a non-toxic acid addition salt thereof formed by an inorganic or organic acid with the amino function in the R₁-position.

### Rifamycins

In certain embodiments, a rifamycin such as rifabutin or an analog thereof can be used in the compositions, methods, and kits of the invention. Rifamycins are antibiotic compounds. The structure of rifabutin, an exemplary rifamycin, is:

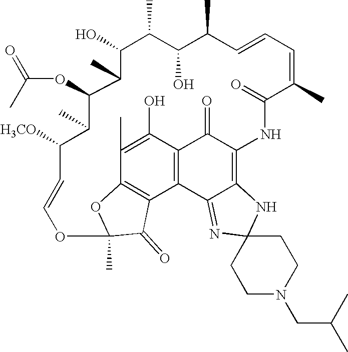

Rifabutin analogs are described, for example, in U.S. Pat. No. 4,219,478, and have the general structure:

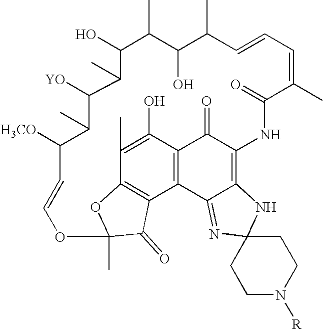

where R is selected from the group consisting of linear alkyl having 4 to 8 carbon atoms, branched alkyl having 4 to 8 carbon atoms, alkenyl having 3 or 4 carbon atoms, cycloalkyl having 3 to 6 carbon atoms, alkoxyalkyl having 3 to 7 carbon atoms, alkyl-furyl having 5 or 6 carbon atoms, alkyl tetrahydrofuryl having 5 or 6 carbon atoms, alkanoyl having 5 or 6 carbon atoms, and monohaloalkanoyl having 2 to 6 carbon atoms, and Y is —H or —COCH₃. Other rifamycins include 16,17-dihydro-17-hydroxyrifamycin S, 16,17-dihydrorifamycin S, 25-deacetoxy-25-hydroxyrifamycin S, 3-((dimethylhydrazono)methyl)rifamycin SV, 3-carbomethoxy rifamycin S, 3-formyl-25-desacetylrifamycin, 3-formylrifamycin SV, 31-homorifamycin W, 4-deoxy-3′-bromopyrido(1′,2′-1,2)imidazo[5,4-c]rifamycin S, AF 013, benzothiazole-rifamycin, C 27, CGP 27557, CGP 29861, CGP 4832, CGP 7040, FCE 22250, FCE 22807, halomicin B, kanglemycin A, KRM 1648, KRM 1657, KRM 1668, KRM 1671, protorifamycin I, R 761, reprimun, rifabutin derivatives (e.g., 17-(allylamino)-17-demethoxygeldanamycin, 25-desacetylrifabutin, and streptovaricin), rifamdin, rifamexil, rifamide, Rifampin or derivatives thereof (e.g., 18,19-dihydrorifampicin, 25-deacetylrifampicin, 25-desacetylrifapentine, CGP 43371, CGS 24565, dehydrorifampicin, DMB-rifampicin, rifampicin N-oxide, rifapentine, Rifaprim, Rifater, and rivicycline), rifamycin B, rifamycin L, rifamycin O, rifamycin P, rifamycin Q, rifamycin S, rifamycin SV, rifamycin Verde, rifaximin, rifazone-82, SPA-S 565, streptovaricin derivatives (e.g., damavaricin C, damavaricin Fc pentyl ether, protostreptovaricin, streptoval C, streptovaricin C, and streptovarone), tolypomycin Y, and tolypomycinone.

### SB-202190

In certain embodiments, SB-202190 or an analog thereof can be used in the compositions, methods, and kits of the invention. SB-202190 is a pyridyl substituted imidazole with selective p38 MAP Kinase (MAPK) inhibitory activity. SB-202190 binds to the ATP binding site on active p38 MAPK. The structure of SB-202190 is:

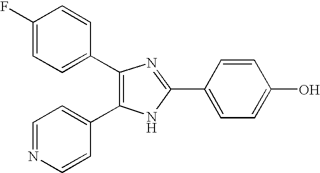

Analogs of SB-202190 are described, for example, in U.S. Pat. No. 6,008,235 and have the structure:

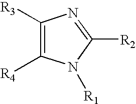

wherein R₁ is a mono- or di-substituted 4-quinolyl, 4-pyridyl, 1-imidazolyl, 1-benzimidazolyl, 4-pyrimidinyl wherein the substituent is independently selected from the group consisting of hydrogen, C₁₋₄ alkyl, halo, O—C₁₋₄ alkyl, S—C₁₋₄ alkyl, or N(Rₐ)₂; Rₐ is hydrogen, C₁₋₆ alkyl, or Rₐ together with the nitrogen, may form a heterocyclic ring of 5 to 7 members, said ring optionally containing an additional heteroatom selected from the group consisting of oxygen, sulfur or nitrogen; R₂ is mono- or di-substituted phenyl wherein the substituents are independently selected from the group consisting of hydrogen, halo, S(O)ₘR₅, OR₆, halo substituted C₁₋₄ alkyl, C₁₋₄ alkyl, or N(R₁₂)₂; R₄ is hydrogen, C₁₋₁₀ alkyl, C₂₋₁₀ alkenyl, C₂₋₁₀ alkynyl, C₃₋₇ cycloalkyl, C₅₋₇ cycloalkenyl, heterocyclic, heterocyclicalkyl, aryl, aryl alkyl, heteroaryl, heteroaryl alkyl; R₃ is (X)ᵣ-(Q)ₛ-(Y)ₜ; X is hydrogen, —(C(R₁₀)₂)ₙ, —NR₁₃, —O—, or S(O)ₘ; r is a number having a value of 0 or 1; m is a number having a value of 0, 1 or 2; Q is alkenyl, alkynyl, cycloalkyl, cycloalkenyl, heterocyclic, heterocyclicalkyl, aryl, arylalkyl, heteroaryl, or heteroarylalkyl; s is a number having a value of 0 or 1; Y is a substituent selected from the group consisting of hydrogen, C₁₋₁₀ alkyl, halo-substituted C₁₋₁₀ alkyl, halogen, —(C(R₁₀)₂)ₙOR₈, —(C(R₁₀)₂)ₙNO₂, —(C(R₁₀)₂)ₙS(O)_{m′}R₁₁, —(C(R₁₀)₂)ₙSR₈, —(C(R₁₀)₂)ₙS(O)_{m′}OR₈, —(C(R₁₀)₂)ₙS(O)_{m′}NR₈R₉, —Xₐ—P(Z)-(XₐR₁₃)₂, —(C(R₁₀)₂)ₙNR₈R₉, —(C(R₁₀)₂)ₙCO₂R₈, —(C(R₁₀)₂)ₙOC(O)—R₈, —(C(R₁₀)₂)ₙCN, —(C(R₁₀)₂)ₙCONR₈R₉, —(C(R₁₀)₂)ₙC(S)NR₈R₉, —(C(R₁₀)₂)ₙNR₁₀C(O)R₈, —(C(R₁₀)₂)ₙNR₁₀C(S)R₈, —(C(R₁₀)₂)ₙNR₁₀C(Z)NR₈R₉, —(C(R₁₀)₂)ₙNR₁₀S(O)ₘR₁₁, —(C(R₁₀)₂)ₙNR₁₀C(═NCN)—S—R₁₁, —(C(R₁₀)₂)ₙNR₁₀C(═NCN)—NR₈R₉, —(C(R₁₀)₂)ₙNR₁₀C(O)C(O)—NR₈R₉, —(C(R₁₀)₂)ₙNR₁₀C(O)C(O)—OR₁₀, —(C(R₁₀)₂)ₙC(═NR₁₀)—NR₈R₉, —(C(R₁₀)₂)ₙ—C(═NR₁₀)-ZR₁₁, —(C(R₁₀)₂)ₙ—OC(Z)-NR₈R₉, —(C(R₁₀)₂)ₙNR₁₀S(O)ₘCF₃, —(C(R₁₀)₂)ₙNR₁₀C(O)OR₁₀; t is an integer having a value of 0, 1, 2, or 3; Xa is independently —(C(R₁₀)₂)ₙ, —NR₈—, —O— or —S—; Z is oxygen or sulfur, m′ is an integer having a value of 1 or 2; n is an integer having a value of 0 to 10; R₅ is hydrogen, C₁₋₄ alkyl, C₂₋₄ alkenyl, C₂₋₄ alkynyl, C₃₋₇ cycloalkyl, C₅₋₇ cycloalkenyl, aryl, or N(R₇)₂; provided that when m is 1 or 2 then R₅ is not hydrogen. R₆ is hydrogen, C₁₋₄ alkyl, halo substituted C₁₋₄ alkyl, C₂₋₄ alkenyl, C₂₋₄ alkynyl, C₃₋₇ cycloalkyl, C₅₋₇ cycloalkenyl, or aryl; R₇ is hydrogen, C₁₋₄ alkyl, C₂₋₄ alkenyl, C₂₋₄ alkynyl, aryl, or may form a heterocyclic ring of 5 to 7 members together with the nitrogen, said ring optionally containing an additional heteroatom selected from the group consisting of oxygen, sulfur or nitrogen; provided that when R₅ is N(R₇)₂ then m is 1 or 2; R₈ is hydrogen, C₁₋₁₀ alkyl, C₂₋₁₀ alkenyl, C₂₋₁₀ alkynyl, C₃₋₇ cycloalkyl, C₅₋₇ cycloalkenyl, heterocyclic, heterocyclic alkyl, aryl, aryl alkyl, heteroaryl, heteroaryl alkyl; R₉ is hydrogen, C₁₋₁₀ alkyl, C₂₋₁₀ alkenyl, C₂₋₁₀ alkynyl, C₃₋₇ cycloalkyl, C₅₋₇ cycloalkenyl, aryl, aryl alkyl, heteroaryl, heteroaryl alkyl or R₈ and R₉ may together form a heterocyclic ring of 5 to 7 members together with the nitrogen, said ring optionally containing an additional heteroatom selected from the group consisting of oxygen, sulfur or nitrogen; R₁₀ is hydrogen, or C₁₋₄ alkyl; R₁₁ is C₁₋₁₀ alkyl, C₂₋₁₀ alkenyl, C₂₋₁₀ alkynyl, C₃₋₇ cycloalkyl, C₅₋₇ cycloalkenyl, aryl, aryl alkyl, heteroaryl, heteroaryl alkyl; R₁₂ is hydrogen, C₁₋₄ alkyl, aryl, or may form a heterocyclic ring of 5 to 7 members together with the nitrogen; R₁₃ is hydrogen, C₁₋₁₀ alkyl, cycloalkyl, heterocylic, aryl, aryl alkyl, heteroaryl, or heteroaryl alkyl.

### Fusidic Acid

In certain embodiments, fusidic acid or a derivative thereof (e.g., sodium fusidate) can be used in the compositions, methods, and kits of the invention. The structure of fusidic acid is:

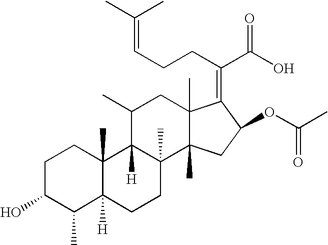

Fusidic acid derivatives are described in U.S. Pat. Nos. 3,352,854, 3,385,869, 3,376,324, 4,004,004, 4,060,606, 4,162,259, 4,315,004, 4,119,717, 6,103,884, and 6,593,319. Derivative include 11-monoketofusidic acid, 16-O-deacetylfusidic acid, 16-O-deacetylfusidic acid lactone, 3,11-diketofusidic acid, diethanolamine fusidate, helvolic acid, and tauro-24,25-dihydrofusidate.

### TOFA

In certain embodiments, 5-(tetradecyloxy)-2-furancarboxylic acid (TOFA) or an analog thereof can be used in the compositions, methods, and kits of the invention. TOFA is an inhibitor of acetyl-CoA carboxylase. The structure of TOFA is:

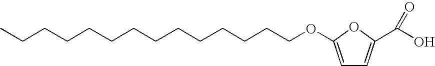

Analogs of TOFA are described, for example, in U.S. Pat. No. 4,382,143 and have the general structure:

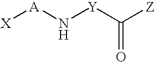

wherein X is selected from the group consisting of hydrogen, C₃-C₈ cycloalkyl, and substituted or unsubstituted aryl; A is a divalent radical selected from the group consisting of branched or unbranched C₆-C₁₉ alkylene, alkenylene, and alkynylene; Y is a 5- or 6-membered heteroaryl ring containing one or more nitrogen, sulfur, or oxygen atoms and optionally unsubstituted or substituted with one fluoro; and Z is selected from the group consisting of hydrogen, hydroxy, loweralkoxy, loweralkoxyloweralkoxy, diloweralkylaminoloweralkoxy, (mono- or polyhydroxy)loweralkoxy, (mono- or polycarboxy)loweralkoxy, (mono- or polycarboxy)hydroxyloweralkoxy, allyloxy, 2,3-epoxypropoxy, substituted or unsubstituted-(phenoxy, benzyloxy, or 3-pyridyloxy), pyridylmethoxy, tetrahydropyranyloxy, (mono- or polyhydroxy)alkylamino, allylamino, propargylamino, 2-sulfoethylamino, (mono- or polycarboxyl)loweralkylamino, loweralkanoylamino, (substituted or unsubstituted)aroylamino, loweralkanesulfonylamino, (substituted or unsubstituted)arenesulfonylamino, loweralkanylhydrazino, hydroxylamino, polymethyleneimino, and (4-carboxy- or 4-carboethoxy)thiazolidino; and the pharmaceutically acceptable acid-addition and cationic salts thereof.

### Tolterodine

In certain embodiments, tolterodine or an analog thereof can be used in the compositions, methods, and kits of the invention. Tolterodine is a competitive muscarinic receptor antagonist. The pharmacologically active agent is the 5-hydroxymethyl derivative. Cholinergic muscarinic receptors mediate urinary bladder contraction. Tolterodine is thus used to treat urinary incontinence. The structure of tolterodine is:

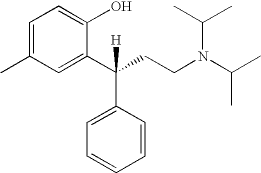

Analogs of tolterodine are described, for example, in U.S. Pat. No. 5,382,600 and have the general structure:

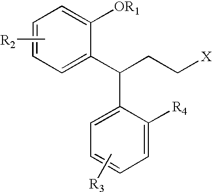

wherein R₁ signifies hydrogen or methyl, R₂, R₃, and R₄ independently signify hydrogen, methyl, methoxy, hydroxy, carbamoyl, sulphanoyl or halogen, and X represents a tertiary amino group (—NR₅R₆) wherein R₅ and R₆ signify non-aromatic hydrocarbol groups, which may be the same or different and which together contain at least three carbon atoms, preferably at least four or five carbon atoms, and where R₅ and R₆ may form a ring together with the amine nitrogen, said ring preferably having no other hetero atom that the amine nitrogen.

### Toremifene

In certain embodiments, toremifene or an analog thereof can be used in the compositions, methods, and kits of the invention. Toremifene is antiestrogen and antineoplastic agent. The structure of toremifene is:

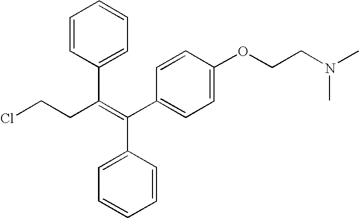

Analogs of toremifene are described, for example, in U.S. Pat. No. 4,696,949 have the general structure:

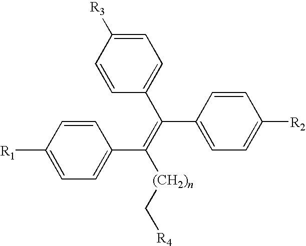

or the structure:

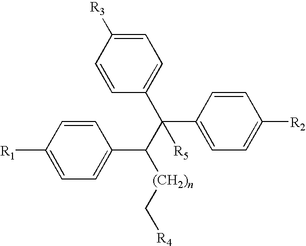

wherein n is 0 to 4, R₁ and R₂, which can be the same or different are H, OH, an alkoxy group of 1 to 4 carbon atoms, benzyloxy or methoxymethoxy; R₃ is H, OH, halogen, alkoxy of 1 to 4 carbon atoms, benzyloxy, methoxymethoxy, 2,3-dihydroxypropoxy or —O(CH₂)ₘCH₂NR₆R₇ wherein m is 1 or 2, R₆ and R₇, which can be the same or different, are H or an alkyl group of 1 to 4 carbon atoms, or —NR₆R₇ can form an N-containing three-, four-, five- or six-membered heterocyclic ring; R₄ is OH, F, Cl, Br, I, mesyloxy, tosyloxy, alkylcarbonyloxy of 1 to 4 carbon atoms, formyloxy or CH₂R₄ is replaced by CHO; R₅ is H or OH; or R₄ and R₅ together form an —O— bridge between the carbon atoms to which they are attached.

### Trequinsin

In certain embodiments, trequinsin or an analog thereof can be used in the compositions, methods, and kits of the invention. Trequinsin is a platelet aggregation inhibitor. The structure of trequinsin is:

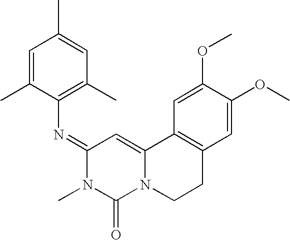

Trequinsin analogs are described, for example, in U.S. Pat. No. 5,141,936 and have the general structure:

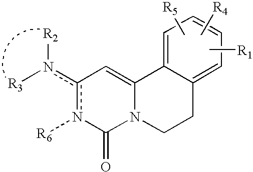

in which R₁, R₄ and R₅, which may be identical or different, may be hydrogen, hydroxyl, lower alkoxy, dialkylphosphinylalkoxy acyloxy or halogen, where two adjacent groups together may denote a methylenedioxy or ethylenedioxy group, and R₂ and R₃, which may be identical or different, may be hydrogen, hydroxyl, lower alkoxy, amino, alkylamino, dialkylamino, arylamino, alkyl, amino or alkyl substituted by a 5- or 6-membered carbon ring which may contain up to 3 heteroatoms from the group comprising N, O or S, cycloalkyl, hydroxyalkyl, alkoxyalkyl, dialkoxyalkyl, haloalkyl, dialkylaminoalkyl, aralkyl, acyl and, optionally substituted, aryl, where aryl is in each case taken to mean an aromatic hydrocarbon having up to 10 carbon atoms, and R₂ denotes an electron pair if R₆ denotes one of the radicals indicated below and R₂ and R₃ together with the nitrogen atom to which they are bonded may denote a part of an optionally substituted nitrogen heterocycle which may contain a further nitrogen atom or an oxygen atom, and R₆ stands for hydrogen, alkyl, cycloalkyl, hydroxyalkyl, alkoxyalkyl, dialkoxyalkyl, haloalkyl, dialkylaminoalkyl, aralkyl, heterocyclic-substituted alkyl, dialkylphosphinylalkyl, acyl and optionally substituted aryl, and also stands for an electron pair if R₂ denotes one of the radicals indicated above, and their acid salts and quaternary ammonium salts.

### Vinorelbine

In certain embodiments, vinorelbine or an analog thereof can be used in the compositions, methods, and kits of the invention. Vinorelbine is an antineoplastic agent that functions by binding microtubular proteins of the mitotic spindle, thereby inhibiting mitosis. The structure of vinorelbine is:

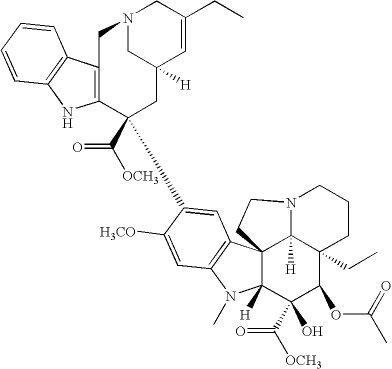

Analogs of vinorelbine are described, for example, in U.S. Pat. No. 4,307,100 and have the general structure:

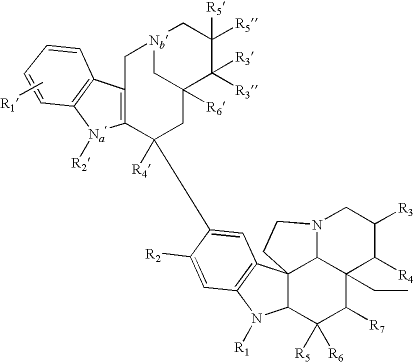

wherein R′₁ represents a hydrogen atom or an alkoxy, acyl, formyl or haloacyl radical; R₁₂ represents a hydrogen atom or an alkyl radical; R′₃ and R″₃ which may be the same or different each represents a hydrogen atom or a hydroxyl radical or an alkanoyloxyl radical or together represent a carbonyl group, or R′₃ and R′₅ together represent an epoxy bridge or a double bond; R₁₄ represent a hydrogen atom or an alkyloxycarbonyl, hydroxymethyl, alkanoyloxymethyl or acetamido radical; R′₅ and R″₅ which may be the same or different each represents a hydrogen atom or a hydroxyl, alkanoyloxyl, ethyl or 2-hydroxyethyl radical; R′₆ represents a hydrogen atom or an ethyl, 2-hydroxyethyl or acetyl radical; R₁ represents a hydrogen atom or an alkyl, formyl, or acyl radical; R₂ represents a hydrogen atom or an alkoxy radical; R₃ represents a hydrogen atom or a hydroxyl or alkanoyloxyl radical, or R₃ and R₄ together represent an epoxy bridge or a double bond; R₄ represents a hydrogen atom or a hydroxyl or alkanoyloxyl radical, or R₄ and R₅ together represent an epoxy bridge; R₆ represents an alkyloxycarbonyl, hydrazido, acetamido, hydroxymethyl or alkanyloxymethyl radical; and R₅ and R₇ represent a hydrogen atom or a hydroxyl or alkanoyloxyl radical. Vinorelbine is a member of the vinblastine compounds, which include 16-O-acetylvindoline, 3′,4′-anhydrovinblastine, 4′-deoxyvinblastine, 4-desacetylvinblastine, 4-desacetylvinblastine hydrazide, 4-O-deacetylvinblastine-3-oic acid, bis(N-ethylidene vindesine)disulfide, catharanthamine, catharinine, desacetylnavelbine, KAR 2, LY 266070, NAPAVIN, ViFuP protocol, vincathicine, vindoline, vindolinine, vinepidine, vinflunine, vinleucinol, vinorelbine, vintriptol, and vintriptol acid.

### Wedelolactone

In certain embodiments, wedelolactone or an analog thereof can be used in the compositions, methods, and kits of the invention. Wedelolactone is IKKα and IKKβ kinase inhibitor and a IkB-α kinase inhibitor. The structure of wedelolactone is:

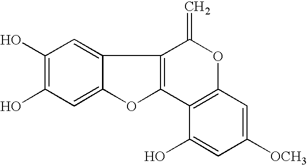

Wedelolactone is a member of the coumarins. Other coumarins include 11,12-dihydroxy-5-methylcoumestan, 11-desacetoxywortmannin, 2″,3″-dihydrogeiparvarin, 2-amino-3-(7-methoxy-4-coumaryl)propionic acid, 2-nitro-6H-dibenzo(b,d)pyran-6-one, 3′-angeloyloxy-4′-acetoxy-3′,4′-dihydroseselin, 3,4-dichloroisocoumarin, 3,4-dihydro-3,4-dibromo-6-bromomethylcoumarin, 3,4-dihydro-3-benzyl-6-chloromethylcoumarin, 3,4-dihydrocoumarin, 3,8-dihydroxy-6H-dibenzo(b,d)pyran-6-one, 3-(2-(N,N-diethyl-N-methylammonium)ethyl)-7-methoxy-4-methylcoumarin, 3-acetylcoumarin, 3-carbethoxypyranocoumarin, 3-carboxylic acid-picumast, 3-cyano-7-ethoxycoumarin, 3-cyano-7-hydroxycoumarin, 3-hydroxy-(28-4-coumaroyloxy)lup-20(29)-en-27-oic acid, 3-hydroxymethyl-picumast, 3-nitro-6H-dibenzo(b,d)pyran-6-one, 3-phenyl-5,6-benzocoumarin, 3H-naphtho(2,1-b)pyran-3-one, 4′-hydroxyasperentin, 4-(diazomethyl)-7-(diethylamino)coumarin, 4-acetylisocoumarin, 4-bromomethyl-6,7-dimethoxycoumarin, 4-bromomethyl-6,7-methylenedioxycoumarin, 4-bromomethyl-7-acetoxycoumarin, 4-chloro-3-ethoxy-7-guanidinoisocoumarin, 4-methyl-7-diethylaminocoumarin, 4-methyl-7-ethoxycoumarin, 4-methyl-N-ethyl pyrrolo[3,2-g]coumarin, 4-nitro-6H-dibenzo(b,d)pyran-6-one, 4-phenyl-3-isocoumarinic acid, 4-phenyl-3-isocoumarinic acid allylamide, 4-trifluoromethylcoumarin phosphate, 5,6-benzocoumarin-3-carboxylic acid ethyl ester, 5,7-dihydroxy-4-imino-2-oxochroman, 5,7-dimethoxycoumarin, 5-iodo-6-amino-1,2-benzopyrone, 5-methyl-8-hydroxycoumarin, 5-methylcoumarin-4-cellobioside, 5-methylcoumarin-4-gentiobioside, 5H-(2)benzopyrano(3,4-g)(1,4)benzodioxin-5-one, 6′-feruloylnodakenin, 6,7-(4-methyl)coumaro-(2.2.2)cryptand, 6,8-dimethoxy-3-methyl-3,4-dihydroisocoumarin, 6-(7-beta-galactosylcoumarin-3-carboxamido)hexylamine, 6-amino-1,2-benzopyrone, 6-amino-4,4,5,7,8-pentamethyldihydrocoumarin, 6-chloro-3,4-dihydroxy-2H-1-benzopyran-2-one, 6-cyano-7-hydroxy-4,8-dimethylcoumarin, 6-hydroxymellein, 6-methoxy-8-hydroxy-3-methyl-3,4-dihydroisocoumarin, 6-methylcoumarin, 6-methylthionecoumarin, 6-nitroso-1,2-benzopyrone, 7,8-dimethoxycoumarin, 7-((N-tosylphenylalanyl)amino)-4-chloro-3-methoxyisocoumarin, 7-(alpha-glutamyl)-4-methylcoumarylamide, 7-(gamma-glutamyl)-4-methylcoumarylamide, 7-(N-benzyloxycarbonyl-beta-benzylaspartyl-prolyl-leucyl)amino-4-methylcoumarin, 7-(N-benzyloxycarbonylglycyl-glycyl-leucyl)amino-4-methylcoumarin, 7-amino-3-(2-bromoethoxy)-4-chloroisocoumarin, 7-amino-4-chloro-3-(3-isothiureidopropoxy)isocoumarin, 7-amino-4-methylcoumarin, 7-amino-4-methylcoumarin-3-acetic acid, 7-amino-4-trifluoromethylcoumarin, 7-aminocoumarin, 7-aminocoumarin-4-methanesulfonic acid, 7-anilino-4-methylcoumarin-3-acetic acid, 7-anilinocoumarin-4-acetic acid, 7-benzylcysteinyl-4-methylcoumarinylamide, 7-benzyloxy-4-trifluoromethylcoumarin, 7-beta-galactopyranosyl-oxycoumarin-4-acetic acid methyl ester, 7-beta-galactopyranosyloxycoumarin-4-acetic acid, 7-diethylamino-3-(4′-isothiocyanatophenyl)-4-methylcoumarin, 7-diethylaminocoumarin-3-carbohydrazide, 7-diethylaminocoumarin-3-carboxylic acid, 7-dimethylamino-4-methylcoumarin, 7-ethenyloxycoumarin, 7-ethoxy-4-trifluoromethylcoumarin, 7-ethoxycoumarin, 7-glycidoxycoumarin, 7-hydroxy-4-phenyl-3-(4-hydroxyphenyl)coumarin, 7-hydroxy-4-trifluoromethylcoumarin, 7-hydroxycoumarin-4-acetic acid, 7-leucylamido-4-methylcoumarin, 7-lysylalanyl-4-methylcoumarinamide, 7-succinylglycyl-prolyl-4-methylcoumaryl-7-amide, 8-(3-(4-phenyl-1-piperazinyl)propoxy)-7-methoxycoumarin, 8-hydroxy-4-methyl-3,4-dihydroxycoumarin, 8-hydroxycoumarin, 9-(3-diethylaminopropyloxy)-3H-naphtho(2,1-b)pyran-3-one, A 1062, Ac-aspartyl-glutamyl-valyl-aspartyl-aminomethylcoumarin, acetyl-aspartyl-glutamyl-valyl-aspartyl-amino-4-methylcoumarin, agrimonolide-6-O-glucopyranoside, AI 77B, alanyl-alanyl-phenylalanyl-7-amino-4-methylcoumarin, amicoumacin A, anomalin, arginine 4-methyl-7-coumarylamide, arnottin I, aspartyl-glutamyl-valyl-aspartyl-7-amino-4-trifluoromethylcoumarin, aurapten, baciphelacin, benzyloxycarbonyl-phenylalanylarginine-4-methylcoumaryl-7-amide, benzyloxycarbonylarginyl-arginine 4-methyloumarin-7-ylamide, bergaptol-O-glucopyranoside, Boc-leucyl-seryl-threonyl-arginine-4-methylcoumaryl-7-amide, byakangelicol, calanolide A, calanolide B, calophyllolid, carbobenzoxycoumarin, Cassella 7657, CGP 13143, chlorobiocic acid, Chromonar, CI 923, cladosporin, clausarin, clausindine, clausmarin, columbianadin, cordatolide A, coumachlor, coumarin, coumarin 3,4-epoxide, coumarin-3-carboxylic acid, coumarin-3-carboxylic acid succinimidyl ester, coumermycins, coumestrol, coumetarol, crenulatin, cytogenin, daphnoretin, dehydroindicolactone, demethylwedelolactone, dicurin, erythrocentaurin, Esculin, esuprone, F 1375, ferujol, ferulenol, folescutol, fraxetin, fraxin, galbanic acid, geiparvarin, gerberinside, glaupadiol, glisoflavone, glutaryl-alanyl-alanyl-phenylalanyl-amidomethylcoumarin, glutaryl-glycyl-arginine-4-methylcoumaryl-7-amide, glycyl-7-amino-4-methylcoumarin-3-acetic acid, glycylprolyl-4-methylcoumaryl-7-amide, GU 7, GUT-70, 4-hydroxycoumarins, hymecromone O,O-diethyl phosphorothioate, iliparcil, inophyllum B, isobyakangelicin angelate, isofraxidin, isorhamnetin 3-O-beta-(4′″-4-coumaroyl-alpha-rhamnosyl(1-6)galactoside), kaempferol-2,4-dicoumaroyl-3-O-glucoside, licopyranocoumarin, LL-N 313, mammein, mammeisin, maoyancaosu, marmesin, marmin, melilot, moellendorffiline, morocromen, moxicoumone, murayalactone, N-(2-(1-maleimidyl)ethyl)-7-(diethylamino)coumarin-3-carboxamide, N-(4-(7-(diethylamino)-4-methylcoumarin-3-yl))maleimide, N-(4-(7-diethylamino 4-methylcoumarin-3-yl)phenyl)iodoacetamide, N-(4-(7-diethylamino-4-methylcoumarin-3-yl)phenyl)maleimide, N-acetyl-alanyl-alanyl-prolyl-alanyl-amidomethylcoumarin, N-benzyloxycarbonylalanyl-arginyl-arginyl-4-trifluoromethyl-7-coumarylamide, N-benzyloxycarbonylglycyl-glycyl-arginine-4-methylcoumarinyl-7-amide, N-carbobenzoxyglycyl-prolyl-4-methylcoumarinyl amide, N-salicylidene-3-aminocoumarin, N-succinimidyl-7-dimethylaminocoumarin-4-acetate, necatorin, neoglycyrol, nitrofarin, nordentatin, notopterol, Ochratoxins, oosponol, oroselol, osthenol, osthol, oxamarine, pargyropyranone, PD 118717, peuarenine, peujaponiside, phebalosin, phellopterin, phyllodulcin, picumast, ponfolin, praeruptorin C, praeruptorin E, Psoralens, psoralidin, pterybinthinone, pteryxin, pyranocoumarins, qianhucoumarin A, qianhucoumarin B, qianhucoumarin C, reticulol, Ro7-AMCA, rubradiric acid A, rubradiric acid B, rubricauloside, sclerin, scoparone, scopolin, serine-7-amino-4-methylcoumarin carbamate, shijiaocaolactone A, soulattrolide, SP500263, succinyl-isoleucyl-isoleucyl-tryptophyl-methylcoumarinamide, succinyl-leucyl-leucyl-valyl-tyrosyl-methylcoumarinamide, succinyl-leucyl-tyrosyl-4-methyl-7-coumarylamide, succinylalanylalanyl-prolyl-phenylalanine-4-methylcoumaryl-7-amide, succinylglycyl-prolyl-leucyl-glycyl-prolyl-4-methylcoumaryl-7-amide, suksdorfin, sulfosuccinimidyl 7-amino-4-methylcoumarin-3-acetate, surangin B, tert-butyloxycarbonyl-leucyl-glycyl-arginine-4-trifluoromethylcoumarin-7-amide, tert-butyloxycarbonyl-norleucyl-glutaminyl-leucyl-glycyl-arginine-7-amino-4-methylcoumarin, tertiary butyloxycarbonylvalyl-leucyl-lysinyl-4-methylcoumarin-7-amide, tertiary-butyloxycarbonyl-isoleucyl-glutamyl-glycyl-arginyl-7-amino-4-methylcoumarin, tertiary-butyloxycarbonyl-phenylalanyl-seryl-arginyl-4-methylcoumarin-7-amide, tertiary-butyloxycarbonyl-valyl-prolyl-arginyl-7-amino-4-methylcoumarin, theo-esberiven, thunberginol A, thunberginol B, thunberginol D, tioclomarol, toddalolactone, tosyl-glycyl-prolyl-arginyl-4-methylcoumaryl-7-amide, ubiquitin C-terminal 7-amido-4-methylcoumarin, Umbelliferones, valyl-leucyl-lysyl-4-aminomethylcoumarin, valyl-leucyl-lysyl-7-amino-4-methylcoumarin, Venalot, W10294A, WS-5995 A, xanthalin, and xanthyletine.

### Telaprevir

In certain embodiments, telaprevir or an analog thereof can be used in the compositions, methods, and kits of the invention. Telaprevir (VX-950) is a hepatitis C therapy. The structure of telaprevir is:

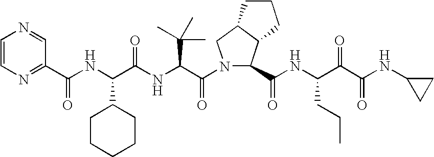

Analogs of telaprevir are described, for example, in U.S. Pat. Application Publication No. 2005/0197299 and can be represented as follows:

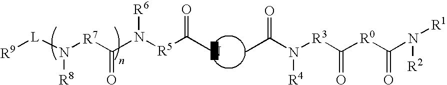

wherein R⁰ is a bond or difluoromethylene; R¹ is hydrogen, optionally substituted aliphatic group, optionally substituted cyclic group or optionally substituted aromatic group; R² and R⁹ are each independently optionally substituted aliphatic group, optionally substituted cyclic group or optionally substituted aromatic group; R³, R⁵, and R⁷ are each independently (optionally substituted aliphatic group, optionally substituted cyclic group or optionally substituted aromatic group) (optionally substituted methylene or optionally substituted ethylene), optionally substituted (1,1- or 1,2-)cycloalkylene or optionally substituted (1,1- or 1,2-)heterocyclylene; R⁴, R⁶, R⁸ and R¹⁰ are each independently hydrogen or optionally substituted aliphatic group;

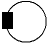

is substituted monocyclic azaheterocyclyl or optionally substituted multicyclic azaheterocyclyl, or optionally substituted multicyclic azaheterocyclenyl wherein the unsaturatation is in the ring distal to the ring bearing the R⁹-L-N(R⁸)—R⁷—C(O)ₙN(R⁶)—R⁵—C(O)—N moiety and to which the —C(O)—N(R⁴)—R³—C(O)—C(O)NR²R¹ moiety is attached; L is —C(O)—, —OC(O)—, —NR¹⁰C(O)—, —S(O)₂—, or —NR¹⁰S(O)₂—; and n is 0 or 1, or a pharmaceutically acceptable salt or prodrug thereof, or a solvate of such a compound, its salt or its prodrug, provided when

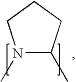

is substituted

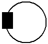

then L is —OC(O)— and R⁹ is optionally substituted aliphatic, or at least one of R³, R⁵ and R⁷ is (optionally substituted aliphatic group, optionally substituted cyclic group or optionally substituted aromatic group) (optionally substituted ethanediyl), or R⁴ is optionally substituted aliphatic.

### HCV-796

In certain embodiments, HCV-796 or an analog thereof can be used in the compositions, methods, and kits of the invention. HCV-796 is a non-nucleoside polymerase inhibitor. The structure of HCV-796 is:

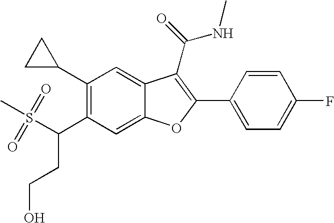

Analogs of HCV-796 are described for example, in U.S. Pat. No. 7,265,152 and have the general structure:

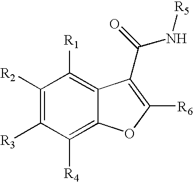

wherein R₁ represents a radical selected from the group consisting of hydrogen, alkyl, halogen, and cyano; R₂ represents a radical selected from the group consisting of hydrogen, a substituted or unsubstituted alkyl radical, a substituted or unsubstituted alkoxy group, hydroxy, cycloalkyl, cycloalkyloxy, polyfluoroalkyl, polyfluoroalkoxy, halogen, amino, monoalkylamino, dialkylamino, cyano, a substituted or unsubstituted benzyloxy group, and a substituted or unsubstituted heterocyclic radical; R₃ represents a radical selected from the group consisting of hydrogen, a substituted or unsubstituted alkyl radical, a substituted or unsubstituted alkoxy group, alkenyl, halogen, hydroxy, polyfluoroalkyl, polyfluoroalkoxy, formyl, carboxyl, alkylcarbonyl, alkoxycarbonyl, hydroxyalkylcarbonyl, amino, a substituted or unsubstituted monoalkylamino, dialkylamino, cyano, amido, alkoxyamido, a substituted or unsubstituted heteroarylamino, acetylsulfonylamino, ureido, carboxamide, sulfonamide, a substituted sulfonamide, a substituted or unsubstituted heterocyclosulfonyl, alkylthio, alkylsulfinyl, alkylsulfonyl, alkylsulfonic acid, a substituted or unsubstituted heterocyclic radical, and —O(CH₂)—C(═O)—R₇; R₄ represents a radical selected from the group consisting of hydrogen, alkyl, halogen, and alkoxy; R₅ represents a radical selected from the group consisting of an alkyl (C₁-C₆) group, cycloalkyl, and cycloalkylalkyl; R₆ represents a radical selected from the group consisting of a substituted or unsubstituted aryl group and a substituted or unsubstituted heteroaryl group; R₇ represents a radical selected from the group consisting of dialkylamino, a substituted or unsubstituted arylamino, a substituted or unsubstituted heteroarylamino, and a substituted or unsubstituted aryl group, said monoalkylamino substituents being one or more radical(s) independently selected from the group consisting of cycloalkyl, hydroxy, alkoxy, and a substituted or unsubstituted heterocyclic radical; said arylamino substituents and said heteroarylamino substituents being one or more radical(s) independently selected from an alkyl group and an alkoxycarbonyl; said sulfonamide substituents being one or more radical(s) independently selected from the group consisting of alkenyl, cycloalkyl, alkoxy, hydroxy, halogen, polyfluoroalkyl, polyfluoroalkoxy, carboxyl, alkylcarbonyl, alkoxycarbonyl, carboxamide, a substituted or unsubstituted aryl group, and a substituted or unsubstituted heterocyclic radical; said heterocyclosulfonyl substituents being one or more radical(s) independently selected from the group consisting of alkoxy and hydroxy; said alkyl radical substituents and said alkoxy group substituents being one or more radical(s) independently selected from the group consisting of alkenyl, amino, monoalkylamino, dialkylamino, alkoxy, cycloalkyl, hydroxy, carboxyl, halogen, cyano, polyfluoroalkyl, polyfluoroalkoxy, sulfonamide, carboxamide, alkylsulfonyl, alkylcarbonyl, alkoxycarbonyl, mercapto, 2,2-dimethyl-4-oxo-4H-benzo[1,3]dioxinyl, a substituted or unsubstituted aryl group, and a substituted or unsubstituted heterocyclic radical; said heterocyclic radical substituents being one or more radical(s) independently selected from the group consisting of alkyl, amino, amido, monoalkylamino, cycloalkyl-alkylamino, dialkylamino, alkoxy, alkoxyalkyl, hydroxy, hydroxyalkyl, cycloalkyl, cycloalkylalkyl, carboxyl, carboxamide, halogen, haloalkyl, cyano, polyfluoroalkyl, polyfluoroalkoxy, alkylsulfonyl, alkylcarbonyl, cycloalkylcarbonyl, alkoxycarbonyl, mercapto, oxo, a substituted or unsubstituted aryl group, arylalkyl, and a substituted or unsubstituted heteroaryl group; said heteroaryl group substituents being one or more radical(s) independently selected from the group consisting of alkyl, amino, alkoxy, alkoxyalkyl, hydroxy, hydroxyalkyl, cycloalkyl, carboxyl, carboxamide, halogen, polyfluoroalkyl, polyfluoroalkoxy, alkylsulfonyl, mercapto, and oxo; said benzyloxy group substituents being one or more radical(s) independently selected from the group consisting of alkyl, alkoxy, polyfluoroalkyl, polyfluoroalkoxy, hydroxy, carboxyl, alkoxycarbonyl, halogen, cyano, alkylsulfonyl, and phenyl; said aryl group substituents being one or more radical(s) independently selected from the group consisting of alkyl, acetylenyl, alkoxy, hydroxy, halogen, polyfluoroalkyl, polyfluoroalkoxy, cyano, amino, monoalkylamino, dialkylamino, aminoalkyl, alkoxyalkoxy, amido, amidoalkyl, carboxyl, alkylsulfonyl, alkylcarbonyl, alkoxycarbonyl, mercapto, and a heterocyclic radical; and pharmaceutically acceptable salts thereof;

### Merimepodib (VX-497)

In certain embodiments, merimepodib or an analog thereof can be used in the compositions, methods, and kits of the invention. Merimepodib is an inhibitor of inosine-5′-monophosphate dehydrogenase (IMPDH) and is used to treat HCV. The structure of merimepodib is:

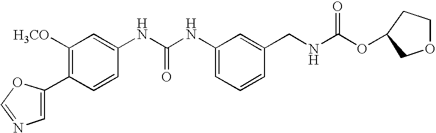

Analogs of merimepodib are described for example, in U.S. Pat. No. 6,541,496 and have the general structure:

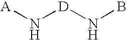

wherein A is selected from (C₁-C₆)-straight or branched alkyl, or (C₂-C₆)-straight or branched alkenyl or alkynyl; and A optionally comprises up to 2 substituents, wherein the first of said substituents, if present, is selected from R¹ or R³, and the second of said substituents, if present, is R¹; B is a saturated, unsaturated or partially saturated monocyclic or bicyclic ring system optionally comprising up to 4 heteroatoms selected from N, O, or S and selected from the formulae:

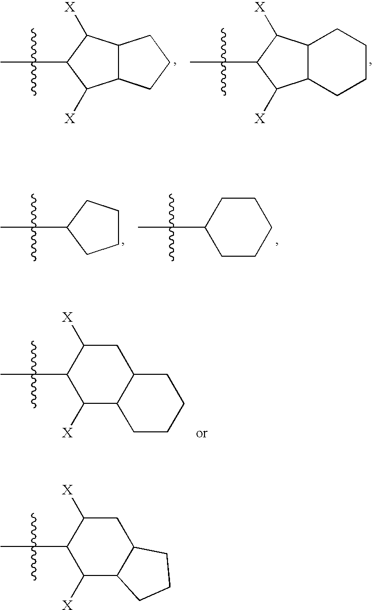

wherein each X is the number of hydrogen atoms necessary to complete proper valence; and B optionally comprises up to 3 substituents, wherein: the first of said substituents, if present, is selected from R¹, R², R⁴ or R⁵, the second of said substituents, if present, is selected from R¹ or R⁴, and the third of said substituents, if present, is R¹; and D is selected from C(O), C(S), or S(O)₂; wherein each R¹ is independently selected from 1,2-methylenedioxy, 1,2-ethylenedioxy, R⁶ or (CH₂)ₙ—Y; wherein n is 0, 1 or 2; and Y is selected from halogen, CN, NO₂, CF₃, OCF₃, OH, SR⁶, S(O)R⁶, SO₂R⁶, NH₂, NHR⁶, N(R⁶)₂, NR⁶R⁸, COOH, COOR⁶ or OR⁶; each R² is independently selected from (C₁-C₄)-straight or branched alkyl, or (C₂-C₄)-straight or branched alkenyl or alkynyl; and each R² optionally comprises up to 2 substituents, wherein the first of said substituents, if present, is selected from R¹, R⁴ and R⁵, and the second of said substituents, if present, is R¹; R³ is selected from a monocyclic or a bicyclic ring system consisting of 5 to 6 members per ring, wherein said ring system optionally comprises up to 4 heteroatoms selected from N, O, or S, and wherein a CH₂ adjacent to any of said N, O, or S heteroatoms is optionally substituted with C(O); and each R³ optionally comprises up to 3 substituents, wherein the first of said substituents, if present, is selected from R¹, R², R⁴ or R⁵, the second of said substituents, if present, is selected from R¹ or R⁴, and the third of said substituents, if present, is R¹; each R⁴ is independently selected from OR⁵, OC(O)R⁶, OC(O)R⁵, OC(O)OR⁶, OC(O)OR⁵, OC(O)N(R⁶)₂, OP(O)(OR⁶)₂, SR⁶, SR⁵, S(O)R⁶, S(O)R⁵, SO₂R⁶, SO₂R⁵, SO₂N(R⁶)₂, SO₂NR⁵R⁶, SO₃R⁶, C(O)R⁵, C(O)OR⁵, C(O)R⁶, C(O)OR⁶, NC(O)C(O)R⁶, NC(O)C(O)R⁵, NC(O)C(O)OR⁶, NC(O)C(O)N(R⁶)₂, C(O)N(R⁶)₂, C(O)N(OR⁶)R⁶, C(O)N(OR⁶)R⁵, C(NOR⁶)R⁶, C(NOR⁶)R⁵, N(R⁶)₂, NR⁶C(O)R⁶, NR⁶C(O)R⁶, NR⁶C(O)R⁵, NR⁶C(O)OR⁶, NR⁶C(O)OR⁵, NR⁶C(O)N(R⁶)₂, NR⁶C(O)NR⁵R⁶, NR⁶SO₂R⁶, NR₆SO₂R⁵, NR₆SO₂N(R⁶)₂, NR⁶SO₂NR⁵R⁶, N(OR⁶)R⁶, N(OR⁶)R⁵, P(O)(OR⁶)N(R⁶)₂, and P(O)(OR⁶)₂; each R⁵ is a monocyclic or a bicyclic ring system consisting of 5 to 6 members per ring, wherein said ring system optionally comprises up to 4 heteroatoms selected from N, O, or S, and wherein a CH₂ adjacent to said N, O or S maybe substituted with C(O); and each R⁵ optionally comprises up to 3 substituents, each of which, if present, is R¹; each R⁶ is independently selected from H, (C₁-C₄)-straight or branched alkyl, or (C₂-C₄) straight or branched alkenyl; and each R⁶ optionally comprises a substituent that is R⁷; R⁷ is a monocyclic or a bicyclic ring system consisting of 5 to 6 members per ring, wherein said ring system optionally comprises up to 4 heteroatoms selected from N, O, or S, and wherein a CH₂ adjacent to said N, O or S maybe substituted with C(O); and each R⁷ optionally comprises up to 2 substituents independently chosen from H, (C₁-C₄)-straight or branched alkyl, (C₂-C₄) straight or branched alkenyl, 1,2-methylenedioxy, 1,2-ethylenedioxy, or (CH₂)ₙ-Z; wherein n is 0, 1 or 2; and Z is selected from halogen, CN, NO₂, CF₃, OCF₃, OH, S(C₁-C₄)-alkyl, SO(C₁-C₄)-alkyl, SO₂(C₁-C₄)-alkyl, NH₂, NH(C₁-C₄)-alkyl, N((C₁-C₄)-alkyl)₂, N((C₁-C₄)-alkyl)R⁸, COOH, C(O)O(C₁-C₄)-alkyl or O(C₁-C₄)-alkyl; and R⁸ is an amino protecting group; and wherein any carbon atom in any A, R² or R⁶ is optionally replaced by O, S, SO, SO₂, NH, or N(C₁-C₄)-alkyl.

### Valopicitabine

In certain embodiments, valopicitabine (NM-283) or an analog thereof can be used in the compositions, methods, and kits of the invention. Valopicitabine is a hepatitis C therapy that acts as a polymerase inhibitor. Valopicitabine is an orally available prodrug of 2′-C-methylcytidine. The structure of valopicitabine is:

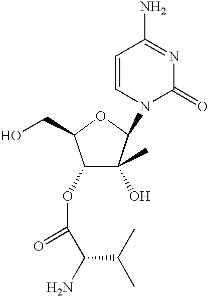

Analogs of valopicitabine are described, for example, in U.S. Pat. Application Publication No. 2007/0015905, which is hereby incorporated by reference.

### Boceprevir (SCH 503034)

In certain embodiments, boceprevir (SCH 503034) or an analog thereof can be used in the compositions, methods, and kits of the invention. Boceprevir is a hepatitis C therapy that acts as a inhibitor of the NS3-serine protease. The structure of boceprevir is:

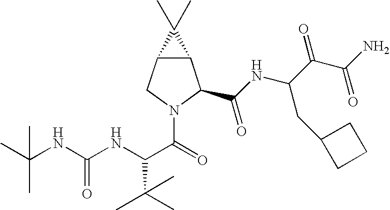

Analogs of boceprevir are described, for example, in U.S. Pat. Application Publication No. 2004/0254117 and have the general structure:

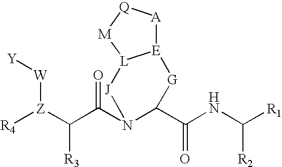

wherein Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y may be optionally substituted with X₁ or X₁₂; X₁₁ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X₁₁ may be additionally optionally substituted with X₁₂; X₁₂ is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X₁₂; R₁ is COR₅ or B(OR)₂, wherein R₅ is H, OH, OR₈, NR₉R₁₀, CF₃, C₂F₅, C₃F₇, CF₂R₆, R₆, or COR₇ wherein R₇ is H, OH, OR₈, CHR₉R₁₀, or NR₉R₁₀, wherein R₆, R₈, R₉ and R₁₀ are independently selected from the group consisting of H, alkyl, aryl, heteroalkyl, heteroaryl, cycloalkyl, cycloalkyl, arylalkyl, heteroarylalkyl, [CH(R₁′)]ₚCOOR₁₁, [CH(R₁′)]ₚCONR₁₂R₁₃, [CH(R₁′)]ₚSO₂R₁₁, [CH(R₁′)]ₚCOR₁₁, [CH(R₁′)]ₚCH(OH)R¹¹, CH(R₁′)CONHCH(R₂′)COOR₁₁, CH(R₁′)CONHCH(R₂′)CON—R₁₂R₁₃, CH(R₁′)CONHCH(R₂′)R₁₁, CH(R₁′)CONHCH(R₂′)CONHCH(R₃′)COOR₁₁, CH(R₁′)CONHCH(R₂′)CONHCH(R₃′)CONR₁₂R₁₃, CH(R₁′)CONHCH(R₂′)CONHCH(R₃′)CONHCH(R₄′)COOR₁₁, CH(R₁′)CONHCH(R₂′)CONHCH(R₃′)CONHCH(R₄′)CONR₁₂R.-sup.13, CH(R₁′)CONHCH(R₂′)CONHCH(R₃′)CONHCH(R₄′)CONHCH—(R₅′)COOR₁₁ and CH(R₁′)CONHCH(R₂′)CONHCH(R₃′)CON—HCH(R₄)CONHCH(R₅′)CONR₁₂R₁₃, wherein R₁′, R₂′, R₃′, R₄′, R₅′, R₁₁, R₁₂, R₁₃, and R′ are independently selected from the group consisting of H, alkyl, aryl, heteroalkyl, heteroaryl, cycloalkyl, alkyl-aryl, alkyl-heteroaryl, aryl-alkyl and heteroaralkyl; Z is selected from O, N, CH or CR; W may be present or absent, and if W is present, W is selected from C═O, C═S, C(═N—CN), or SO₂; Q may be present or absent, and when Q is present, Q is CH, N, P, (CH₂)ₚ, (CHR)ₚ, (CRR′)ₚ, O, NR, S, or SO₂; and when Q is absent, M may be present or absent; when Q and M are absent, A is directly linked to L; A is O, CH₂, (CHR)ₚ, (CHR—CHR′)ₚ, (CRR′)ₚ, NR, S, SO₂ or a bond; E is CH, N, CR, or a double bond towards A, L or G; G may be present or absent, and when G is present, G is (CH₂)ₚ, (CHR)ₚ, or (CRR′)ₚ; and when G is absent, J is present and E is directly connected to the carbon atom in Formula I as G is linked to; J maybe present or absent, and when J is present, J is (CH₂)ₚ, (CHR)ₚ, or (CRR′)ₚ, SO₂, NH, NR or O; and when J is absent, G is present and E is directly linked to N shown in Formula I as linked to J; L may be present or absent, and when L is present, L is CH, CR, O, S or NR; and when L is absent, then M may be present or absent; and if M is present with L being absent, then M is directly and independently linked to E, and J is directly and independently linked to E; M may be present or absent, and when M is present, M is O, NR, S, SO₂, (CH₂)ₚ, (CHR)ₚ(CHR—CHR′)ₚ, or (CRR′)ₚ; p is a number from 0 to 6; and R, R′, R₂, R₃ and R₄ are independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen; (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl; wherein said alkyl, heteroalkyl, alkenyl, heteroalkenyl, aryl, heteroaryl, cycloalkyl and heterocycloalkyl moieties may be optionally and chemically-suitably substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate; further wherein said unit N—C-G-E-L-J-N represents a five-membered or six-membered cyclic ring structure with the proviso that when said unit N—C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of the cyclic ring.

### Interferons

In certain embodiments, an interferon or an analog thereof can be used in the compositions, methods, and kits of the invention. Intefereons includes interferon-α, interferon alfa-2a, interferon alfa-2b, interfereon alfa-2c, interferon alfacon-1, interferon alfa-n1, interferon alfa-n3, interferon-β, interferon β-1a, interferon β-1b, interferon-γ, interferon γ-1a, interferon γ-1b, and pegylated forms thereof.

### Miscellaneous Agents

Albendazole analogs are described in U.S. Pat. Nos. 5,468,765, 5,432,187, 4,299,837, 4,156,006, and 4,136,174. Amitraz analogs are described in U.S. Pat. No. 3,781,355. Betaxolol analogs are described in U.S. Pat. No. 4,252,984. Bromhexine analogs are described in U.S. Pat. Nos. 3,408,446 and 4,191,780 and Belgian patent BE625002. Bromocriptine analogs are described in U.S. Pat. No. 4,145,549. Capsaicin analogs are described in U.S. Pat. No. 4,812,446. Carbaryl analogs are described in U.S. Pat. No. 2,903,478. Chloroquine analogs are described in U.S. Pat. No. 2,233,970. Cladribine (2-chloro-2′-deoxyadenosine) analogs are described in U.S. Pat. Nos. 4,760,137, 5,208,327, 6,252,061, 6,596,858, and 6,884,880. Clomiphene analogs are described in U.S. Pat. No. 2,914,563. Cyclocytidine analogs are described in U.S. Pat. No. 3,463,850. Dibucaine analogs are described in U.S. Pat. No. 1,825,623. Dicyclomine analogs are described in U.S. Pat. No. 2,474,796. Dilazep analogs are described in U.S. Pat. No. 3,532,685. Diphenidol analogs are described in U.S. Pat. No. 2,411,664. Donepezil analogs are described in U.S. Pat. No. 4,895,841. Emetine analogs are described in U.S. Pat. No. 3,102,118. Exemestane analogs are described in U.S. Pat. No. 4,808,616. Ezetimibe analogs are described in U.S. Pat. No. 5,767,115. Fenbendazole analogs are described in U.S. Pat. No. 3,954,791. Fenretinide analogs are described in U.S. Pat. No. 4,190,594. Fenvalerate analogs are described in U.S. Pat. No. 3,996,244. Flubendazole analogs are described in U.S. Pat. No. 3,657,267 and German patent DE2029637. Fludarabine analogs are described in U.S. Pat. No. 5,034,518. Fluorouracil analogs are described in U.S. Pat. Nos. 2,802,005, 2,885,396, 4,092,313, and 4,080,455. Ifenprodil analogs are described in U.S. Pat. No. 3,509,164. Indocyanine green analogs are described in U.S. Pat. No. 2,895,955. Iophenoxic acid analogs are described in British patent GB726987. Isosulfan blue analogs include sulfan blue. Mycophenolic acid analogs are described in U.S. Pat. Nos. 3,705,894, 3,903,071, 4,686,234, 4,725,622, 4,727,069, 4,753,935, 4,786,637, 4,808,592, 4,861,776, 4,868,153, 4,948,793, 4,952,579, 4,959,387, 4,992,467, 5,247,083, 5,380,879, 5,441,953, 5,444,072, 5,493,030, 5,538,969, 5,512,568, 5,525,602, 5,554,612, 5,633,279, 6,399,773, 6,420,403, 6,624,184, 6,916,809, 6,919,335, 7,053,111, and U.S. patent application Ser. No. 07/927,260. Narasin analogs are described in U.S. Pat. Nos. 4,035,481, 4,038,384, 4,141,907, 4,174,404, 4,204,039, and 5,541,224. Oxeladin analogs are described in U.S. Pat. No. 2,885,404. Oxfendazole analogs are described in U.S. Pat. No. 3,929,821. Oxibendazole analogs are described in U.S. Pat. No. 3,574,845. Perospirone analogs are described in U.S. Pat. No. 4,745,117. Picotamide analogs are described in French patent FR2100850. Pramoxine analogs are described in U.S. Pat. No. 2,870,151. Quinacrine analogs are described in U.S. Pat. Nos. 2,113,357, 1,782,727, and 1,889,704. Repaglinide analogs are described in International Application Publication No. WO 93/00337. Rifaximin analogs are described in U.S. Pat. No. 4,341,785. Silver sulfadiazine analogs are described in U.S. Pat. Nos. 2,407,966 2,410,793. Terconazole analogs are described in U.S. Pat. Nos. 4,144,346 and 4,223,036. Tioxolone analogs are described in U.S. Pat. Nos. 2,332,418 and 2,886,488. Tirapazamine analogs are described in U.S. Pat. No. 3,868,371. Tiratricol analogs are described in British patent Nos. GB803149 GB805761. Toremifene analogs are described in U.S. Pat. No. 4,696,949. Vincristine analogs are described in U.S. Pat. No. 4,144,237. Zafirlukast analogs are described in U.S. Pat. No. 4,859,692.

### Conjugates

If desired, the agents used in any of the combinations described herein may be covalently attached to one another to form a conjugate of formula I.

(A)-(L)-(B) (I)

In formula I, (A) is a drug listed on Table 1, Table 2, or Table 3 covalently tethered via a linker (L) to (B), a second drug listed on Table 1, Table 2, Table 3, Table 4, or Table 5.

Conjugates of the invention can be administered to a subject by any route and for the treatment of viral hepatitis (e.g., those described herein).

The conjugates of the invention can be prodrugs, releasing drug (A) and drug (B) upon, for example, cleavage of the conjugate by intracellular and extracellular enzymes (e.g., amidases, esterases, and phosphatases). The conjugates of the invention can also be designed to largely remain intact in vivo, resisting cleavage by intracellular and extracellular enzymes. The degradation of the conjugate in vivo can be controlled by the design of linker (L) and the covalent bonds formed with drug (A) and drug (B) during the synthesis of the conjugate.

Conjugates can be prepared using techniques familiar to those skilled in the art. For example, the conjugates can be prepared using the methods disclosed in G. Hermanson, Bioconjugate Techniques, Academic Press, Inc., 1996. The synthesis of conjugates may involve the selective protection and deprotection of alcohols, amines, ketones, sulfhydryls or carboxyl functional groups of drug (A), the linker, and/or drug (B). For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994. Additional synthetic details are provided below.

### Linkers

The linker component of the invention is, at its simplest, a bond between drug (A) and drug (B), but typically provides a linear, cyclic, or branched molecular skeleton having pendant groups covalently linking drug (A) to drug (B).

Thus, linking of drug (A) to drug (B) is achieved by covalent means, involving bond formation with one or more functional groups located on drug (A) and drug (B). Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of drug (A) and drug (B) may be effected using a linker which contains reactive moieties capable of reaction with such functional groups present in drug (A) and drug (B). For example, an amine group of drug (A) may react with a carboxyl group of the linker, or an activated derivative thereof, resulting in the formation of an amide linking the two.

Examples of moieties capable of reaction with sulfhydryl groups include α-haloacetyl compounds of the type XCH₂CO— (where X=Br, Cl, or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulfide bridges.

Examples of reactive moieties capable of reaction with amino groups include, for example, alkylating and acylating agents. Representative alkylating agents include:

(i) α-haloacetyl compounds, which show specificity towards amino groups in the absence of reactive thiol groups and are of the type XCH₂CO— (where X=Br, Cl, or I), for example, as described by Wong Biochemistry 24:5337 (1979);

(ii) N-maleimide derivatives, which may react with amino groups either through a Michael type reaction or through acylation by addition to the ring carbonyl group, for example, as described by Smyth et al., J. Am. Chem. Soc. 82:4600 (1960) and Biochem. J. 91:589 (1964);

(iii) aryl halides such as reactive nitrohaloaromatic compounds;

(iv) alkyl halides, as described, for example, by McKenzie et al., J. Protein Chem. 7:581 (1988);

(v) aldehydes and ketones capable of Schiff's base formation with amino groups, the adducts formed usually being stabilized through reduction to give a stable amine;

(vi) epoxide derivatives such as epichlorohydrin and bisoxiranes, which may react with amino, sulfhydryl, or phenolic hydroxyl groups;

(vii) chlorine-containing derivatives of s-triazines, which are very reactive towards nucleophiles such as amino, sufhydryl, and hydroxyl groups;

(viii) aziridines based on s-triazine compounds detailed above, e.g., as described by Ross, J. Adv. Cancer Res. 2:1 (1954), which react with nucleophiles such as amino groups by ring opening;

(ix) squaric acid diethyl esters as described by Tietze, Chem. Ber. 124:1215 (1991); and

(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:

(i) isocyanates and isothiocyanates, particularly aromatic derivatives, which form stable urea and thiourea derivatives respectively;

(ii) sulfonyl chlorides, which have been described by Herzig et al., Biopolymers 2:349 (1964);

(iii) acid halides;

(iv) active esters such as nitrophenylesters or N-hydroxysuccinimidyl esters;

(v) acid anhydrides such as mixed, symmetrical, or N-carboxyanhydrides;

(vi) other useful reagents for amide bond formation, for example, as described by M. Bodansky, Principles of Peptide Synthesis, Springer-Verlag, 1984;

(vii) acylazides, e.g., wherein the azide group is generated from a preformed hydrazide derivative using sodium nitrite, as described by Wetz et al., Anal. Biochem. 58:347 (1974); and

(viii) imidoesters, which form stable amidines on reaction with amino groups, for example, as described by Hunter and Ludwig, J. Am. Chem. Soc. 84:3491 (1962).

Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

It will be appreciated that functional groups in drug (A) and/or drug (B) may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of drug (A) with a reactive chemical group of drug (B) without introducing additional linking material may, if desired, be used in accordance with the invention.

More commonly, however, the linker will include two or more reactive moieties, as described above, connected by a spacer element. The presence of such a spacer permits bifunctional linkers to react with specific functional groups within drug (A) and drug (B), resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between drug (A) and drug (B).

Spacer elements in the linker typically consist of linear or branched chains and may include a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl.

In some instances, the linker is described by formula (II):

G¹-(Z¹)ₒ-(Y¹)ᵤ-(Z²)ₛ-(R₃₀)-(Z³)ₜ-(Y²)ᵥ-(Z⁴)ₚ-G² (II)

In formula (II), G¹ is a bond between drug (A) and the linker; G² is a bond between the linker and drug (B); Z¹, Z², Z³, and Z⁴ each, independently, is selected from O, S, and NR₃₁; R₃₁ is hydrogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; Y¹ and Y² are each, independently, selected from carbonyl, thiocarbonyl, sulphonyl, or phosphoryl; o, p, s, t, u, and v are each, independently, 0 or 1; and R₃₀ is a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl, or a chemical bond linking G¹-(Z¹)ₒ-(Y¹)ᵤ-(Z²)ₛ- to -(Z³)ₜ—(Y²)ᵥ-(Z⁴)ₚ-G².

Examples of homobifunctional linkers useful in the preparation of conjugates of the invention include, without limitation, diamines and diols selected from ethylenediamine, propylenediamine and hexamethylenediamine, ethylene glycol, diethylene glycol, propylene glycol, 1,4-butanediol, 1,6-hexanediol, cyclohexanediol, and polycaprolactone diol.

### Formulation of Pharmaceutical Compositions

The compositions, methods, and kits of the invention can include formulation(s) of compound(s) that, upon administration to a subject, result in a concentration of the compound(s) that treats a viral hepatitis infection. The compound(s) may be contained in any appropriate amount in any suitable carrier substance, and are generally present in an amount of 1-95% by weight of the total weight of the composition. The composition may be provided in a dosage form that is suitable for the oral, parenteral (e.g., intravenously or intramuscularly), rectal, determatological, cutaneous, nasal, vaginal, inhalant, skin (patch), ocular, intrathecal, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions according to the invention or used in the methods of the invention may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agent(s) of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the combination to a particular target cell type. Administration of compound(s) in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound(s) are formulated with appropriate excipients into a pharmaceutical composition that, upon administration, releases the compound(s) in a controlled manner. Examples include single or multiple unit tablet or capsule compositions, oil solutions, suspensions, emulsions, microcapsules, molecular complexes, microspheres, nanoparticles, patches, and liposomes.

### Delivery of Compound(s)

It is not intended that administration of compounds be limited to a single formulation and delivery method for all compounds of a combination. The combination can be administered using separate formulations and/or delivery methods for each compound of the combination using, for example, any of the above-described formulations and methods. In one example, a first agent is delivered orally, and a second agent is delivered intravenously.

### Dosages

The dosage of a compound or a combination of compounds depends on several factors, including: the administration method, the type of viral hepatitis to be treated, the severity of the infection, whether dosage is designed to treat or prevent a viral hepatitis infection, and the age, weight, and health of the patient to be treated.

For combinations that include an anti-viral agent in addition to a compound identified herein (e.g., a compound of Table 1, Table 2, or Table 3), the recommended dosage for the anti-viral agent is can be less than or equal to the recommended dose as given in the Physician's Desk Reference, 60^{th} Edition (2006).

As described above, the compound in question may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied. The correct dosage of a compound can be determined by examining the efficacy of the compound in viral replication assays, as well as its toxicity in humans.

An antiviral agent is usually given by the same route of administration that is known to be effective for delivering it as a monotherapy. For example, when used in combination therapy with a compound of Table 1, Table 2, or Table 3 according to the methods of this invention, an agent of Table 4 or Table 5 is dosed in amounts and frequencies equivalent to or less than those that result in its effective monotherapeutic use.

### Additional Applications

If desired, the compounds of the invention may be employed in mechanistic assays to determine whether other combinations, or single agents, are as effective as the combinations of the invention in inhibiting a viral disease (e.g., those described herein) using assays generally known in the art. For example, candidate compounds may be tested, alone or in combination (e.g., with an agent that inhibits viral replication, such as those described herein) and applied to cells (e.g., hepatic cells such as Huh7, Huh2, Huh 8, Sk-Hep-1, Huh7 lunet, HepG2, WRL-68, FCA-1, LX-1, and LX-2). After a suitable time, viral replication or load of these cells is examined. A decrease in viral replication or viral load identifies a candidate compound or combination of agents as an effective agent for treating a viral disease.

The agents of the invention are also useful tools in elucidating mechanistic information about the biological pathways involved in viral diseases. Such information can lead to the development of new combinations or single agents for treating, preventing, or reducing a viral disease. Methods known in the art to determine biological pathways can be used to determine the pathway, or network of pathways affected by contacting cells (e.g., hepatic cells) infected with a virus with the compounds of the invention. Such methods can include, analyzing cellular constituents that are expressed or repressed after contact with the compounds of the invention as compared to untreated, positive or negative control compounds, and/or new single agents and combinations, or analyzing some other activity of the cell or virus such as an enzymatic-activity, nutrient uptake, and proliferation. Cellular components analyzed can include gene transcripts, and protein expression. Suitable methods can include standard biochemistry techniques, radiolabeling the compounds of the invention (e.g., ¹⁴C or ³H labeling), and observing the compounds binding to proteins, e.g., using 2D gels, gene expression profiling. Once identified, such compounds can be used in in vivo models (e.g., knockout or transgenic mice) to further validate the tool or develop new agents or strategies to treat viral disease.

### Exemplary Candidate Compounds

Peptide Moieties

Peptides, peptide mimetics, and peptide fragments (whether natural, synthetic or chemically modified) are suitable for use in the methods of the invention. Exemplary inhibitors include compounds that reduce the amount of a target protein or RNA levels (e.g., antisense compounds, dsRNA, ribozymes) and compounds that compete with viral reproduction machinery (e.g., dominant negative proteins or polynucleotides encoding the same).

Antisense Compounds

The biological activity of any protein that increases viral replication, viral RNA or DNA replication, viral RNA translation, viral protein processing or activity, or viral packaging can be reduced through the use of an antisense compound directed to RNA encoding the target protein. Antisense compounds can be identified using standard techniques. For example, accessible regions of the target the mRNA of the target enzyme can be predicted using an RNA secondary structure folding program such as MFOLD (M. Zuker, D. H. Mathews & D. H. Turner, Algorithms and Thermodynamics for RNA Secondary Structure Prediction: A Practical Guide. In: RNA Biochemistry and Biotechnology, J. Barciszewski & B. F. C. Clark, eds., NATO ASI Series, Kluwer Academic Publishers, (1999)). Sub-optimal folds with a free energy value within 5% of the predicted most stable fold of the mRNA are predicted using a window of 200 bases within which a residue can find a complimentary base to form a base pair bond. Open regions that do not form a base pair are summed together with each suboptimal fold and areas that are predicted as open are considered more accessible to the binding to antisense nucleobase oligomers. Other methods for antisense design are described, for example, in U.S. Pat. No. 6,472,521, Antisense Nucleic Acid Drug Dev. 19977:439-444, Nucleic Acids Res. 28:2597-2604, 2000, and Nucleic Acids Res. 31:4989-4994, 2003.

RNA Interference

The biological activity of a molecule involved in a viral infection or viral replication can be reduced through the use of RNA interference (RNAi), employing, e.g., a double stranded RNA (dsRNA) or small interfering RNA (siRNA) directed to the signaling molecule in question (see, e.g., Miyamoto et al., Prog. Cell Cycle Res. 5:349-360, 2003; U.S. Pat. Application Publication No. 20030157030). Methods for designing such interfering RNAs are known in the art. For example, software for designing interfering RNA is available from Oligoengine (Seattle, Wash.).

### Dominant Negative Proteins

One skilled in the art would know how to make dominant negative proteins to the molecules involved in a viral infection or viral replication. Such dominant negative proteins are described, for example, in Gupta et al., J. Exp. Med., 186:473-478, 1997; Maegawa et al., J. Biol. Chem. 274:30236-30243, 1999; Woodford-Thomas et al., J. Cell Biol. 117:401-414, 1992).

The following example is intended to illustrate rather than limit the invention. Unless stated otherwise, the data shown in the Examples was generated using the HCV replicon assay.

### Example 1

### HCV Replicon Assay

The HCV replicon assay enables screening of compounds with antiviral activity against HCV viral RNA replication. Huh7 cells expressing a subgenomic RNA replicon of Con1 (genotype 1b) sequence origin and expressing the reporter enzyme luciferase were obtained from ReBLikon, GmBH. In order to perform the assay, seed replicon cells on a 384-well plate at 4,000 cells/well in a total volume of 30 uL/well. The plated cells are incubated at 37° C., 5% CO₂. Pre-diluted compounds are added at a 10× concentration to each well to achieve the desired final concentration. Plates are centrifuged at 900×g, 1 minute following the addition of compounds. Incubate cells an additional 48 hours or 72 hours at 37° C., 5% CO₂. Remove plates from the incubator 30 minutes to 1 hour prior to the addition of 25 μL/well of SteadyLite luciferase assay reagent from Perkin Elmer in order to equilibrate plates to room temperature. Following the addition of SteadyLite reagent, allow cells to incubate for 10 minutes prior to collecting data with a luminometer. Antiviral activity is quantified by the inhibition of luciferase activity.

In order to confirm that a decrease in luciferase activity correlates with inhibition of HCV replicon replication and not an increase in cell death, a counter screen is run in tandem. Huh7 parental cells which do not express HCV replicon RNA are treated similarly to the above replicon cells; briefly, seed cells on a 384-well plate at 4,000 cells/well as described above. Compounds are added the following day and, after a subsequent 48-hour incubation at 37° C., 5% CO₂, 15 μl/well of ATPlite (Perkin Elmer) is added after plates have been equilibrated at room temperature. The ATPlite assay provides a quantitative measure of the levels of ATP in the cell cultures in each well, where higher levels of ATP correlate with greater cellular viability. Thus, a compound with antiviral activity is expected to inhibit the levels of luciferase measured by the SteadyLite assay without any or minimal effect on the ATP levels measured by the ATPlite assay.

Using the screen described above or a similar screen, we identified the agents listed in Tables 1, 2, and 3 and the combinations of agents listed in Table 9. For screens involving a combination of compounds, a synergy score was calculated by the formula s=log f_{X} log f_{Y}ΣI_{data}(I_{data}−I_{Loewe}), summed over all non-single-agent concentration pairs, and where log f_{X,Y} are the natural logarithm of the dilution factors used for each single agent. This effectively calculates a volume between the measured and Loewe additive response surfaces, weighted towards high inhibition and corrected for varying dilution factors. The synergy score indicates that the combination of the two agents provides greater antiviral activity than would be expected based on the protection provided by each agent of the combination individually. The following ranges of concentrations of agents were used to generate the synergy scores in Table 12: sertraline (0.105-13 μM); simvastatin (0.175-22 μM); fluvastatin (0.22-28 μM); lovastatin (0.06-7.9 μM); rosuvastatin (0.19-24 μM); and hydroxyzine (0.21-27 μM). These data were generated following a 48-hour cell incubation.

TABLE 12
Combinations of compounds

| Compound 1 | Compound 2 | Synergy Score |
|---|---|---|
| Sertraline hydrochloride | Fluvastatin | 4.7305 |
| Sertraline hydrochloride | Lovastatin | 3.6093 |
| Sertraline hydrochloride | Rosuvastatin calcium | 4.4640 |
| Sertraline hydrochloride | Simvastatin | 3.0251 |
| Sertraline hydrochloride | Hydroxyzine hydrochloride | 1.4113 |

Synergy scores were also identified for the following combination of compounds (Tables 13 and 14). These data were also generated after a 48-hour cell incubation.

TABLE 13

| | | Synergy |
|---|---|---|
| Compound A | Compound B | Score |
| Amorolfine Hydrochloride | Sertraline Hydrochloride | 5.202 |
| Fluvastatin | Sertraline Hydrochloride | 4.729 |
| Rosuvastatin calcium | Sertraline Hydrochloride | 4.481 |
| Fulvestrant | Satraplatin | 3.562 |
| Amorolfine Hydrochloride | Mebeverine Hydrochloride | 3.527 |
| Amorolfine Hydrochloride | Satraplatin | 3.414 |
| Ifenprodil tartrate | Sertraline Hydrochloride | 3.344 |
| Amorolfine Hydrochloride | Tolterodine Tartrate | 3.156 |
| Atorvastatin | Sertraline Hydrochloride | 3.136 |
| Amorolfine Hydrochloride | Irinotecan Hydrochloride | 3.059 |
| Lovastatin | Sertraline Hydrochloride | 3.022 |
| Cytarabine | Triciribine | 2.970 |
| Artesunate | Wortmannin | 2.964 |
| Sertraline Hydrochloride | Simvastatin Hydroxy Acid, | 2.955 |
| | Ammonium Salt | |
| Amorolfine Hydrochloride | Cytarabine | 2.944 |
| Sertraline Hydrochloride | Simvastatin | 2.930 |
| Octyl Methoxycinnamate | Suberohydroxamic Acid | 2.840 |
| 1,5-Bis(4-aminophenoxy)- | Amorolfine Hydrochloride | 2.756 |
| pentane | | |
| (S,S)-N-Desmethyl Sertraline, | Simvastatin | 2.737 |
| Hydrochloride | | |
| Artemisinin | SB-202190 | 2.689 |
| Interferon Alfa-2a | Sirolimus | 2.678 |
| Amorolfine Hydrochloride | Indocyanine Green | 2.623 |
| TOFA | Triciribine | 2.606 |
| 3,3′-(Penta- | Artemisinin | 2.602 |
| methylenedioxy)dianiline | | |
| Artemisinin | Wortmannin | 2.599 |
| 3,3″-(Penta | Artemisinin | 2.554 |
| methylenedioxy)diacetanilide | | |
| Amorolfine Hydrochloride | Benzamil HCL | 2.549 |
| Artemisinin | Triciribine | 2.495 |
| 2,2′-(Penta- | Amorolfine Hydrochloride | 2.494 |
| methylenedioxy)dianiline | | |
| (S,S)-N-Desmethyl Sertraline, | Simvastatin Hydroxy Acid, | 2.475 |
| Hydrochloride | Ammonium Salt | |
| Levothyroxine Sodium | Wedelolactone | 2.417 |
| 1,5-Bis(4-aminophenoxy)- | Artemisinin | 2.390 |
| pentane | | |
| Benzamil HCL | Dextrothyroxine Sodium | 2.353 |
| Amorolfine Hydrochloride | Trifluperidol | 2.321 |
| Artemisinin | Indocyanine Green | 2.311 |
| Dihydroartemisinin | Wortmannin | 2.243 |
| Flupentixol Dihydrochloride | Sertraline Hydrochloride | 2.185 |
| Benzamil HCL | Levothyroxine Sodium | 2.131 |
| Amorolfine Hydrochloride | Meclizine | 2.093 |
| Pravastatin Sodium | Sertraline Hydrochloride | 2.033 |
| 1,5-Bis(4-aminophenoxy)- | Indocyanine Green | 2.030 |
| pentane | | |
| 2-Hydroxyflavanone | Amorolfine Hydrochloride | 1.990 |
| Ritonavir | Vinorelbine | 1.989 |
| Benoxinate Hydrochloride | Dehydroepiandrosterone | 1.975 |
| Ifenprodil tartrate | Indocyanine Green | 1.930 |
| Amorolfine Hydrochloride | Arbidol | 1.911 |
| 3,3′-(Penta- | Indocyanine Green | 1.905 |
| methylenedioxy)dianiline | | |
| Fulvestrant | Vinorelbine | 1.902 |
| Amorolfine Hydrochloride | Ezetimibe | 1.890 |
| Amorolfine Hydrochloride | Evans Blue | 1.885 |
| Amorolfine Hydrochloride | Gefitinib (Base) | 1.838 |
| Amorolfine Hydrochloride | Topotecan Hydrochloride | 1.810 |
| 2′,2″-(Penta- | Artemisinin | 1.798 |
| methylenedioxy)diacetanilide | | |
| Amorolfine Hydrochloride | Wedelolactone | 1.770 |
| 3,3′-(Penta | Amorolfine Hydrochloride | 1.746 |
| methylenedioxy)dianiline | | |
| Simvastatin | rac-cis-N-Desmethyl Sertraline, | 1.744 |
| | Hydrochloride | |
| Adefovir Dipivoxil | Triciribine | 1.741 |
| Cytarabine | Evans Blue | 1.714 |
| Artemisinin | Evans Blue | 1.664 |
| Fluphenazine Hydrochloride | Sertraline Hydrochloride | 1.647 |
| Benzamil HCL | SB-202190 | 1.643 |
| Artemisinin | Rifabutin | 1.627 |
| Fluphenazine Hydrochloride | Tolterodine Tartrate | 1.603 |
| Interferon Alfa-2a | Melphalan | 1.537 |
| Amorolfine Hydrochloride | Melphalan | 1.535 |
| Artemisinin | Fulvestrant | 1.477 |
| Ifenprodil tartrate | Quinacrine | 1.466 |
| Simvastatin Hydroxy Acid, | rac-cis-N-Desmethyl Sertraline, | 1.456 |
| Ammonium Salt | Hydrochloride | |
| Flupentixol Dihydrochloride | Tolterodine Tartrate | 1.440 |
| Triciribine | Wortmannin | 1.439 |
| Loratadine | Vinorelbine | 1.423 |
| Meclizine | Sertraline Hydrochloride | 1.358 |
| Budesonide | Vinorelbine | 1.356 |
| 2-Hydroxyflavanone | Indocyanine Green | 1.308 |
| Hydroxyzine Hydrochloride | Sertraline Hydrochloride | 1.293 |
| 2,2′-(Penta- | Artemisinin | 1.281 |
| methylenedioxy)dianiline | | |
| Amorolfine Hydrochloride | Flupentixol Dihydrochloride | 1.259 |
| Artemisinin | Chlorophyllin | 1.256 |
| Ezetimibe | Fluphenazine Hydrochloride | 1.240 |
| Benzamil HCL | Fluphenazine Hydrochloride | 1.237 |
| Artemisinin | Wedelolactone | 1.228 |
| Cytarabine | Dydrogesterone | 1.215 |
| Artemisinin | Benzamil HCL | 1.205 |
| 3,3′-(Penta- | Artemether | 1.169 |
| methylenedioxy)dianiline | | |
| Tolterodine Tartrate | Trifluperidol | 1.146 |
| Artesunate | Fluvastatin | 1.102 |
| Artemisinin | Trifluridine | 1.095 |
| Adefovir Dipivoxil | Amorolfine Hydrochloride | 1.069 |
| Interferon Alfa-2a | Trifluridine | 1.066 |
| Fulvestrant | Triciribine | 1.032 |
| Artesunate | Dydrogesterone | 1.032 |
| Artesunate | LY 294002 | 1.006 |
| Mosapride Citrate | TOFA | 0.986 |
| Bromocriptine Mesylate | Wedelolactone | 0.978 |
| Artemisinin | Sodium Fusidate | 0.968 |
| Celgosivir | Interferon Alfa-2a | 0.966 |
| Amorolfine Hydrochloride | Dextrothyroxine Sodium | 0.960 |
| Andrographis | Fulvestrant | 0.944 |
| 2′-C-Methylcytidine | Artemisinin | 0.937 |
| Amorolfine Hydrochloride | Gemcitabine Hydrochloride | 0.923 |
| Oxeladin | Sertraline Hydrochloride | 0.909 |
| Artemisinin | Parthenolide | 0.903 |
| Artemisinin | Ribavirin | 0.899 |
| Dehydroepiandrosterone | Tyrphostin Ag 1478 | 0.880 |
| Sertraline Hydrochloride | Toremifene | 0.879 |
| Dihydroartemisinin | Fulvestrant | 0.863 |
| 2-Hydroxyflavanone | TOFA | 0.860 |
| Artesunate | Repaglinide | 0.854 |
| Mofebutazone | Wedelolactone | 0.842 |
| Artesunate | Simvastatin | 0.841 |
| 2,2′-(Penta | Artesunate | 0.821 |
| methylenedioxy)dianiline | | |
| Artemisinin | Gemcitabine Hydrochloride | 0.820 |
| Dihydroartemisinin | Ezetimibe | 0.812 |
| Chlorophyllin | Cytarabine | 0.811 |

TABLE 14

| | | Synergy |
|---|---|---|
| Compound A | Compound B | Score |
| Interferon Alfa-2a | Sirolimus | 2.678 |
| Suberohydroxamic Acid | VX-497 | 2.113 |
| Artemisinin | VX-497 | 2.103 |
| Artesunate | VX-497 | 1.692 |
| Tolterodine Tartrate | VX-950 | 1.689 |
| Artemisinin | HCV-796 | 1.683 |
| Artemisinin | NM-283 | 1.681 |
| NM-283 | Wedelolactone | 1.667 |
| Artemisinin | SCH 503034 | 1.654 |
| Cytarabine | SCH 503034 | 1.562 |
| SCH 503034 | Triciribine | 1.549 |
| Interferon Alfa-2a | Melphalan | 1.537 |
| Benoxinate Hydrochloride | VX-950 | 1.432 |
| HCV-796 | Sirolimus | 1.412 |
| Benoxinate Hydrochloride | SCH 503034 | 1.401 |
| Melphalan | VX-950 | 1.397 |
| Ritonavir | VX-950 | 1.388 |
| VX-950 | VX-497 | 1.354 |
| Artemisinin | VX-950 | 1.343 |
| Triciribine | VX-950 | 1.305 |
| Suberohydroxamic Acid | VX-950 | 1.277 |
| HCV-796 | Suberohydroxamic Acid | 1.259 |
| Sirolimus | VX-950 | 1.245 |
| Melphalan | SCH 503034 | 1.224 |
| SCH 503034 | Wortmannin | 1.212 |
| SCH 503034 | Tolterodine Tartrate | 1.188 |
| Ritonavir | SCH 503034 | 1.160 |
| Ezetimibe | VX-950 | 1.160 |
| HCV-796 | VX-497 | 1.146 |
| Chlorophyllin | VX-497 | 1.144 |
| HCV-796 | Melphalan | 1.143 |
| Capsaicin | NM-283 | 1.112 |
| SCH 503034 | Sirolimus | 1.105 |
| LY 294002 | SCH 503034 | 1.073 |
| Adefovir Dipivoxil | SCH 503034 | 1.072 |
| Interferon Alfa-2a | Trifluridine | 1.066 |
| HCV-796 | Trifluridine | 1.065 |
| GW 5074 | NM-283 | 1.061 |
| Mosapride Citrate | VX-950 | 1.057 |
| Interferon Alfa-2a | VX-497 | 1.017 |
| NM-283 | Trequinsin Hydrochloride | 0.990 |
| Cytarabine | HCV-796 | 0.989 |
| Adefovir Dipivoxil | VX-950 | 0.961 |
| Cytarabine | VX-950 | 0.956 |
| SCH 503034 | Saquinavir Mesylate | 0.948 |
| VX-950 | Wortmannin | 0.941 |
| Capsaicin | VX-950 | 0.938 |
| 2-Hydroxyflavanone | NM-283 | 0.935 |
| Bromhexine | VX-950 | 0.935 |
| HCV-796 | Wortmannin | 0.915 |
| Artemisinin | Ribavirin | 0.899 |
| VX-950 | Verapamil | 0.895 |
| SCH 503034 | Verapamil | 0.880 |
| SCH 503034 | Topotecan Hydrochloride | 0.879 |
| HCV-796 | Topotecan Hydrochloride | 0.875 |
| Trifluperidol | VX-950 | 0.866 |
| Irinotecan Hydrochloride | SCH 503034 | 0.864 |
| Artesunate | SCH 503034 | 0.849 |
| Repaglinide | SCH 503034 | 0.845 |
| Topotecan Hydrochloride | VX-950 | 0.839 |
| Repaglinide | VX-950 | 0.825 |
| Arbidol | VX-950 | 0.821 |
| Chlorophyllin | HCV-796 | 0.813 |
| Benzydamine hydrochloride | VX-950 | 0.800 |
| NM-283 | Trifluperidol | 0.798 |
| Capsaicin | HCV-796 | 0.755 |
| NM-283 Hydrochloride | Phenazopyridine | 0.692 |
| NM-283 | Trifluridine | 0.688 |
| Adefovir Dipivoxil | HCV-796 | 0.672 |

Synergy scores and were also determined for combinations of sertraline analogs with simvastatin (Table 15). IC₅₀, Maximal effect, CC₅₀ and the therapeutic index (TI) (CC₅₀/IC₅₀) for the sertraline analogs are shown in Table 16. These data were generated after a 48-hour cell incubation.

TABLE 15

| Compound + Simvastatin | Synergy Score | Std. Dev. |
|---|---|---|
| Sertraline Hydrochloride | 2.76 | 0.35 |
| (1S,4S)-Desmethyl Sertraline, Hydrochloride | 2.14 | 0.33 |
| Sertraline B-Ring Para-Trifluoromethane | 1.93 | 0.47 |
| rac-cis-N-Desmethyl Sertraline, | 1.51 | 0.52 |
| Hydrochloride | | |
| Sertraline A-Ring Ethanol | 1.46 | 0.2 |
| Dimethyl Sertraline Reverse Sulfonamide | 1.22 | 0.35 |
| N,N-Dimethyl Sertraline | 0.89 | 0.4 |
| Sertraline B-Ring Ortho-Methoxy | 0.81 | 0.11 |
| Sertraline (Reverse) Methanesulfonamide | 0.69 | 0.33 |
| (1R,4R)-Desmethyl Sertraline | 0.57 | 0.4 |
| Sertraline Reverse Sulfonamide (CH₂ linker) | 0.55 | 0.02 |
| Sertraline N,N-Dimethylsulfonamide | 0.53 | 0.08 |
| Sertraline Nitrile | 0.52 | 0.2 |
| Sertraline A-Ring Carboxylic Acid | 0.52 | 0.08 |
| Sertraline A-Ring Methyl Ester | 0.51 | 0.24 |
| Sertraline Sulfonamide NH₂ | 0.49 | 0.05 |
| Sertraline Sulfonamide | 0.43 | 0.28 |
| Sertraline B-Ring Para-Phenoxy | 0.38 | 0.04 |

TABLE 16

| | | Max | | TI |
|---|---|---|---|---|
| Compound | IC₅₀ | Effect | CC₅₀ | (CC₅₀/IC₅₀) |
| Dimethyl Sertraline Reverse | n/a | 44% | >13.39 | n/a |
| Sulfonamide | | | | |
| Sertraline B-Ring | n/a | 24% | >22.32 | n/a |
| Ortho-Methoxy | | | | |
| Sertraline A-Ring | 2.88 | 89% | 9.81 | 3.405 |
| Carboxylic Acid | | | | |
| Sertraline B-Ring Para-Phenoxy | 3.75 | 99% | 7.46 | 1.987 |
| Sertraline A-Ring Ethanol | 4.48 | 97% | 11.1 | 2.472 |
| Sertraline Hydrochloride | 5.08 | 99% | 8.83 | 1.738 |
| (1S,4S)-Desmethyl Sertraline, | 5.35 | 98% | 6.68 | 1.247 |
| Hydrochloride | | | | |
| Sertraline Reverse Sulfonamide | 5.97 | 100% | 10.5 | 1.761 |
| (CH₂ linker) | | | | |
| Sertraline Sulfonamide NH₂ | 6.02 | 91% | >13.39 | >2.226 |
| rac-cis-N-Desmethyl Sertraline, | 6.26 | 99% | 7.84 | 1.252 |
| Hydrochloride | | | | |
| Sertraline A-Ring Methyl Ester | 6.42 | 99% | 8.01 | 1.248 |
| Sertraline (Reverse) | 6.43 | 95% | 12.9 | 2.010 |
| Methanesulfonamide | | | | |
| Sertraline Sulfonamide | 7.26 | 94% | 11.6 | 1.605 |
| Sertraline N,N- | 7.30 | 91% | 7.85 | 1.076 |
| Dimethylsulfonamide | | | | |
| (1R,4R)-Desmethyl Sertraline | 7.32 | 81% | >8.83 | >1.206 |
| Sertraline Nitrile | 7.46 | 89% | >13.39 | >1.796 |
| N,N-Dimethyl Sertraline | 7.77 | 70% | >13.41 | >1.727 |
| Sertraline B-Ring Para- | 12.22 | 99% | 15.9 | 1.299 |
| Trifluoromethane | | | | |

Activity data for sertraline analogs was also generated following a 72-hour cell incubation, as shown in Table 17.

TABLE 17

| | | | | Max Effect | |
|---|---|---|---|---|---|
| | | | | @CC30 | |
| Name | IC₅₀ (uM) | CC₅₀ (uM) | TI | (inhibition) | IC90 |
| Sertraline HCL | 4.72 ± 0.93 | 11.78 ± 2.09 | 2.55 | 62 ± 11.15% | |
| (1S,4S)-Desmethyl Sertraline | 7.5 ± 0.4 | 8.98 ± 0.1 | 1.2 | 0% | |
| (1R,4R)-Desmethyl Sertraline | 8.70 | 8.1 | 0.93 | 0% | |
| Sertraline Sulfonamide | 6.87 ± 0.2 | 13.04 ± 0.1 | 1.9 | 40.5 ± 5% | |
| Sertraline (Reverse) | 3.99 ± 0.8 | 12.89 ± 0.3 | 3.23 | 83.3 ± 20.2% | |
| Methanesulfonamide | | | | | |
| 1R,4R Sertraline Enantiomer | 12.80 | 14.32 | 1.12 | 42% | |
| N,N-Dimethyl Sertraline | 5.22 ± 2.2 | 72.2 | 13.83 | 99% | 17.06 ± 2.93 |
| Nitro Sertraline | 6.46 | 8.03 | 1.24 | 27% | |
| Sertraline Aniline | 6 | 8.72 | 1.45 | 50% | |
| Sertraline Iodide | 6.57 | 8.92 | 1.34 | 25% | |
| Sertraline Sulfonamide NH₂ | 3.65 ± 2.3 | 9.99 ± 4.4 | 2.74 | 63.5 ± 6.4% | |
| Sertraline Sulfonamide Ethanol | 8.13 | 13.75 | 1.69 | 80% | |
| Sertraline Nitrile | 4.81 | 14.82 | 3.08 | 87% | |
| Sertraline-CME | n/a | n/a | n/a | | |
| Dimethyl Sertraline Reverse | 3.45 | 16.46 | 4.77 | 70% | |
| Sulfonamide | | | | | |
| Sertraline Reverse | 3.28 ± 1.5 | 11.24 ± 0.3 | 3.45 | 59.7 ± 3.5% | |
| Sulfonamide (CH₂ linker) | | | | | |
| Sertraline B-ring Ortho | n/a | 58.63 | n/a | n/a | |
| Methoxy | | | | | |
| Sertraline A-ring Methyl Ester | 9.13 | 13.69 | 1.5 | 78% | |
| Sertraline A-Ring Ethanol | 2.85 ± 1.21 | 14.18 ± 0.3 | 4.97 | 92 ± 5.6% | 11.32 ± 2.91 |
| Sertraline N,N- | 6.69 | 8.87 | 1.33 | 38% | |
| dimethylsulfonamide | | | | | |
| Sertraline A-ring carboxylic | 1.8 ± 0.18 | 16.22 ± 0.57 | 9 | 94.5 ± 0.7% | 5.4 ± 1.81 |
| acid | | | | | |
| Sertraline B-ring para-Phenoxy | 5.15 | 6.8 | 1.32 | 65% | |
| Sertraline B-Ring para- | 13.38 | 14.18 | 1.06 | 0% | |
| Trifluoromethane | | | | | |
| N,N-Dimethyl Sertraline B-Ring | n/a | n/a | n/a | n/a | |
| Para-Trifluoromethane | | | | | |
| UK-416244 | 1.63 | 327(>100) | 200 | 97% | 9.86 |

### Example 2

### In-Vitro Activity of Combinations in H5N1 Influenza Stimulated Macrophages

Monocytes purified from blood mononuclear cell preparation were differentiated to macrophages (14 days) in 5% autologous serum. Macrophages were then infected with an A/VN/3212/04 (H5N1) virus at a MOI of two. Cells were incubated with the combination, one hour prior to the infection. During the infection, the drug was washed off for 30 minutes and reintroduced for 3 hours. RT-PCR analysis of mRNA in virus infected macrophages was carried out for the following cytokines: TNF-alpha, IFN-beta, IP-10, IL-6, IL-8, H5N₁ matrix gene (Lee et. al., J. Virol., 79:10147-10154, 2005). Cytotoxicity was evaluated visually and by Beta-actin gene expression. Fifteen combinations of agents were tested at three concentrations each.

From these experiments, the RT-PCR data was analyzed and calculated as a percentage inhibition versus a DMSO-treated control. The percent inhibition data is show in Table 18 below.

TABLE 18

| Test Combination | TNF-α | IFN-β | IP-10 | IL-6 | IL-8 | MCP-1 | M gene |
|---|---|---|---|---|---|---|---|
| Amoxapine 0.3 μM + | ++ | ++ | ++++ | − | + | +++ | + |
| Prednisolone 0.03 μM | | | | | | | |
| Amoxapine 3 μM + | +++ | + | ++ | ++++ | ++ | +++ | − |
| Prednisolone 0.3 μM | | | | | | | |
| Amoxapine 30 μM + | ++++ | +++ | +++++ | +++++ | + | ++++ | − |
| Prednisolone 3 μM | | | | | | | |
| Paroxetine HCl 0.17 μM + | ++ | + | +++ | ++++ | − | ++ | + |
| Prednisolone 0.0062 μM | | | | | | | |
| Paroxetine HCl 1.7 μM + | +++ | + | ++++ | ++++ | + | ++++ | − |
| Prednisolone 0.062 μM | | | | | | | |
| Paroxetine HCl 17 μM + | ++++ | +++ | +++++ | +++++ | − | +++ | + |
| Prednisolone 0.62 μM | | | | | | | |
| Amoxapine 0.2 μM + | − | + | ++ | + | − | − | − |
| Dipyridamole 0.5 μM | | | | | | | |
| Amoxapine 2 μM + | + | + | ++ | − | − | ++ | − |
| Dipyridamole 5 μM | | | | | | | |
| Amoxapine 20 μM + | ++++ | ++++ | ++++ | +++++ | − | ++++ | + |
| Dipyridamole 50 μM | | | | | | | |
| Budesonide 0.00012 μM + | + | + | ++ | ++ | 3 | + | + |
| Nortriptyline HCl | | | | | | | |
| 0.41 μM | | | | | | | |
| Budesonide 0.0012 μM + | ++ | + | +++ | +++++ | +++ | ++++ | − |
| Nortriptyline HCl 4.1 μM | | | | | | | |
| Budesonide 0.012 μM + | +++ | +++ | ++++ | +++++ | − | +++ | + |
| Nortriptyline HCl 41 μM | | | | | | | |
| Dipyridamole 0.0032 μM + | ++ | − | ++++ | +++ | ++ | ++ | − |
| Budesonide 0.0017 | | | | | | | |
| Dipyridamole 0.032 μM + | ++ | − | ++++ | ++++ | + | ++ | − |
| Budesonide 0.017 | | | | | | | |
| Dipyridamole 0.32 μM + | +++ | + | ++++ | ++++ | ++ | +++ | − |
| Budesonide 0.17 | | | | | | | |
| Nortriptyline HCl 0.25 μM + | − | − | ++++ | ++++ | − | − | − |
| Prednisolone 0.062 μM | | | | | | | |
| Nortriptyline HCl 2.5 μM + | ++ | − | ++++ | ++ | − | +++ | − |
| Prednisolone 0.0062 μM | | | | | | | |
| Nortriptyline HCl 25 μM + | ++++ | + | ++++ | ++ | − | +++ | − |
| Prednisolone 0.62 μM | | | | | | | |
| Paroxetine HCl 0.4 μM + | − | − | ++++ | ++ | − | ++ | − |
| Dipyridamole 0.24 μM | | | | | | | |
| Paroxetine HCl 4 μM + | + | + | +++ | ++++ | − | ++ | − |
| Dipyridamole 2.4 μM | | | | | | | |
| Paroxetine HCl 40 μM + | +++ | +++ | +++++ | +++++ | − | + | ++++ |
| Dipyridamole 24 μM | | | | | | | |
| Dipyridamole 0.06 μM + | − | − | ++ | ++ | − | + | − |
| Ibudilast 0.025 μM | | | | | | | |
| Dipyridamole 0.6 μM + | − | − | +++ | − | − | − | − |
| Ibudilast 0.25 μM | | | | | | | |
| Dipyridamole 6 μM + | ++ | + | +++ | ++ | + | ++ | + |
| Ibudilast 2.5 μM | | | | | | | |
| Epinastine 0.22 μM + | + | − | ++++ | +++ | − | + | − |
| Prednisolone 0.0062 μM | | | | | | | |
| Epinastine 2.2 μM + | ++ | − | ++++ | ++++ | ++ | ++ | − |
| Prednisolone 0.062 μM | | | | | | | |
| Epinastine 22 μM + | +++ | ++ | ++++ | + | + | +++ | − |
| Prednisolone 0.62 μM | | | | | | | |
| Bufexamac 0.28 μM + | + | − | +++ | ++ | − | − | − |
| Prednisolone 0.0016 μM | | | | | | | |
| Bufexamac 2.8 μM + | ++ | − | ++++ | +++ | − | ++ | − |
| Prednisolone 0.016 μM | | | | | | | |
| Bufexamac 28 μM + | +++ | + | ++++ | ++++ | ++ | +++ | − |
| Prednisolone 0.16 μM | | | | | | | |
| Sertraline 0.38 μM + | ++ | + | ++ | ++++ | − | ++++ | − |
| Prednisolone 0.025 μM | | | | | | | |
| Sertraline 3.8 μM + | +++ | + | +++++ | +++ | − | − | − |
| Prednisolone 0.25 μM | | | | | | | |
| Sertraline 38 μM + | ++++ | + | +++++ | ++ | − | − | +++++ |
| Prednisolone 2.5 μM | | | | | | | |
| Desloratidine 0.2 μM + | − | − | +++ | +++++ | − | − | − |
| Cyclosporine 0.004 μM | | | | | | | |
| Desloratidine 2 μM + | − | − | +++ | +++ | − | + | − |
| Cyclosporine 0.04 μM | | | | | | | |
| Desloratidine 20 μM + | ++ | + | +++ | ++++ | + | +++ | − |
| Cyclosporine 0.4 μM | | | | | | | |
| CME-Amoxapine 0.17 μM + | + | − | ++++ | − | − | − | − |
| Prednisolone | | | | | | | |
| 0.0063 μM | | | | | | | |
| CME-Amoxapine 1.7 μM + | +++ | + | ++++ | ++++ | + | ++ | − |
| Prednisolone 0.063 μM | | | | | | | |
| CME-Amoxapine 17 μM + | +++ | − | ++++ | + | +++ | +++ | − |
| Prednisolone 0.63 μM | | | | | | | |
| Desloratidine 5.3 μM + | + | − | ++++ | + | − | + | − |
| Nortriptyline HCl 0.73 μM | | | | | | | |
| Desloratidine 16 μM + | +++ | − | +++++ | +++ | − | +++ | − |
| Nortriptyline HCl 2.2 μM | | | | | | | |
| Desloratidine 48 μM + | ++++ | ++ | +++++ | +++ | − | ++ | +++ |
| Nortriptyline HCl 6.6 μM | | | | | | | |
| Desloratidine 5.3 μM + | − | − | ++ | ++ | − | − | − |
| Fluoxetine 0.15 μM | | | | | | | |
| Desloratidine 16 μM + | + | − | ++++ | +++ | − | ++ | − |
| Fluoxetine 0.45 μM | | | | | | | |
| Desloratidine 48 μM + | +++ | ++ | +++++ | ++++ | − | +++ | − |
| Fluoxetine 1.35 μM | | | | | | | |

No inhibition −
0%-20% inhibition +
21%-40% inhibition ++
41%-60% inhibition +++
61%-80% inhibition ++++
81%-100% inhibition +++++

### Example 3

### Activity of Sertraline and Combinations Containing Sertraline in Influenza Mouse Model

We also tested the effectiveness of sertraline and combinations containing sertraline in an influenza mouse model. Mouse adapted influenza A/PR/8/34 was procured from American Type Culture Collection (ATCC) and propagated in Madin-Darby Canine Kidney (MDCK) cells. The virus stock was titrated in MDCK cells to give a 10⁸TCID₅₀/mL, prior to use in mice. The virus stock was diluted in phosphate buffered saline (PBS) such that the working concentration was 10^{4.5} TCID₅₀ of virus per 50 μL.

Specific pathogen free, male C57/BL6 mice weighing 20-25 g were procured from Biological Resource Centre (BRC) and housed in groups of 3, in cages with Corncob bedding (Harlan-Teklad, U.K.). Experiments were conducted in Animal Bio-safety level 3 (ABSL-3) rooms. Cages were placed in isolator maintained at −100 pa pressure and supply of HEPA filtered air. Mice were provided with commercial rodent diet (Harlan-Teklad, U.K.) and distilled water ad libitum.

Mice were orally administered with respective treatments starting 4 hours before virus inoculation daily for five days. At the time of virus inoculation mice were anesthetized with Ketamine (75 mg/kg)+Xylazine (50 mg/kg). 50 μL of 10^{4.5} TCID₅₀ virus suspension was administered intranasally to each mouse. Previous experiments have shown that 10^{4.5} TCID₅₀/mouse of virus is lethal and produces 100% mortality in C57/BL6 mice (data not shown). Mice were weighed daily, and the weights were used for dose adjustment. Sertraline and prednisolone were suspended in 0.5% HPMC and administered once daily while oseltamivir was dissolved in distilled water and administered twice daily. Sertraline, sertraline+prednisolone combination, oseltamivir, and vehicle were orally administered for 5 days starting 4 hr before virus inoculation. The survival rate of animals was monitored for 10 days after infection.

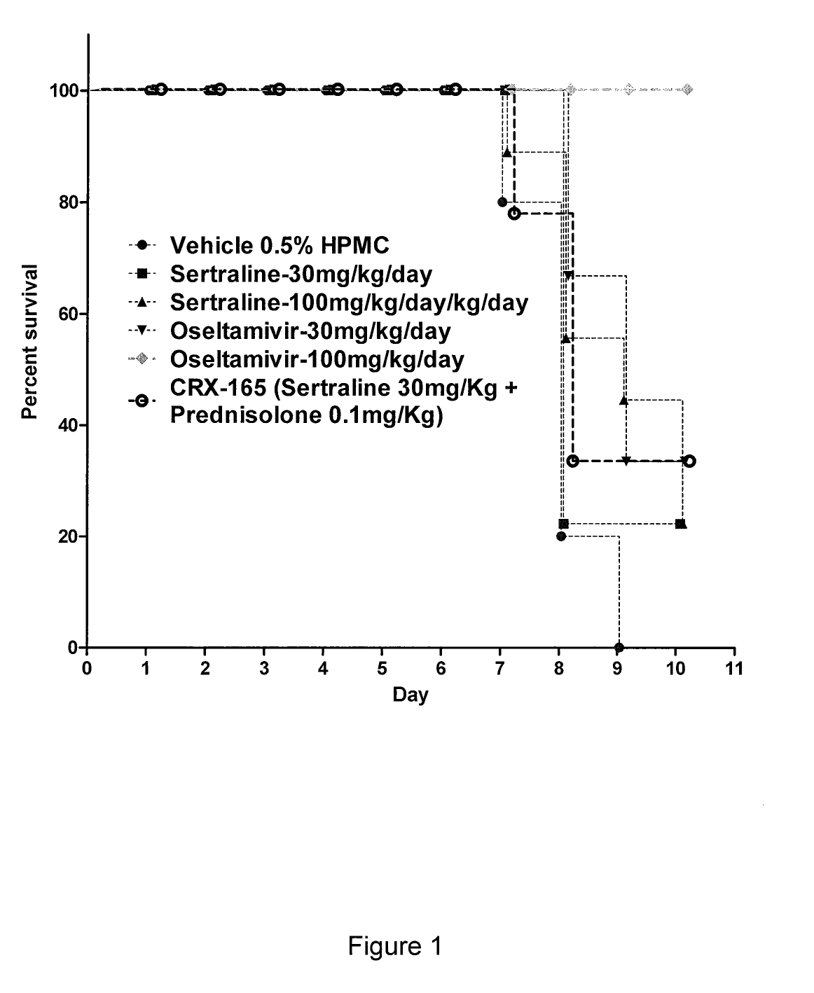

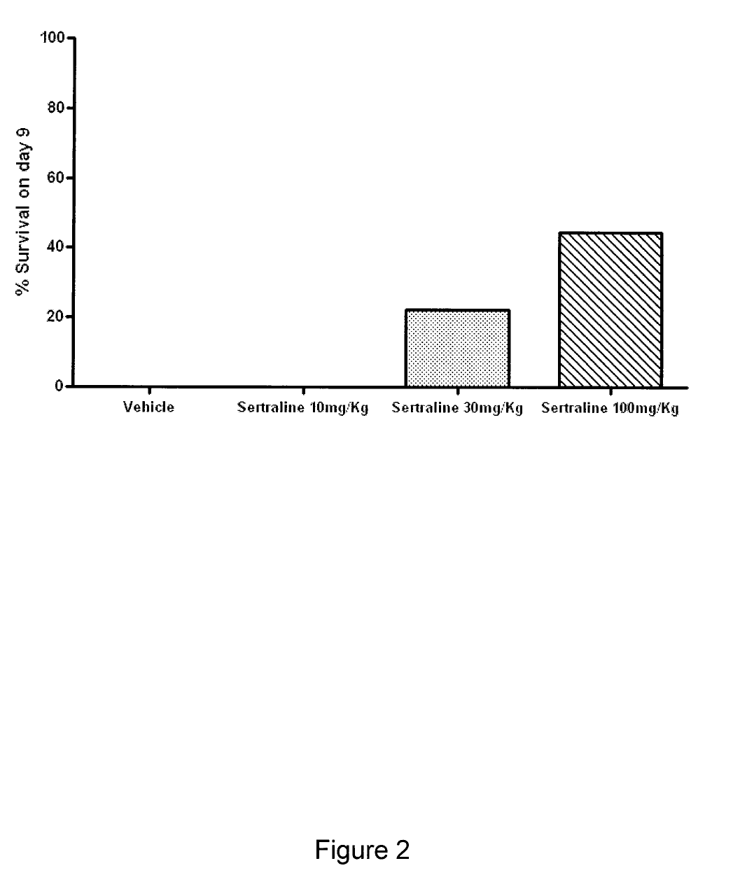

From these experiments, vehicle treated mice began to die on day 7 and their survival rate on day 9 was 0%. The survival rate of mice receiving sertraline at a dose of 30 mg/kg/day was 22.2% on day 10. In mice treated with sertraline at 100 mg/kg/day, the survival rate was 55.5% on day 8, 44.4% on day 9, and 22.2% on day 10. Thus, sertraline shows dose dependant increase in survival rate by day 9 by which vehicle treated group shows 100% mortality (FIGS. 1 and 2).

Mice treated with a combination of sertraline 30 mg/kg/day and prednisolone 0.1 mg/kgday showed 30% survival on day 10. Oseltamivir was used as a positive control and the survival rates for 30 mg/kg/day and 100 mg/kg/day were 33.3% and 100% respectively on day 10. Sertraline alone or in combination with prednisolone improves survival rate of C57/BL6 mice infected with lethal dose of influenza A/8/PR/34.

### Example 4

### Sertraline, UK-416244, and Analoges thereof

Characterization of sertraline, UK-416244, and analoges thereof is shown in Table 19.

TABLE 19

| | | | | | BBB/Neurotransmitter | | |
|---|---|---|---|---|---|---|---|
| | SA IC50 | Max effect | IC90 | | Binding (nM) | | |
| Compound | (uM)/SI | @CC30 (%) | (uM) | Pharmcokinetics | SRI | DRI | NRI |
| UK-416244 | 1.41 ± 0.20/ | 97.2 ± 1.4 | 6.17 ± 3.23 | Optimal PK, Liver | <5 nM | | |
| | >35 | | | levels lower than | | | |
| | | | | sertraline, 511 fold over | | | |
| | | | | IC50, Brain levels much | | | |
| | | | | lower than sertraline | | | |
| Sertraline A-ring Methyl | 1.46 ± 0.23/ | 97.9 ± 1.1 | 5.13 ± 0.16 | | 340 | 89 | 450 |
| Sulfoxide (CH₂ Linker) | 8.6 | | | | | | |
| Sertraline A-ring | 1.8 ± 0.18/9 | 94.5 ± 0.7 | 5.4 ± 1.81 | Optimal PK. Liver | 2 | 170 | 410 |
| carboxamide | | | | levels lower than | | | |
| | | | | sertraline, 450 fold over | | | |
| | | | | IC50, Brain levels lower | | | |
| | | | | than sertraline. | | | |
| Compound 48 | 2.33/17 | 95.49 | 10.02 | | | | |
| Sertraline A-Ring Reverse | 0.91 ± 0.08/ | 83.03 ± 6.55 | N.A. | | | | |
| Carboxamide | 13.14 | | | | | | |
| Sertraline A-Ring | 1.59/2.32 | 85.93 | NA | | | | |
| Methanamine | | | | | | | |
| Sertraline A-Ring | 1.68/7.72 | 84.79 | NA | | | | |
| Sulfonylmethane (CH₂- | | | | | | | |
| Linker) | | | | | | | |
| Sertraline (Reverse) | 3.99 ± 0.8/ | 83.3 ± 20.2 | NA | Optimal PK, Liver levels | 3 | 55 | 60 |
| Methanesulfonamide | 3.23 | | | much greater | | | |
| | | | | Sertraline, 1300 fold over | | | |
| | | | | IC50, Brain levels much | | | |
| | | | | lower sertraline | | | |
| Sertraline | 4.22 ± 1.05/ | 62.6 ± 21.5 | NA | Liver levels 1083 folds | 3 | 310 | 825 |
| | 2.77 | | | over IC50, Brain:plasma | | | |
| | | | | ratio of 31:1 | | | |
| Sertraline A-ring Thiophene | 8.27 ± 0.46/ | 80.8 ± 2.55 | NA | | | | |
| | 2.28 | | | | | | |

Characterization of additional sertraline analogs of the formula,

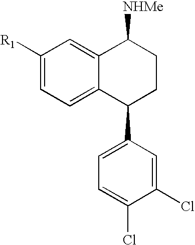

is shown in Table 20 below. Sertraline is shown in bold.

TABLE 20

| R₁ | IC50 | TI | IC90 |
|---|---|---|---|
| NHCOMe | 0.91 | 13.1 | |
| CH₂S(O)CH₃ | 1.46 | 8.6 | 5.13 |
| CH₂NH₂ | 1.6 | 2.32 | |
| CH₂SO₂CH₃ | 1.68 | 7.72 | |
| CONH₂ | 1.8 | 9 | 5.4 |
| CH₂OH | 2.85 | 4.97 | 11.32 |
| NHCOPh | 2.94 | 1.25 | |
| CH₂NHSO₂Me | 3.28 | 3.45 | |
| NHSO₂Ph | 3.48 | 1.13 | |
| NMe₂ | 3.52 | 1.77 | |
| SO₂NH₂ | 3.65 | 2.74 | |
| NHCOBu | 3.68 | 1.17 | |
| NHSO₂Me | 3.99 | 3.23 | |
| NHCOcyclopentyl | 4.02 | 1.74 | |
| H | 4.23 | 2.75 | |
| CN | 4.81 | 3.08 | |
| NHSO₂cyclopropyl | 5.63 | 1.32 | |
| NH₂ | 6 | 1.45 | |
| NO₂ | 6.46 | 1.24 | |
| I | 6.57 | 1.34 | |
| SO₂NMe₂ | 6.69 | 1.33 | |
| SO₂NHMe | 6.87 | 1.9 | |
| SO₂NHCH₂CH₂OH | 8.13 | 1.69 | |
| CO₂Me | 9.13 | 1.5 | |
| NHSO₂Bu | NA | NA | toxic |

Characterization of sertraline analogs of the formula:

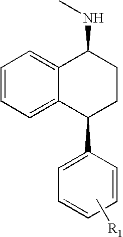

is shown in Table 21. Sertraline is shown in bold.

TABLE 21

| R₁ | IC50 | TI |
|---|---|---|
| p-OPh | 4.2 | 1.95 |
| 3,4-di-Cl | 4.23 | 2.75 |
| P—CF₃ | 13.38 | 1.06 |
| p-OMe | 21 | 3.3 |
| m-OMe | 28.77 | >1.74 |
| o-OMe | NA | NA |

Characterization of sertraline analogs of the formula:

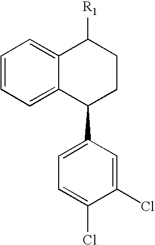

are shown in Table 22. Sertraline is noted in bold.

TABLE 22

| R₁ | IC50 | TI |
|---|---|---|
| NHMe | 4.23 | 2.75 |
| OMe-P1 | 22.67 | >2.2 |
| OH—P1 | 24.47 | >2.04 |
| OMe-P2 | 26.4 | >1.89 |
| ═O | 32.57 | >1.53 |
| OH—P2 | >50 | NA |

Characterization of sertraline analogs of the formula:

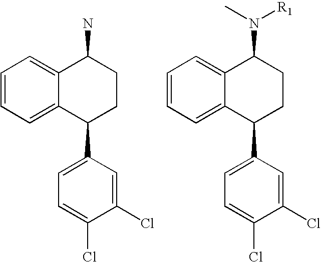

is shown in Table 23. Sertraline is shown in bold.

TABLE 23

| R₁ | IC50 | TI | IC90 |
|---|---|---|---|
| H | 4.23 | 2.75 | |
| Me | 6.06 | 10.7 | 18.13 |
| CH2CH2OH | 7.8 | 6.38 | 21.92 |
| cyclopropyl | 14.5 | >3.4 | 34.2 |
| CH2COOH | NA | NA | NA |

Characterization of additional sertraline analogs is shown in Table 24. Sertraline is in bold.

TABLE 24

| | | | | Max | | |
|---|---|---|---|---|---|---|
| | IC₅₀ (uM) | CC₅₀ (uM) | Therapeutic | Effect@CC₃₀ | | n |
| Name | [SG] | [SG] | index | (inhibition) | IC90 | [SG] |
| Sertraline HCL | 4.22 ± 1.05 | 11.7 ± 2.03 | 2.77 | 62.62 ± 21.53% | | 14 |
| (1S,4S)-Desmethyl Sertraline | *7.5 ± 0.4 | 8.98 ± 0.1 | 1.2 | 0% | | 2 |
| (1R,4R)-Desmethyl Sertraline | *8.70 | 8.1 | 0.93 | 0% | | 1 |
| Sertraline Sulfonamide | 6.87 ± 0.2 | 13.04 ± 0.1 | 1.9 | 40.5 ± 5% | | 2 |
| Sertraline (Reverse) | 3.99 ± 0.8 | 12.89 ± 0.3 | 3.23 | 83.3 ± 20.2% | | 3 |
| Methanesulfonamide | | | | | | |
| 1R,4R Sertraline Enantiomer | 10.9 ± 2.67 | 14.58 ± 0.37 | 1.34 | 58.7 ± 23.6% | | 2 |
| N,N-Dimethyl Sertraline | 6.07 ± 2.2 | 64.7 ± 10.61 | 10.6 | 99% | 18.13 ± 2.77 | 3 |
| Nitro Sertraline | **6.46 | 8.03 | 1.24 | 27% | | 1 |
| Sertraline Aniline | 6 | 8.72 | 1.45 | 50% | | 1 |
| Sertraline Iodide | **6.57 | 8.92 | 1.34 | 25% | | 1 |
| Sertraline Sulfonamide NH₂ | 3.65 ± 2.3 | 9.99 ± 4.4 | 2.74 | 63.5 ± 6.4% | | 2 |
| Sertraline Sulfonamide Ethanol | 8.13 | 13.75 | 1.69 | 80% | | 1 |
| Sertraline Nitrile | 4.81 | 14.82 | 3.08 | 87% | | 1 |
| Sertraline-CME | NA | NA | NA | | | 1 |
| Dimethyl Sertraline Reverse | 3.45 | 16.46 | 4.77 | 70% | | 1 |
| Sulfonamide | | | | | | |
| Sertraline Reverse Sulfonamide | 3.28 ± 1.5 | 11.24 ± 0.3 | 3.45 | 59.7 ± 3.5% | | 3 |
| (CH₂ linker) | | | | | | |
| Sertraline B-ring Ortho Methoxy | NA | 58.63 | NA | NA | | 2 |
| Sertraline A-ring Methyl Ester | 9.13 | 13.69 | 1.5 | 78% | | 1 |
| Sertraline A-Ring Ethanol | 2.85 ± 1.21 | 14.18 ± 0.3 | 4.97 | 92 ± 5.6% | 11.32 ± 2.91 | 2 |
| Sertraline N,N- | 6.69 | 8.87 | 1.33 | 38% | | 1 |
| dimethylsulfonamide | | | | | | |
| Sertraline A-ring carboxamide | 1.8 ± 0.18 | 16.22 ± 0.57 | 9 | 94.5 ± 0.7% | 5.4 ± 1.81 | 2 |
| Sertraline B-ring para-Phenoxy | 4.02 ± 1.6 | 7.85 ± 1.48 | 1.95 | 81 ± 22.6% | | 2 |
| Sertraline B-Ring para- | 13.38 | 14.18 | 1.06 | 0% | | 1 |
| Trifluoromethane | | | | | | |
| N,N-Dimethyl Sertraline B-Ring | NA | NA | NA | NA | | 1 |
| Para-Trifluoromethane | | | | | | |
| Sertraline B-ring 2-Thiophene | 52.3 | 88.55 | 1.69 | 42% | | 1 |
| Sertraline without B-Ring | NA | NA | NA | NA | | 1 |
| N-Ethanol Sertraline | 7.8 | 49.8 | 6.38 | 93% | 21.92 | 1 |
| N-Cyclopropyl Sertraline | 14.5 | >50 | >3.4 | 99% | 34.2 | 1 |
| (1S,4R) Sertraline Hydrochloride | 5.96 ± 0.78 | 7.51 ± 0.8 | 1.26 | 50 ± 17.4% | | 2 |
| (1R,4S) Sertraline Hydrochloride | 8.02 ± 0.71 | 14.2 ± 0.16 | 1.77 | 90.8 ± 1.6% | 12.09 ± 0.83 | 2 |
| Sertraline B-Ring Para-Methoxy | 26.8 | 51.1 | 1.9 | 30.40% | | 1 |
| P1 | | | | | | |
| Sertraline B-Ring Para-Methoxy | 21 | 68.7 | 3.3 | 50.10% | | 1 |
| P2 | | | | | | |
| Sertraline A-ring Thiophene | 8.27 ± 0.46 | 18.84 ± 0.93 | 2.28 | 80.8 ± 2.55% | | 2 |
| Sertraline A-ring Methyl | 1.34 ± 0.26 | 14.85 ± 0.97 | 11.08 | 96.47 ± 2.6% | 5.17 ± 0.14 | 3 |
| Sulfoxide (CH₂ Linker) | | | | | | |
| N,N-dimethyl Sertraline A-ring | NA | NA | NA | NA | | 2 |
| Carboxylic acid | | | | | | |
| Sertraline B-ring m-Methoxy | 28.77 | >50 | >1.74 | 27.70% | | 1 |
| N,N-Dimethyl Sertraline A-Ring | 2.62 ± 0.48 | 21.73 ± 1.44 | 8.29 | 94.3 ± 0.7% | 8.77 ± 0.05 | 2 |
| Carboxamide | | | | | | |
| Sertraline A-Ring Reverse | 0.91 ± 0.08 | 11.96 ± 1.77 | 13.14 | 83.03 ± 6.55% | | 2 |
| Carboxamide | | | | | | |
| 4S-Sertraline Ketone | 32.57 | >50 | >1.54 | 77% | | 1 |
| Sertraline A-Ring Butane Reverse | 6.07 | 3.63 | 0.6 | NA | | 1 |
| Sulfonamide | | | | | | |
| Sertraline A-Ring Reverse | 3.67 | 4.32 | 1.18 | 33.08% | | 1 |
| Pentanamide | | | | | | |
| Sertraline A-Ring Methanamine | 1.59 | 3.7 | 2.32 | 85.93% | | 1 |
| Alcohol Sertraline-P1 | 24.47 | >50 | >2.04 | 89.20% | | 1 |
| Alcohol Sertraline-P2 | 54.8 | >50 | >0.9 | 43.45% | | 1 |
| Sertraline A-Ring Cyclopropane | 5.62 | 7.45 | 1.32 | 58.82% | | 1 |
| Reverse Sulfonamide | | | | | | |
| Sertraline A-Ring Benzene | 3.48 | 3.91 | 1.12 | NA | | 1 |
| Reverse Sulfonamide | | | | | | |
| Sertraline A-Ring Reverse | 2.94 | 3.67 | 1.25 | 62.17% | | 1 |
| Benzamide | | | | | | |
| Sertraline A-Ring N,N- | 3.52 | 6.22 | 1.76 | 35.11% | | 1 |
| Dimethylamine | | | | | | |
| Methoxy 4S-Sertraline-P1 | 22.67 | >50 | >2.2 | 93.98% | 46.15 | 1 |
| Methoxy 4S-Sertraline-P2 | 26.4 | 54.58 | 2.07 | 47.76% | | 1 |
| Sertraline A-Ring | 1.68 | 12.97 | 7.72 | 84.79% | | 1 |
| Sulfonylmethane (CH₂-Linker) | | | | | | |
| Sertraline A-Ring Reverse | 4.02 | 7 | 1.74 | 22.89% | | 1 |
| Cyclopentanecarboxamide | | | | | | |
| Sertraline A-Ring | 7.76 | 13.62 | 1.75 | 20.18% | | |
| Methylimidazole Reverse | | | | | | |
| Sulfonamide | | | | | | |
| Sertraline A-Ring Methylsulfide | 4.18 | 6.18 | 1.48 | 14.75% | | |
| No-N Sertraline | NA | NA | NA | NA | | |
| Isopropyl Sertraline | 9.25 ± 0.95 | >50 | >5.40 | 97.96 ± 0.06% | | |
| N,N-Dimethyl Sertraline A-Ring | 4.45 | 19.07 | 4.28 | 91.96% | | |
| Reverse Carboxamide (CH2- | | | | | | |
| Linker) | | | | | | |
| Sertraline A-ring N-methyl reverse | 3.79 | 14.6 | 3.86 | 91.70% | | |
| carboxamide | | | | | | |
| N,N-Dimethyl Sertraline A-Ring | 9.41 | 12.51 | 1.33 | NA | | |
| Reverse Benzamide | | | | | | |
| Sertraline A-ring Pyridine | 4.43 | 7.05 | 1.59 | 61.28% | | |
| Carboxamide | | | | | | |
| Sertraline A-Ring Benzamide | 2.73 | 3.89 | 1.42 | 48.62% | | |
| Sertraline A-Ring Cyclopropyl | 2.75 | 8.87 | 3.22 | 87.96% | | |
| Carboxamide | | | | | | |
| Sertraline A-Ring Methyl | 2.82 | 11.08 | 3.93 | 81.20% | | |
| Carboxamide | | | | | | |

### Example 5

### Characterization of UK-416244 and Analogs thereof

Characterization of analogs of UK-416244 having the formula:

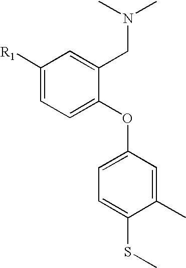

is shown in Table 25. UK-416244 is shown in bold.

TABLE 25

| R₁ | IC50 | TI | IC90 |
|---|---|---|---|
| SO2NH2 | 1.41 | >35 | 6.17 |
| SO₂NHMe | 5.8 | 7.8 | 14.76 |
| CN | 6.4 | >7.8 | 11.54 |
| H | 12.19 | 2.95 | |
| Br | 14.44 | 3.52 | |
| CONH₂ | 26.85 | >1.86 | |
| COOH | NA | NA | NA |

Characterization of analogs of UK-416244 having the formula:

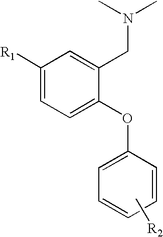

is shown in Table 26. UK-416244 is shown in bold.

TABLE 26

| R₁ | R₂ | IC50 | TI | IC90 |
|---|---|---|---|---|
| SO2NH2 | 3-Me, 4-SMe | 1.41 | >35 | 6.17 |
| H | 3-Me, 4-SMe | 12.19 | 2.95 | |
| Br | 3-Me, 4-SMe | 14.52 | 3.46 | |
| Br | 4-SMe | 25.81 | >1.94 | 28.75 |
| SO₂NH₂ | 4-SMe | 26.33 | >1.9 | |
| H | 4-Br | 29.86 | >1.67 | |
| Br | 4-OMe | 30.76 | >1.63 | |
| H | 4-CF₃ | NA | NA | |
| Br | 3-OMe | NA | NA | |
| H | 4-SMe | NA | NA | |

Characterization of analogs of UK-416244 having the formula:

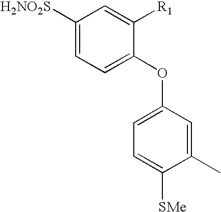

is shown in Table 27. UK-416244 is shown in bold.

TABLE 27

| R₁ | IC50 | TI | IC90 |
|---|---|---|---|
| CH2NMe2 | 1.41 | >35 | 6.17 |
| CH₂NHMe | 2.33 | 16.98 | 10 |
| CH₂OH | 18.14 | >2.76 | |
| CONHMe | NA | NA | |

Characterization of additional UK-416244 analogs is described in Table 28.

TABLE 28

| | | | | | | n |
|---|---|---|---|---|---|---|
| Name | IC₅₀ (uM) [SG] | CC₅₀ (uM) [SG] | TI | Max Effect@CC30 (inhibition) | IC90 | [SG] |
| UK-416244 | 1.41 ± 0.20 | >50 | >35 | 97.2 ± 1.4% | 6.17 ± 3.23 | 3 |
| Compound 6 | 5.8 | 45.3 | 7.8 | 93.20% | 14.76 | 1 |
| Compound 30 | 13.19 | 26.37 | 2 | 26% | | 1 |
| Compound 31 | 7.51 | 13.51 | 1.8 | 18% | | 1 |
| Compound 9 | 14.52 | 54.8 | 3.44 | 79.00% | | 1 |
| Compound 11 | NA | NA | NA | NA | | 1 |
| Compound 7 | 6.4 | >50 | >7.8 | 96.70% | 11.54 | 1 |
| Compound 10 | 26.85 | >50 | >1.86 | 80.70% | | 1 |
| Compound 8 | 12.19 | 35.97 | 2.95 | 52.30% | | 1 |
| Compound 55 | 29.86 | >50 | >1.67 | 77.20% | | 1 |
| Compound 32 | 31.17 | >50 | >1.60 | 30% | | 1 |
| Compound 33 | 30.17 | >50 | >1.65 | 72.30% | | 1 |
| Compound 34 | 24.57 | 35.51 | 1.44 | 72.60% | | 1 |
| Compound 35 | NA | NA | NA | 48.00% | | 1 |
| Compound 36 | NA | NA | NA | NA | | 1 |
| Compound 37 | 27.69 | 46.86 | 1.69 | 12.40% | | 1 |
| Compound 38 | NA | NA | NA | 42% | | 1 |
| Compound 39 | 37.5 | >50 | >1.3 | 91.70% | 50 | 1 |
| Compound 40 | NA | NA | NA | NA | | 1 |
| Compound 41 | 25.81 | >50 | >1.94 | 94.70% | 28.75 | 1 |
| Compound 42 | 30.76 | >50 | >1.63 | 51.70% | | 1 |
| Compound 43 | 32.69 | >50 | >1.53 | 73.70% | | 1 |
| Compound 44 | NA | NA | NA | 32.10% | | 1 |
| Compound 45 | NA | NA | NA | 10.33% | | 1 |
| Compound 46 | 26.3 | >50 | >1.90 | 83.22% | | 1 |
| Compound 47 | 18.14 | >50 | >2.76 | 63.55% | | 1 |
| Compound 48 | 2.33 | 39.57 | 16.98 | 95.49% | 10.02 | 1 |
| Compound 49 | 6.53 | 10.81 | 1.65 | 15.28% | | |
| Compound 5 | NA | NA | NA | NA | | |
| Compound 3 | NA | NA | NA | NA | | |
| Compound 14 | 11.43 | 16.9 | 1.48 | 59.00% | | |
| Compound 50 | 6.04 | 26.08 | 4.32 | 90.00% | 11.36 | |
| Compound 51 | NA | NA | NA | NA | | |
| Compound 52 | NA | NA | NA | NA | | |
| Compound 53 | 22.63 | >50 | >2.21 | 91.54% | 45.71 | |
| Compound 54 | 6.18 | >50 | >8.1 | 97.88% | 7.07 | |
| Compound 16 | NA | NA | NA | NA | | |

### Example 6

### Further Sertraline and UK-416244 Analog Characterization

Additional characterization of sertraline and UK-416244 analogs is provided in Tables 29-32 below.

TABLE 29

| | | | | | Max Effect |
|---|---|---|---|---|---|
| | | CC | Therapeutic | | @CC30 |
| Name | IC 50 (uM) | 50 (uM) | index | IC 90 (uM) | (inhibition) |
| Sertraline | 3.76 | 7.98 | 2.12 | 11.32 | 68.47% |
| Compound 45 | NA | >50 | NA | NA | NA |
| A-Ring Butane Reverse Sulfonamide | NA | 3.63 | NA | 6.14 | NA |
| Sertraline A-Ring Reverse Pentanamide | 3.68 | 4.32 | 1.17 | 5.03 | 33.08% |
| Sertraline A-Ring Methanamine | 1.60 | 3.70 | 2.32 | 3.28 | 85.93% |
| Alcohol Sertraline P1 | 24.47 | >50 | >2.04 | 54.12 | 87.44% |
| Compound 46 | 26.33 | >50 | >1.90 | 60.90 | 83.22% |
| N,N-Dimethyl Sertraline A-Ring Carboxamide | 2.96 | 20.71 | 6.99 | 8.73 | 94.78% |
| Alcohol Sertraline P2 | >50 | >50 | >1 | 78.57 | 43.45% |
| Sertraline A-Ring Reverse Carboxamide | 0.85 | 10.71 | 12.55 | 7.26 | 87.66 |
| Tocotrienol sample 1 | 22.66 | >50 | >2.21 | 39.49 | 98.00% |
| Tocotrienol sample 2 | 15.65 | 34.63 | 2.21 | 28.98 | 97.13% |
| Tocotrienol sample 3 | 17.05 | 48.31 | 2.83 | 26.87 | 90.32% |
| Tocotrienol sample 4 | 19.66 | 33.35 | 1.70 | 27.79 | 83.10% |

TABLE 30

| | | | | | Max |
|---|---|---|---|---|---|
| | IC 50 | CC 50 | Therapeutic | | Effect@CC30 |
| Name | (uM) | (uM) | index | IC 90 (uM) | (inhibition) |
| Sertraline | 3.46 | 7.70 | 2.22 | 9.65 | 75.12% |
| Sertraline A-Ring Cyclopropane Reverse | 5.63 | 7.45 | 1.32 | 10.65 | 56.82% |
| Sulfonamide | | | | | |
| Sertraline A-Ring Benzene Reverse | 3.48 | 3.91 | 1.13 | 3.83 | NA |
| Sulfonamide | | | | | |
| Sertraline A-Ring Reverse Benzamide | 2.94 | 3.67 | 1.25 | 3.88 | 62.17% |
| Sertraline A-Ring N,N-Dimethylamine | 3.52 | 6.22 | 1.77 | 5.82 | 35.11% |
| Methoxy 4S-Sertraline | 22.67 | >50 | >2.21 | 46.15 | 93.98% |
| Methoxy 4S-Sertraline | 26.40 | >50 | >1.89 | 46.9 | 47.76% |
| Sertraline A-Ring Sulfonylmethane (CH2- | 1.68 | 12.97 | 7.72 | 7.89 | 84.79% |
| Linker) | | | | | |
| Compound 48 | 2.33 | 39.57 | 16.98 | 10.02 | 95.49% |
| Compound 47 | 18.14 | >50 | >2.76 | NA | 63.55% |
| Sertraline A-Ring Reverse | 4.02 | 7.0 | 1.74 | 5.89 | 22.89% |
| Cyclopentanecarboxamide | | | | | |
| Sertraline A-ring Methyl Sulfoxide | 1.11 | 14.23 | 12.81 | 5.27 | 93.61% |

Additional characterization of sertraline analogs is provided in Table 31.

TABLE 31

| | | IC₅₀ | CC₅₀ | |
|---|---|---|---|---|
| | | (uM) | (uM) | |
| Name | Systematic Name | [SG] | [SG] | IC90 |
| Sertraline HCL | Sertraline HCl | 4.48 | 11.26 | NA |
| (1S,4S)-Desmethyl | (1S,4S)-4-(3,4-dichlorophenyl)-1,2,3,4- | 7.5 | 8.98 | NA |
| Sertraline | tetrahydronaphthalen-1-amine | | | |
| (1R,4R)-Desmethyl | (1R,4R)-4-(3,4-dichlorophenyl)-1,2,3,4- | 8.7 | 8.1 | NA |
| Sertraline | tetrahydronaphthalen-1-amine | | | |
| Sertraline | (5S,8S)-5-(3,4-dichlorophenyl)-N-methyl-8- | 6.87 | 13.04 | NA |
| Sulfonamide | (methylamino)-5,6,7,8-tetrahydronaphthalene-2- | | | |
| | sulfonamide | | | |
| Sertraline (Reverse) | N-((5S,8S)-5-(3,4-dichlorophenyl)-8-(methylamino)- | 3.99 | 12.89 | NA |
| Methanesulfonamide | 5,6,7,8-tetrahydronaphthalen-2- | | | |
| | yl)methanesulfonamide | | | |
| 1R,4R Sertraline | (1R,4R)-4-(3,4-dichlorophenyl)-N-methyl-1,2,3,4- | 10.9 | 14.58 | NA |
| Enantiomer | tetrahydronaphthalen-1-amine | | | |
| N,N-Dimethyl | (1S,4S)-4-(3,4-dichlorophenyl)-N,N-dimethyl- | 6.07 | 64.7 | 18.13 ± 2.77 |
| Sertraline | 1,2,3,4-tetrahydronaphthalen-1-amine | | | |
| Nitro Sertraline | (1S,4S)-4-(3,4-dichlorophenyl)-N-methyl-7-nitro- | 6.46 | 8.03 | NA |
| | 1,2,3,4-tetrahydronaphthalen-1-amine | | | |
| Sertraline Aniline | (1S,4S)-4-(3,4-dichlorophenyl)-N1-methyl-1,2,3,4- | 6 | 8.72 | NA |
| | tetrahydronaphthalene-1,7-diamine | | | |
| Sertraline Iodide | (1S,4S)-4-(3,4-dichlorophenyl)-7-iodo-N-methyl- | 6.57 | 8.92 | NA |
| | 1,2,3,4-tetrahydronaphthalen-1-amine | | | |
| Sertraline | (5S,8S)-5-(3,4-dichlorophenyl)-8-(methylamino)- | 3.65 | 9.99 | NA |
| Sulfonamide NH2 | 5,6,7,8-tetrahydronaphthalene-2-sulfonamide | | | |
| Sertraline | (5S,8S)-5-(3,4-dichlorophenyl)-N-(2-hydroxyethyl)- | 8.13 | 13.75 | NA |
| Sulfonamide | 8-(methylamino)-5,6,7,8-tetrahydronaphthalene-2- | | | |
| Ethanol | sulfonamide | | | |
| Sertraline Nitrile | (5S,8S)-5-(3,4-dichlorophenyl)-8-(methylamino)- | 4.81 | 14.82 | NA |
| | 5,6,7,8-tetrahydronaphthalene-2-carbonitrile | | | |
| Sertraline-CME | 2-(((1S,4S)-4-(3,4-dichlorophenyl)-1,2,3,4- | NA | NA | NA |
| | tetrahydronaphthalen-1-yl)(methyl)amino)acetic acid | | | |
| Dimethyl Sertraline | N-((5S,8S)-5-(3,4-dichlorophenyl)-8- | 3.45 | 16.46 | NA |
| Reverse | (dimethylamino)-5,6,7,8-tetrahydronaphthalen-2- | | | |
| Sulfonamide | yl)methanesulfonamide | | | |
| Sertraline Reverse | N-(((5S,8S)-5-(3,4-dichlorophenyl)-8- | 3.28 | 11.24 | NA |
| Sulfonamide (CH2 | (methylamino)-5,6,7,8-tetrahydronaphthalen-2- | | | |
| linker) | yl)methyl)methanesulfonamide | | | |
| Sertraline B-ring | 4-(2-methoxyphenyl)-N-methyl-1,2,3,4- | NA | >50 | NA |
| Ortho Methoxy | tetrahydronaphthalen-1-amine | | | |
| Sertraline A-ring | (5S,8S)-methyl 5-(3,4-dichlorophenyl)-8- | 9.13 | 13.69 | NA |
| Methyl Ester | (methylamino)-5,6,7,8-tetrahydronaphthalene-2- | | | |
| | carboxylate | | | |
| Sertraline A-Ring | ((5S,8S)-5-(3,4-dichlorophenyl)-8-(methylamino)- | 2.85 | 14.18 | 1132% |
| Ethanol | 5,6,7,8-tetrahydronaphthalen-2-yl)methanol | | | |
| Sertraline N,N- | (5S,8S)-5-(3,4-dichlorophenyl)-N,N-dimethyl-8- | 6.69 | 8.87 | NA |
| dimethylsulfonamide | (methylamino)-5,6,7,8-tetrahydronaphthalene-2- | | | |
| | sulfonamide | | | |
| Sertraline A ring | (5S,8S)-5-(3,4-dichlorophenyl)-8-(methylamino)- | 1.8 | 16.22 | 540% |
| carboxamide | 5,6,7,8-tetrahydronaphthalene-2-carboxamide | | | |
| Sertraline B-ring | N-methyl-4-(4-phenoxyphenyl)-1,2,3,4- | 4.02 | 7.85 | NA |
| para-Phenoxy | tetrahydronaphthalen-1-amine | | | |
| Sertraline B-Ring | N-methyl-4-(4-(trifluoromethyl)phenyl)-1,2,3,4- | 13.38 | 14.18 | NA |
| para- | tetrahydronaphthalen-1-amine | | | |
| Trifluoromethane | | | | |
| N,N-Dimethyl | N,N-dimethyl-4-(4-(trifluoromethyl)phenyl)-1,2,3,4- | NA | NA | NA |
| Sertraline B-Ring | tetrahydronaphthalen-1-amine | | | |
| Para- | | | | |
| Trifluoromethane | | | | |
| Sertraline B ring 2- | N-methyl-4-(thiophen-2-yl)-1,2,3,4- | NA | NA | NA |
| Thiophene | tetrahydronaphthalen-1-amine | | | |
| Sertraline without B- | N-methyl-1,2,3,4-tetrahydronaphthalen-1-amine | NA | NA | NA |
| Ring | | | | |
| N-Ethanol Sertraline | 2-(((1S,4S)-4-(3,4-dichlorophenyl)-1,2,3,4- | 7.8 | 49.8 | 21.92 |
| | tetrahydronaphthalen-1-yl)(methyl)amino)ethanol | | | |
| N-Cyclopropyl | (1S,4S)—N-cyclopropyl-4-(3,4-dichlorophenyl)-N- | 14.5 | >50 | 34.2 |
| Sertraline | methyl-1,2,3,4-tetrahydronaphthalen-1-amine | | | |
| (1S,4R) Sertraline | (1S,4R) Sertraline Hydrochloride | 5.96 | 7.51 | NA |
| Hydrochloride | | | | |
| (1R,4S) Sertraline | (1R,4S) Sertraline Hydrochloride | 8.02 | 14.2 | 12.09 |
| Hydrochloride | | | | |
| Sertraline B-Ring | 4-(4-methoxyphenyl)-N-methyl-1,2,3,4- | 26.8 | 51.1 | NA |
| Para-Methoxy | tetrahydronaphthalen-1-amine | | | |
| Sertraline A ring | 4-(3,4-dichlorophenyl)-N-methyl-4,5,6,7- | 8.27 | 18.84 | NA |
| Thiophene | tetrahydrobenzo[b]thiophen-7-amine | | | |
| Sertraline A-ring | (1S,4S)-4-(3,4-dichlorophenyl)-N-methyl-7- | 1.55 | 15.35 | 5.15 |
| Methyl Sulfoxide | (methylsulfinylmethyl)-1,2,3,4- | | | |
| (CH2 Linker) | tetrahydronaphthalen-1-amine | | | |
| N,N-dimethyl | (5S,8S)-5-(3,4-dichlorophenyl)-8-(dimethylamino)- | NA | NA | NA |
| Sertraline A ring | 5,6,7,8-tetrahydronaphthalene-2-carboxylic acid | | | |
| Carboxylic acid | | | | |
| Sertraline B ring m- | 4-(3-methoxyphenyl)-N-methyl-1,2,3,4- | 28.77 | >50 | NA |
| Methoxy | tetrahydronaphthalen-1-amine | | | |
| N,N-Dimethyl | (5S,8S)-5-(3,4-dichlorophenyl)-8-(dimethylamino)- | 3.18 | 23.72 | 8.77 |
| Sertraline A-Ring | 5,6,7,8-tetrahydronaphthalene-2-carboxamide | | | |
| Carboxamide | | | | |
| Sertraline A-Ring | N-((5S,8S)-5-(3,4-dichlorophenyl)-8-(methylamino)- | 0.91 | 11.96 | NA |
| Reverse | 5,6,7,8-tetrahydronaphthalen-2-yl)acetamide | | | |
| Carboxamide | | | | |
| 4S-Sertraline Ketone | (S)-4-(3,4-dichlorophenyl)-3,4-dihydronaphthalen- | 32.57 | >50 | NA |
| | 1(2H)-one | | | |
| Sertraline A-Ring | N-((5S,8S)-5-(3,4-dichlorophenyl)-8-(methylamino)- | 6.07 | 3.63 | NA |
| Butane Reverse | 5,6,7,8-tetrahydronaphthalen-2-yl)butane-1- | | | |
| Sulfonamide | sulfonamide | | | |
| Sertraline A-Ring | N-((5S,8S)-5-(3,4-dichlorophenyl)-8-(methylamino)- | 3.67 | 4.32 | NA |
| Reverse | 5,6,7,8-tetrahydronaphthalen-2-yl)pentanamide | | | |
| Pentanamide | | | | |
| Sertraline A-Ring | (1S,4S)-7-(aminomethyl)-4-(3,4-dichlorophenyl)-N- | 1.67 | 3.68 | NA |
| Methanamine | methyl-1,2,3,4-tetrahydronaphthalen-1-amine | | | |
| Alcohol Sertraline- | (S)-4-(3,4-dichlorophenyl)-1,2,3,4- | 24.47 | >50 | NA |
| P1 | tetrahydronaphthalen-1-ol | | | |
| Sertraline A-Ring | N-((5S,8S)-5-(3,4-dichlorophenyl)-8-(methylamino)- | 5.62 | 7.45 | NA |
| Cyclopropane | 5,6,7,8-tetrahydronaphthalen-2- | | | |
| Reverse | yl)cyclopropanesulfonamide | | | |
| Sulfonamide | | | | |
| Sertraline A-Ring | N-((5S,8S)-5-(3,4-dichlorophenyl)-8-(methylamino)- | 3.48 | 3.91 | NA |
| Benzene Reverse | 5,6,7,8-tetrahydronaphthalen-2- | | | |
| Sulfonamide | yl)benzenesulfonamide | | | |
| Sertraline A-Ring | N-((5S,8S)-5-(3,4-dichlorophenyl)-8-(methylamino)- | 2.94 | 3.67 | NA |
| Reverse Benzamide | 5,6,7,8-tetrahydronaphthalen-2-yl)benzamide | | | |
| Sertraline A-Ring | (1S,4S)-4-(3,4-dichlorophenyl)-N1,N7,N7-trimethyl- | 3.52 | 6.22 | NA |
| N,N-Dimethylamine | 1,2,3,4-tetrahydronaphthalene-1,7-diamine | | | |
| Methoxy 4S- | (S)-1-(3,4-dichlorophenyl)-4-methoxy-1,2,3,4- | 22.67 | >50 | 46.15 |
| Sertraline-P1 | tetrahydronaphthalene | | | |
| Sertraline A-Ring | (1S,4S)-4-(3,4-dichlorophenyl)-N-methyl-7- | 2.33 | 14.22 | NA |
| Sulfonylmethane | (methylsulfonylmethyl)-1,2,3,4- | | | |
| (CH2-Linker) | tetrahydronaphthalen-1-amine | | | |
| Sertraline A-Ring | N-((5S,8S)-5-(3,4-dichlorophenyl)-8-(methylamino)- | 4.02 | 7 | NA |
| Reverse | 5,6,7,8-tetrahydronaphthalen-2- | | | |
| Cyclopentanecarboxamide | yl)cyclopentanecarboxamide | | | |
| Sertraline A-Ring | N-((5S,8S)-5-(3,4-dichlorophenyl)-8-(methylamino)- | 7.76 | 13.62 | NA |
| Methylimidazole | 5,6,7,8-tetrahydronaphthalen-2-yl)-1-methyl-1H- | | | |
| Reverse | imidazole-4-sulfonamide | | | |
| Sulfonamide | | | | |
| Sertraline A-Ring | (1S,4S)-4-(3,4-dichlorophenyl)-N-methyl-7- | 4.18 | 6.18 | NA |
| Methylsulfide | (methylthiomethyl)-1,2,3,4-tetrahydronaphthalen-1- | | | |
| | amine | | | |
| No-N Sertraline | 1-(3,4-dichlorophenyl)-1,2,3,4- | NA | NA | NA |
| | tetrahydronaphthalene | | | |
| Isopropyl Sertraline | 1-(3,4-dichlorophenyl)-4-isopropyl-1,2,3,4- | 9.25 | >50 | 32.13 |
| | tetrahydronaphthalene | | | |
| N,N-Dimethyl | N-(((5S,8S)-5-(3,4-dichlorophenyl)-8- | 4.45 | 19.07 | 9.72 |
| Sertraline A-Ring | (dimethylamino)-5,6,7,8-tetrahydronaphthalen-2- | | | |
| Reverse | yl)methyl)acetamide | | | |
| Carboxamide (CH2- | | | | |
| Linker) | | | | |
| Sertraline A ring N- | N-((5S,8S)-5-(3,4-dichlorophenyl)-8-(methylamino)- | 3.79 | 14.6 | 10.7 |
| methyl reverse | 5,6,7,8-tetrahydronaphthalen-2-yl)-N- | | | |
| carboxamide | methylacetamide | | | |
| N,N-Dimethyl | N-((5S,8S)-5-(3,4-dichlorophenyl)-8- | 9.41 | 12.51 | NA |
| Sertraline A-Ring | (dimethylamino)-5,6,7,8-tetrahydronaphthalen-2- | | | |
| Reverse Benzamide | yl)benzamide | | | |
| Sertraline A-ring | (5S,8S)-5-(3,4-dichlorophenyl)-8-(methylamino)-N- | 4.43 | 7.05 | NA |
| Pyridine | (pyridin-2-yl)-5,6,7,8-tetrahydronaphthalene-2- | | | |
| Carboxamide | carboxamide | | | |
| Sertraline A-Ring | (5S,8S)-5-(3,4-dichlorophenyl)-8-(methylamino)-N- | 2.73 | 3.89 | NA |
| Benzamide | phenyl-5,6,7,8-tetrahydronaphthalene-2-carboxamide | | | |
| Sertraline A-Ring | (5S,8S)—N-cyclopropyl-5-(3,4-dichlorophenyl)-8- | 2.75 | 8.87 | NA |
| Cyclopropyl | (methylamino)-5,6,7,8-tetrahydronaphthalene-2- | | | |
| Carboxamide | carboxamide | | | |
| Sertraline A-Ring | (5S,8S)-5-(3,4-dichlorophenyl)-N-methyl-8- | 2.82 | 11.08 | NA |
| Methyl | (methylamino)-5,6,7,8-tetrahydronaphthalene-2- | | | |
| Carboxamide | carboxamide | | | |
| Sertraline A-Ring | methyl 2-((1S,4S)-1-(3,4-dichlorophenyl)-4- | 6.69 | 22.9 | NA |
| Methyl Acetate | (methylamino)-1,2,3,4-tetrahydronaphthalene-6- | | | |
| Carboxamide | carboxamido)acetate | | | |
| Sertraline A-Ring | 2-((1S,4S)-1-(3,4-dichlorophenyl)-4-(methylamino)- | NA | NA | NA |
| Acetic Acid | 1,2,3,4-tetrahydronaphthalene-6-carboxamido)acetic | | | |
| Carboxamide | acid | | | |
| Sertraline A-Ring | (5S,8S)-5-(3,4-dichlorophenyl)-8-(methylamino)- | 7.19 | 13.25 | NA |
| Thiocarboxamide | 5,6,7,8-tetrahydronaphthalene-2-carbothioamide | | | |
| Sertraline A-Ring | (1S,4S)-4-(3,4-dichlorophenyl)-N-methyl-7- | 6.24 | 6.7 | NA |
| Phenyl Sulfone | (phenylsulfonylmethyl)-1,2,3,4- | | | |
| (CH2-Linker) | tetrahydronaphthalen-1-amine | | | |
| N,N-Dimethyl | (5S,8S)—N-cyclopropyl-5-(3,4-dichlorophenyl)-8- | 12.95 | 26.31 | NA |
| Sertraline A-Ring N- | (dimethylamino)-N-methyl-5,6,7,8- | | | |
| Methyl | tetrahydronaphthalene-2-carboxamide | | | |
| Cyclopropane | | | | |
| Carboxamide | | | | |
| N-Ethanol Sertraline | N-((5S,8S)-5-(3,4-dichlorophenyl)-8-((2- | 13.16 | 44.89 | NA |
| A-Ring Reverse | hydroxyethyl)(methyl)amino)-5,6,7,8- | | | |
| Carboxamide | tetrahydronaphthalen-2-yl)acetamide | | | |
| N-Ethanol Sertraline | 2-(((1S,4S)-4-(3,4-dichlorophenyl)-7- | 24.2 | >50 | NA |
| A-Ring Methyl | (methylsulfinylmethyl)-1,2,3,4- | | | |
| Sulfoxide (CH2- | tetrahydronaphthalen-1-yl)(methyl)amino)ethanol | | | |
| Linker) | | | | |
| N,N-Dimethyl | (5S,8S)—N-cyclopropyl-5-(3,4-dichlorophenyl)-8- | 7.3 | 25.41 | NA |
| Sertraline A-Ring | (dimethylamino)-5,6,7,8-tetrahydronaphthalene-2- | | | |
| Cyclopropane | carboxamide | | | |
| Carboxamide | | | | |
| Isopropyl Sertraline | N-(5-(3,4-dichlorophenyl)-8-isopropyl-5,6,7,8- | 15.81 | >50 | 37.74 |
| A-Ring Reverse | tetrahydronaphthalen-2-yl)acetamide | | | |
| Carboxamide | | | | |
| N,N-Dimethyl | (5S,8S)—N-cyclobutyl-5-(3,4-dichlorophenyl)-8- | 5.78 | 13.39 | NA |
| Sertraline A-Ring | (dimethylamino)-5,6,7,8-tetrahydronaphthalene-2- | | | |
| Cyclobutyl | carboxamide | | | |
| Carboxamide | | | | |
| Isopropyl alkene | (S)-5-(3,4-dichlorophenyl)-8-isopropyl-5,6- | 21.49 | >50 | NA |
| Sertraline A-Ring | dihydronaphthalene-2-carboxamide | | | |
| Carboxamide | | | | |
| Isopropyl Sertraline | (S)-5-(3,4-dichlorophenyl)-8-isopropyl-5,6,7,8- | 38.93 | >50 | NA |
| A-Ring | tetrahydronaphthalene-2-carboxamide | | | |
| Carboxamide | | | | |
| N,N-Dimethyl | (5S,8S)—N-(cyclopropylmethyl)-5-(3,4- | 5.61 | 19.42 | 12.25 |
| Sertraline A-Ring | dichlorophenyl)-8-(dimethylamino)-5,6,7,8- | | | |
| Cyclopropylmethyl | tetrahydronaphthalene-2-carboxamide | | | |
| Carboxamide | | | | |
| N,N-Dimethyl | (5S,8S)—N-cyclopropyl-5-(3,4-dichlorophenyl)-8- | 8.63 | 18.17 | NA |
| Sertraline A-Ring | (dimethylamino)-5,6,7,8-tetrahydronaphthalene-2- | | | |
| Reverse | carboxamide | | | |
| Cyclopropane | | | | |
| Carboxamide | | | | |

Additional characterization of UK-416244 analogs is provided in Table 32.

TABLE 32

| | | IC₅₀ | CC₅₀ | |
|---|---|---|---|---|
| Compound | | (uM) | (uM) | |
| Name | Systematic Name | [SG] | [SG] | IC90 |
| UK-416244 | 3-((dimethylamino)methyl)-4-(3-methyl-4- | 1.74 | >50 | 6.55 |
| | (methylthio)phenoxy)benzenesulfonamide | | | |
| Compound 1 | 3-((2-hydroxyethylamino)methyl)-4-(3-methyl-4- | 4.31 | >50 | 14.96 |
| | (methylthio)phenoxy)benzenesulfonamide | | | |
| Compound 2 | 3-(((2-hydroxyethyl)(methyl)amino)methyl)-4-(3- | 5.58 | >50 | 20.08 |
| | methyl-4-(methylthio)phenoxy)benzenesulfonamide | | | |
| Compound 3 | 3-((dimethylamino)methyl)-4-(m- | NA | NA | NA |
| | tolyloxy)benzenesulfonamide | | | |
| Compound 5 | 3-((dimethylamino)methyl)-4-(3-methyl-4- | NA | NA | NA |
| | (methylsulfinyl)phenoxy)benzenesulfonamide | | | |
| Compound 6 | 3-((dimethylamino)methyl)-N-methyl-4-(3-methyl- | 5.8 | 45.3 | 14.76 |
| | 4-(methylthio)phenoxy)benzenesulfonamide | | | |
| Compound 7 | 3-((dimethylamino)methyl)-4-(3-methyl-4- | 6.4 | >50 | 11.54 |
| | (methylthio)phenoxy)benzonitrile | | | |
| Compound 8 | N,N-dimethyl-1-(2-(3-methyl-4- | 12.19 | 35.97 | NA |
| | (methylthio)phenoxy)phenyl)methanamine | | | |
| Compound 9 | (5-bromo-2-(3-methyl-4- | 14.52 | >50 | NA |
| | (methylthio)phenoxy)phenyl)-N,N- | | | |
| | dimethylmethanamine | | | |
| Compound 10 | 3-((dimethylamino)methyl)-4-(3-methyl-4- | 26.85 | >50 | NA |
| | (methylthio)phenoxy)benzamide | | | |
| Compound 11 | 3-((dimethylamino)methyl)-4-(3-methyl-4- | NA | NA | NA |
| | (methylthio)phenoxy)benzoic acid | | | |
| Compound 12 | N-butyl-3-((dimethylamino)methyl)-4-(3-methyl-4- | 13.43 | 18.41 | NA |
| | (methylthio)phenoxy)benzenesulfonamide | | | |
| Compound 13 | N-cyclopropyl-3-((dimethylamino)methyl)-4-(3- | 25.52 | 41.8 | NA |
| | methyl-4-(methylthio)phenoxy)benzenesulfonamide | | | |
| Compound 14 | N-benzyl-3-((dimethylamino)methyl)-4-(3-methyl- | 11.43 | 16.9 | NA |
| | 4-(methylthio)phenoxy)benzenesulfonamide | | | |
| Compound 15 | 3-((dimethylamino)methyl)-N,N-dimethyl-4-(3- | 23.01 | 38.7 | NA |
| | methyl-4-(methylthio)phenoxy)aniline | | | |
| Compound 16 | N-(3-((dimethylamino)methyl)-4-(3-methyl-4- | NA | NA | NA |
| | (methylthio)phenoxy)phenylsulfonyl)acetamide | | | |
| Compound 17 | N-(3-((dimethylamino)methyl)-4-(3-methyl-4- | 22.73 | >50 | NA |
| | (methylthio)phenoxy)benzyl)methanesulfonamide | | | |
| Compound 24 | 3-((dimethylamino)methyl)-4-(3-methyl-4- | NA | NA | NA |
| | (methylthio)phenoxy)-N-(prop-2- | | | |
| | ynyl)benzenesulfonamide | | | |
| Compound 25 | 3-((dimethylamino)methyl)-N-methyl-4-(3-methyl- | 20.02 | 46.94 | NA |
| | 4-(methylthio)phenoxy)aniline | | | |
| Compound 26 | 2-(3-methyl-4-(methylthio)phenoxy)-5- | NA | NA | NA |
| | sulfamoylbenzoic acid | | | |
| Compound 28 | 3-((dimethylamino)methyl)-4-(3-methoxy-4- | 10.05 | >50 | 33.59 |
| | (methylthio)phenoxy)benzenesulfonamide | | | |
| Compound 28 | 3-isobutyl-4-(3-methyl-4- | 37.07 | >50 | NA |
| | (methylthio)phenoxy)benzenesulfonamide | | | |
| Compound 30 | N-methyl-1-(2-(3-methyl-4- | 13.19 | 26.37 | NA |
| | (methylthio)phenoxy)phenyl)methanamine | | | |
| Compound 31 | (5-bromo-2-(3-methyl-4- | 7.51 | 13.51 | NA |
| | (methylthio)phenoxy)phenyl)-N- | | | |
| | methylmethanamine | | | |
| Compound 33 | N-methyl(2-(4- | 30.17 | >50 | NA |
| | (methylthio)phenoxy)phenyl)methanamine | | | |
| Compound 34 | (5-bromo-2-(4-(methylthio)phenoxy)phenyl)-N- | 24.57 | 35.51 | NA |
| | methylmethanamine | | | |
| Compound 35 | N,N-dimethyl(2-(4- | NA | NA | NA |
| | (methylthio)phenoxy)phenyl)methanamine | | | |
| Compound 36 | (5-bromo-2-(4-methoxyphenoxy)phenyl)-N- | NA | NA | NA |
| | methylmethanamine | | | |
| Compound 37 | (5-bromo-2-(3-methoxyphenoxy)phenyl)-N- | 27.69 | 46.86 | NA |
| | methylmethanamine | | | |
| Compound 38 | (5-bromo-2-(3-methoxyphenoxy)phenyl)-N,N- | NA | NA | NA |
| | dimethylmethanamine | | | |
| Compound 39 | N-methyl-1-(2-(4- | 37.5 | >50 | 50 |
| | (trifluoromethyl)phenoxy)phenyl)methanamine | | | |
| Compound 40 | N,N-dimethyl(2-(4- | NA | NA | NA |
| | (trifluoromethyl)phenoxy)phenyl)methanamine | | | |
| Compound 41 | 1-(5-bromo-2-(4-(methylthio)phenoxy)phenyl)- | 25.81 | >50 | 28.75 |
| | N,N-dimethylmethanamine | | | |
| Compound 42 | (5-bromo-2-(4-methoxyphenoxy)phenyl)-N,N- | 30.76 | >50 | NA |
| | dimethylmethanamine | | | |
| Compound 43 | 3-((methylamino)methyl)-4-(4- | 32.69 | >50 | NA |
| | (methylthio)phenoxy)benzenesulfonamide | | | |
| Compound 44 | 4-(4-bromophenoxy)-3- | NA | NA | NA |
| | ((methylamino)methyl)benzenesulfonamide | | | |
| Compound 45 | N-methyl-2-(3-methyl-4-(methylthio)phenoxy)-5- | NA | NA | NA |
| | sulfamoylbenzamide | | | |
| Compound 46 | 3-((dimethylamino)methyl)-4-(4- | 26.3 | >50 | NA |
| | (methylthio)phenoxy)benzenesulfonamide | | | |
| Compound 47 | 3-(hydroxymethyl)-4-(3-methyl-4- | 18.14 | >50 | NA |
| | (methylthio)phenoxy)benzenesulfonamide | | | |
| Compound 48 | 4-(3-methyl-4-(methylthio)phenoxy)-3- | 2.26 ± 0.11 | >50 | 8.26 ± 2.49 |
| | ((methylamino)methyl)benzenesulfonamide | | | |
| Compound 49 | 3-((dimethylamino)methyl)-4-(3-methyl-4- | 6.53 | 10.81 | NA |
| | (methylthio)phenoxy)-N- | | | |
| | phenylbenzenesulfonamide | | | |
| Compound 50 | N-methyl-1-(2-(3-methyl-4-(methylthio)phenoxy)- | 6.04 | 26.08 | 11.36 |
| | 5-nitrophenyl)methanamine | | | |
| Compound 51 | N-(3-((dimethylamino)methyl)-4-(3-methyl-4- | NA | NA | NA |
| | (methylthio)phenoxy)phenyl)methanesulfonamide | | | |
| Compound 52 | (2-(4-bromophenoxy)phenyl)-N- | 31.17 | >50 | NA |
| | methylmethanamine | | | |
| Compound 52 | N-(2-(3-methyl-4-(methylthio)phenoxy)-5- | NA | NA | NA |
| | sulfamoylbenzyl)acetamide | | | |
| Compound 53 | 3-((dimethylamino)methyl)-4-(3-methyl-4- | 22.63 | >50 | 45.71 |
| | (methylthio)phenoxy)aniline | | | |
| Compound 54 | N,N-dimethyl-1-(2-(3-methyl-4- | 6.18 | >50 | 7.07 |
| | (methylthio)phenoxy)-5-nitrophenyl)methanamine | | | |
| Compound 55 | (2-(4-bromophenoxy)phenyl)-N,N- | 29.86 | >50 | NA |
| | dimethylmethanamine | | | |
| Compound 57 | N-(4-bromophenyl)-3-((dimethylamino)methyl)-4- | 10.95 | 8.17 | NA |
| | (3-methyl-4- | | | |
| | (methylthio)phenoxy)benzenesulfonamide | | | |
| Compound 58 | 3-amino-4-(3-methyl-4- | 15.31 | >50 | NA |
| | (methylthio)phenoxy)benzenesulfonamide | | | |
| Compound 59 | 3-((dimethylamino)methyl)-N-(4-methoxyphenyl)- | 6.39 | 8.32 | NA |
| | 4-(3-methyl-4- | | | |
| | (methylthio)phenoxy)benzenesulfonamide | | | |
| Compound 60 | 1-(4-bromo-2-(3-methyl-4- | 12.62 | 28.71 | NA |
| | (methylthio)phenoxy)phenyl)-N,N- | | | |
| | dimethylmethanamine | | | |
| Compound 61 | 4-(4-bromo-3-methylphenoxy)-3- | 12.12 | >50 | NA |
| | ((dimethylamino)methyl)benzenesulfonamide | | | |
| Compound 62 | 4-(3-methyl-4-(methylthio)phenoxy)-3- | NA | >50 | NA |
| | (methylamino)benzenesulfonamide | | | |
| Compound 63 | 4-(3-methyl-4-(methylthio)phenoxy)-3-((4- | 4.82 | >50 | 10.49 |
| | methylpiperazine-1-yl)methyl)benzenesulfonamide | | | |
| Compound 64 | 4-((dimethylamino)methyl)-3-(3-methyl-4- | 22.51 | >50 | NA |
| | (methylthio)phenoxy)benzonitrile | | | |
| Compound 65 | 3-isobutyl-4-(m-tolyloxy)benzenesulfonamide | 30.67 | >50 | 50 |
| Compound 66 | (S)-3-((3-hydroxypyrrolidin-1-yl)methyl)-4-(3- | 2.43 | >50 | 6.78 |
| | methyl-4-(methylthio)phenoxy)benzenesulfonamide | | | |
| Compound 67 | (R)-3-((3-hydroxypyrrolidin-1-yl)methyl)-4-(3- | 2.26 | >50 | 7.4 |
| | methyl-4-(methylthio)phenoxy)benzenesulfonamide | | | |
| Compound 68 | (R)-3-((2-hydroxypropylamino)methyl)-4-(3- | 4.34 | >50 | 15.28 |
| | methyl-4-(methylthio)phenoxy)benzenesulfonamide | | | |
| Compound 69 | (S)-3-((2-hydroxypropylamino)methyl)-4-(3- | 3.54 | >50 | 10.44 |
| | methyl-4-(methylthio)phenoxy)benzenesulfonamide | | | |
| Compound 70 | (R)-3-(((2-hydroxypropyl)(methyl)amino)methyl)- | 7.01 | >50 | 23.95 |
| | 4-(3-methyl-4- | | | |
| | (methylthio)phenoxy)benzenesulfonamide | | | |
| Compound 71 | (S)-3-(((2-hydroxypropyl)(methyl)amino)methyl)- | 4.96 | >50 | 13.39 |
| | 4-(3-methyl-4- | | | |
| | (methylthio)phenoxy)benzenesulfonamide | | | |
| Compound 72 | 2-(5-bromo-2-((dimethylamino)methyl)phenoxy)-4- | 16.33 | 44.99 | NA |
| | methyl-5-(methylthio)benzenesulfonamide | | | |
| Compound 73 | 4-(3-methyl-4-(methylthio)phenoxy)-3-(piperidin- | 4.7 | 37.91 | 13.6 |
| | 1-ylmethyl)benzenesulfonamide | | | |
| Compound 74 | 3-((4-methyl-1,4-diazepan-1-yl)methyl)-4-(3- | 7.31 | 35.92 | 16.1 |
| | methyl-4-(methylthio)phenoxy)benzenesulfonamide | | | |
| Compound 75 | 4-(3-methoxy-4-(methylthio)phenoxy)-3- | 11.94 | >50 | NA |
| | ((methylamino)methyl)benzenesulfonamide | | | |
| Compound 76 | 4-(3-methyl-4-nitrophenoxy)-3- | 30.96 | >50 | NA |
| | ((methylamino)methyl)benzenesulfonamide | | | |
| Compound 77 | 3-((dimethylamino)methyl)-4-(naphthalen-1- | NA | NA | NA |
| | yloxy)benzenesulfonamide | | | |
| Compound 78 | 6-bromo-3-methyl-1-(3-methyl-4- | 13.73 | 24.94 | 15.48 |
| | (methylthio)phenyl)-1,2,3,4-tetrahydroquinazoline | | | |
| Compound 79 | 3-((3-(dimethylamino)propylamino)methyl)-4-(3- | 3.44 | 17.75 | 9.66 |
| | methyl-4-(methylthio)phenoxy)benzenesulfonamide | | | |
| Compound 80 | 3-((methylamino)methyl)-4-(naphthalen-1- | NA | NA | NA |
| | yloxy)benzenesulfonamide | | | |
| Compound 81 | 4-(4-fluoro-3-methylphenoxy)-3- | NA | NA | NA |
| | ((methylamino)methyl)benzenesulfonamide | | | |
| Compound 82 | 4-(3-methyl-4-(methylthio)phenoxy)-3- | 20.75 | >50 | NA |
| | (morpholinomethyl)benzenesulfonamide | | | |
| Compound 83 | 3-cyano-4-(3-methyl-4- | NA | NA | NA |
| | (methylthio)phenoxy)benzenesulfonamide | | | |
| Compound 84 | 4-(3,4-dimethylphenoxy)-3- | 28.13 | 46.67 | 39.61 |
| | ((methylamino)methyl)benzenesulfonamide | | | |
| Compound 85 | (S)-3-((3-fluoropyrrolidin-1-yl)methyl)-4-(3- | 7.22 | >50 | NA |
| | methyl-4-(methylthio)phenoxy)benzenesulfonamide | | | |
| Compound 86 | 4-(3-methyl-4-(methylthio)phenoxy)-3-(2- | 34.76 | >50 | NA |
| | methylprop-1-enyl)benzenesulfonamide | | | |
| Compound 87 | 3-((4-(2-fluorophenyl)piperazin-1-yl)methyl)-4-(3- | 23.47 | 30.21 | NA |
| | methyl-4-(methylthio)phenoxy)benzenesulfonamide | | | |
| Compound 88 | (S)-3-((3-(dimethylamino)pyrrolidin-1-yl)methyl)- | 4.15 | 39.37 | 13.19 |
| | 4-(3-methyl-4- | | | |
| | (methylthio)phenoxy)benzenesulfonamide | | | |
| Compound 89 | 4-(3-methyl-4-(methylthio)phenoxy)-3-((4- | 6.62 | >50 | 21.26 |
| | morpholinopiperidin-1- | | | |
| | yl)methyl)benzenesulfonamide | | | |
| Compound 90 | 4-(3-methyl-4-(methylthio)phenoxy)-3-((4-(1- | 2.03 | 9.1 | 7.48 |
| | methylpiperidin-4-yl)piperazin-1- | | | |
| | yl)methyl)benzenesulfonamide | | | |
| Compound 91 | 4-(3-methyl-4-(methylthio)phenoxy)-3-((4- | 4.91 | 9.86 | NA |
| | (pyridin-4-yl)piperazin-1- | | | |
| | yl)methyl)benzenesulfonamide | | | |
| Compound 92 | 3-((4-(4-chlorophenyl)piperazin-1-yl)methyl)-4-(3- | 30.01 | 44.42 | NA |
| | methyl-4-(methylthio)phenoxy)benzenesulfonamide | | | |
| Compound 93 | 4-(3-methyl-4-(methylthio)phenoxy)-3-((4- | 20.15 | 43.12 | NA |
| | phenylpiperazin-1-yl)methyl)benzenesulfonamide | | | |
| Compound 94 | 3-(((3- | 3.26 | 14.98 | 7.38 |
| | (dimethylamino)propyl)(methyl)amino)methyl)-4- | | | |
| | (3-methyl-4- | | | |
| | (methylthio)phenoxy)benzenesulfonamide | | | |
| Compound 95 | 3-((4-(2-hydroxyethyl)piperazin-1-yl)methyl)-4-(3- | 6.5 | >50 | 16.96 |
| | methyl-4-(methylthio)phenoxy)benzenesulfonamide | | | |
| Compound 96 | 3-((2-(dimethylamino)ethylamino)methyl)-4-(3- | 2.77 | 19.39 | 5.89 |
| | methyl-4-(methylthio)phenoxy)benzenesulfonamide | | | |
| Compound 97 | 3-(((2- | 12.42 | 43.28 | 14 |
| | (dimethylamino)ethyl)(methyl)amino)methyl)-4-(3- | | | |
| | methyl-4-(methylthio)phenoxy)benzenesulfonamide | | | |
| Compound 98 | 4-Bromo-2-((dimethylamino)methyl)-N-methyl-N- | 24.96 | >50 | 26.75 |
| | (3-methyl-4-(methylthio)phenyl)aniline | | | |
| Compound 99 | 3-(methoxymethyl)-N-methyl-4-(3-methyl-4- | NA | NA | NA |
| | (methylthio)phenoxy)benzenesulfonamide | | | |
| Compound 100 | (R)-3-((3-(dimethylamino)pyrrolidin-1-yl)methyl)- | 5.03 | 35.62 | 10.55 |
| | 4-(3-methyl-4- | | | |
| | (methylthio)phenoxy)benzenesulfonamide | | | |
| Compound 101 | 4-(3-methyl-4-(methylthio)phenoxy)-3-(pyrrolidin- | 1.74 | 40.57 | 4.74 |
| | 1-ylmethyl)benzenesulfonamide | | | |
| Compound 102 | (R)-3-((2-(hydroxymethyl)pyrrolidin-1-yl)methyl)- | 6.76 | >50 | 17.64 |
| | 4-(3-methyl-4- | | | |
| | (methylthio)phenoxy)benzenesulfonamide | | | |
| Compound 103 | (S)-3-((2-(hydroxymethyl)pyrrolidin-1-yl)methyl)- | 6.88 | >50 | 17.63 |
| | 4-(3-methyl-4- | | | |
| | (methylthio)phenoxy)benzenesulfonamide | | | |
| Compound 104 | (S)-3-((2-(methoxymethyl)pyrrolidin-1-yl)methyl)- | 27.78 | >50 | NA |
| | 4-(3-methyl-4- | | | |
| | (methylthio)phenoxy)benzenesulfonamide | | | |
| Compound 105 | (S)-4-(3-methyl-4-(methylthio)phenoxy)-3-(((2- | 6.38 | 22.22 | 6.86 |
| | pyrrolidin-1-ylmethyl)pyrrolidin-1- | | | |
| | yl)methyl)benzensulfonamide | | | |
| Compound 106 | 3-((4-isopropylpiperazin-1-yl)methyl)-4-(3-methyl- | 3.57 | 24.3 | 8.21 |
| | 4-(methylthio)phenoxy)benzenesulfonamide | | | |
| Compound 107 | 3-methyl-1-(3-methyl-4-(methylthio)phenyl)- | 4.84 | >50 | 12.78 |
| | 1,2,3,4-tetrahydroquinazoline-6-sulfonamide | | | |
| Compound 108 | 3-((dimethylamino)methyl)-4-(3-methyl-4- | 2.64 | 44.88 | 16.99 |
| | (methylthio)phenylamino)benzensulfonamide | | | |
| Compound 109 | (R)-4-(3-methyl-4-(methylthio)phenoxy)-3- | 4.9 | >50 | 16.34 |
| | (((tetrahydrofuran-2- | | | |
| | yl)methylamino)methyl)benzenesulfonamide | | | |
| Compound 110 | 3-(((2S,4R)-4-hydroxy-2- | 13.07 | >50 | NA |
| | (hydroxymethyl)pyrrolidin-1-yl)methyl)-4-((3- | | | |
| | methyl-4-methylthio)phenoxy)benzenesulfonamide | | | |
| Compound 111 | 4-(3-methyl-4-(methylthio)phenoxy)-3-(piperazin- | 7.12 | 46.28 | 22.6 |
| | 1-ylmethyl)benzenesulfonamide | | | |

### Synthesis of the Sertraline and UK-416244 Analogs

Synthesis of exemplary sertraline analogs is described in Examples 7-64 below. Other sertraline analogs can be made using the methods described in Welch et al., J. Med. Chem. 27:1508-1515, 1984, and PCT Publication Nos. WO 00/51972 WO 02/18333, and WO 01/72687. In the Examples below, the starting materials were purchased from Aldrich, Tee Hai, and Atomax. Merck silica gel 60 (230-400 mesh) was used for chromatography. ¹H NMR spectra were recorded on Bruker 400 MHz spectrometers. MS was obtained on Agilent 1200 LC/MS system. The HPLC separations were achieved on shimadzu HPLC system.

### ABBREVIATIONS

The following abbreviations are used in the Examples below. 6 chemical shift; Ac: acetyl; Ar: aromatic; Boc: t-Butoxycarbonyl; d: doublet; DCM: dichloromethane; DIPEA: N,N-diisopropylethylamine; DMF: N,N-dimethylformamide; DMSO: Dimethylsulfoxide; HATU: 2-(1H-7-Azabenzotriazol-1-yl)-1,1,3,3-tetramethyl uronium hexafluorophosphate Methanaminium; HPLC: High pressure liquid chromatography; LAH: lithium aluminum hydride; Me: methyl; MS: mass spectrum; NMP: N-methylmorpholine; NMR: nuclear magnetic resonance; m/z: mass spectrum peak; Pd/C: palladium on activated charcoal, 10% Pd; q: quartet; s: singlet; t: triplet; TBAI: tetrabutylammonium Iodide; TEA: triethyl amine; THF: tetrahydrofuran;

### Example 7

### Synthesis of 3-((dimethylamino)methyl)-4-(3-methyl-4-(methylthio)phenoxy)-N-phenylbenzenesulfonamide

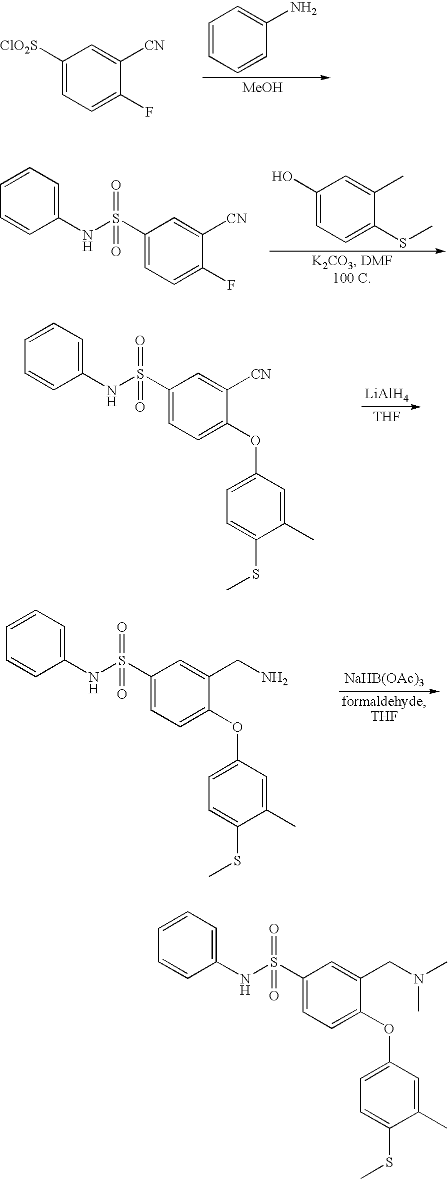

### 3-cyano-4-fluoro-N-phenylbenzenesulfonamide

A mixture of 3-cyano-4-fluorobenzene-1-sulfonyl chloride (1 g, 4.55 mmol) and aniline (4.1 ml, 45.5 mmol) in methanol (15 ml) was stirred at room temperature for 15 min. The mixture was quenched with 2N HCl. pH was adjusted to 1. The mixture was extracted 3 times with ethyl acetate. The organic layer was separated, dried over anhydrous Na₂SO₄, and concentrated in vacuo. The residue (1.25 g) was directly used for the next step. ¹H NMR (CD₃OD, 400 Mhz) generated the following peaks: δ 8.10 (dd, 1H), 7.99-8.03 (m, 1H), 7.47 (t, 1H), 7.23-7.27 (m, 2H), 7.07-7.13 (m, 3H).

### 3-cyano-4-(3-methyl-4-(methylthio)phenoxy)-N-phenylbenzenesulfonamide

A mixture of 3-cyano-4-fluoro-N-phenylbenzenesulfonamide (1.25 g, 4.55 mmol), 3-methyl-4-(methylthio)phenol (772 mg, 5.00 mmol) and K₂CO₃ (660 mg, 4.78 mmol) in DMF (20 ml) was stirred in an 100° C. oil bath for 4 hours. The mixture was then cooled to 0° C. and acidified to pH 1 using 2 N HCl. The mixture was extracted 3 times with diethyl ether. The organic layer was separated, dried over anhydrous Na₂SO₄, and concentrated in vacuo. The residue was purified by flash chromatography eluted with ethyl acetate:Hexane (30-50%) to yield 1.4 g of the desired product, 3-cyano-4-(3-methyl-4-(methylthio)phenoxy)-N-phenylbenzenesulfonamide. ¹H NMR (CD₃OD, 400 Mhz) generated the following peaks: δ 8.04 (d, 1H), 7.83 (dd, 1H), 7.23-7.30 (m, 3H), 7.07-7.11 (m, 3H), 6.97-6.99 (m, 2H), 6.85 (d, 1H), 2.47 (s, 3H), 2.30 (s, 3H).

### 3-(aminomethyl)-4-(3-methyl-4-(methylthio)phenoxy)-N-phenylbenzenesulfonamide

3-cyano-4-(3-methyl-4-(methylthio)phenoxy)-N-phenylbenzenesulfonamide (200 mg, 0.48 mmol) was dissolved in 3 ml anhydrous THF and cooled to 0° C., followed by drop-wise addition of 2 ml of LAH (1.0 M in THF). The mixture was then warmed to room temperature and stirred overnight. The mixture was quenched by addition of 2 N NaOH, water, and 10% Rochelle's salt solution. The mixture was extracted 3 times with ethyl acetate. The resulting organic layer was separated, dried over anhydrous Na₂SO₄, and concentrated in vacuo. The residue was purified by flash chromatography and eluted with MeOH:DCM (1-10%) to yield 100 mg of the desired product, 3-(aminomethyl)-4-(3-methyl-4-(methylthio)phenoxy)-N-phenylbenzenesulfonamide. ¹H NMR (CD₃OD, 400 Mhz) generated the following peaks: δ 7.81 (d, 1H), 7.55 (dd, 1H), 7.18-7.26 (m, 3H), 7.01-7.10 (m, 3H), 6.88-6.89 (m, 2H), 6.71 (d, 1H), 3.86 (s, 2H), 2.44 (s, 3H), 2.29 (s, 3H). Mass spectrometry showed m/z=415.0 (M+H⁺).

### 3-((dimethylamino)methyl)-4-(3-methyl-4-(methylthio)phenoxy)-N-phenylbenzenesulfonamide

3-(aminomethyl)-4-(3-methyl-4-(methylthio)phenoxy)-N-phenylbenzenesulfonamide (100 mg, 0.24 mmol) was dissolved in 3 ml THF and was added to 37% formaldehyde (27 μl, 0.36 mmol). The mixture was stirred at room temperature for 30 minutes, followed by addition of NaHB(OAc)₃ (153 mg, 0.72 mmol). The mixture was then stirred overnight, quenched by addition of saturated sodium bicarbonate solution, and extracted 3 times with ethyl acetate. The organic layer was separated and dried over anhydrous Na₂SO₄ and concentrated in vacuo. The residue was purified by flash chromatography eluted with MeOH:DCM (1-5%) twice to yield 18 mg of the desired product, 3-((dimethylamino)methyl)-4-(3-methyl-4-(methylthio)phenoxy)-N-phenylbenzenesulfonamide. ¹H NMR (CDCl₃, 400 Mhz) generated the following peaks: δ 7.96 (d, 1H), 7.54 (dd, 1H), 7.15-7.26 (m, 3H), 7.07-7.09 (m, 3H), 6.78-6.81 (m, 2H), 6.67 (d, 1H), 3.58 (s, 2H), 2.45 (s, 3H), 2.31 (s, 3H), 2.25 (s, 6H). Mass spectrometry showed m/z=443.1 (M+H⁺).

### Example 8

### Synthesis of N-benzyl-3-((dimethylamino)methyl)-4-(3-methyl-4-(methylthio)phenoxy)benzenesulfonamide

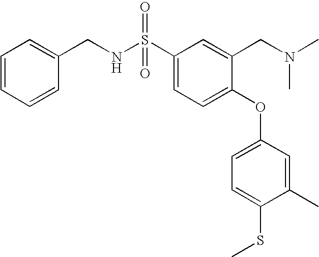

This compound was prepared a manner analogous to 3-((dimethylamino)methyl)-4-(3-methyl-4-(methylthio)phenoxy)-N-phenylbenzenesulfonamide, as described in Example 7. ¹H NMR (CDCl₃, 400 Mhz) generated the following peaks: δ 8.01 (d, 1H), 7.66 (dd, 1H), 7.23-7.30 (m, 3H), 7.18-7.20 (m, 3H), 6.81-6.84 (m, 3H), 4.16 (s, 2H), 3.58 (s, 2H), 2.47 (s, 3H), 2.35 (s, 3H), 2.30 (s, 6H). Mass spectrometry showed m/z=457.1 (M+H⁺)

### Example 9

### Synthesis of 1-(3,4-dichlorophenyl)-4-isopropyl-1,2,3,4-tetrahydronaphthalene

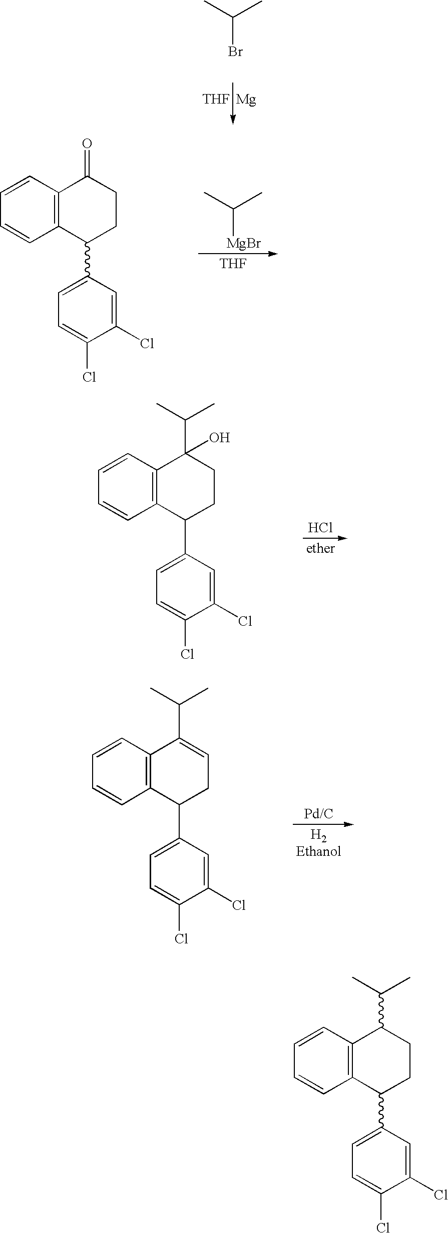

### 4-(3,4-dichlorophenyl)-1-isopropyl-1,2,3,4-tetrahydronaphthalen-1-ol

Magnesium (80 mg, 3.28 mmol), 2-bromopropane (0.324 ml, 3.46 mmol) and THF (5 ml) were added to a flame-dried round bottom flask. The mixture was stirred for 30 minutes until all of the magnesium was consumed. 4-(3,4-dichlorophenyl)-3,4-dihydronaphthalen-1(2H)-one (503 mg, 1.73 mmol) in 5 ml THF was then added to the mixture at 0° C. After stirring for two hours, the reaction mixture was diluted using saturated NH₄Cl. The mixture was extracted 3 times with diethyl ether. The organic layer was separated and dried over anhydrous Na₂SO₄, and concentrated in vacuo. The residue was purified by flash chromatography eluted with ethyl acetate:Hexane (10%) to yield 174 mg (cis+trans) of the desired product, 4-(3,4-dichlorophenyl)-1-isopropyl-1,2,3,4-tetrahydronaphthalen-1-ol.

### 1-(3,4-dichlorophenyl)-4-isopropyl-1,2-dihydronaphthalene

4-(3,4-dichlorophenyl)-1-isopropyl-1,2,3,4-tetrahydronaphthalen-1-ol (174 mg, 0.52 mmol) was dissolved in 20 ml 1.0 M HCl in diethyl ether and the mixture was stirred overnight. The mixture was concentrated in vacuo, resulting in a yellow residue. The residue was purified by flash chromatography eluted with hexane to yield 80.4 mg of the desired product, 1-(3,4-dichlorophenyl)-4-isopropyl-1,2-dihydronaphthalene. ¹H NMR (CDCl₃, 400 Mhz) generated the following peaks: δ 7.42 (d, 1H), 7.32 (d, 1H), 7.27 (t, 1H), 7.22 (d, 1H), 7.14 (t, 1H), 6.98 (dd, 1H), 6.89 (d, 1H), 5.74 (t, 1H), 3.98 (t, 1H), 2.98-3.02 (m, 1H), 2.45-2.67 (m, 2H), 1.16 (d, 6H).

### 1-(3,4-dichlorophenyl)-4-isopropyl-1,2,3,4-tetrahydronaphthalene

1-(3,4-dichlorophenyl)-4-isopropyl-1,2-dihydronaphthalene (80.4 mg, 0.25 mmol) was dissolved in 5 ml ethanol. The mixture was purged with N₂ before 150 mg Pd/C was added. H₂ then was allowed to bubble through the solution until all of the starting material was consumed. The mixture was passed through Celite and concentrated in vacuo, resulting in a yellow residue. The residue was purified by flash chromatography eluted with hexane to yield 53.3 mg of the desired product, 1-(3,4-dichlorophenyl)-4-isopropyl-1,2,3,4-tetrahydronaphthalene. ¹H NMR (CDCl₃, 400 Mhz) generated the following peaks: δ 7.32 (d, 1H), 7.25-7.27 (m, 1H), 7.14-7.20 (m, 2H), 7.06-7.08 (m, 2H), 6.86-6.89 (m, 1H), 4.09-4.17 (m, 1H), 2.67-2.74 (m, 1H), 2.31-2.37 (m, 1H), 2.03-2.08 (m, 2H), 1.61-1.71 (m, 2H), 1.04 (d, 3H), 0.80 (d, 3H).

### Example 10

### Synthesis of 4-(3,4-dichlorophenyl)-N-methyl-4,5,6,7-tetrahydrobenzo[b]thiophen-7-amine

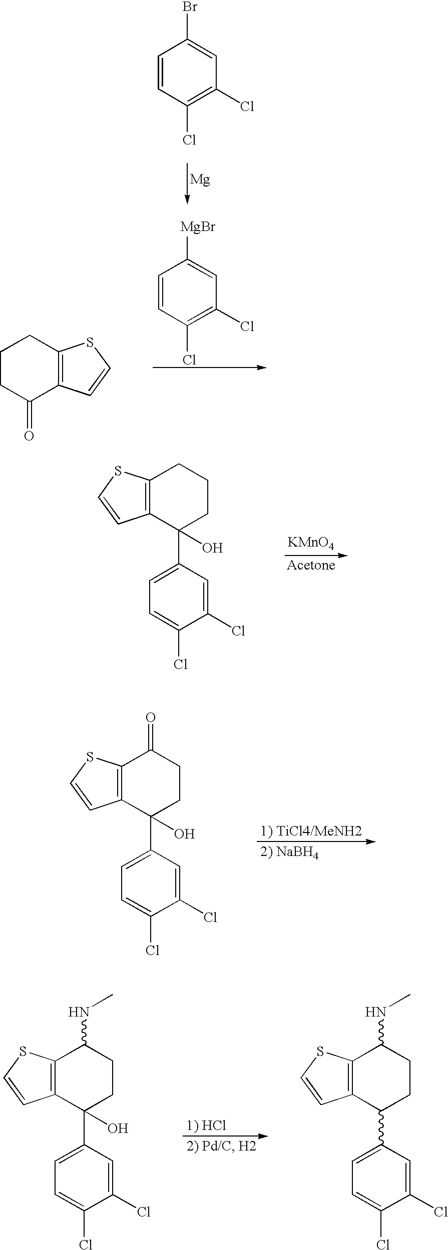

### 4-(3,4-dichlorophenyl)-4,5,6,7-tetrahydrobenzo[b]thiophen-4-ol

Magnesium (323 mg, 13.28 mmol), 4-bromo-1,2-dichlorobenzene (3 g, 13.28 mmol), and THF (10 ml) were added to a flame-dried round bottom flask. The mixture was allowed to stir until all of magnesium was consumed. Then 4-(3,4-dichlorophenyl)-3,4-dihydronaphthalen-1(2H)-one (1.11 g, 7.3 mmol) in 4 ml THF was added to the mixture at 0° C. After stirring for two hours, the reaction mixture was diluted with saturated NH₄Cl. The mixture was then extracted 3 times with diethyl ether. The organic layer was separated, dried over anhydrous Na₂SO₄, and concentrated in vacuo. The residue was purified by flash chromatography eluted with ethyl acetate:hexane (4-7%) to yield 1.5 g of the desired product, 4-(3,4-dichlorophenyl)-4,5,6,7-tetrahydrobenzo[b]thiophen-4-ol. ¹H NMR (CDCl₃, 400 Mhz) generated the following peaks: δ 7.50 (d, 1H), 7.36 (d, 1H), 7.14 (dd, 1H), 7.07 (d, 1H), 6.59 (d, 1H), 2.84-2.97 (m, 2H), 1.86-2.17 (m, 4H).

### 4-(3,4-dichlorophenyl)-4-hydroxy-5,6-dihydrobenzo[b]thiophen-7(4H)-one

4-(3,4-dichlorophenyl)-4,5,6,7-tetrahydrobenzo[b]thiophen-4-ol (1.5 g, 5.01 mmol) was dissolved in acetone/H₂O (170.6 ml/3.5 ml) and KMnO₄ (11.88 g, 75.2 nmol) was added to the solution. The mixture was heated in a 60° C. oil bath overnight. The mixture was passed through Celite and concentrated in vacuo, resulting in a yellow residue. The residue was dissolved in ethyl Acetate\H₂O mixture. The aqueous layer was extracted twice using ethyl acetate. The organic layer was separated and dried over anhydrous Na₂SO₄, and concentrated in vacuo. The residue was purified by flash chromatography eluted with Ethyl Acetate:Hexane (20%) to yield 225 mg of the desired product, 4-(3,4-dichlorophenyl)-4-hydroxy-5,6-dihydrobenzo[b]thiophen-7(4H)-one. ¹H NMR (CDCl₃, 400 Mhz) generated the following peaks: δ 7.69 (d, 1H), 7.51 (d, 1H), 7.41 (d, 1H), 7.11 (dd, 1H), 6.87 (d, 1H), 2.88-2.91 (m, 1H), 2.42-2.56 (m, 3H).

### 4-(3,4-dichlorophenyl)-7-(methylamino)-4,5,6,7-tetrahydrobenzo[b]thiophen-4-ol

4-(3,4-dichlorophenyl)-4-hydroxy-5,6-dihydrobenzo[b]thiophen-7(4H)-one (225 mg, 0.72 mmol) was dissolved in 1 ml THF, and 5.4 ml 2 M MeNH₂ in THF was added to the solution. The mixture was placed in an ice bath, and TiCl₄ (144 mg, 0.43 mmol) was slowly added. After stirring for 3 hours, the mixture was passed through Celite and concentrated in vacuo, generating a white foam. The foam was dissolved in 3 ml anhydrous methanol and followed by addition of NaBH₄ (54 mg, 1.44 mmol). The mixture was stirred for 1 hour, diluted with ethyl acetate, and washed using water and brine. The organic layer was separated, dried over anhydrous Na₂SO₄, and concentrated in vacuo. The residue was purified by flash chromatography and eluted with DCM:methanol:NH₃ (90:10:1) to yield 200 mg of the desired product, 4-(3,4-dichlorophenyl)-7-(methylamino)-4,5,6,7-tetrahydrobenzo[b]thiophen-4-ol. ¹H NMR (CDCl₃, 400 Mhz) generated the following peaks: δ 7.52 (d, 1H), 7.37 (d, 1H), 7.13-7.16 (m, 2H), 6.55 (d, 1H), 3.88-3.91 (m, 1H), 2.60 (s, 3H), 2.17-2.27 (m, 2H), 1.82-2.00 (m, 2H).

### 4-(3,4-dichlorophenyl)-N-methyl-4,5,6,7-tetrahydrobenzo[b]thiophen-7-amine

4-(3,4-dichlorophenyl)-7-(methylamino)-4,5,6,7-tetrahydrobenzo[b]thiophen-4-ol (200 mg, 0.61 mmol) was dissolved in 3 ml 10% methanol in DCM. 5 ml 2.0 M HCl in diethyl ether was added to the solution. The mixture was then stirred for 1 hour, concentrated in vacuo, and basified with saturated sodium bicarbonate solution. The mixture was extracted 3 times with ethyl acetate. The organic layer was separated, dried over anhydrous Na₂SO₄, and concentrated in vacuo to give 180 mg of brown oil. The residue was dissolved in 10 ml ethanol. N₂ was used to purge the mixture prior to adding 200 mg Pd/C. H₂ then was allowed to bubble through the solution until all of the starting material was consumed. The mixture was passed through Celite and concentrated in vacuo to give yellow residue, which was purified by flash chromatography eluted with ethyl acetate:hexane:triethylamine (40:60:1) to yield 84.8 mg of the desired product, 4-(3,4-dichlorophenyl)-N-methyl-4,5,6,7-tetrahydrobenzo[b]thiophen-7-amine. ¹H NMR (CDCl₃, 400 Mhz) generated the following peaks: δ 7.32-7.36 (m, 1H), 7.18-7.21 (m, 1H), 7.11-7.16 (m, 1H), 6.91-6.96 (m, 1H), 6.43-6.51 (m, 1H), 3.85-3.97 (m, 2H), 2.57 (d, 3H), 1.73-2.29 (m, 4H). Mass spectrometry showed m/z=312.0 (M+H⁺).

### Example 11

### Synthesis of 2-(((1S,4S)-4-(3,4-dichlorophenyl)-1,2,3,4-tetrahydronaphthalen-1-yl) (methyl)amino)ethanol

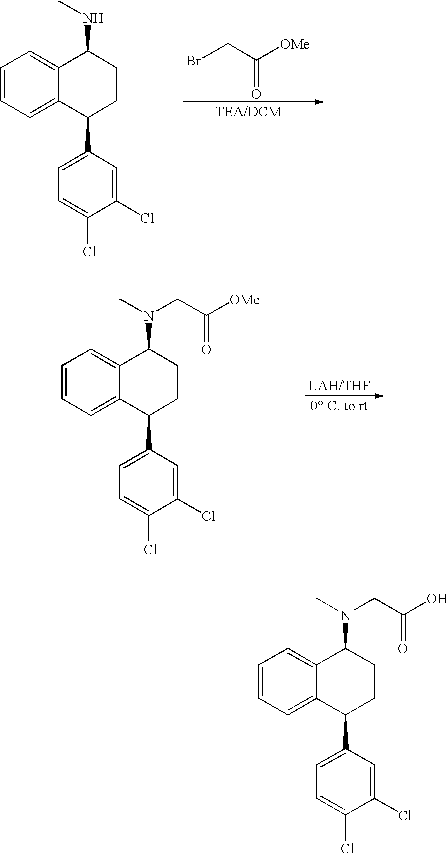

### Methyl 2-(((1S,4S)-4-(3,4-dichlorophenyl)-1,2,3,4-tetrahydronaphthalen-1-yl)(methyl)amino)acetate

Triethyl amine (2.87 ml, 20.52 mmol) was added to sertraline (3 g, 9.80 mmol) in DCM (40 ml), followed addition of methyl bromoacetate (1.1 ml, 11.75 mmol) at 0° C. The mixture was stirred overnight and then washed with water. The aqueous layer was extracted 3 times with DCM. The organic layer was separated, dried over anhydrous MgSO₄, and concentrated in vacuo. The residue was purified by flash chromatography and eluted with ethyl acetate:hexane (5%-10%) to yield 1.38 g of the desired product, methyl 2-(((1S,4S)-4-(3,4-dichlorophenyl)-1,2,3,4-tetrahydronaphthalen-1-yl)(methyl)amino)acetate. ¹H NMR (CDCl₃, 400 MHz) generated the following peaks: δ 7.81 (d, 1H), 7.32 (d, 1H), 7.26 (t, 1H), 7.12-7.17 (m, 2H), 6.89 (d, 1H), 6.81 (dd, 1H), 4.07-4.14 (m, 1H), 3.93-3.97 (m, 1H), 3.31 (q, 2H), 2.41 (s, 3H), 2.02-2.14 (m, 2H), 1.62-1.76 (m, 2H).

### 2-(((1S,4S)-4-(3,4-dichlorophenyl)-1,2,3,4-tetrahydronaphthalen-1-yl) (methyl)amino)ethanol

1 M LAH (5 ml) in THF was added to methyl 2-(((1S,4S)-4-(3,4-dichlorophenyl)-1,2,3,4-tetrahydronaphthalen-1-yl)(methyl)amino) acetate (0.28 g, 0.74 mmol) in THF (5 ml) at 0° C. The mixture was stirred overnight and was quenched with water. The aqueous layer was extracted 3 times with ethyl acetate. The organic layer was then separated, dried over anhydrous MgSO₄, and concentrated in vacuo. The residue was purified by flash chromatography eluted with ethyl acetate:hexane (10%-30%) to yield 150 mg of the desired product, 2-(((1S,4S)-4-(3,4-dichlorophenyl)-1,2,3,4-tetrahydronaphthalen-1-yl)(methyl)amino)ethanol. ¹H NMR (CDCl₃, 400 Mhz) generated the following peaks: δ 7.70 (d, 1H), 7.32 (d, 1H), 7.28 (t, 1H), 7.16 (t, 1H), 7.11 (d, 1H), 6.91 (d, 1H), 6.82 (dd, 1H), 4.11-4.14 (m, 1H), 3.93 (t, 1H), 3.64-3.67 (m, 2H), 2.71 (t, 2H), 2.41 (s, 3H), 1.99-2.24 (m, 2H), 1.68-1.73 (m, 2H). Mass spectrometry revealed m/z=350.1 (M+H⁺).

### Example 12

### Synthesis of (5S,8S)—N-cyclopropyl-5-(3,4-dichlorophenyl)-8-(methylamino)-5,6,7,8-tetrahydronaphthalene-2-carboxamide

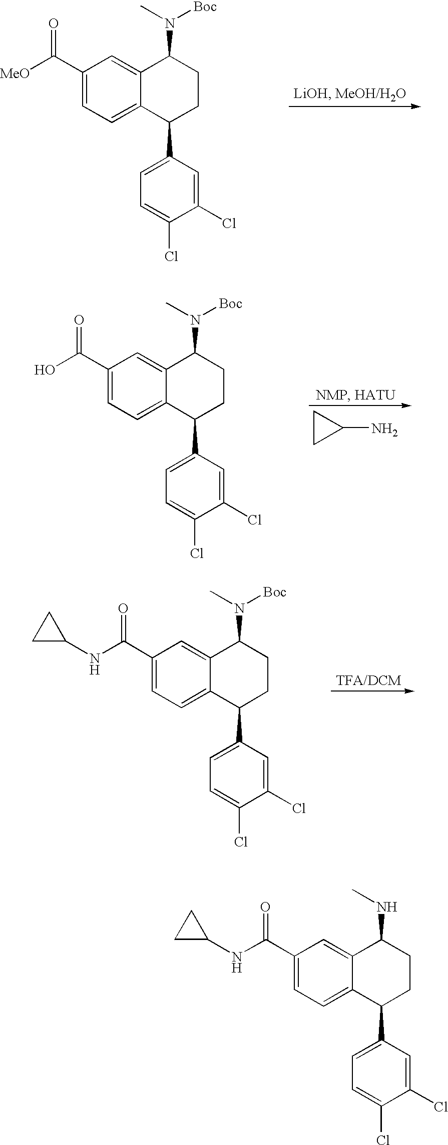

### (5S,8S)-8-(tert-butoxycarbonyl(methyl)amino)-5-(3,4-dichlorophenyl)-5,6,7,8-tetrahydronaphthalene-2-carboxylic acid

Lithium hydroxide (64 mg, 2.69 mmol) was added to (5S,8S)-methyl 8-(tert-butoxycarbonyl(methyl)amino)-5-(3,4-dichlorophenyl)-5,6,7,8-tetra-hydronaphthalene-2-carboxylate (Preparation 1 (Example 23), 250 mg, 0.538 mmol) in 10 ml MeOH/H₂O (9:1). The mixture was stirred at room temperature overnight and then acidified using 1 N HCl to pH 3-4. The resulting mixture was extracted 3 times with ethyl acetate. The organic layer was separated, dried over anhydrous Na₂SO₄, and concentrated in vacuo. The residue was purified by flash chromatography and eluted with MeOH:DCM (5%-20%) to yield 200 mg of the desired product, (5S,8S)-8-(tert-butoxycarbonyl(methyl)amino)-5-(3,4-dichlorophenyl)-5,6,7,8-tetrahydrona-phthalene-2-carboxylic acid.

### tert-butyl (1S,4S)-7-(cyclopropylcarbamoyl)-4-(3,4-dichlorophenyl)-1,2,3,4-tetrahydro-naphthalen-1-yl(methyl)carbamate

To (5S,8S)-8-(tert-butoxycarbonyl(methyl)amino)-5-(3,4-dichlorophenyl)-5,6,7,8-tetrahydrona-phthalene-2-carboxylic acid (120 mg, 0.27 mmol) in 2 ml DMF/DCM (1:1) was added HATU (0.186 g, 0.49 mmol), followed by cyclopropylamine (33 μl, 0.49 mmol) and 4-methyl morpholine (0.14 ml, 1.33 mmol). The mixture was stirred over night and diluted with water. The resulting mixture was extracted 3 times with ethyl acetate. The organic layer was separated and dried over anhydrous Na₂SO₄, and concentrated in vacuo. The residue was purified by flash chromatography and eluted with ethyl acetate:hexane (20%) to yield 100 mg desired product, tert-butyl (1S,4S)-7-(cyclopropylcarbamoyl)-4-(3,4-dichlorophenyl)-1,2,3,4-tetrahydro-naphthalen-1-yl(methyl)carbamate. Mass spectrometry resulted in m/z=511.1 (M+Na⁺).

### (5S,8S)—N-cyclopropyl-5-(3,4-dichlorophenyl)-8-(methylamino)-5,6,7,8-tetrahydronaphthalene-2-carboxamide

Trifluoroacetic acid (0.5 ml) was added to (5S,8S)-8-(tert-butoxycarbonyl(methyl)amino)-5-(3,4-dichlorophenyl)-5,6,7,8-tetrahydrona-phthalene-2-carboxylic acid (100 mg, 0.20 mmol) in 2 ml DCM. The mixture was stirred for 1 hour, diluted with DCM, and washed using saturated sodium bicarbonate solution and brine. The organic layer was separated, dried over anhydrous Na₂SO₄, and concentrated in vacuo. The residue was purified by flash chromatography eluted with MeOH:DCM:triethyl amine (2:98:1 to 10:90:1) to yield 25 mg of the desired product, (5S,8S)—N-cyclopropyl-5-(3,4-dichlorophenyl)-8-(methylamino)-5,6,7,8-tetrahydronaphthalene-2-carboxamide. ¹H NMR (CDCl₃, 400 Mhz) generated the following peaks: δ 7.77 (d, 1H), 7.45 (dd, 1H), 7.34 (d, 1H), 7.20 (d, 1H), 6.93 (dd, 1H), 6.85 (d, 1H), 6.23 (s, 1H), 3.99 (t, 1H), 3.74-3.76 (m, 1H), 2.87-2.92 (m, 1H), 2.54 (s, 3H), 1.99-2.08 (m, 2H), 1.79-1.85 (m, 2H), 0.85-0.90 (m, 2H), 0.60-0.64 (m, 2H). Mass spectrometry showed m/z=389.0 (M+H⁺).

### Example 13

### Synthesis of N-benzyl-3-((dimethylamino)methyl)-4-(3-methyl-4-(methylthio)phenoxy)benzenesulfonamide

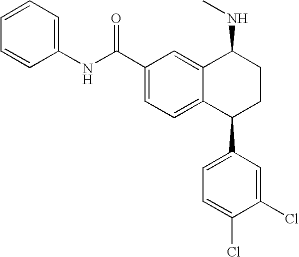

This compound was prepared in an analogous fashion to (5S,8S)—N-cyclopropyl-5-(3,4-dichlorophenyl)-8-(methylamino)-5,6,7,8-tetrahydronaphthalene-2-carboxamide, as described in example 12. ¹H NMR (CDCl₃, 400 Mhz) generated the following peaks: δ 7.94 (d, 2H), 7.64-7.66 (m, 3H), 7.35-7.39 (m, 3H), 7.23 (d, 1H), 7.15 (t, 1H), 6.91-6.97 (m, 2H), 4.01 (t, 1H), 3.79-3.81 (m, 1H), 2.56 (s, 3H), 1.87-2.10 (m, 4H). Mass spectrometry showed m/z=425.0 (M+H⁺).

### Example 14

### Synthesis of (5S,8S)-5-(3,4-dichlorophenyl)-8-(methylamino)-N-(pyridin-2-yl)-5,6,7,8-tetrahydronaphthalene-2-carboxamide

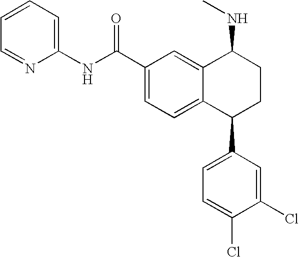

This compound was prepared in an analogous fashion to (5S,8S)—N-cyclopropyl-5-(3,4-dichlorophenyl)-8-(methylamino)-5,6,7,8-tetrahydronaphthalene-2-carboxamide, as described in example 12. ¹H NMR (CDCl₃, 400 Mhz) generated the following peaks: δ 8.61 (s, 1H), 8.39 (d, 1H), 8.30-8.32 (m, 1H), 7.97 (d, 1H), 7.74-7.79 (m, 1H), 7.67 (dd, 1H), 7.37 (d, 1H), 7.24 (d, 1H), 7.07-7.10 (m, 1H), 6.93-6.97 (m, 2H), 4.00-4.04 (m, 1H), 3.77-3.79 (m, 1H), 2.56 (s, 3H), 1.85-2.12 (m, 4H). Mass spectrometry showed m/z=426.0 (M+H⁺).

### Example 15

### Synthesis of (5S,8S)—N-cyclobutyl-5-(3,4-dichlorophenyl)-8-(dimethylamino)-5,6,7,8-tetrahydronaphthalene-2-carboxamide

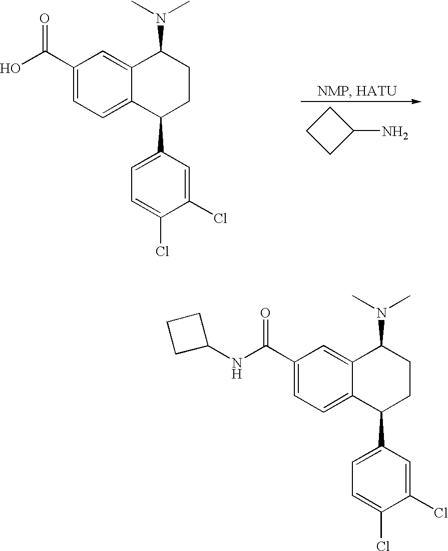

To (5S,8S)-5-(3,4-dichlorophenyl)-8-(dimethylamino)-5,6,7,8-tetrahydronaphthalene-2-carboxylic acid (made from N,N-dimethyl sertraline; see PCT Publication No. WO 00/51972 for details, 100 mg, 0.27 mmol) in 2 ml DMF/DCM (1:1) was added HATU (163 mg, 0.43 mmol), followed by cyclobutylamine (36 μl, 0.43 mmol) and 4-methyl morpholine (0.18 ml, 1.65 mmol). The mixture was stirred overnight and diluted with water. The resulting mixture was extracted 3 times with ethyl acetate. The organic layer was separated and dried over anhydrous Na₂SO₄, and concentrated in vacuo. The residue was purified by flash chromatography and eluted with ethyl acetate:hexane (20%) to yield 100 mg desired product, (5S,8S)—N-cyclobutyl-5-(3,4-dichlorophenyl)-8-(dimethylamino)-5,6,7,8-tetrahydronaphthalene-2-carboxamide. ¹H NMR (CDCl₃, 300 Mhz) generated the following peaks: δ 8.06 (s, 1H), 7.60 (d, 1H), 7.31 (d, 1H), 7.08 (s, 1H), 6.96 (d, 1H), 6.81 (d, 1H), 6.31 (b, 1H), 4.51-4.62 (m, 1H), 4.11-4.14 (m, 1H), 3.75-3.80 (t, 1H), 2.42-2.48 (m, 2H), 2.30 (s, 6H), 1.95-2.11 (m, 4H), 1.56-1.82 (m, 4H). Mass spectrometry showed m/z=417.1 (M+H⁺).

### Example 16

### Synthesis of (5S,8S)—N-cyclopropyl-5-(3,4-dichlorophenyl)-8-(dimethylamino)-5,6,7,8-tetrahydronaphthalene-2-carboxamide

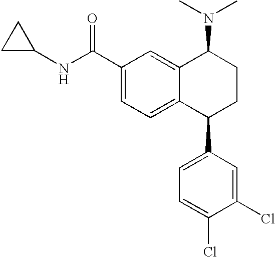

This compound was prepared in an analogous fashion to (5S,8S)—N-cyclobutyl-5-(3,4-dichlorophenyl)-8-(dimethylamino)-5,6,7,8-tetrahydronaphthalene-2-carboxamide, as described in example 15. ¹H NMR (CDCl₃, 300 Mhz) generated the following peaks: δ 8.03 (s, 1H), 7.61 (d, 1H), 7.31 (d, 1H), 7.07 (s, 1H), 6.96 (d, 1H), 6.80 (d, 1H), 6.34 (b, 1H), 4.11-4.14 (m, 1H), 3.75-3.80 (t, 1H), 2.89-2.93 (m, 1H), 2.29 (s, 6H), 2.00-2.14 (m, 2H), 1.69-1.73 (m, 2H), 0.84-0.95 (m, 2H), 0.55-0.64 (m, 2H). Mass spectrometry showed m/z 403.1 (M+H⁺).

### Example 17

### Synthesis of (5S,8S)—N-(cyclopropylmethyl)-5-(3,4-dichlorophenyl)-8-(dimethylamino)-5,6,7,8-tetrahydronaphthalene-2-carboxamide

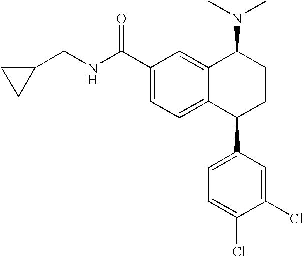

This compound was prepared in an analogous fashion to (5S,8S)—N-cyclobutyl-5-(3,4-dichlorophenyl)-8-(dimethylamino)-5,6,7,8-tetrahydronaphthalene-2-carboxamide, as described in example 15. ¹H NMR (CDCl₃, 300 Mhz) generated the following peaks: δ 8.01 (s, 1H), 7.62 (d, 1H), 7.32 (d, 1H), 7.09 (s, 1H), 6.97 (d, 1H), 6.82 (d, 1H), 6.31 (b, 1H), 4.09-4.16 (m, 1H), 3.77-3.82 (t, 1H), 3.30-3.36 (m, 2H), 2.31 (s, 6H), 2.04-2.14 (m, 2H), 1.68-1.73 (m, 2H), 1.04-1.14 (m, 1H), 0.55-0.65 (m, 2H), 0.24-0.34 (m, 2H). Mass spectrometry showed m/z=417.0 (M+H⁺).

### Example 18

### Synthesis of (5S,8S)-5-(3,4-dichlorophenyl)-8-(methylamino)-N-(pyridin-2-yl)-5,6,7,8-tetrahydronaphthalene-2-carboxamide

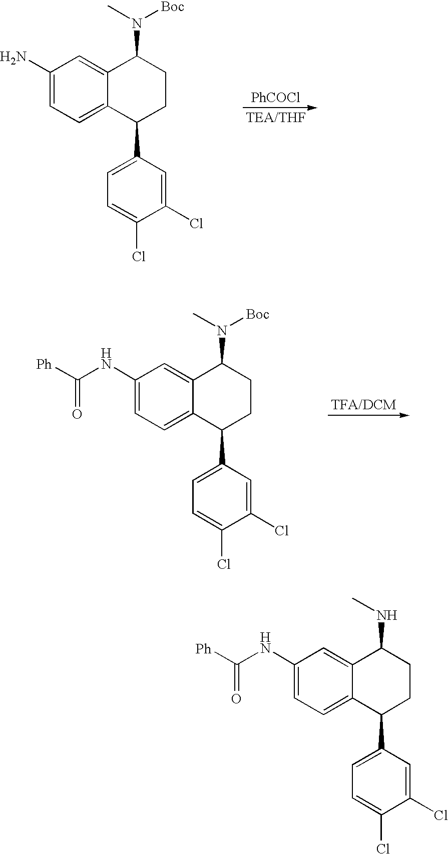

### tert-butyl (1S,4S)-7-benzamido-4-(3,4-dichlorophenyl)-1,2,3,4-tetrahydronaphthalen-1-yl (methyl)carbamate

To tert-butyl (1S,4S)-7-amino-4-(3,4-dichlorophenyl)-1,2,3,4-tetrahydronaphthalen-1-yl(methyl) carbamate (see PCT Publication No. WO 00/51972 for details, 100 mg, 0.24 mmol) in 5 ml THF was added benzoyl chloride (48 μl, 0.47 mmol) and triethyl amine (99 μl, 0.71 mmol). The mixture was stirred at room temperature overnight and diluted with water. The resulting mixture was extracted 3 times with ethyl acetate. The organic layer was separated, dried over anhydrous MgSO₄, and concentrated in vacuo. The residue was purified by flash chromatography eluted with ethyl acetate:hexane (5-20%) to yield 121 mg of the desired product, tert-butyl (1S,4S)-7-benzamido-4-(3,4-dichlorophenyl)-1,2,3,4-tetrahydrona-phthalen-1-yl(methyl)carbamate. Mass spectrometry showed m/z=547.1 (M+Na⁺).

### N-((5S,8S)-5-(3,4-dichlorophenyl)-8-(methylamino)-5,6,7,8-tetrahydronaphthalen-2-yl)benzamide

Trifluoroacetic acid (0.25 ml) was added to tert-butyl (1S,4S)-7-benzamido-4-(3,4-dichlorophenyl)-1,2,3,4-tetrahydrona-phthalen-1-yl(methyl)carbamate (121 mg, 0.23 mmol) in 2 ml DCM, and the mixture was stirred for 3 hours. The mixture was then diluted with DCM and washed with saturated sodium bicarbonate solution and brine. The organic layer was separated, dried over anhydrous Na₂SO₄, and concentrated in vacuo. The residue was purified by flash chromatography eluted with methanol:DCM (6%) to yield 85.4 mg of the desired product, N-((5S,8S)-5-(3,4-dichlorophenyl)-8-(methylamino)-5,6,7,8-tetrahydronaphthalen-2-yl)benzamide. ¹H NMR (CDCl₃, 400 Mhz) generated the following peaks: δ 7.85-7.87 (m, 3H), 7.76 (d, 1H), 7.48-7.55 (m, 3H), 7.33-7.36 (m, 2H), 7.24 (d, 1H), 6.97 (d, 1H), 6.81 (d, 1H), 3.96-4.00 (m, 1H), 3.74-3.76 (m, 1H), 2.55 (s, 3H), 1.83-2.04 (m, 4H). Mass spectrometry showed m/z=425.0 (M+H⁺).

### Example 19

### Synthesis of N-((5S,8S)-5-(3,4-dichlorophenyl)-8-(methylamino)-5,6,7,8-tetrahydronaphthalen-2-yl)cyclopentanecarboxamide

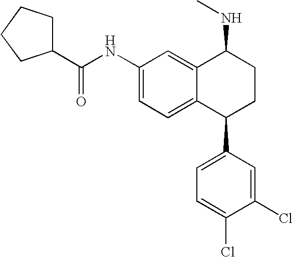

This compound was prepared in fashion analogous to that of (5S,8S)-5-(3,4-dichlorophenyl)-8-(methylamino)-N-(pyridin-2-yl)-5,6,7,8-tetrahydronaphthalene-2-carboxamide (Example 18). ¹H NMR (MeOH-d₄, 400 Mhz) generated the following peaks: δ 7.62 (s, 1H), 7.42 (d, 1H), 7.37 (d, 1H), 7.25 (dd, 1H), 7.13 (dd, 1H), 6.72 (d, 1H), 4.02 (t, 1H), 3.76-3.78 (m, 1H), 2.76-2.80 (m, 1H), 2.51 (s, 3H), 1.77-2.04 (m, 10H), 1.62-1.65 (m, 2H). Mass spectrometry showed m/z=417.1 (M+H⁺)

### Example 20

### Synthesis of N-((5S,8S)-5-(3,4-dichlorophenyl)-8-(dimethylamino)-5,6,7,8-tetrahydronaphthalen-2-yl)benzamide

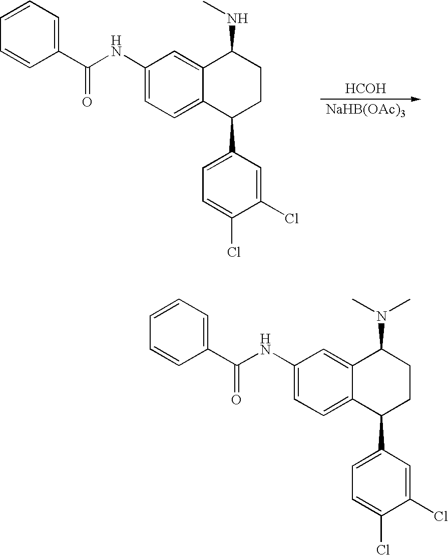

(5S,8S)-5-(3,4-dichlorophenyl)-8-(methylamino)-N-(pyridin-2-yl)-5,6,7,8-tetrahydronaphthalene-2-carboxamide (85.4 mg, 0.20 mmol), produced as described in Example 18, was dissolved in 1 ml DCM and was added 37% formaldehyde (14.7 μl, 0.18 mmol). The mixture was stirred at room temperature for 60 minutes, followed by addition of NaHB(OAc)₃ (153 mg, 0.72 mmol). The mixture was stirred overnight, quenched by addition of saturated sodium bicarbonate solution, and was extracted 3 times with ethyl acetate. The organic layer was separated, dried over anhydrous Na₂SO₄, and concentrated in vacuo. The residue was purified by flash chromatography and eluted with ethyl acetate:hexane (20-50%) to yield 25 mg of the desired product, N-((5S,8S)-5-(3,4-dichlorophenyl)-8-(dimethylamino)-5,6,7,8-tetrahydronaphthalen-2-yl)benzamide. ¹H NMR (CDCl₃, 400 Mhz) generated the following peaks: δ 7.87-7.89 (m, 3H), 7.79 (dd, 1H), 7.71 (d, 1H), 7.47-7.58 (m, 3H), 7.31 (d, 1H), 7.13 (d, 1H), 6.93 (d, 1H), 6.84 (dd, 1H), 4.09-4.14 (m, 1H), 3.78-3.82 (m, 1H), 2.32 (s, 6H), 1.62-2.14 (m, 4H). Mass spectrometry showed m/z=439.0 (M+H⁺).

### Example 21

### Synthesis of N-((5S,8S)-5-(3,4-dichlorophenyl)-8-(methylamino)-5,6,7,8-tetrahydro-naphthalen-2-yl)-1-methyl-1H-imidazole-4-sulfonamide

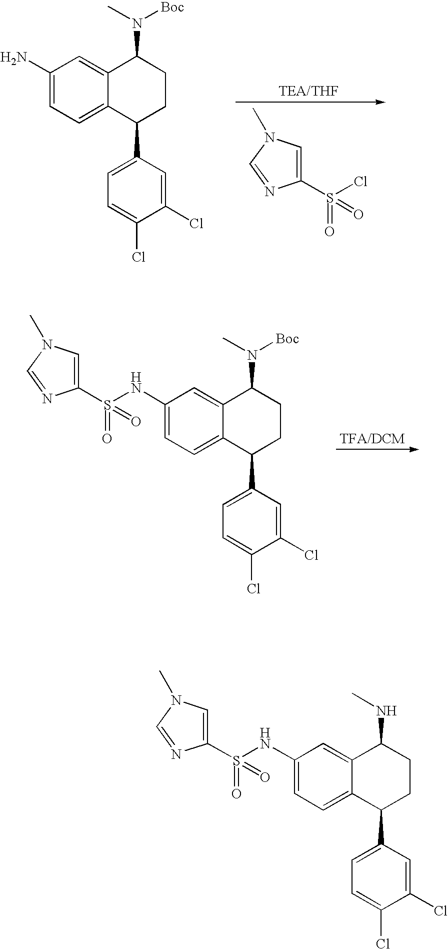

### tert-butyl (1S,4S)-4-(3,4-dichlorophenyl)-7-(1-methyl-1H-imidazole-4-sulfonamido)-1,2,3,4-tetrahydronaphthalen-1-yl(methyl)carbamate

1-methyl-1H-imidazole-4-sulfonyl chloride (186 μl, 1.43 mmol) and triethyl amine (298 μl, 2.14 mmol) was added to tert-butyl (1S,4S)-7-amino-4-(3,4-dichlorophenyl)-1,2,3,4-tetrahydronaphthalen-1-yl(methyl) carbamate (150 mg, 0.36 mmol, see PCT Publication No. WO 00/51972 for details) in 6 ml THF. The mixture was stirred at room temperature overnight and diluted with water, which was then extracted 3 times with ethyl acetate. The organic layer was separated, dried over anhydrous MgSO₄, and concentrated in vacuo. The residue was purified using flash chromatography eluted with ethyl acetate:hexane (40-60%) to yield 181 mg of the desired product, tert-butyl (1S,4S)-4-(3,4-dichlorophenyl)-7-(1-methyl-1H-imidazole-4-sulfonamido)-1,2,3,4-tetrahydronaphthalen-1-yl(methyl)carbamate. Mass spectrometry showed m/z=587.0 (M+Na⁺).

### N-((5S,8S)-5-(3,4-dichlorophenyl)-8-(methylamino)-5,6,7,8-tetrahydronaphthalen-2-yl)-1-methyl-1H-imidazole-4-sulfonamide

Trifluoroacetic acid (0.4 ml) was added to tert-butyl (1S,4S)-4-(3,4-dichlorophenyl)-7-(1-methyl-1H-imidazole-4-sulfonamido)-1,2,3,4-tetrahydronaphthalen-1-yl(methyl)carbamate (181 mg, 0.32 mmol) in 5 ml DCM. The mixture was stirred for 3 hours, diluted with DCM, and washed with saturated sodium bicarbonate solution and brine. The organic layer was separated, dried over anhydrous Na₂SO₄, and concentrated in vacuo. The residue was purified by flash chromatography eluted with MeOH:ethyl acetate (20%) to yield 93 mg of the desired product, N-((5S,8S)-5-(3,4-dichlorophenyl)-8-(methylamino)-5,6,7,8-tetrahydronaphthalen-2-yl)-1-methyl-1H-imidazole-4-sulfonamide. ¹H NMR (CDCl₃, 400 Mhz) generated the following peaks: δ 7.52 (d, 1H), 7.29-7.35 (m, 3H), 7.13 (dd, 1H), 7.02 (dd, 1H), 6.91 (dd, 1H), 6.63 (d, 1H), 3.88 (t, 1H), 3.65-3.67 (m, 4H), 2.44 (s, 3H), 1.79-1.99 (m, 4H). Mass spectrometry showed m/z=465.0 (M+H⁺).

### Example 22

### Synthesis of N-((5S,8S)-5-(3,4-dichlorophenyl)-8-(methylamino)-5,6,7,8-tetrahydronaphthalen-2-yl)benzenesulfonamide

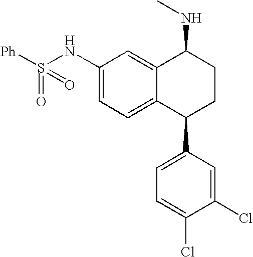

This compound was prepared in a manner analogous to N-((5S,8S)-5-(3,4-dichlorophenyl)-8-(methylamino)-5,6,7,8-tetrahydro-naphthalen-2-yl)-1-methyl-1H-imidazole-4-sulfonamide, as described in Example 21. ¹H NMR (MeOH-d₄, 400 Mhz) generated the following peaks: δ 8.50 (s, 1H), 7.78 (dd, 2H), 7.57 (t, 1H), 7.44-7.50 (m, 3H), 7.33 (dd, 2H), 7.13 (dd, 1H), 6.96 (dd, 1H), 6.74 (d, 1H), 4.30 (t, 1H), 4.06-4.10 (m, 1H), 2.71 (s, 3H), 1.83-2.21 (m, 4H). Mass spectrometry showed m/z=461.0 (M+H⁺).

### Example 23

### Synthesis of N-((5S,8S)-5-(3,4-dichlorophenyl)-8-(methylamino)-5,6,7,8-tetrahydronaphthalen-2-yl)cyclopropanesulfonamide

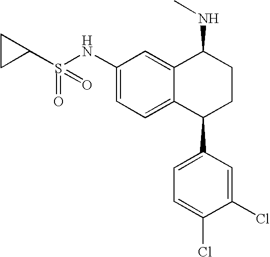

This compound was prepared in a manner analogous to N-((5S,8S)-5-(3,4-dichlorophenyl)-8-(methylamino)-5,6,7,8-tetrahydro-naphthalen-2-yl)-1-methyl-1H-imidazole-4-sulfonamide, as described in Example 21. ¹H NMR (MeOH-d₄, 400 Mhz) generated the following peaks: δ 8.33 (s, 1H), 7.40 (d, 1H), 7.28 (d, 2H), 7.13 (d, 1H), 7.03 (d, 1H), 6.89 (d, 1H), 4.28-4.30 (m, 1H), 4.08-4.10 (m, 1H), 2.71 (s, 3H), 2.51-2.59 (m, 1H), 1.80-2.10 (m, 4H), 0.92-0.94 (m, 4H). Mass spectrometry showed m/z=425.0 (M+H⁺).

### Example 24

### Synthesis of (5S,8S)-5-(3,4-dichlorophenyl)-8-(methylamino)-5,6,7,8-tetrahydronaphthalene-2-carbothioamide

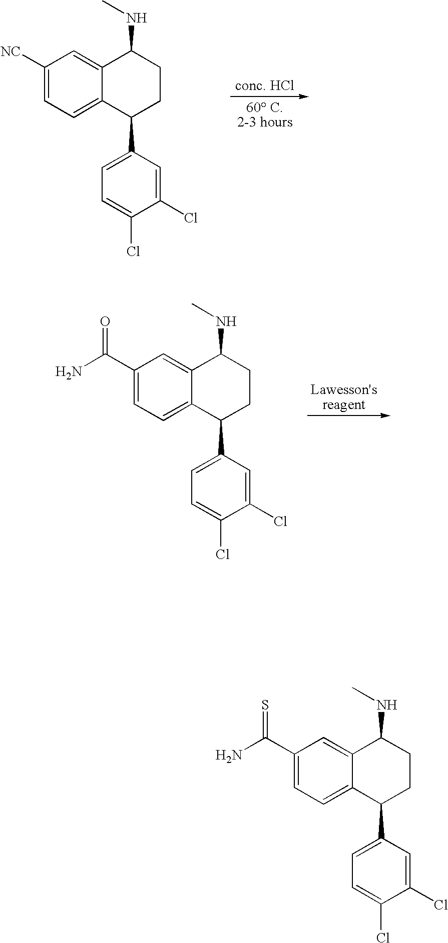

### (5S,8S)-5-(3,4-dichlorophenyl)-8-(methylamino)-5,6,7,8-tetrahydronaphthalene-2-carboxamide

(5S,8S)-5-(3,4-dichlorophenyl)-8-(methylamino)-5,6,7,8-tetrahydronaphthalene-2-carbonitrile (1 g, 3.01 mmol, described in PCT Publication No. WO 00/51972) was added to 20 ml concentrated HCl and the mixture was stirred in a 60° C. oil bath for 2-3 hours. The mixture was then cooled to room temperature and treated with sodium bicarbonate in an ice bath. The resulting mixture was extracted 3 times with ethyl acetate. The organic layer was separated, dried over anhydrous Na₂SO₄, and concentrated in vacuo. The residue was purified by flash chromatography eluted with MeOH: DCM (10-20%) to yield 404 mg of the desired product, (5S,8S)-5-(3,4-dichlorophenyl)-8-(methylamino)-5,6,7,8-tetrahydro-naphthalene-2-carboxamide. ¹H NMR (MeOH-d₄, 400 Mhz) generated the following peaks: δ 7.92 (d, 1H), 7.64 (dd, 1H), 7.45 (d, 1H), 7.39 (d, 1H), 7.15 (dd, 1H), 6.89 (d, 1H), 4.12 (t, 1H), 3.91-3.94 (m, 1H), 2.56 (s, 3H), 1.91-2.16 (m, 4H). Mass spectrometry showed m/z=349.0 (M+H⁺).

### (5S,8S)-5-(3,4-dichlorophenyl)-8-(methylamino)-5,6,7,8-tetrahydronaphthalene-2-carbothioamide

Lawesson's reagent (76 mg, 0.18 mmol) was added to (5S,8S)-5-(3,4-dichlorophenyl)-8-(methylamino)-5,6,7,8-tetrahydro-naphthalene-2-carbox-amide (65 mg, 0.19 mmol) in 3 ml THF. The mixture was stirred in a 55° C. oil bath for 6 hours. After being cooled to room temperature, the mixture was diluted with water, and the resulting mixture was extracted 3 times with ethyl acetate. The organic layer was separated, dried over anhydrous MgSO₄, and concentrated in vacuo. The residue was purified by flash chromatography and was eluted with MeOH:DCM (5-10%) to yield 20 mg of product which was further purified by HPLC (33% acetonitrile with 0.1% formic acid). The resulting formic acid salt was treated with NaOH and was extracted 3 times with ethyl acetate. The organic layer was separated and dried over anhydrous MgSO₄, and concentrated in vacuo, resulting in 8 mg of the desired product, (5S,8S)-5-(3,4-dichlorophenyl)-8-(methylamino)-5,6,7,8-tetrahydronaphthalene-2-carbothioamide. ¹H NMR (MeOH-d₄, 400 Mhz) generated the following peaks: δ 7.93 (d, 1H), 7.65 (dd, 1H), 7.44 (d, 1H), 7.39 (d, 1H), 7.15 (dd, 1H), 6.81 (d, 1H), 4.08 (t, 1H), 3.81-3.83 (m, 1H), 2.52 (s, 3H), 1.88-2.12 (m, 4H). Mass spectrometry showed m/z=365.0 (M+H⁺).

### Example 25

### Synthesis of (1S,4S)-4-(3,4-dichlorophenyl)-N-methyl-7-(phenylsulfonylmethyl)-1,2,3,4-tetrahydronaphthalen-1-amine

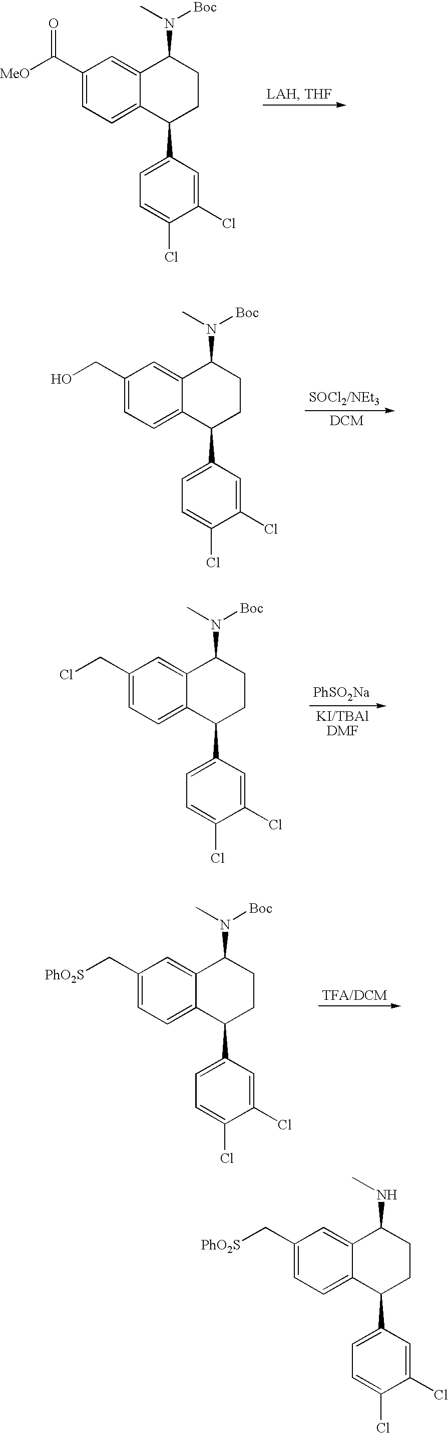

### tert-butyl (1S,4S)-4-(3,4-dichlorophenyl)-7-(hydroxymethyl)-1,2,3,4-tetrahydronaphthalen-1-yl(methyl)carbamate

1.0 M LAH solution (1.26 ml, 1.26 mmol) was added to (5S,8S)-methyl 8-(tert-butoxycarbonyl(methyl)amino)-5-(3,4-dichlorophenyl)-5,6,7,8-tetra-hydronaphthalene-2-carboxylate (Preparation 1 (Example 23), 450 mg, 0.969 mmol) in 10 ml THF. The mixture was stirred at room temperature overnight and then quenched by Rochelle solution and 1 N NaOH. The resulting mixture was extracted 3 times with ethyl acetate. The organic layer was separated and dried over anhydrous Na₂SO₄, and concentrated in vacuo. The residue was purified by flash chromatography and eluted with ethyl acetate:hexane (30-40%) to yield 350 mg of the desired product, tert-butyl (1S,4S)-4-(3,4-dichlorophenyl)-7-(hydroxymethyl)-1,2,3,4-tetrahydronaphthalen-1-yl (methyl)carbamate. ¹H NMR (CDCl₃, 400 Mhz) generated the following peaks: δ 7.32 (d, 1H), 7.19 (d, 2H), 7.08 (s, 1H), 6.95 (d, 1H), 6.82 (d, 1H), 5.28-5.50 (m, 1H), 4.69 (d, 2H), 4.16-4.18 (m, 1H), 2.62 (s, 3H), 1.73-2.27 (m, 4H), 1.52 (s, 9H).

### tert-butyl (1S,4S)-7-(chloromethyl)-4-(3,4-dichlorophenyl)-1,2,3,4-tetrahydronaphthalen-1-yl(methyl)carbamate

Triethyl amine (0.56 ml, 4.01 mmol) was added to tert-butyl (1S,4S)-4-(3,4-dichlorophenyl)-7-(hydroxymethyl)-1,2,3,4-tetrahydronaphthalen-1-yl (methyl)carbamate (350 mg, 0.80 mmol) in 15 ml DCM. The mixture was stirred at room temperature for 10 minutes and thionyl chloride (0.13 ml, 1.60 mmol) was added at 0° C. After being stirred at room temperature for 2 hours, the reaction mixture was quenched by saturated sodium bicarbonate solution. The resulting mixture was extracted 3 times with ethyl acetate. The organic layer was separated, dried over anhydrous Na₂SO₄, and concentrated in vacuo. The residue was purified by flash chromatography eluted with ethyl acetate:hexane (5-20%) to yield 250 mg of the desired product, tert-butyl (1S,4S)-7-(chloromethyl)-4-(3,4-dichlorophenyl)-1,2,3,4-tetrahydronaphthalen-1-yl(methyl)carbamate. ¹H NMR (CDCl₃, 400 Mhz) generated the following peaks: δ 7.33 (d, 1H), 7.21 (d, 2H), 7.09 (s, 1H), 6.95 (d, 1H), 6.80 (d, 1H), 5.28-5.50 (m, 1H), 4.58 (s, 2H), 4.16-4.18 (m, 1H), 2.62 (s, 3H), 1.73-2.27 (m, 4H), 1.52 (s, 9H).

### tert-butyl (1S,4S)-4-(3,4-dichlorophenyl)-7-(phenylsulfonylmethyl)-1,2,3,4-tetrahydro-naphthalen-1-yl(methyl)carbamate

Benzenesulfinic sodium salt (72 mg, 0.44 mmol), potassium iodide (37 mg, 0.22 mmol) and TBAI (16 mg, 0.044 mmol) was added to tert-butyl (1S,4S)-7-(chloromethyl)-4-(3,4-dichlorophenyl)-1,2,3,4-tetrahydronaphthalen-1-yl(methyl)carbamate (100 mg, 0.22 mmol) in 6 ml DMF was added. The mixture was stirred at room temperature for 1 hour, and the reaction mixture was then diluted with water. The resulting mixture was extracted 3 times with ethyl acetate. The organic layer was separated, dried over anhydrous Na₂SO₄, and concentrated in vacuo. The residue was purified by flash chromatography eluted with ethyl acetate:hexane (10-40%) to yield 50 mg of the desired product, tert-butyl (1S,4S)-4-(3,4-dichlorophenyl)-7-(phenylsulfonylmethyl)-1,2,3,4-tetrahydro-naphthalen-1-yl(methyl)carbamate. ¹H NMR (CDCl₃, 400 Mhz) generated the following peaks: δ 7.59-7.69 (m, 3H), 7.46-7.52 (m, 2H), 7.35 (d, 1H), 6.81-6.95 (m, 5H), 5.15-5.35 (m, 1H), 4.30 (s, 2H), 4.14-4.16 (m, 1H), 2.45 (d, 3H), 1.63-2.27 (m, 4H), 1.51 (d, 9H).

### (1S,4S)-4-(3,4-dichlorophenyl)-N-methyl-7-(phenylsulfonylmethyl)-1,2,3,4-tetrahydro-naphthalen-1-amine

Trifluoroacetic acid (0.8 ml) was added to tert-butyl (1S,4S)-4-(3,4-dichlorophenyl)-7-(phenylsulfonylmethyl)-1,2,3,4-tetrahydro-naphthalen-1-yl(methyl)carbamate (50 mg, 0.089 mmol) in 3 ml DCM. The mixtures was stirred for 1 hour in an ice bath, diluted with DCM, and washed with saturated sodium bicarbonate solution and brine. The organic layer was separated, dried over anhydrous Na₂SO₄, and concentrated in vacuo. The residue was purified by flash chromatography eluted with MeOH:DCM (4%) to yield 22 mg of the desired product, (1S,4S)-4-(3,4-dichlorophenyl)-N-methyl-7-(phenylsulfonylmethyl)-1,2,3,4-tetrahydro-naphthalen-1-amine. ¹H NMR (CDCl3, 400 Mhz) generated the following peaks: δ 7.59-7.67 (m, 3H), 7.46-7.50 (m, 2H), 7.35 (d, 1H), 7.15-7.17 (m, 2H), 6.96 (dd, 1H), 6.84 (dd, 1H), 6.71 (d, 1H), 4.27 (s, 2H), 3.93-3.97 (m, 1H), 3.72 (b, 1H), 2.47 (d, 3H), 1.80-2.01 (m, 4H). Mass spectrometry showed m/z=460.0 (M+H⁺).

### Example 26

### Synthesis of (S)-5-(3,4-dichlorophenyl)-8-isopropyl-5,6,7,8-tetrahydronaphthalene-2-carboxamide

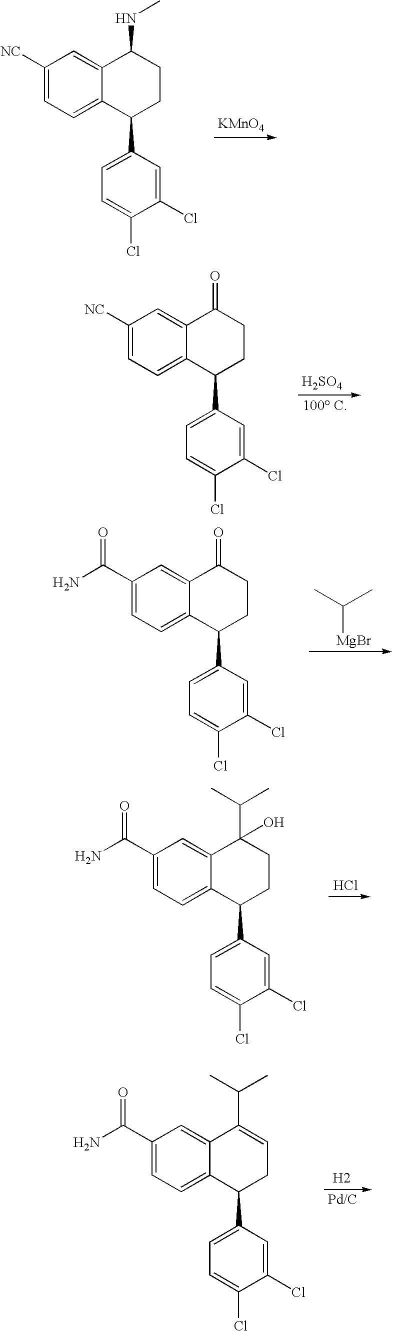

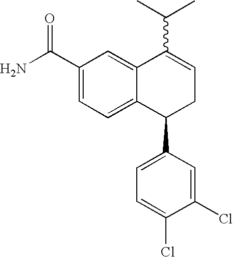

### (S)-5-(3,4-dichlorophenyl)-8-oxo-5,6,7,8-tetrahydronaphthalene-2-carbonitrile

(5S,8S)-5-(3,4-dichlorophenyl)-8-(methylamino)-5,6,7,8-tetrahydronaphthalene-2-carbonitrile (1.50 g, 4.53 mmol, described in PCT Publication No. WO 00/51972) was dissolved in acetone (37 ml). A solution of KMnO₄ (1.22 g, 7.70 mmol) in 37 ml water was added dropwise over 20 minutes. After stirring for 1 hour, the solids were filtered off and washed thoroughly with acetone and EA. The filtrate was concentrated in vacuo and brought to pH 1 using concentrated HCl. The mixture was warmed on a steam bath for 45 minutes. The cooled suspension was extracted 2 times with chloroform. The combined organic layers were dried over anhydrous Na₂SO₄, filtered and concentrated in vacuo. The residue was purified by flash chromatography eluted with EA: Hexane (10-20%) to yield 910 mg of the desired product, (S)-5-(3,4-dichlorophenyl)-8-oxo-5,6,7,8-tetrahydronaphthalene-2-carbonitrile. ¹H NMR (CDCl₃, 400 MHz) generated the following peaks: δ 8.41 (d, 1H), 7.70 (dd, 1H), 7.44 (d, 1H), 7.21 (d, 1H), 7.09 (d, 1H), 6.93 (dd, 1H), 4.30 (m, 1H), 2.65-2.82 (m, 2H), 2.47-2.52 (m, 1H), 2.26-2.32 (m, 1H).

### (S)-5-(3,4-dichlorophenyl)-8-oxo-5,6,7,8-tetrahydronaphthalene-2-carboxamide

(S)-5-(3,4-dichlorophenyl)-8-oxo-5,6,7,8-tetrahydronaphthalene-2-carbonitrile (910 mg, 2.88 mmol) was dissolved in concentrated H₂SO₄ (29.5 ml) and heated at 100° C. for 70 minutes. The cooled reaction was poured into water and neutralized with 2 N NaOH solution until pH 7. The mixture was extracted 3 times with EA. The combined organic layers were dried over anhydrous Na₂SO₄, filtered and concentrated in vacuo. The residue was dissolved in a little MeOH and filtered to yield 700 mg of the desired product, (S)-5-(3,4-dichlorophenyl)-8-oxo-5,6,7,8-tetrahydronaphthalene-2-carboxamide. ¹H NMR ((CD₃)₂SO, 300 MHz) generated the following peaks: δ 8.46 (s, 1H), 8.15 (s, 1H), 8.99 (d, 1H), 7.61 (d, 1H), 7.51 (s, 1H), 7.45 (s, 1H), 7.15 (d, 1H), 6.99 (d, 1H), 4.50 (t, 1H), 2.32-2.68 (m, 4H).

### (5S)-5-(3,4-dichlorophenyl)-8-hydroxy-8-isopropyl-5,6,7,8-tetrahydronaphthalene-2-carboxamide

A small amount of 2-bromopropane (1.97 ml, 20.95 mmol), in anhydrous THF (10 ml) was stirred with magnesium (458 mg, 18.85 mmol) at 55° C. until a reaction is started. The rest of the solution was added and stirred for 1 hour at 55° C. under nitrogen atmosphere until all the magnesium was consumed. A solution of (S)-5-(3,4-dichlorophenyl)-8-oxo-5,6,7,8-tetrahydronaphthalene-2-carboxamide (700 mg, 2.09 mmol) in anhydrous THF (10 ml) was slowly added to the Grignard preparation at 0° C. The mixture was warmed to room temperature and stirred for 3 hours under nitrogen atmosphere. The mixture was diluted with water and 10% NH₄Cl and extracted 3 times with diethyl ether. The combined organic layers were dried over anhydrous Na₂SO₄, filtered and concentrated in vacuo. The residue was purified by flash chromatography and eluted with EA:Hexane (20-40%) to yield 150 mg of the desired product, (5S)-5-(3,4-dichlorophenyl)-8-hydroxy-8-isopropyl-5,6,7,8-tetrahydronaphthalene-2-carboxamide.

### (S)-5-(3,4-dichlorophenyl)-8-isopropyl-5,6-dihydronaphthalene-2-carboxamide

(5S)-5-(3,4-dichlorophenyl)-8-hydroxy-8-isopropyl-5,6,7,8-tetrahydronaphthalene-2-carboxamide (150 mg, 0.40 mmol) was dissolved in 20 ml of 1 M HCl in diethyl ether. The mixture was stirred overnight. The mixture was diluted with NaHCO₃ and extracted 3 times with EA. The combined organic layers were dried over anhydrous Na₂SO₄, filtered and concentrated in vacuo. The residue was purified by flash chromatography and eluted with EA:Hexane (30-50%) to yield 92 mg of the desired product, (S)-5-(3,4-dichlorophenyl)-8-isopropyl-5,6-dihydronaphthalene-2-carboxamide. ¹H NMR (CDCl₃, 300 MHz) generated the following peaks: δ 7.91 (s, 1H), 7.51 (dd, 1H), 7.34 (d, 1H), 7.20 (d, 1H), 6.97 (d, 2H), 5.82 (t, 1H), 4.03 (t, 1H), 3.04-3.14 (m, 1H), 2.52-2.72 (m, 2H), 1.17 (d, 6H). Mass spectrometry showed m/z=360.1 (M+H⁺).

### (S)-5-(3,4-dichlorophenyl)-8-isopropyl-5,6,7,8-tetrahydronaphthalene-2-carboxamide

N-(5-(3,4-dichlorophenyl)-8-isopropyl-5,6-dihydronaphthalen-2-yl)acetamide (87 mg, 0.24 mmol) was dissolved in methanol. The reaction vessel was purged with nitrogen before Pd/C (200 mg) was added. Hydrogen gas was allowed to bubble through the solution for 2 hours. The mixture was filtered over celite and concentrated in vacuo. The residue was purified by flash chromatography and eluted with EA:Hexane (50%) to yield 36 mg of the desired product, (S)-5-(3,4-dichlorophenyl)-8-isopropyl-5,6,7,8-tetrahydronaphthalene-2-carboxamide. ¹H NMR (CDCl₃, 400 MHz) generated the following peaks: δ 7.82 (s, 1H), 7.44 (dd, 1H), 7.18-7.30 (m, 3H), 7.04 (dd, 2H), 6.99 (d, 1H), 4.17 (t, 1H), 2.72-2.75 (m, 1H), 2.37-2.39 (m, 1H), 2.00-2.08 (m, 2H), 1.70-1.74 (m, 2H), 1.05 (d, 3H), 0.84 (d, 3H).

### Example 27

### Synthesis of N-(5-(3,4-dichlorophenyl)-8-isopropyl-5,6,7,8-tetrahydronaphthalen-2-yl)acetamide

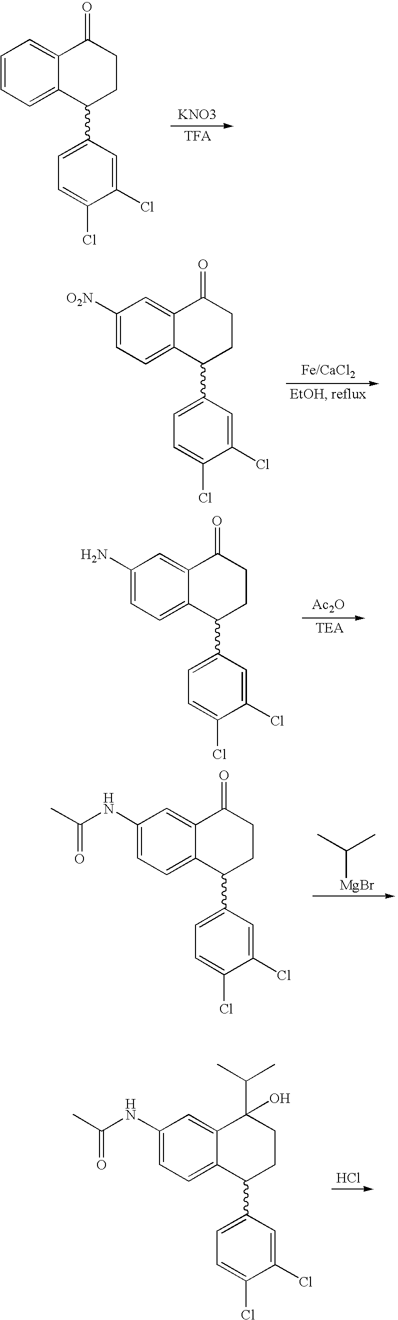

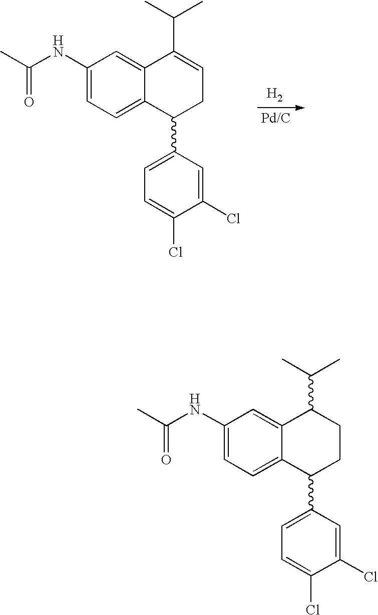

### 4-(3,4-dichlorophenyl)-7-nitro-3,4-dihydronaphthalen-1(2H)-one

A solution of 4-(3,4-dichlorophenyl)-3,4-dihydronaphthalen-1(2H)-one (5.00 g, 17.17 mmol) in TFA (52.5 ml) was cooled to 0° C. Trifluoromethanesulfonic acid (5.25 ml) was added followed by potassium nitrate (1.73 g, 17.17 mmol). The mixture was stirred for 1.5 hours under nitrogen atmosphere. The reaction was poured into a mixture of ice and ammonia solution. The mixture was extracted 3 times with EA. The combined organic layers were dried over anhydrous Na₂SO₄, filtered and concentrated in vacuo. The residue was purified by flash chromatography eluted with EA:Hexane (10%) to yield 1.70 g of the desired product, 4-(3,4-dichlorophenyl)-7-nitro-3,4-dihydronaphthalen-1(2H)-one. ¹H NMR (CDCl₃, 300 MHz) generated the following peaks: δ 7.53 (d, 1H), 6.81-6.93 (m, 4H), 6.32 (d, 1H), 3.72-3.77 (m, 1H), 2.05-2.09 (m, 2H), 1.87-1.95 (m, 1H), 1.63-1.72 (m, 1H).

### 7-amino-4-(3,4-dichlorophenyl)-3,4-dihydronaphthalen-1(2H)-one

4-(3,4-dichlorophenyl)-7-nitro-3,4-dihydronaphthalen-1(2H)-one (1.64 g, 4.88 mmol) was dissolved in 35 ml of 85% ethanol. Fe powder (2.45 g, 43.91 mmol) and CaCl₂ (271 mg, 2.44 mmol) were added and the mixture was reflux at 90° C. overnight under nitrogen atmosphere. The reaction was cooled and filtered over celite. The filtrate was dried over MgSO₄, filtered and concentrated in vacuo. The residue was purified by flash chromatography and eluted with EA:Hexane (10-25%) to yield 1.09 g of the desired product, 7-amino-4-(3,4-dichlorophenyl)-3,4-dihydronaphthalen-1(2H)-one. ¹H NMR (CDCl₃, 300 MHz) generated the following peaks: δ 8.11 (d, 1H), 7.35-7.50 (m, 2H), 7.01 (d, 1H), 6.66 (d, 1H), 6.34 (d, 1H), 4.13-4.16 (m, 1H), 2.21-2.77 (m, 4H).

### N-(5-(3,4-dichlorophenyl)-8-oxo-5,6,7,8-tetrahydronaphthalen-2-yl)acetamide

To a solution of 7-amino-4-(3,4-dichlorophenyl)-3,4-dihydronaphthalen-1(2H)-one (1.09 g, 3.56 mmol) in DCM (15 ml) was added triethylamine (2.5 ml) followed by acetic anhydride (1.3 ml). The mixture was stirred for 2 days. The mixture was diluted with water and extracted 3 times with EA. The combined organic layers were dried over anhydrous Na₂SO₄, filtered and concentrated in vacuo. The residue was purified by flash chromatography and eluted with EA:Hexane (30%) to yield 900 mg of the desired product, N-(5-(3,4-dichlorophenyl)-8-oxo-5,6,7,8-tetrahydronaphthalen-2-yl)acetamide. ¹H NMR (CDCl₃, 300 MHz) generated the following peaks: δ 8.28 (s, 1H), 8.12 (d, 1H), 7.69 (s, 1H), 7.36-7.50 (m, 2H), 6.97 (d, 1H), 6.86 (d, 1H), 4.27-4.31 (m, 1H), 2.24-2.77 (m, 4H), 2.24 (s, 3H). Mass spectrometry showed m/z=348.0 (M+H⁺).

### N-(5-(3,4-dichlorophenyl)-8-hydroxy-8-isopropyl-5,6,7,8-tetrahydronaphthalen-2-yl)acetamide

A small amount of 2-bromopropane (809 ul, 8.62 mmol), in anhydrous THF (8 ml) was stirred with magnesium (188 mg, 7.75 mmol) at 35° C. until a reaction is started. The rest of the solution was added and stirred for 30 minutes at 55° C. under nitrogen atmosphere until all the magnesium was consumed. A solution of N-(5-(3,4-dichlorophenyl)-8-oxo-5,6,7,8-tetrahydronaphthalen-2-yl)acetamide (300 mg, 0.86 mmol) in anhydrous THF (8 ml) was slowly added to the Grignard preparation at 0° C. The mixture was warmed to room temperature and stirred for 3 hours under nitrogen atmosphere. The mixture was diluted with water and 10% NH₄Cl and extracted 3 times with diethyl ether. The combined organic layers were dried over anhydrous Na₂SO₄, filtered and concentrated in vacuo. The residue was purified by flash chromatography and eluted with EA:Hexane (20-40%) to yield 60 mg of the desired product, N-(5-(3,4-dichlorophenyl)-8-hydroxy-8-isopropyl-5,6,7,8-tetrahydronaphthalen-2-yl)acetamide.

### N-(5-(3,4-dichlorophenyl)-8-isopropyl-5,6-dihydronaphthalen-2-yl)acetamide

N-(5-(3,4-dichlorophenyl)-8-hydroxy-8-isopropyl-5,6,7,8-tetrahydronaphthalen-2-yl)acetamide (60 mg, 0.15 mmol) was dissolved in 10 ml of 1 M HCl in diethyl ether. The mixture was stirred overnight. The mixture was diluted with NaHCO₃ and extracted 3 times with EA. The combined organic layers were dried over anhydrous Na₂SO₄, filtered and concentrated in vacuo. The residue was purified by flash chromatography and eluted with EA:Hexane (15-30%) to yield 20 mg of the desired product, N-(5-(3,4-dichlorophenyl)-8-isopropyl-5,6-dihydronaphthalen-2-yl)acetamide. Mass spectrometry showed m/z=374.1 (M+H⁺).

### N-(5-(3,4-dichlorophenyl)-8-isopropyl-5,6,7,8-tetrahydronaphthalen-2-yl)acetamide

N-(5-(3,4-dichlorophenyl)-8-isopropyl-5,6-dihydronaphthalen-2-yl)acetamide (20 mg, 0.05 mmol) was dissolved in methanol. The reaction vessel was purged with nitrogen before Pd/C (150 mg) was added. Hydrogen gas was allowed to bubble through the solution for 2 hours. The mixture was filtered over celite and concentrated in vacuo. The residue was purified by flash chromatography and eluted with EA:Hexane (30%) to yield 8 mg of the desired product, N-(5-(3,4-dichlorophenyl)-8-isopropyl-5,6,7,8-tetrahydronaphthalen-2-yl)acetamide. ¹H NMR (CDCl₃, 400 MHz) generated the following peaks: δ 7.46 (d, 1H), 7.15-7.31 (m, 2H), 7.05-7.09 (m, 2H), 6.91 (d, 1H), 6.80 (d, 1H), 4.11-4.14 (m, 1H), 2.68-2.70 (m, 1H), 2.31-2.33 (m, 1H), 2.14 (s, 3H), 1.95-2.09 (m, 2H), 1.67-1.71 (m, 2H), 1.04 (d, 3H), 0.82 (d, 3H).

### Example 28

### Synthesis of N-((5S,8S)-5-(3,4-dichlorophenyl)-8-((2-hydroxyethyl)(methyl)amino)-5,6,7,8-tetrahydronaphthalen-2-yl)acetamide

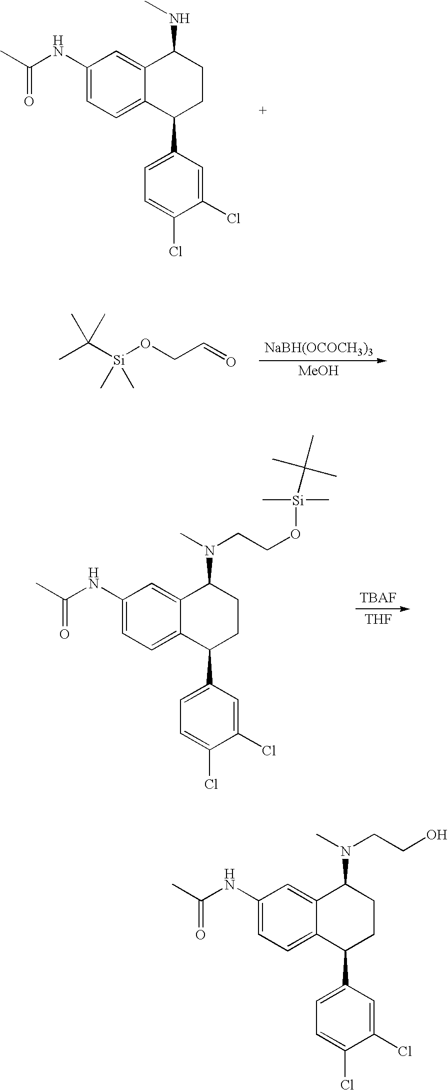

### N-((5S,8S)-8-((2-(tert-butyldimethylsilyloxy)ethyl)(methyl)amino)-5-(3,4-dichlorophenyl)-5,6,7,8-tetrahydronaphthalen-2-yl)acetamide

N-(5-(3,4-dichlorophenyl)-8-((2-hydroethyl)(methyl)amino)-5,6,7,8-tetrahydronaphthalen-2-yl)acetamide (43.5 mg, 0.12 mmol, described in PCT Publication No. WO 00/51972) in methanol (1 mL) was mixed with (tert-butyldimethylsilyloxy)acetaldehyde (15.8 μL, 0.083 mmol). The reaction mixture was stirred at room temperature for 1 hour. Sodium triacetoxyborohydride (70 mg, 0.33 mmol) was added to the mixture, and it was allowed to stir overnight at room temperature. The mixture was concentrated in vacuo before subsequent dilution with dichloromethane and extraction with water. The organic fractions were washed with brine and dried over anhydrous MgSO₄. The product was purified by silica gel flash chromatography using 10% ethyl acetate in hexane to yield 33 mg of the desired product. Mass spectrometry showed m/z=521.2 (M+H⁺).

### N-((5S,8S)-5-(3,4-dichlorophenyl)-8-((2-hydroxyethyl)(methyl)amino)-5,6,7,8-tetrahydronaphthalen-2-yl)acetamide

N-((5S,8S)-8-((2-(tert butyldimethylsilyloxy)ethyl)(methyl)amino)-5-(3,4-dichloro-phenyl)-5,6,7,8-tetrahydronaphthalen-2 yl)acetamide (33 mg, 0.063 mmol) in THF (0.5 mL) was added to 1M tetrabutylammonium fluoride in THF (0.095 mL, 0.095 mmol). The reaction mixture was stirred at room temperature for 1 hour. The reaction mixture was quenched with water and extracted with ethyl acetate. The organic fractions were washed with brine and dried over anhydrous MgSO4. The product was purified by silica gel flash chromatography using 65% ethyl acetate in hexane to yield 9.9 mg of the desired product. 1H NMR (MeOD, 400 MHz) δ 7.90 (d, 1H), 7.40 (dd, 1H), 7.13 (d, 1H), 6.94 (dd, 1H), 6.85 (d, 1H), 4.16 (t, 1H), 3.88 (s, 1H), 3.69-3.65 (m, 2H), 2.71-2.58 (m, 2H), 2.31 (s, 3H), 2.15-2.11 (m, 4H), 2.04-2.00 (m, 1H), 1.74-1.60 (m, 2H). Mass spectrometry showed m/z=407.1 (M+H⁺).

### Example 29

### Synthesis of 2-(((1S,4S)-4-(3,4-dichlorophenyl)-7-(methylsulfinylmethyl)-1,2,3,4-tetrahydronaphthalen-1-yl)(methyl)amino)ethanol

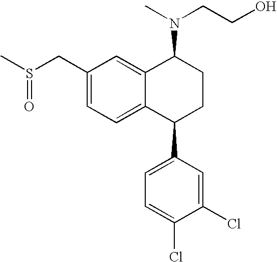

This compound was prepared in a manner analogous to N-((5S,8S)-5-(3,4-dichlorophenyl)-8-((2-hydroxyethyl)(methyl)amino)-5,6,7,8-tetrahydronaphthalen-2-yl)acetamide, as described in Example 28. ¹H NMR (MeOH-d₄, 400 Mhz) generated the following peaks: δ 7.84 (s, 1H), 7.39 (d, 1H), 7.14-7.17 (m, 2H), 6.94 (dd, 2H), 4.13-4.21 (m, 2H), 3.99-4.04 (m, 2H), 3.67 (t, 2H), 2.62-2.70 (m, 2H), 2.58 (s, 3H), 2.35 (s, 3H), 1.98-2.06 (m, 2H), 1.64-1.77 (m, 2H). Mass spectrometry showed m/z=426.1 (M+H⁺).

### Example 30

### Synthesis of (5S,8S)-methyl 8-(tert-butoxycarbonyl(methyl)amino)-5-(3,4-dichlorophenyl)-5,6,7,8-tetrahydronaphthalene-2-carboxylate (Preparation 1)

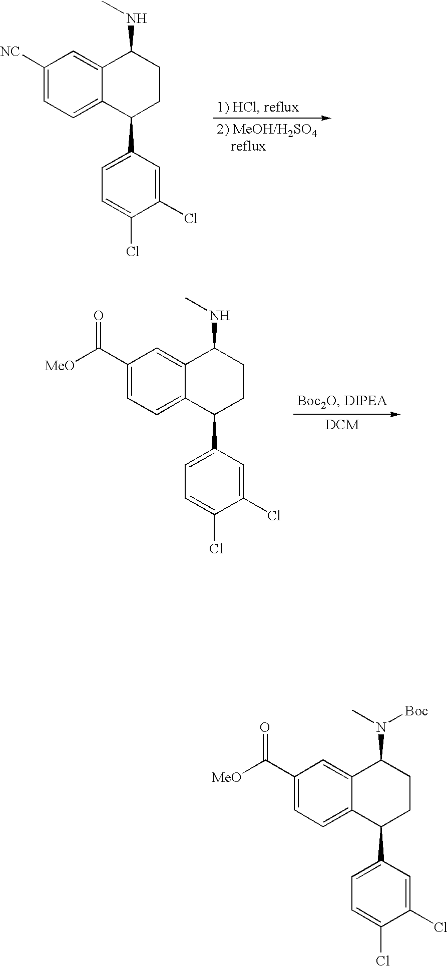

### (5S,8S)-methyl 5-(3,4-dichlorophenyl)-8-(methylamino)-5,6,7,8-tetrahydronaphthalene-2-carboxylate

(5S,8S)-5-(3,4-dichlorophenyl)-8-(methylamino)-5,6,7,8-tetrahydronaphthalene-2-carbonitrile (2.1 g, 6.34 mmol, WO 00/51972) was added to 40 ml concentrated HCl and the mixture was refluxed overnight. After being cooled to room temperature white solid was filtered, washed with cold water, and dried in vacuum. The white solid was dissolved in methanol (60 ml), to which 2 ml of concentrated H₂SO₄ was added. The mixture was refluxed overnight and then the reaction was quenched by sodium bicarbonate in ice bath. The resulting mixture was extracted 3 times with ethyl acetate. The organic layer was separated and dried over anhydrous Na₂SO₄, and concentrated in vacuo. The residue was purified by flash chromatography eluted with ethyl acetate:hexane (50%) to yield 1.5 g desired product, (5S,8S)-methyl 5-(3,4-dichlorophenyl)-8-(methylamino)-5,6,7,8-tetrahydronaphthalene-2-carboxylate. Mass spectrometry showed m/z=364.0 (M+H⁺).

### (5S,8S)-methyl 8-(tert-butoxycarbonyl(methyl)amino)-5-(3,4-dichlorophenyl)-5,6,7,8-tetrahydronaphthalene-2-carboxylate

DIPEA (1.12 ml, 8.24 mmol) was added to (5S,8S)-methyl 5-(3,4-dichlorophenyl)-8-(methylamino)-5,6,7,8-tetrahydronaphthalene-2-carboxylate (1.5 g, 4.12 mmol) in DCM (30 ml) and followed by addition of Boc₂O (1.35 g, 6.18 mmol) at 0° C. The mixture was stirred at room temperature for 3 hours, diluted with DCM, and washed with water and brine. The organic layer was separated and dried over anhydrous Na₂SO₄, and concentrated in vacuo. The residue was purified by flash chromatography and eluted with ethyl acetate:hexane (10%-40%) to yield 1.86 g desired product, (5S,8S)-methyl 8-(tert-butoxycarbonyl(methyl)amino)-5-(3,4-dichlorophenyl)-5,6,7,8-tetrahydronaphthalene-2-carboxylate. Mass spectrometry showed m/z=364.0 (M+H⁺-Boc).

### Example 31

### Synthesis of 3-((2-hydroxyethylamino)methyl)-4-(3-methyl-4-(methylthio)phenoxy)-benzenesulfonamide

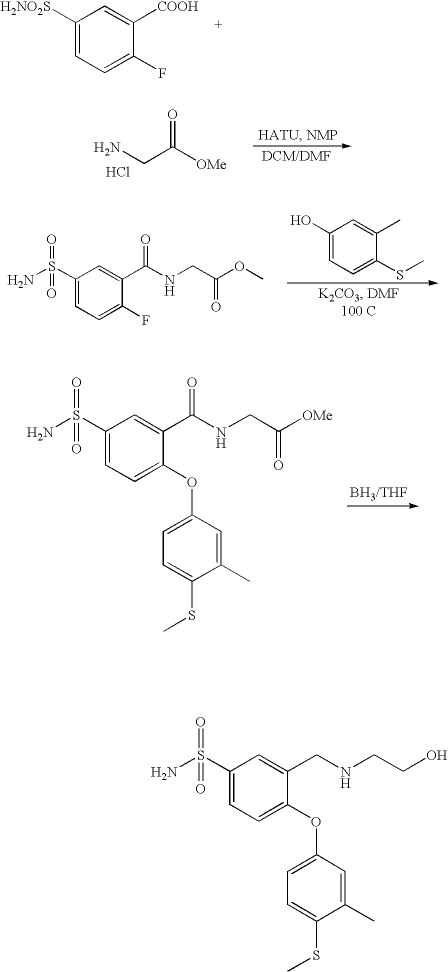

### Methyl 2-(2-fluoro-5-sulfamoylbenzamido)acetate

To 2-fluoro-5-sulfamoylbenzoic acid (250 mg, 1.14 mmol) in 3 mL DMF/DCM (1:1) was added HATU (542 mg, 1.42 mmol) and followed by Glycine methyl ester, hydrochloride (179 mg, 1.42 mmol) and 4-methyl morpholine (0.627mL, 5.70 mmol). The mixture was stirred at room temperature for 2 hours and diluted with water. The resulting mixture was extracted 3 times with ethyl acetate. The organic layer was separated and dried over anhydrous MgSO₄, and concentrated in vacuo. The residue was purified by flash chromatography eluted with Ethyl Acetate:Hexane (40%) to yield 280 mg desired product, methyl 2-(2-fluoro-5-sulfamoylbenzamido)acetate.

### Methyl 2-(2-(3-methyl-4-(methylthio)phenoxy)-5-sulfamoylbenzamido)acetate

A mixture of methyl 2-(2-fluoro-5-sulfamoylbenzamido)acetate (280 mg, 0.96 mmol), 3-methyl-4-(methylthio)phenol (164 mg, 1.06 mmol) and K₂CO₃ (160 mg, 1.15 mmol) in DMF (4 ml) was allowed to stir at 100° C. oil bath for 2 hours. Then the mixture was cooled to 0° C. then acidified to pH=1 by 2N HCl. The mixture was extracted 3 times with ethyl acetate. The organic layer was separated and dried over anhydrous MgSO₄, and concentrated in vacuo. The residue was purified by flash chromatography eluted with ethyl acetate:Hexane (50%) to yield 267 mg desired product, Methyl 2-(2-(3-methyl-4-(methylthio)phenoxy)-5-sulfamoylbenzamido)acetate. Mass spectrometry showed m/z=425.0 (M+H⁺).

### 3-((2-hydroxyethylamino)methyl)-4-(3-methyl-4-(methylthio)phenoxy)benzene-sulfonamide

Methyl 2-(2-(3-methyl-4-(methylthio)phenoxy)-5-sulfamoylbenzamido)acetate (247 mg, 0.58 mmol) was dissolved in 5 mL of anhydrous THF and cooled to 0° C., followed by dropwise addition of 17.5 mL of Borane THF complex (1.0M in THF). Then the mixture was warmed to room temperature and then refluxed for two days. The mixture was quenched by addition of 10N HCl (1.75 mL), and the mixture was refluxed for 1 hour. Then the solution was basified by K₂CO₃ until pH=9-10. The mixture was extracted 3 times with ethyl acetate. The organic layer was separated and dried over anhydrous MgSO₄, and concentrated in vacuo. The residue was purified by flash chromatography eluted with MeOH: DCM (10%) to yield 77.5 mg desired product, 3-((2-hydroxyethylamino)methyl)-4-(3-methyl-4-(methylthio)phenoxy)benzenesulfon-amide. ¹H NMR (DMSO-d₆, 400 MHz) generated the following peaks: δ 7.98 (d, 1H), 7.66 (dd, 1H), 7.24-7.28 (m, 3H), 6.85-6.95 (m, 3H), 4.59 (b, 1H), 3.86 (s, 2H), 3.48-3.53 (m, 2H), 2.66 (t, 2H), 2.46 (s, 3H), 2.25 (s, 3H). Mass spectrometry showed m/z=383.1 (M+H⁺).

### Example 32

### Synthesis of 3-(((2-hydroxyethyl)(methyl)amino)methyl)-4-(3-methyl-4-(methylthio) phenoxy)benzenesulfonamide

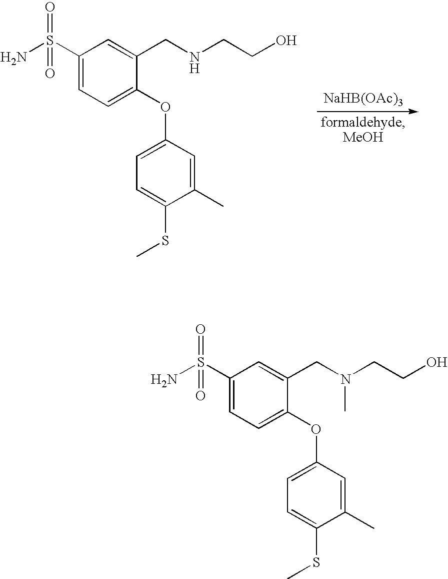

3-((2-hydroxyethylamino)methyl)-4-(3-methyl-4-(methylthio)phenoxy)benzenesulfon-amide (Example 29, 67 mg, 0.17 mmol) was dissolved in 1 mL MeOH and was added to 37% formaldehyde (12.8 uL, 0.16 mmol). The mixture was stirred at room temperature for 1 hour and followed by addition of NaHB(OAc)₃ (148 mg, 0.70 mmol). Then the mixture was stirred for 4 hours. The mixture was quenched by addition of water. The mixture was extracted 3 times with ethyl acetate. The organic layer was separated and dried over anhydrous MgSO₄, and concentrated in vacuo. The residue was purified by flash chromatography eluted with ethyl acetate:Hexane (80%) and ethyl acetate:Hexane:triethylamine (90:10:1) to yield 16.7 mg desired product, 3-(((2-hydroxyethyl)(methyl)amino)methyl)-4-(3-methyl-4-(methylthio)phenoxy)benzenesul-fonamide. ¹H NMR (CDCl₃, 300 MHz) generated the following peaks: δ 8.02 (d, 1H), 7.74 (dd, 1H), 7.18 (d, 1H), 6.83-6.86 (m, 3H), 3.73 (s, 2H), 3.64 (t, 2H), 2.66 (t, 2H), 2.46 (s, 3H), 2.33 (d, 6H). Mass spectrometry showed m/z=397.1 (M+H⁺).

### Example 33

### Synthesis of 2-(3-methyl-4-(methylthio)phenoxy)-5-sulfamoylbenzoic acid (Preparation 2)

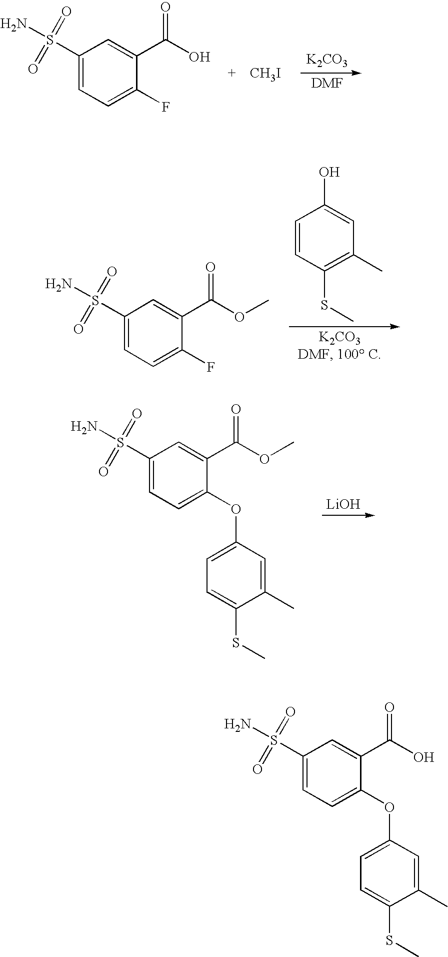

### Methyl 2-fluoro-5-sulfamoylbenzoate

2-fluoro-5-sulfamoyl benzoic acid (3.5 g, 16 mmol) in anhydrous DMF (30 mL) were added to iodomethane (1.09 mL, 17.6 mmol) and potassium carbonate (2.65 g, 19.2 mmol). The reaction was then stirred overnight at room temperature and quenched with water. The organic layer was separated, the aqueous fraction was extracted with ethyl acetate, and the organic layers were combined and dried with anhydrous MgSO₄. The desired product (2.57 g) was purified by silica gel flash chromatography using 50% ethyl acetate in hexanes. ¹H NMR (CDCl₃, 300 MHz) generated the following peaks: δ 8.53 (dd, 1H), 8.05-8.11 (m, 1H), 7.31 (d, 1H), 5.14 (s, 2H), 3.96 (s, 3H).

### Methyl 2-(3-methyl-4-(methylthio)phenoxy)-5-sulfamoylbenzoate

Methyl 2-fluoro-5-sulfamoylbenzoate (2.57 g, 11 mmol) in anhydrous DMF (15 mL) were added to 3-methyl-4-(methylthio)phenol (1.87 g, 12.1 mmol) and potassium carbonate (1.82 g, 13.2 mmol). The reaction was then stirred overnight at 100° C. and acidified with 1M HCl to pH 1. The organic layer was separated, the aqueous fraction was extracted with ethyl acetate, and the organic layers were combined and dried with anhydrous MgSO₄. The desired product (2.00 g) was purified by silica gel flash chromatography using 50% ethyl acetate in hexane. ¹H NMR (CDCl₃, 300 MHz) generated the following peaks: δ 8.45 (d, 1H), 7.91 (dd, 1H), 7.20 (d, 1H), 6.88-6.93 (m, 3H), 4.88 (s, 2H), 3.91 (s, 3H), 2.47 (s, 3H), 2.34 (s, 3H).

### 2-(3-methyl-4-(methylthio)phenoxy)-5-sulfamoylbenzoic acid

Methyl 2-(3-methyl-4-(methylthio)phenoxy)-5-sulfamoylbenzoate (2.00 g, 5.44 mmol) in 10% H₂O in methanol was added to lithium hydroxide (1.96 g, 81.6 mmol). The reaction was then stirred overnight at room temperature and quenched with 0.1N HCl. The organic layer was separated. The aqueous fraction was extracted with ethyl acetate and the organic layers were combined and dried with anhydrous MgSO₄. The desire product (1.36 g) was purified by silica gel flash chromatography using 20% methanol in ethyl acetate. ¹H NMR (CD₃OD, 300 MHz) generated the following peaks: δ 8.16 (d, 1H), 7.78 (dd, 1H), 7.24 (d, 1H), 6.87-6.91 (m, 3H), 2.43 (s, 3H), 2.29 (s, 3H); Mass spectrometry showed m/z=354.2 (M+H⁺).

### Example 34

### Synthesis of (S)-3-((2-hydroxypropylamino)methyl)-4-(3-methyl-4-(methylthio) phenoxy)benzenesulfonamide

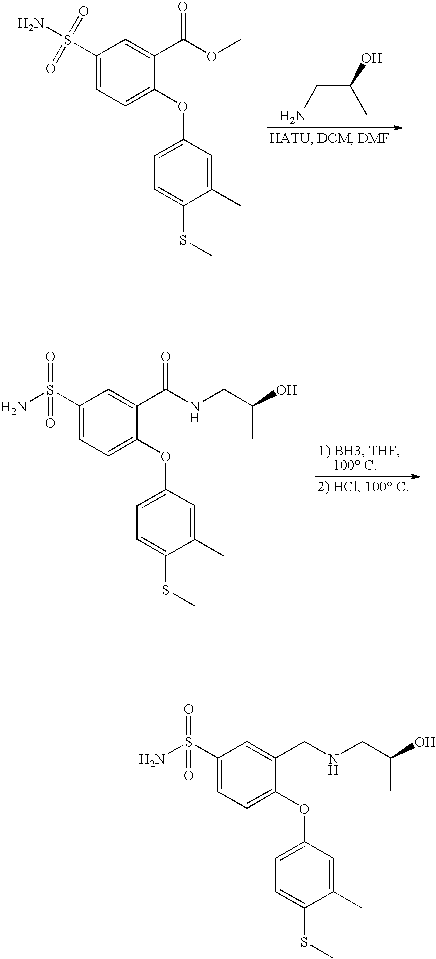

### (S)—N-(2-hydroxypropyl)-2-(3-methyl-4-(methylthio)phenoxy)-5-sulfamoylbenzamide

To a mixture of 2-(3-methyl-4-(methylthio)phenoxy)-5-sulfamoylbenzoic acid (100 mg, 0.28 mmol, Preparation 2) and HATU (161 mg, 0.42 mmol) in 1:1 anhydrous DCM: DMF (3 ml) was added to (S)-1-aminopropan-2-ol (25 ul, 0.31 mmol) and 4-methylmorpholine (124 ul, 1.13 mmol). The mixture was stirred overnight. The mixture was diluted with water and extracted 3 times with EA. The combined organic layers were dried over anhydrous Na₂SO₄, filtered and concentrated in vacuo. The residue was purified by flash chromatography eluted with MeOH:DCM (2%) to yield 96 mg of the desired product, (S)—N-(2-hydroxypropyl)-2-(3-methyl-4-(methylthio)phenoxy)-5-sulfamoylbenzamide. Mass spectrometry showed m/z=411.0 (M+H⁺).

### (S)-3-((2-hydroxypropylamino)methyl)-4-(3-methyl-4-(methylthio)phenoxy)benzenesulfonamide

A solution of (S)—N-(2-hydroxypropyl)-2-(3-methyl-4-(methylthio)phenoxy)-5-sulfamoylbenzamide (96 mg, 0.23 mmol) in THF (3 ml) was treated with 1 M BH₃.THF complex (935 ul, 0.94 mmol) at room temperature. The mixture was refluxed at 100° C. for 5 hours. The mixture was then cooled to room temperature and treated cautiously with 6 M HCl solution (2 ml). The resulting mixture was refluxed at 100 for 30 minutes. The mixture was cooled to room temperature, diluted with water and basified by the cautious addition of K₂CO₃ to pH 9. The mixture was extracted 3 times with EA. The combined organic layers were dried over anhydrous Na₂SO₄, filtered and concentrated in vacuo. The residue was purified by flash chromatography eluted with MeOH:EA (0-10%) to yield 50 mg of the desired product, (S)-3-((2-hydroxypropylamino)methyl)-4-(3-methyl-4-(methylthio)phenoxy)benzenesulfonamide. ¹H NMR (CD₃OD, 300 MHz) generated the following peaks: δ 7.95 (d, 1H), 7.74 (dd, 1H), 7.29 (d, 1H), 6.84-6.94 (m, 3H), 3.86-3.40 (m, 3H), 2.55-2.61 (m, 2H), 2.46 (s, 3H), 2.32 (s, 3H), 1.15 (d, 3H). Mass spectrometry showed m/z=397.1 (M+H⁺).

### Example 35-57

Example 35-57 (Table 33) were prepared in a manner analogous to (S)-3-((2-hydroxypropylamino)methyl)-4-(3-methyl-4-(methylthio)phenoxy)benzenesulfonamide, as described in Example 34.

TABLE 33

| Example | | |
|---|---|---|
| number | Compound | Data |
| 35 | 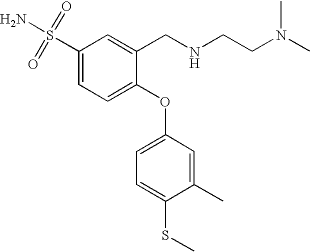 | δ_{H} (DMSO-d₆, 300 MHz) 7.95(d, 1 H), 7.65(dd, 1 H), 7.24-7.27(m, 3 H), 6.86-6.93(m, 3 H), 3.79(s, 2 H), 2.60(t, 2 H), 2.45(s, 3 H), 2.32(t, 2 H), 2.25(s, 3 H), 2.09(s, 6 H); MS m/z 410.1 (M + H⁺) |
| 36 | 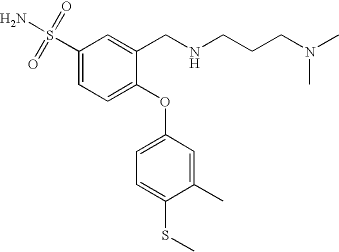 | δ_{H} (DMSO-d₆, 300 MHz) 7.97(d, 1 H), 7.65(dd, 1 H), 7.24-7.28(m, 3 H), 6.87-6.93(m, 3 H), 3.76(s, 2 H), 2.49-2.56(m, 2 H), 2.45(s, 3 H), 2.19-2.25(m, 5 H), 2.08(s, 6 H), 1.52-1.57(m, 2 H); MS m/z 424.1 (M + H⁺) |
| 37 | 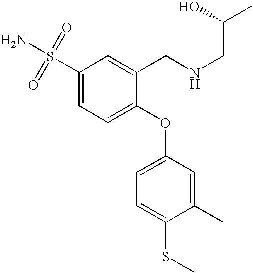 | δ_{H} (DMSO-d₆, 300 MHz) 7.97(d, 1 H), 7.65 (dd, 1 H), 7.24-7.28(m, 3 H), 6.85-6.93 (m, 3 H), 4.50(d, 1 H), 3.80 (s, 2 H), 3.65-3.75(m, 1 H), 2.45-2.28 (m, 5 H), 2.25 (s, 3 H), 0.82 (d, 3 H); MS m/z 397.0 (M + H⁺). |
| 38 | 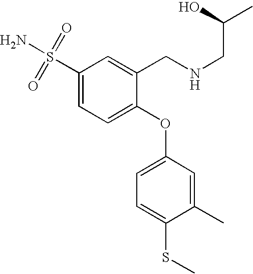 | δ_{H} (CD₃OD, 300 MHz) 7.95(d, 1 H), 7.74 (dd, 1 H), 7.28(d, 1 H), 6.84-6.93 (m, 3 H), 3.86-4.00 (m, 3 H), 2.55-2.61 (m, 2 H), 2.46(s, 3 H), 2.32 (s, 3 H), 1.12 (d, 3 H); MS m/z 397.0 (M + H⁺). |
| 39 | 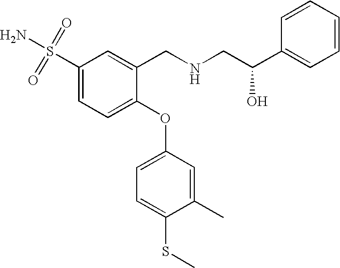 | δ_{H} (DMSO-d₆, 400 MHz) 7.97(s, 1 H), 7.65 (dd, 1 H), 7.21-7.33(m, 8 H), 6.84-6.91 (m, 3 H), 5.33(d, 1 H)), 4.64-4.67(m, 1 H), 4.36(t, 1 H), 3.84 (s, 2 H), 2.62-2.67(m, 2 H), 2.45(s, 3 H), 2.24 (s, 3 H); MS m/z 459.1 (M + H⁺). |
| 40 | 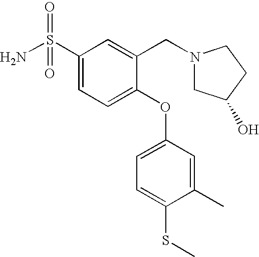 | δ_{H} (CD₃OD, 300 MHz) 8.03(d, 1 H), 7.73(dd, 1 H), 7.28(d, 1 H), 6.85-6.90(m, 3 H), 4.34-4.38(m, 1 H), 3.81(s, 2 H), 2.79-2.91(m, 2 H), 2.54-2.66(m, 2 H), 2.46(s, 3 H), 2.32(s, 3 H), 2.11-2.22(m, 1 H), 1.70-1.75(m, 1 H); MS m/z 409.0 (M + H⁺) |
| 41 | 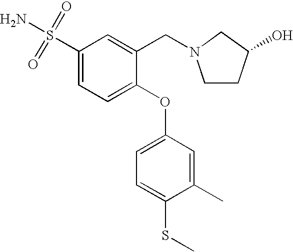 | δ_{H} (CD₃OD, 300 MHz) 8.03(d, 1 H), 7.73 (dd, 1 H), 7.27(d, 1 H), 6.85-6.89 (m, 3 H), 4.33-4.36(m, 1 H), 3.81 (s, 2 H), 2.79-2.89(m, 2 H), 2.56-2.66 (m, 2 H), 2.45 (s, 3 H), 2.32 (s, 3 H), 2.10-2.19 (m, 1 H), 1.73-1.76(m, 1 H); MS m/z 409.0 (M + H⁺). |
| 42 | 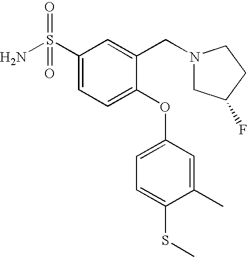 | δ_{H} (CDCl₃, 300 MHz) 8.08(d, 1 H), 7.72(dd, 1 H), 7.17-7.20(m, 1 H), 6.82-6.85(m, 3 H), 5.28 and 5.10(2 m, 1 H), 4.87(s, 2 H), 3.83(s, 2 H), 2.53-3.00(m, 4 H), 2.47(s, 3 H), 2.34(s, 3 H), 2.04-2.23(m, 2 H); MS m/z 411.0 (M + H⁺) |
| 43 | 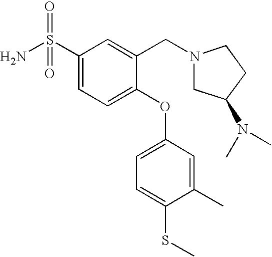 | δ_{H} (CD₃OD, 400 MHz) 8.00(d, 1 H), 7.73(dd, 1 H), 7.26(d, 1 H), 6.85-6.90(m, 3 H), 3.78(d, 2 H), 2.77-2.90(m, 3 H), 2.61-2.64(m, 1 H), 2.46-2.50(m, 1 H), 2.44(s, 3 H), 2.31(s, 3 H), 2.26(s, 6 H), 2.00-2.10(m, 1 H), 1.72-1.80(m, 1 H); MS m/z 436.1 (M + H⁺) |
| 44 | 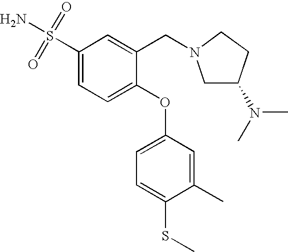 | δ_{H} (CD₃OD, 400 MHz) 8.00(d, 1 H), 7.73(dd, 1 H), 7.26(d, 1 H), 6.85-6.88(m, 3 H), 3.78(d, 2 H), 2.78-2.89(m, 3 H), 2.61-2.64(m, 1 H), 2.43-2.45(m, 4 H), 2.31(s, 3 H), 2.21(s, 6 H), 1.98-2.06(m, 1 H), 1.72-1.78(m, 1 H); MS m/z 436.1 (M + H⁺) |
| 45 | 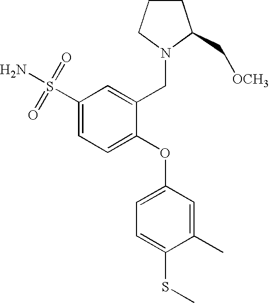 | δ_{H} (DMSO-d₆, 300 MHz) 7.92 (d, 1 H), 7.64 (dd, 1 H), 7.30 (s, 2 H),7.24 (d, 1 H), 6.85-6.92(m, 3 H), 4.07 (d, 1 H), 3.55 (d, 1 H), 3.31-3.38 (m, 1 H), 3.19-3.21 (m, 4 H), 2.88-2.94 (m, 1 H), 2.70-2.78 (m, 1 H), 2.44 (s, 3 H), 2.24 (s, 3 H), 2.18 (q, 1 H), 1.85-1.90(m, 1 H), 1.64-1.69(m, 2 H), 1.52-1.56(m, 1 H); MS m/z 437.1 (M + H⁺). |
| 46 | 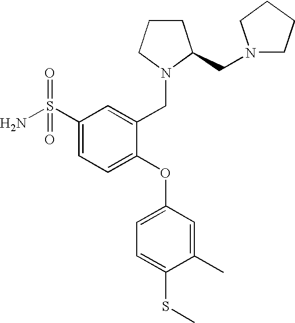 | δ_{H} (CD₃OD, 400 MHz) 8.01(d, 1 H), 7.74(dd, 1 H), 7.27(d, 1 H), 6.86-6.89(m, 3 H), 4.17(d, 1 H), 3.62(d, 1 H), 2.65-3.07(m, 8 H), 2.45(s, 3 H), 2.3 1(s, 3 H), 1.65-2.12(m, 9 H); MS m/z 476.1 (M + H⁺) |
| 47 | 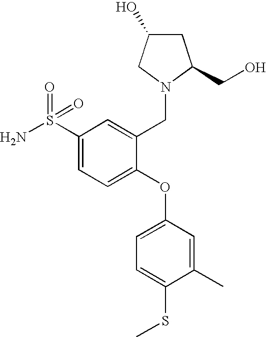 | δ_{H} (DMSO-d₆, 400 MHz) 7.91 (d, 1 H), 7.64 (dd, 1 H), 7.29 (s, 2 H), 7.24 (d, 1 H), 6.86-6.91 (m, 3 H), 4.69 (d, 1 H), 4.35 (t, 1 H), 4.05-4.13 (m, 2 H), 3.59 (d, 1 H), 3.41-3.46 (m, 1 H), 3.13-3.16 (m, 1 H), 2.85-2.88 (m, 1 H), 2.44 (s, 3 H), 2.24 (s, 3 H), 2.10-2.14 (m, 1 H), 1.70-1.73 (m, 2 H); MS m/z 439.1 (M + H⁺). |
| 48 | 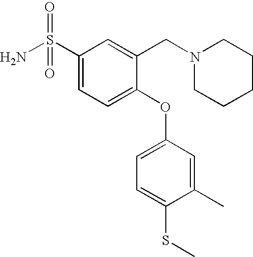 | δ_{H} (CD₃OD, 300 MHz) 8.01(d, 1 H), 7.74(dd, 1 H), 7.27(d, 1 H), 6.86-6.89(m, 3 H), 3.66(s, 2 H), 2.2.46-2.58(m, 4 H), 2.45(s, 3 H), 2.32(s, 3 H), 1.59-1.63(m, 4 H), 1.45-1.48(m, 2 H); MS m/z 407.1 (M + H⁺) |
| 49 | 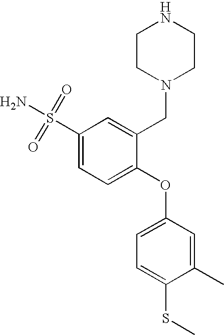 | δ_{H} (DMSO-d₆, 400 MHz) 7.91 (d, 1 H), 7.66 (dd, 1 H), 7.30 (s, 2 H), 7.24 (d, 1 H), 6.84-6.91 (m, 3 H), 3.51 (s, 2 H), 2.67 (b, 4 H), 2.44 (s, 3 H), 2.33 (b, 4 H), 2.18 (s, 3 H); MS m/z 408.1 (M + H⁺). |
| 50 | 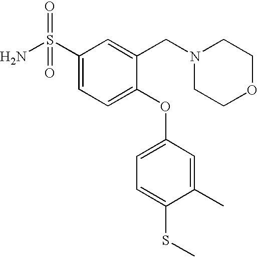 | δ_{H} (CDCl₃, 400 MHz) 8.07(d, 1 H), 7.71(dd, 1 H), 7.17-7.21(m, 1 H), 6.80-6.84 (m, 3 H), 3.67-3.70(m, 4 H), 3.64(s, 2 H), 3.10-3.13(m, 4 H), 2.50-2.53(m, 4 H), 2.45(s, 3 H), 2.32(s, 3 H); MS m/z 409.0 (M + H⁺) |
| 51 | 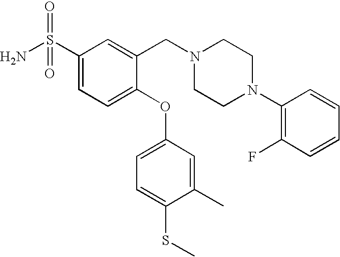 | δ_{H} (CDCl₃, 300 MHz) 8.11(d, 1 H), 7.73(dd, 1 H), 7.17-7.21(m, 1 H), 6.85-7.08(m, 7 H), 4.84(s, 2 H), 3.75(s, 2 H), 3.10-3.13(m, 4 H), 2.73-2.75(m, 4 H), 2.47(s, 3 H), 2.35(s, 3 H); MS m/z 502.1 (M + H⁺) |
| 52 | 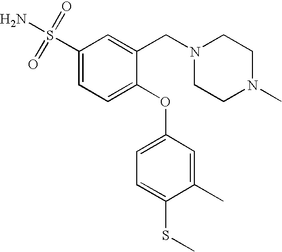 | δ_{H} (CDCl₃, 400 MHz) 8.06(d, 1 H), 7.71 (dd, 1 H), 7.18(d, 1 H), 6.81-6.85 (m, 3 H), 3.68 (s, 2 H), 2.59 (b, 4 H), 2.49(b, 4 H), 2.46 (s, 3 H), 2.33 (s, 3 H), 2.30 (s, 3 H); MS m/z 422.1 (M + H⁺). |
| 53 | 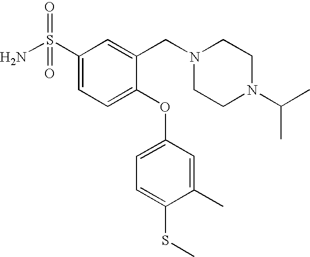 | δ_{H} (CD₃OD, 400 MHz) 7.98(d, 1 H), 7.75(dd, 1 H), 7.26(d, 1 H), 6.84-6.92 (m, 3 H), 3.72(s, 2 H), 2.68-2.98(m, 9 H), 2.44(s, 3 H), 2.31(s, 3 H), 1.20(d, 6 H); MS m/z 450.1 (M + H⁺) |
| 54 | 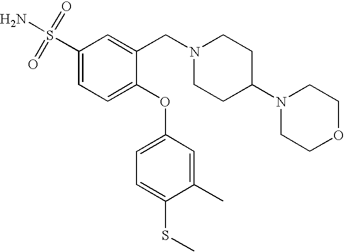 | δ_{H} (CD₃OD, 400 MHz) 8.00(d, 1 H), 7.74 (dd, 1 H), 7.26 (d, 1 H), 6.83-6.89 (m, 3 H), 3.65-3.69 (m, 6 H), 2.99-3.02 (m, 2 H), 2.53-2.56 (m, 4 H), 2.44 (s, 3 H), 2.31 (s, 3 H), 2.09-2.20 (m, 3 H), 1.85-1.90(m, 2 H), 1.49-1.56 (m, 2 H); MS m/z 492.1 (M + H⁺). |
| 55 | 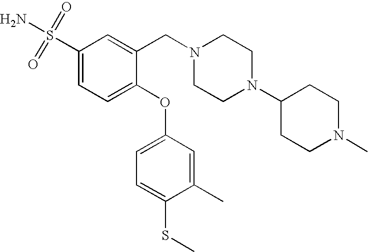 | δ_{H} (CD₃OD, 400 MHz) 7.99(d, 1 H), 7.74 (dd, 1 H), 7.26 (d, 1 H), 6.84-6.90 (m, 3 H), 3.68 (s, 2 H), 3.02-3.05 (m, 2 H), 2.61 (b, 8 H), 2.44 (s, 3 H), 2.37 (s, 3 H), 2.20-2.34 (m, 6 H), 1.90-1.94(m, 2 H), 1.56-1.60 (m, 2 H); MS m/z 505.2 (M + H⁺). |
| 56 | 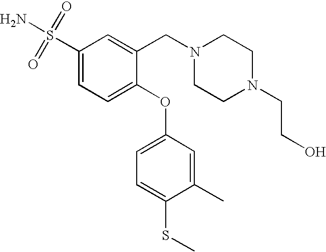 | δ_{H} (DMSO-d₆, 300 MHz) 7.91 (d, 1 H), 7.67 (dd, 1 H), 7.31 (s, 2 H), 7.25 (d, 1 H), 6.86-6.93 (m, 3 H), 4.34 (t, 1 H), 3.56 (s, 2 H), 3.46-3.50 (m, 2 H), 2.34-2.45 (m, 13 H), 2.25 (s, 3 H); MS m/z 452.1 (M + H⁺). |
| 57 | 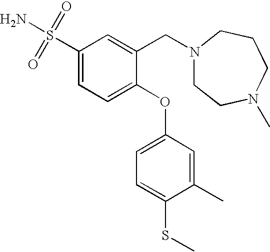 | δ_{H} (CD₃OD, 400 MHz) 8.05(d, 1 H), 7.75 (dd, 1 H), 7.26(d, 1 H), 6.83-6.91 (m, 3 H), 3.82 (s, 2 H), 2.96-3.06 (m, 4 H), 2.80-2.89 (m, 4 H), 2.60(s, 3 H), 2.44(s, 3 H), 2.31 (s, 3 H), 1.90-1.95 (m, 2 H); MS m/z 436.1 (M + H⁺). |

### Example 58-61

Example 58-61 (Table 34) were prepared in a manner analogous to 3-(((2-hydroxyethyl)(methyl)amino)methyl)-4-(3-methyl-4-(methylthio) phenoxy)benzenesulfonamide, as described in Example 32.

TABLE 34

| Example | Prepare | | |
|---|---|---|---|
| number | from | Structure | Data |
| 58 | 35 | 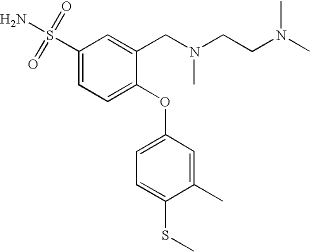 | δ_{H} (DMSO-d₆, 400 MHz) 7.93(d, 1 H), 7.65(dd, 1 H), 7.29(s, 2 H), 7.24 (d, 1 H), 6.84-6.90(m, 3 H), 3.58(s, 2 H), 2.48(t, 2 H), 2.45(s, 3 H), 2.33-2.36(m, 2 H), 2.24(s, 3 H), 2.18(s, 3 H), 2.08(s, 6 H); MS m/z 424.1 (M + H⁺) |
| 59 | 36 | 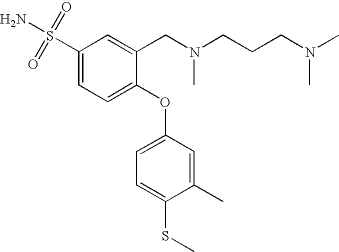 | δ_{H} (DMSO-d₆, 300 MHz) 7.94(d, 1 H), 7.66(dd, 1 H), 7.23-7.30(m, 3 H), 6.85-6.91(m, 3 H), 3.54(s, 2 H), 2.44(s, 3 H), 2.38(t, 2 H), 2.16-2.25(m, 8 H), 2.07(s, 6 H), 1.52-1.59(m, 2 H); MS m/z 438.1 (M + H⁺) |
| 60 | 37 | 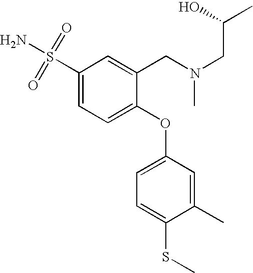 | δ_{H} (DMSO-d₆, 300 MHz) 7.97(d, 1 H), 7.66 (dd, 1 H), 7.23-7.28(m, 3 H), 6.86-6.91 (m, 3 H), 4.27(d, 1 H), 3.79(b, 1 H), 3.61 (s, 2 H), 2.45 (s, 3 H), 2.20-2.36 (m, 8 H), 1.04(d, 3 H); MS m/z 411.1 (M + H⁺). |
| 61 | 38 | 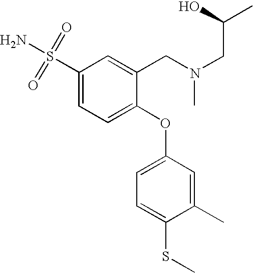 | δ_{H} (DMSO-d₆, 300 MHz) 7.97(d, 1 H), 7.66 (dd, 1 H), 7.23-7.29(m, 3 H), 6.86-6.90 (m, 3 H), 4.27(d, 1 H), 3.79(b, 1 H), 3.61 (s, 2 H), 2.45 (s, 3 H), 2.35(t, 2 H), 2.25(s, 3 H), 2.20 S(s, 3 H), 1.04(d, 3 H); MS m/z 411.1 (M + H⁺). |

### Example 62

### Synthesis of 3-isobutyl-4-(3-methyl-4-(methylthio)phenoxy)benzenesulfonamide

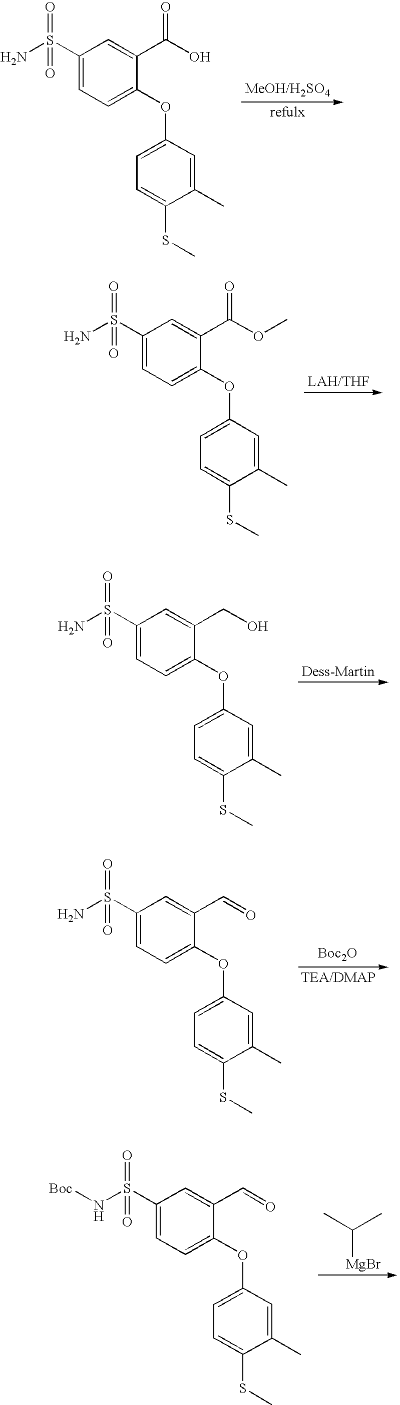

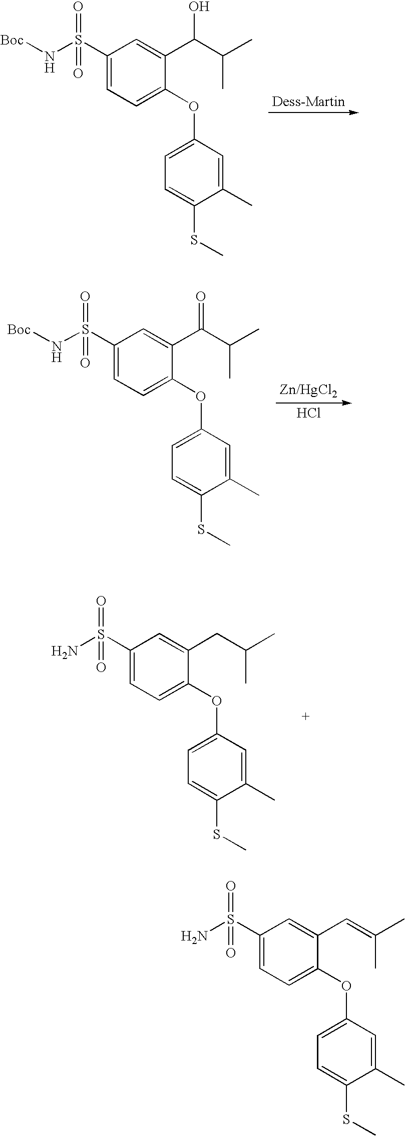

### Methyl 2-(3-methyl-4-(methylthio)phenoxy)-5-sulfamoylbenzoate

A solution of 2-(3-methyl-4-(methylthio)phenoxy)-5-sulfamoylbenzoic acid (500 mg, 1.41 mmol, Preparation 2) in MeOH (40 ml) and concentrated sulfuric acid (0.5 ml) was refluxed at 80° C. overnight. The mixture was concentrated in vacuo. The residue was purified by flash chromatography eluted with EA:Hexane (30-50%) to yield 500 mg of the desired product, methyl 2-(3-methyl-4-(methylthio)phenoxy)-5-sulfamoylbenzoate.

### 3-(hydroxymethyl)-4-(3-methyl-4-(methylthio)phenoxy)benzenesulfonamide

Methyl 2-(3-methyl-4-(methylthio)phenoxy)-5-sulfamoylbenzoate (500 mg, 1.36 mmol) was dissolved in anhydrous THF (10 ml) and cooled to 0° C., followed by drop-wise addition of 1 M LAH in THF (2.7 ml). The mixture was then warmed to room temperature and stirred for 2 hours under nitrogen atmosphere. The mixture was quenched by addition of water at 0° C., and then acidified to pH 1 using 2% HCl solution. The aqueous layer was extracted 3 times with DCM. The combined organic layers were dried over anhydrous Na₂SO₄, filtered and concentrated in vacuo. The residue was purified by flash chromatography and eluted with EA:Hexane (50%) to yield 400 mg of the desired product, 3-(hydroxymethyl)-4-(3-methyl-4-(methylthio)phenoxy)benzene-sulfonamide.

### 3-formyl-4-(3-methyl-4-(methylthio)phenoxy)benzenesulfonamide

To a solution of 3-(hydroxymethyl)-4-(3-methyl-4-(methylthio)phenoxy)benzenesul-fonamide (400 mg, 1.18 mmol) in DCM (10 ml) was added Dess-Martin periodinane (600 mg, 1.41 mmol). The mixture was stirred for 2 hours. The mixture was diluted with 10% sodium thiosulfate and saturated NaHCO₃. The aqueous layer was extracted 3 times with DCM. The combined organic layers were dried over anhydrous Na₂SO₄, filtered and concentrated in vacuo. The residue was purified by flash chromatography and eluted with EA:Hexane (20-60%) to yield 324 mg of the desired product, 3-formyl-4-(3-methyl-4-(methylthio)phenoxy)benzenesulfonamide.

### tert-butyl 3-formyl-4-(3-methyl-4-methylthio)phenoxy)phenylsulfonylcarbamate

To a solution of 3-formyl-4-(3-methyl-4-(methylthio)phenoxy)benzenesulfonamide (324 mg, 0.96 mmol) in DCM (10 ml) was added di-tert-butyl dicarbonate (241 mg, 1.10 mmol), triethylamine (147 ul, 1.06 mmol) and DMAP (12 mg, 0.10 mmol). The mixture was stirred overnight. The mixture was diluted with water and extracted 3 times with DCM. The combined organic layers were dried over anhydrous Na₂SO₄, filtered and concentrated in vacuo. The residue was purified by flash chromatography and eluted with EA:Hexane (30-60%) to yield 305 mg of the desired product, tert-butyl 3-formyl-4-(3-methyl-4-(methylthio)phenoxy)phenylsulfonylcarbamate.

### tert-butyl 3-(1-hydroxy-2-methylpropyl)-4-(3-methyl-4-(methylthio)phenoxy)phenylsulfonylcarbamate

A small amount of 2-bromopropane (655 ul, 6.97 mmol), in anhydrous THF (8 ml) was stirred with magnesium (136 mg, 5.58 mmol) at 35° C. until a reaction is started. The rest of the solution was added and stirred for 30 minutes at 35° C. under nitrogen atmosphere until all the magnesium was consumed. A solution of tert-butyl 3-formyl-4-(3-methyl-4-(methylthio)phenoxy)phenylsulfonylcarbamate (305 mg, 0.70 mmol) in anhydrous THF (8 ml) was slowly added to the Grignard preparation at 0° C. The mixture was warmed to room temperature and stirred for 30 minutes under nitrogen atmosphere. The mixture was diluted with water and 10% NH₄Cl and extracted 3 times with diethyl ether. The combined organic layers were dried over anhydrous Na₂SO₄, filtered and concentrated in vacuo. The residue was purified by flash chromatography and eluted with EA:Hexane (30%) to yield 90 mg of the desired product, tert-butyl 3-(1-hydroxy-2-methylpropyl)-4-(3-methyl-4-(methylthio)phenoxy)phenylsulfonylcarbamate. ¹H NMR (CDCl₃, 400 MHz) generated the following peaks: δ 8.11 (d, 1H), 7.81 (dd, 1H), 7.20 (d, 1H), 6.78-6.91 (m, 3H), 4.87 (d, 1H), 2.48 (s, 3H), 2.35 (s, 3H), 2.15 (m, 2H), 1.40 (s, 9H), 0.96 (dd, 6H).

### tert-butyl 3-isobutyryl-4-(3-methyl-4-(methylthio)phenoxy)phenylsulfonyl-carbamate

To a solution of tert-butyl 3-(1-hydroxy-2-methylpropyl)-4-(3-methyl-4-(methylthio) phenoxy)phenylsulfonylcarbamate (90 mg, 0.19 mmol) in DCM (3 ml) was added Dess-Martin periodinane (95 mg, 0.22 mmol). The mixture was stirred for 2 hours. The mixture was diluted with 10% sodium thiosulfate and saturated NaHCO₃. The aqueous layer was extracted 3 times with DCM. The combined organic layers were dried over anhydrous Na₂SO₄, filtered and concentrated in vacuo. The residue was purified by flash chromatography and eluted with EA:Hexane (20-30%) to yield 62 mg of the desired product, tert-butyl 3-isobutyryl-4-(3-methyl-4-(methylthio)phenoxy)phenylsulfonyl-carbamate. ¹H NMR (CDCl₃, 300 MHz) generated the following peaks: δ 8.22 (d, 1H), 7.98 (dd, 1H), 7.22 (d, 1H), 6.88-6.94 (m, 3H), 3.51 (m, 1H), 2.49 (s, 3H), 2.36 (s, 3H), 1.42 (s, 9H), 1.20 (d, 6H).

### 3-isobutyl-4-(3-methyl-4-(methylthio)phenoxy)benzenesulfonamide and 4-(3-methyl-4-(methylthio)phenoxy)-3-(2-methylprop-1-enyl)benzenesulfonamide

A mixture of zinc (169 mg, 2.59 mmol), mercury(II) chloride, concentrated HCl (1 drop) and water (1 ml) was stirred for 5 minutes. The solution was decanted and to it was added water (2.4 ml), concentrated HCl (0.6 ml), tert-butyl 3-isobutyryl-4-(3-methyl-4-(methylthio)phenoxy)phenylsulfonylcarbamate (62 mg, 0.13 mmol) in toluene (0.9 ml) and glacial acetic acid (1 drop) sequentially. The mixture was heated at 100° C. overnight. The reaction was diluted with water and extracted 3 times with diethyl ether. The combined organic layers were dried over anhydrous Na₂SO₄, filtered and concentrated in vacuo. The residue was purified by flash chromatography eluted with EA:Hexane (10-30%) to yield 10 mg of a mixture of products. The crude product was further purified using reverse phase HPLC to yield 5 mg of the desired product, 3-isobutyl-4-(3-methyl-4-(methylthio)phenoxy)benzenesulfonamide. ¹H NMR (CDCl₃, 300 MHz) generated the following peaks: δ 7.77 (d, 1H), 7.65 (dd, 1H), 7.19 (d, 1H), 6.80-6.84 (m, 3H), 4.77 (s, 2H), 2.61 (d, 2H), 2.47 (s, 3H), 2.35 (s, 3H), 2.01 (m, 1H), 0.94 (d, 6H). Mass spectrometry showed m/z=366.1 (M+H⁺).

5 mg of a side product, 4-(3-methyl-4-(methylthio)phenoxy)-3-(2-methylprop-1-enyl)benzenesulfonamide was obtained. ¹H NMR (CDCl₃, 300 MHz) generated the following peaks: δ 7.84 (d, 1H), 7.66 (dd, 1H), 7.18 (d, 1H), 6.82-6.85 (m, 3H), 6.30 (s, 1H), 4.78 (s, 2H), 2.46 (s, 3H), 2.34 (s, 3H), 1.88 (d, 6H). Mass spectrometry showed m/z=364.0 (M+H⁺).

### Example 63

### Synthesis of 3-(((((2R,4R)-4-hydroxypyrrolidin-2-yl)methyl)(methyl)amino)methyl)-4-(3-methyl-4-(methylthio)phenoxy)benzenesulfonamide

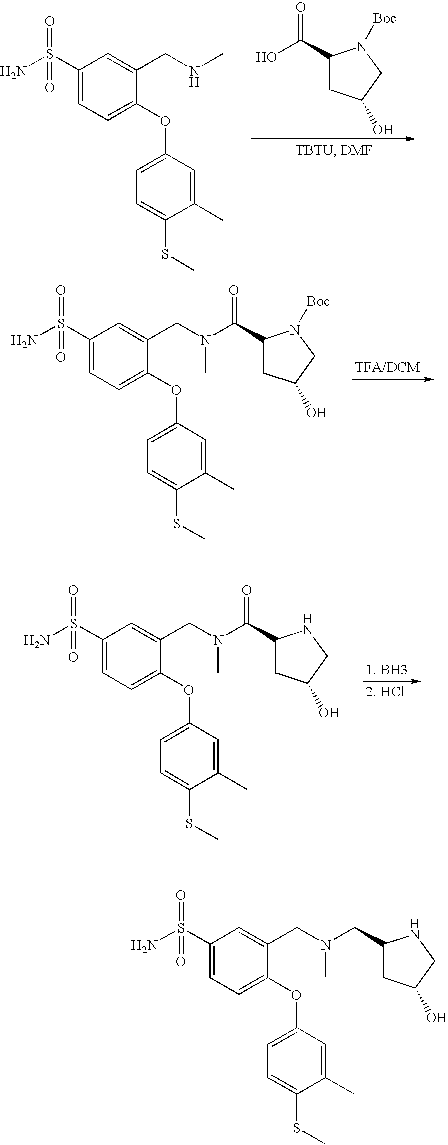

### (2R,4R)-tert-butyl 4-hydroxy-2-(methyl(2-(3-methyl-4-(methylthio)phenoxy)-5-sulfamoylbenzyl)carbamoyl)pyrrolidine-1-carboxylate

To a mixture of (2R,4R)-1-(tert-butoxycarbonyl)-4-hydroxypyrrolidine-2-carboxylic acid (108 mg, 0.47 mmol) and TBTU (163 mg, 0.51 mmol) in DMF (2 ml) was added 4-(3-methyl-4-(methylthio)phenoxy)-3-((methylamino)methyl)benzenesulfonamide (149 mg, 0.42 mmol, WO 00/51972) and N-ethyl-N-isopropylpropan-2-amine (295 ul, 1.69 mmol). The mixture was stirred overnight. The mixture was diluted with water and extracted 3 times with EA. The combined organic layers were dried over anhydrous Na₂SO₄, filtered and concentrated in vacuo. The residue was purified by flash chromatography eluted with EA:Hexane (50-100%) to yield 120 mg of the desired product, (2R,4R)-tert-butyl 4-hydroxy-2-(methyl(2-(3-methyl-4-(methylthio)phenoxy)-5-sulfamoylbenzyl)carbamoyl)pyrrolidine-1-carboxylate.

### (2R,4R)-4-hydroxy-N-methyl-N-(2-(3-methyl-4-(methylthio)phenoxy)-5-sulfamoyl-benzyl)pyrrolidine-2-carboxamide

To a solution of (2R,4R)-4-hydroxy-N-methyl-N-(2-(3-methyl-4-(methylthio)phenoxy)-5-sulfamoylbenzyl)pyrrolidine-2-carboxamide (120 mg, 0.21 mmol) in DCM (3 ml) was added TFA (3 ml). The mixture was stirred for 30 minutes. The mixture was concentrated in vacuo. Te residue was diluted with saturated NaHCO₃ and neutralized with NH₄Cl to pH 8. The mixture was extracted 3 times with EA. The combined organic layers were dried over anhydrous Na₂SO₄, filtered and concentrated in vacuo. The residue was purified by flash chromatography eluted with MeOH:EA (8-20%) to yield 75 mg of the desired product, (2R,4R)-4-hydroxy-N-methyl-N-(2-(3-methyl-4-(methylthio)phenoxy)-5-sulfamoylbenzyl)pyrrolidine-2-carboxamide. Mass spectrometry showed m/z=466.1 (M+H⁺).

### 3-(((((2R,4R)-4-hydroxypyrrolidin-2-yl)methyl)(methyl)amino)methyl)-4-(3-methyl-4-(methylthio)phenoxy)benzenesulfonamide

A solution of (2R,4R)-4-hydroxy-N-methyl-N-(2-(3-methyl-4-(methylthio)phenoxy)-5-sulfamoylbenzyl)pyrrolidine-2-carboxamide (75 mg, 0.16 mmol) in THF (2 ml) was treated with 1 M BH₃.THF complex (1.6 ml, 1.61 mmol) at room temperature. The mixture was refluxed at 100° C. for 5 hours. The mixture was then cooled to room temperature and treated cautiously with 6 M HCl solution (2 ml). The resulting mixture was refluxed at 100 for 30 minutes. The mixture was cooled to room temperature, diluted with water and basified by the cautious addition of K₂CO₃ to pH 9. The mixture was extracted 3 times with EA. The combined organic layers were dried over anhydrous Na₂SO₄, filtered and concentrated in vacuo. The residue was purified by flash chromatography eluted with MeOH:DCM (5-20%) to yield 20 mg of the desired product, 3-(((((2R,4R)-4-hydroxypyrrolidin-2-yl)methyl)(methyl)amino)methyl)-4-(3-methyl-4-(methylthio)phenoxy)benzenesulfonamide. ¹H NMR ((CD₃)₂SO, 400 MHz) generated the following peaks: δ 7.98 (d, 1H), 7.67 (dd, 1H), 7.28 (s, 2H), 7.24 (d, 1H), 6.85-6.92 (m, 3H), 4.92 (s, 1H), 4.23 (s, 1H), 4.10 (s, 1H), 3.31 (s, 4H), 3.06 (dd, 1H), 2.76 (d, 1H), 2.44 (s, 3H), 2.24 (s, 3H), 2.20 (s, 3H), 1.78-1.82 (m, 1H), 1.48-1.51 (m, 1H). Mass spectrometry showed m/z=452.1 (M+H⁺).

### Example 64

### Synthesis of 3-methyl-1-(3-methyl-4-(methylthio)phenyl)-1,2,3,4-tetrahydroquinazoline-6-sulfonamide

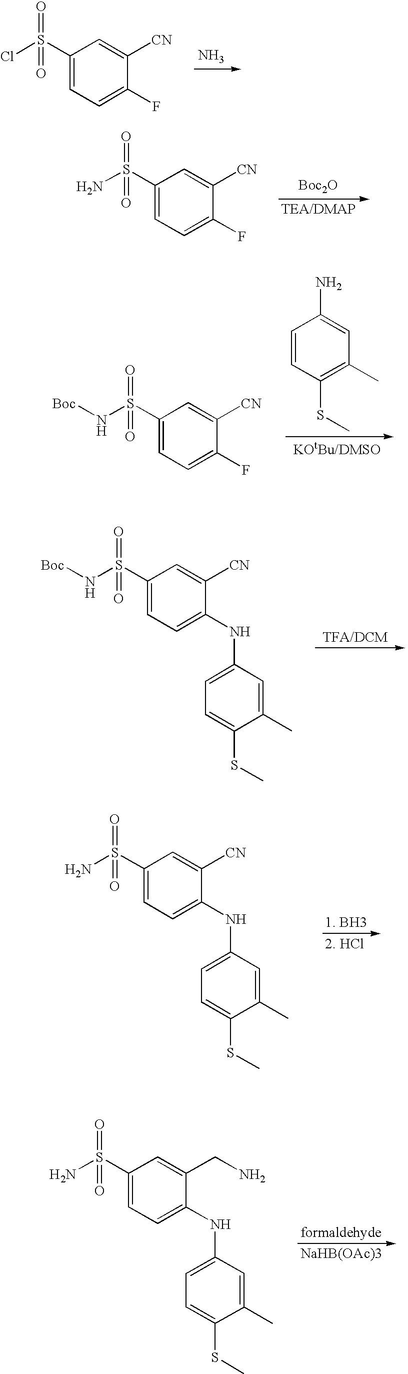

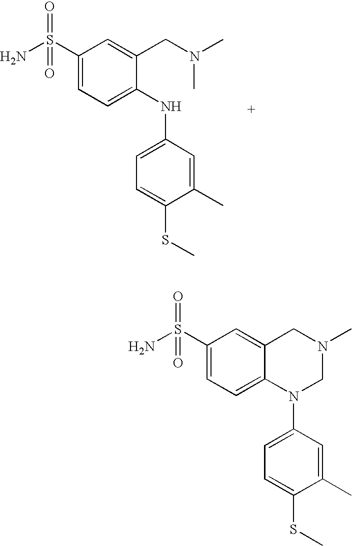

### 3-cyano-4-fluorobenzenesulfonamide

2 M Ammonia in ethanol (54 ml) was added to 3-cyano-4-fluorobenzenesulfonyl chloride (4.70 g, 21.40 mmol). The suspension was stirred for 15 minutes under nitrogen atmosphere. The mixture was diluted with 2 N HCl solution and extracted 3 times with diethyl ether. The combined organic layers were dried over anhydrous Na₂SO₄, filtered and concentrated in vacuo. The residue was purified by flash chromatography and eluted with EA:Hexane (50%) to yield 3.00 g of the desired product, 3-cyano-4-fluorobenzenesulfonamide. ¹H NMR ((CD₃)₂SO, 300 MHz) generated the following peaks: δ 8.32 (dd, 1H), 8.16-8.20 (m, 1H), 7.76 (t, 1H), 7.62 (s, 2H).

### tert-butyl 3-cyano-4-fluorophenylsulfonylcarbamate

To a solution of 3-cyano-4-fluorobenzenesulfonamide (1.00 g, 5.00 mmol) in anhydrous DCM (20 ml) was added di-tert-butyl dicarbonate (1.25 g, 5.74 mmol), triethylamine (766 ul, 5.49 mmol) and DMAP (61 mg, 0.50 mmol). The mixture was stirred for 5 hours. The mixture was diluted with water and extracted 3 times with DCM. The combined organic layers were dried over anhydrous Na₂SO₄, filtered and concentrated in vacuo. The residue was purified by flash chromatography and eluted with MeOH:DCM (2-10%) to yield 1.27 g of the desired product, tert-butyl 3-cyano-4-fluorophenylsulfonylcarbamate. ¹H NMR ((CD₃)₂SO, 400 MHz) generated the following peaks: δ 8.16 (dd, 1H), 8.07-8.11 (m, 1H), 7.61 (t, 1H), 1.23 (s, 9H).

### tert-butyl 3-cyano-4-(3-methyl-4-(methylthio)phenylamino)phenylsulfonyl-carbamate

The prepared aniline (367 mg, 2.40 mmol) was added to potassium tert-butoxide (1.12 g, 9.99 mmol) in anhydrous DMSO (8 ml). The mixture was stirred for 10 minutes and cooled to 0° C. tert-butyl 3-cyano-4-fluorophenylsulfonylcarbamate (600 mg, 2.00 mmol) was added and the mixture stirred overnight at room temperature under nitrogen atmosphere. The mixture was diluted with saturated NH₄Cl solution and extracted 3 times with diethyl ether. The combined organic layers were dried over anhydrous Na₂SO₄, filtered and concentrated in vacuo. The residue was purified by flash chromatography and eluted with MeOH:DCM (2-10%) to yield 400 mg of the desired product, tert-butyl 3-cyano-4-(3-methyl-4-(methylthio)phenylamino)phenylsulfonyl-carbamate, contaminated with a side product. Sufficient clean fractions were obtained for ¹H NMR (CDCl₃, 400 MHz) which generated the following peaks: δ 8.13 (d, 1H), 7.88 (dd, 1H), 7.20 (d, 1H), 7.01-7.10 (m, 3H), 6.74 (s, 1H), 2.50 (s, 3H), 2.35 (s, 3H), 1.43 (s, 9H).

### 3-cyano-4-(3-methyl-4-(methylthio)phenylamino)benzenesulfonamide

To a solution of impure tert-butyl 3-cyano-4-(3-methyl-4-(methylthio)phenylamino) phenylsulfonylcarbamate (400 mg, 0.92 mmol) in DCM (15 ml) was added TFA (5 ml). The mixture was stirred for 30 minutes. The mixture was concentrated in vacuo. The residue was purified by flash chromatography eluted with EA:Hexane (40%) to yield 190 mg of the desired product, 3-cyano-4-(3-methyl-4-(methylthio)phenylamino)benzene-sulfonamide. ¹H NMR (CD₃OD, 400 MHz) generated the following peaks: δ 7.99 (d, 1H), 7.79 (dd, 1H), 7.26 (d, 1H), 7.06-7.12 (m, 3H), 2.46 (s, 3H), 2.32 (s, 3H).

### 3-(aminomethyl)-4-(3-methyl-4-(methylthio)phenylamino)benzenesulfonamide

A solution of 3-cyano-4-(3-methyl-4-(methylthio)phenylamino)benzenesulfonamide (190 mg, 0.57 mmol) in THF (4 ml) was treated with 1 M BH₃.THF complex (5.7 ml) at room temperature. The mixture was refluxed at 100° C. for 5 hours. The mixture was then cooled to room temperature and treated cautiously with MeOH (5.7 ml). The mixture was concentrated in vacuo. The residue was treated with 6 M HCl solution (5.7 ml) and the resulting mixture was refluxed at 100 for 1 hour. The mixture was cooled to room temperature, diluted with water and basified with 2 N NaOH solution until pH 9 was reached. The mixture was extracted 3 times with EA. The combined organic layers were dried over anhydrous Na₂SO₄, filtered and concentrated in vacuo. The residue was purified by flash chromatography eluted with MeOH:EA (0-5%) to yield 140 mg of the desired product, 3-(aminomethyl)-4-(3-methyl-4-(methylthio)phenylamino)benzenesulfonamide. ¹H NMR (CD₃OD, 400 MHz) generated the following peaks: δ 7.74 (d, 1H), 7.61 (dd, 1H), 7.18-7.23 (m, 2H), 6.97-6.99 (m, 2H), 3.90 (s, 2H), 2.41 (s, 3H), 2.31 (s, 3H).

### 3-((dimethylamino)methyl)-4-(3-methyl-4-(methylthio)phenylamino)benzenesul-fonamide and 3-methyl-1-(3-methyl-4-(methylthio)phenyl)-1,2,3,4-tetrahydro-quinazoline-6-sulfonamide

37% Formaldehyde in MeOH (10 ul, 0.13 mmol) was added to 3-(aminomethyl)-4-(3-methyl-4-(methylthio)phenylamino)benzenesulfonamide (50 mg, 0.15 mmol) in anhydrous DCM (3 ml) and stirred for 30 minutes. NaHB(OAc)₃ (126 mg, 0.59 mmol) was added and the mixture stirred overnight. Another portion of formaldehyde and NaHB(OAc)₃ were added and the mixture stirred for another 5 hours. The mixture was basified with saturated NaHCO₃ and extracted 3 times with ethyl acetate. The combined organic layers were dried over anhydrous Na₂SO₄, filtered and concentrated in vacuo. The residue was purified by flash chromatography eluted with EA:Hexane (30-80%) to yield 10 mg of the desired product, 3-methyl-1-(3-methyl-4-(methylthio)phenyl)-1,2,3,4-tetrahydroquinazoline-6-sulfonamide. ¹H NMR (CDCl₃, 400 MHz) generated the following peaks: δ 7.49 (m, 2H), 7.20 (d, 1H), 7.00-7.06 (m, 2H), 6.59 (d, 1H), 4.62 (s, 2H), 4.38 (s, 2H), 3.99 (s, 2H), 2.55 (s, 3H), 2.49 (s, 3H), 2.34 (s, 3H). Mass spectrometry showed m/z=364.1 (M+H⁺).

10 mg of side product, 3-((dimethylamino)methyl)-4-(3-methyl-4-(methylthio)phenyl-amino)benzenesulfonamide was also obtained. ¹H NMR (CDCl₃, 400 MHz) generated the following peaks: δ 9.19 (s, 1H), 7.66 (dd, 1H), 7.61 (d, 1H), 7.20 (m, 2H), 6.97 (m, 2H), 4.65 (s, 2H), 3.52 (s, 2H), 2.45 (s, 3H), 2.36 (s, 3H), 2.26 (s, 6H). Mass spectrometry showed m/z=366.1 (M+H⁺).

### Other Embodiments

All publications, patent applications, including U.S. Provisional Application Nos. 60/844,463, filed Sep. 14, 2006, 60/874,061 filed Dec. 11, 2006, and 61/069,917, filed Mar. 19, 2008, 61/070,047, filed Mar. 19, 2008, and U.S. patent application Ser. No. 11/900,893, filed Sep. 13, 2007, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of molecular biology, medicine, immunology, pharmacology, virology, or related fields are intended to be within the scope of the invention.

What is claimed is:

1. A composition comprising (a) sertraline, a sertraline analog, UK-416244, or a UK-416244 analog and (b) an HMG-CoA reductase inhibitor.

2. The composition of claim 1, wherein said sertraline analog has a structure shown in Table 9 or said UK-416244 analog has a structure shown in Table 10 or Table 11.

3. The composition of claim 1, wherein said HMG-CoA reductase inhibitor is fluvastatin, simvastatin, lovastatin, or rosuvastatin.

4. A composition comprising sertraline, a sertraline analog, UK-416244, or a UK-416244 analog; and an antihistamine.

5. The composition of claim 4, wherein said antihistamine is hydroxyzine.

6. The composition of claim 5, wherein said sertraline analog has a structure shown in Table 9 or said UK-416244 analog has a structure shown in Table 10 or Table 11.

7. A compound having the formula: 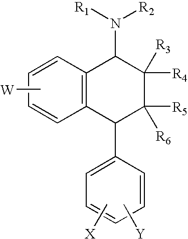
wherein
R₁ and R₂ are independently selected from the group consisting of H, optionally substituted C₁₋₆ alkyl (CH₂)ₓCOOH, or CH₂CHOH(CH₂)ₓ, (CH₂)ₓN(CH₃)₂, where x is 1, 2, 3, 4, or 5, and optionally substituted C₁₋₇ heteroalkyl;
R₃, R₄, R₅, and R₆ are independently H or optionally substituted C₁₋₆ alkyl; X and Y are each selected from the group consisting of H, F, Cl, Br, CF₃, C₁₋₆ alkoxy, and cyano; and
W is selected from the group consisting of H, F, Cl, Br, CF₃, C₁₋₃ alkoxy, COOH, CH₂CH₂OH, NHCOH, NHCOCH₃, CH₂NH₂, CH₂S(O)ₙCH₃, CONH₂, CH₂OH, NHCOPh, CH₂NHS(O)ₙCH₃, NHS(O)ₙPh, N(CH₃)₂, S(O)ₙNH₂, NHCOBu, NHS(O)ₙCH₃, NHCOcyclopropyl, NHCOcyclopentyl, CN, NHS(O)ₙcyclopropyl, NH₂, NO₂, I, SO₂N(CH₃)₂, SO₂NHMe, SO₂NHCH₂CH₂OH, CO₂Me, NHSO₂Bu, CONHCH₃, CH₂NHCOCH₃, CONHPh, 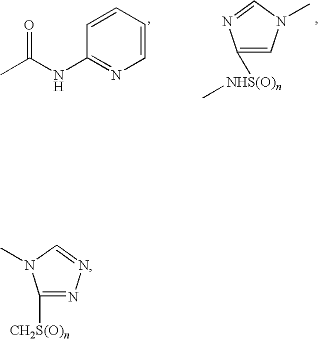
CONHcylopropyl, C(S)NH₂, NHC(S)CH₃, CONHCH₂COOCH₃, CONHCH₂COOH, CONHCH₂cyclopropyl, CONHcyclobutyl, N(CH₃)COCH₃, and CH₂S(O)ₙR₁₁, where n is 0, 1, or 2 and R₁₁ is phenyl, C₂₋₆ heterocyclyl, or optionally substituted C₁₋₈ alkyl (e.g., C₄₋₈ unsubstituted alkyl such as Bu or C₃₋₈ substituted alkyl), wherein said compound is not sertraline or an isomer thereof.

8. The compound of claim 7 having the formula: 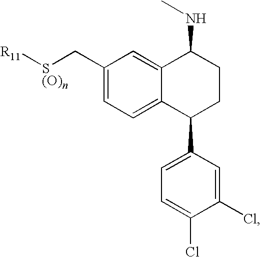
wherein n is 0, 1, or 2; and R₁₁ is phenyl, C₂₋₆ heterocyclyl, C₄₋₈ unsubstituted alkyl, or C₃₋₈ substituted alkyl.

9. A composition comprising the compound of claim 7 and a pharmaceutically acceptable carrier.

10. A compound having a structure selected from the group consisting of the compounds of Table 9, wherein said compound is not sertraline or an isomer thereof.

11. A compound having the formula: 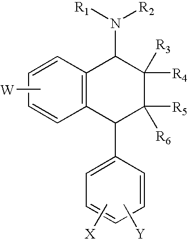
wherein R₁ is C₁₋₆ alkyl and R₂ is CH₂CH(OH)R₈, or CH₂CH(R₈)NR₉R₁₀, where R₈, R₉, and R₁₀ are independently H or C₁₋₆ alkyl; R₃, R₄, R₅, and R₆ are independently H or optionally substituted C₁₋₆ alkyl; X and Y are each selected from the group consisting of H, F, Cl, Br, CF₃, C₁₋₆ alkoxy, and cyano; and W is selected from the group consisting of H, F, Cl, Br, CF₃, C₁₋₃ alkoxy, COOH, CH₂CH₂OH, NHCOH, NHCOCH₃, CH₂NH₂, CH₂S(O)ₙCH₃, CONH₂, CH₂OH, NHCOPh, CH₂NHS(O)ₙCH₃, NHS(O), Ph, N(CH₃)₂, S(O)ₙNH₂, NHCOBu, NHS(O)ₙCH₃, NHCOcyclopropyl, NHCOcyclopentyl, CN, NHS(O)ₙcyclopropyl, NH₂, NO₂, I, SO₂N(CH₃)₂, SO₂NHMe, SO₂NHCH₂CH₂OH, CO₂Me, NHSO₂Bu, CONHCH₃, CH₂NHCOCH₃, CONHPh, 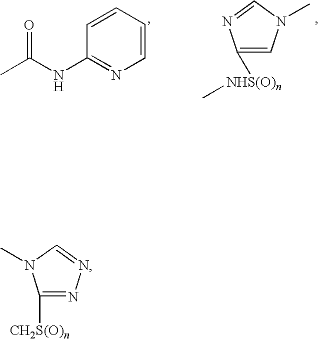
CONHcylopropyl, C(S)NH₂, NHC(S)CH₃, CONHCH₂COOCH₃, CONHCH₂COOH, CONHCH₂cyclopropyl, CONHcyclobutyl, and CH₂S(O)ₙR₁₁, where n is 0, 1, or 2 and R₁₁ is phenyl, C₂₋₆ heterocyclyl, or optionally substituted C₁₋₈ alkyl (e.g., C₄₋₈ unsubstituted alkyl such as Bu or C₃₋₈ substituted alkyl), wherein said compound is not sertraline or an isomer thereof.

12. A compound of claim 11 having a formula selected from the group consisting of: 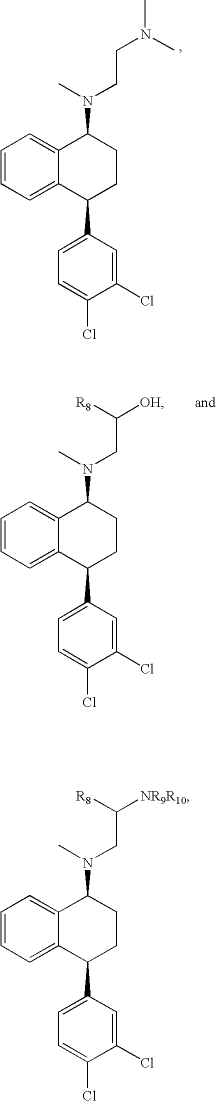
wherein R₈, R₉, and R₁₀ are independently C₁₋₈ optionally substituted alkyl, alkoxy or heteroalkyl.

13. A compound having the formula: 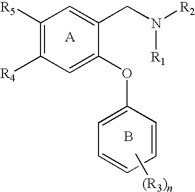
wherein R₁ and R₂ are independently H, C₁₋₆ alkyl, (CH₂)ₘ(C₃₋₆ cycloalkyl) where m is 0, 1, 2, or 3, or R₁ and R₂ together with the nitrogen to which they are attached form an azetidine ring; each R₃ is independently H, I, Br, F, Cl, C₁₋₆ alkyl, CF₃, CN, OCF₃, C₁₋₄ alkylthio, C₁₋₄ alkoxy, aryloxy, or CONR₆R₇; n is 1, 2, or 3; where one of R₄ and R₅ is A-X, where A is —CH═CH— or —(CH₂)ₚ— where p is 0, 1, or 2; X is H, F, Cl, Br, I, CONR₆R₇, SO₂NR₆R₇, SO₂NHC(═O)R₆, OH, C₁₋₄ alkoxy, NR₈SO₂R₉, NO₂, NR₁₁, CN, CO₂R₁₀, CHO, SR₁₀, S(O)R₉ or SO₂R₁₀; R₆, R₇, R₈ and R₁₀ independently are H, C₁₋₆ alkyl, C₆₋₁₂ aryl optionally substituted independently by one or more R₁₂, or C₁₋₆ alkyl-aryl optionally substituted, and the other of R₄ and R₅ is SNHPh, SONHPh, or SO₂NHPh, where the phenyl is optionally substituted by one or more R₁₂; R₉ is C₁₋₆ alkyl optionally substituted independently by one or more R₁₂; R₁₁ is H, C₁₋₆ alkyl optionally substituted independently by one or more R₁₂, C(O)R₆, CO₂R₉, C(O)NHR₆, or SO₂NR₆R₇; R₁₂ is F (preferably up to 3), OH, CO₂H, C₃₋₆ cycloalkyl, NH₂, CONH₂, C₁₋₆ alkoxy, C₁₋₆ alkoxycarbonyl, or a 5- or 6-membered heterocyclic ring containing 1, 2, or 3 heteroatoms selected from N, S, and O optionally substituted independently by one or more R₁₃; or R₆ and R₇, together with the nitrogen to which they are attached, form a 4-, 5-, or 6-membered heterocyclic ring optionally substituted independently by one or more R₁₃; or a 5- or 6-membered heterocyclic ring containing 1, 2, or 3 heteroatoms selected from N, S, and O optionally substituted independently by one or more R₁₃; where R₁₃ is hydroxy, C₁₋₄ alkoxy, F, C₁₋₆ alkyl, haloalkyl, haloalkoxy, —NH₂, —NH(C₁₋₆ alkyl), or —N(C₁₋₆ alkyl)₂—, wherein said compound is not UK-416244.

14. A compound having the structure: 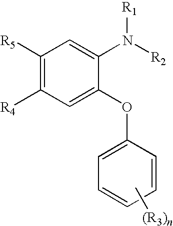
where R₁ is H or C₁₋₆ alkyl and R₂ is C₁₋₆ alkyl substituted with OH or is CH₂XR₁₄ or CH₂CH₂XR₁₄, where X is N, O, or S, and R₁₄ is H, C₁₋₆ alkyl, optionally substituted C₁₋₆ heteroalkyl, or (CH₂)_{q}(C₃₋₆ cycloalkyl) where q is 0, 1, 2, or 3, and R₃ is independently H, I, Br, F, Cl, C₁₋₆ alkyl, CF₃, CN, OCF₃, C₁₋₄ alkylthio, C₁₋₄ alkoxy, aryloxy, or CONR₆R₇; n is 1, 2, or 3; and R₄ and R₅ are independently A-X, where A is —CH═CH— or —(CH₂)ₚ— where p is 0, 1, or 2; X is H, F, Cl, Br, I, CONR₆R₇, SO₂NR₆R₇, SO₂NHC(═O)R₆, OH, C₁₋₄ alkoxy, NR₈SO₂R₉, NO₂, NR₆R₁₁, CN, CO₂R₁₀, CHO, SR₁₀, S(O)R₉, or SO₂R₁₀; R₆, R₇, R₈, and R₁₀ are independently H or C₁₋₆ alkyl, C₆₋₁₂ aryl optionally substituted independently by one or more R₁₂, or C₁₋₆ alkyl-aryl optionally substituted; R₉ is C₁₋₆ alkyl optionally substituted independently by one or more R₁₂; R₁₁ is H, C₁₋₆ alkyl optionally substituted independently by one or more R₁₂, C(O)R₆, CO₂R₉, C(O)NHR₆, or SO₂NR₆R₇; R₁₂ is F (preferably up to 3), OH, CO₂H, C₃₋₆ cycloalkyl, NH₂, CONH₂, C₁₋₆ alkoxy, C₁₋₆ alkoxycarbonyl or a 5- or 6-membered heterocyclic ring containing 1, 2, or 3 heteroatoms selected from N, S, and O optionally substituted independently by one or more R₁₃; or R₆ and R₇, together with the nitrogen to which they are attached, form a 4-, 5-, or 6-membered heterocyclic ring optionally substituted independently by one or more R₁₃; or a 5- or 6-membered heterocyclic ring containing 1, 2, or 3 heteroatoms selected from N, S, and O optionally substituted independently by one or more R₁₃; where R₁₃ is hydroxy, C₁₋₄ alkoxy, F, C₁₋₆ alkyl, haloalkyl, haloalkoxy, —NH₂, —NH(C₁₋₆ alkyl) or —N(C₁₋₆ alkyl)₂, wherein said compound is not UK-416244.

15. The compound of claim 14, wherein R₁ is H, CH₃, or CH₂CH₃ and R₂ is CH₂CH₂OH, CH(OH)CH₃, CH₂CH₂CH₂OH, CH(CH₂)CH₂OH, and CH₂CH₂CH₂CH₂OH, CH(OH)CH₂CH₂CH₃, CH₂CH(OH)CH₂CH₃, and CH₂CH₂CH(OH)CH₃.

16. The compound of claim 14, wherein said compound has the structure: 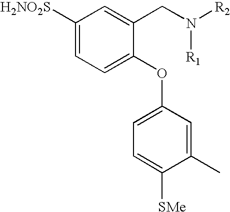
where R₁ is H or C₁₋₆ alkyl and R₂ is C₁₋₆ alkyl substituted with OH.

17. The compound of claim 16, wherein R₁ is H, CH₃, or CH₂CH₃ and R₂ is CH₂CH₂OH, CH(OH)CH₃, CH₂CH₂CH₂OH, CH(CH₂)CH₂OH, CH₂CH₂CH₂CH₂OH, CH(OH)CH₂CH₂CH₃, CH₂CH(OH)CH₂CH₃, or CH₂CH₂CH(OH)CH₃.

18. The compound of claim 17, wherein the compound has the structure:

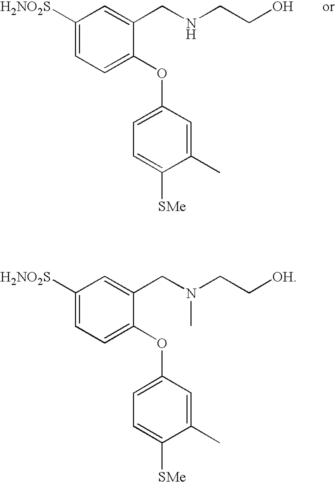

19. A compound having the structure: 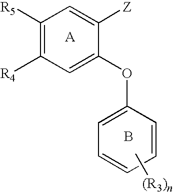
where
R₃ is independently H, I, Br, F, Cl, C₁₋₆ alkyl, CF₃, CN, OCF₃, C₁₋₄ alkylthio, C₁₋₄ alkoxy, aryloxy, or CONR₆R₇ and n is 1, 2, or 3;
R₄ and R₅ are independently A-X, where A is —CH═CH— or —(CH₂)ₚ— where p is 0, 1, or 2; X is H, F, Cl, Br, I, CONR₆R₇, SO₂NR₆R₇, SO₂NHC(═O)R₆, OH, C₁₋₄ alkoxy, NR₈SO₂R₉, NO₂, NR₆R₁₁, CN, CO₂R₁₀, CHO, SR₁₀, S(O)R₉, or SO₂R₁₀; R₆, R₇, R₈, and R₁₀ are independently H or C₁₋₆ alkyl, C₆₋₁₂ aryl optionally substituted independently by one or more R₁₂, or C₁₋₆ alkyl-aryl optionally substituted; R₉ is C₁₋₆ alkyl optionally substituted independently by one or more R₁₂; R₁₁ is H, C₁₋₆ alkyl optionally substituted independently by one or more R₁₂, C(O)R₆, CO₂R₉, C(O)NHR₆, or SO₂NR₆R₇; R₁₂ is F (preferably up to 3), OH, CO₂H, C₃₋₆ cycloalkyl, NH₂, CONH₂, C₁₋₆ alkoxy, C₁₋₆ alkoxycarbonyl or a 5- or 6-membered heterocyclic ring containing 1, 2, or 3 heteroatoms selected from N, S, and O optionally substituted independently by one or more R₁₃; or R₆ and R₇, together with the nitrogen to which they are attached, form a 4-, 5-, or 6-membered heterocyclic ring optionally substituted independently by one or more R₁₃; or a 5- or 6-membered heterocyclic ring containing 1, 2, or 3 heteroatoms selected from N, S, and O optionally substituted independently by one or more R₁₃; where R₁₃ is hydroxy, C₁₋₄ alkoxy, F, C₁₋₆ alkyl, haloalkyl, haloalkoxy, —NH₂, —NH(C₁₋₆ alkyl) or —N(C₁₋₆ alkyl)₂; and
Z is NH₂, optionally substituted optionally hetero C₁₋₈ alkyl, or is selected from the group consisting of: 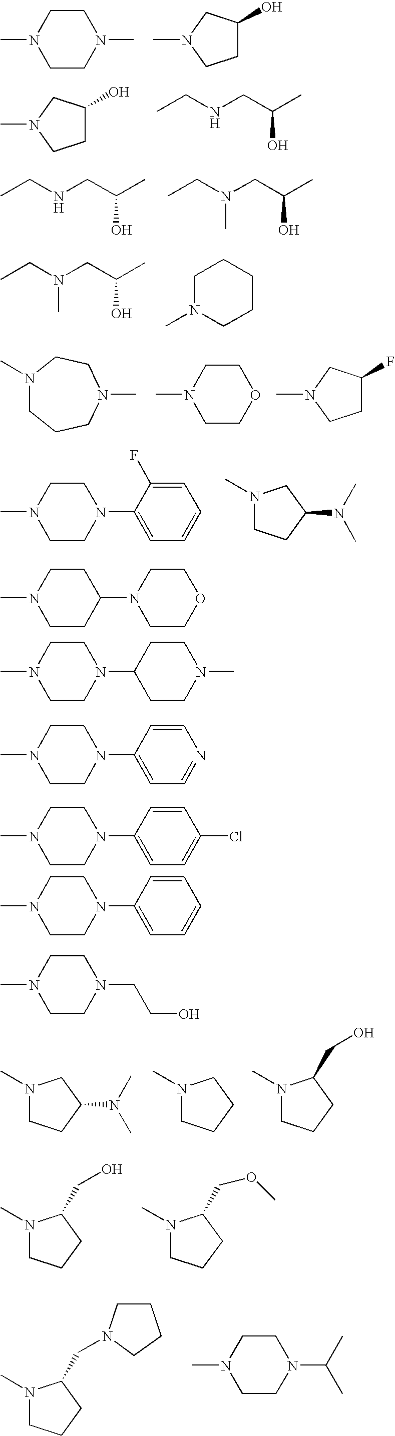 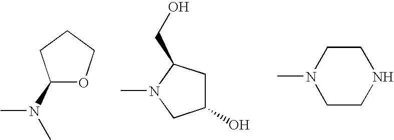
wherein said compound is not UK-416244.

20. The compound of claim 19, wherein Z is NH₂, CH₂NHCH₃, CN, CH₂CH(CH₃)₂, CH₂OCH₃, CH₂N(CH₃)CH₂CH₂OH, N(CH₃)₂, CH₂N(CH₃)₂, COOH, CH₂NHCH₃, CH₂OH, CH₂NHCOCH₃, CONHCH₃, CH₂NH(CH₂)₂N(CH₃)₂, CH₂NH(CH₂)₃N(CH₃)₂, CHC(CH₃)₂, CH₂N(CH₃)(CH₂)₂N(CH₃)₂, CH₂N(CH₃)(CH₂)₃N(CH₃)₂, or CH₂CH(CH₃)₂.

21. The compound of claim 19, wherein R₄ is H and R₅ is S(O₂)NH₂.

22. A compound having a structure shown in Table 10 or Table 11.

23. A method for treating a patient having a viral disease, said method comprising administering to said patient sertraline, a sertraline analog, UK-416244, or a UK-416244 analog.

24. The method of claim 23, wherein said sertraline analog is an analog set forth in Table 9 or said UK-416244 analog is set forth in Table 10 or Table 11.

25. The method of claim 23, wherein said patient has not been diagnosed with or does not suffer from depression, major depressive disorder, obsessive-compulsive disorder, panic disorder, posttraumatic stress disorder, social anxiety disorder, generalized anxiety disorder, or premenstrual dysphoric disorder.

26. The method of claim 23, wherein said viral disease is hepatitis C.

27. The method of claim 23, wherein said patient is a human.

28. A method for treating a patient having a viral disease, said method comprising administering to said patient (a) sertraline, a sertraline analog, UK-416244, or a UK-416244 analog and (b) an HMG-CoA reductase inhibitor, wherein said two agents are administered within 28 days of each other in amounts that together are effective to treat said patient.

29. The method of claim 28, wherein said sertraline analog is an analog set forth in Table 9 or said UK-416244 analog is set forth in Table 10 or Table 11.

30. The method of claim 28, wherein said patient has not been diagnosed with or does not suffer from depression, major depressive disorder, obsessive-compulsive disorder, panic disorder, posttraumatic stress disorder, social anxiety disorder, generalized anxiety disorder, or premenstrual dysphoric disorder.

31. The method of claim 28, wherein said HMG-CoA reductase inhibitor is fluvastatin, simvastatin, lovastatin, or rosuvastatin.

32. The method of claim 28, wherein said patient has not been diagnosed with or does not suffer from hypercholesterolemia, primary familial hypercholesterolemia (heterozygous variant), mixed hyperlipidaemia (corresponding to type Ia and IIb of the Fredrickson classification), or coronary artery disease.

33. The method of claim 28, wherein said patient has not had a myocardial infarction, a cerebrovascular event, an coronary bypass surgery, or a translumen percutaneous coronary angioplasty.

34. A method for treating a patient having a viral disease, said method comprising administering to said patient sertraline, or an analog thereof, and an antihistamine wherein said two agents are administered within 28 days of each other in amounts that together are effective to treat said patient.

35. The method of claim 34, wherein said sertraline analog is an analog set forth in Table 9 or said UK-416244 analog is set forth in Table 10 or Table 11.

36. The method of claim 34, wherein said patient has not been diagnosed with or does not suffer from depression, major depressive disorder, obsessive-compulsive disorder, panic disorder, posttraumatic stress disorder, social anxiety disorder, generalized anxiety disorder, or premenstrual dysphoric disorder.

37. The method of claim 34, wherein said antihistamine is hydroxyzine.

38. The method of claim 34, wherein said viral disease is hepatitis C.

39. The method of claim 34, wherein said patient is a human.
